(19) United States
(12) Patent Application Publication
(10) Pub. No.: US 2025/0382277 A1
(43) Pub. Date: Dec. 18, 2025

(54) SMALL MOLECULE INHIBITORS OF DYRK/CLK AND USES THEREOF

(71) Applicants: Arizona Board of Regents on Behalf of the University of Arizona, Tucson, AZ (US); University of Dundee, Dundee (GB)

(72) Inventors: Christopher Hulme, Tucson, AZ (US); Alessandra Fistrovich, Tucson, AZ (US); Arthur Shaw, Tucson, AZ (US); Tim Chavez, Tucson, AZ (US); Sourav Banerjee, Dundee (GB); Vasudha Tandon, Dundee (GB)

(21) Appl. No.: 18/877,543

(22) PCT Filed: Jun. 22, 2023
(86) PCT No.: PCT/US2023/025972

§ 371 Date: Dec. 20, 2024

Related U.S. Application Data
(60) Provisional application No. 63/354,495, filed on Jun. 22, 2022.

(51) Int. Cl. C07D 401/04; C07B 59/00; C07D 239/74; C07D 401/14; C07D 405/14; C07D 413/14; C07D 471/04; C07D 471/10; C07D 487/04; C07D 487/08; C07D 491/107; C07D 498/08

(52) U.S. Cl. CPC C07D 401/04; C07B 59/002; C07D 239/74; C07D 401/14; C07D 405/14; C07D 413/14; C07D 471/04; C07D 471/10; C07D 487/04; C07D 487/08; C07D 491/107; C07D 498/08

(57) ABSTRACT

This invention is in the field of medicinal chemistry. In particular, the invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

### STATEMENT OF GOVERNMENT SUPPORT

This invention was made with government support under Grant No. AG067926 awarded by National Institutes of Health. The government has certain rights in the invention.

### FIELD OF THE INVENTION

This invention is in the field of medicinal chemistry. In particular, the invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

### INTRODUCTION

It's estimated that 2 million new cases of cancer will be shortly diagnosed per year with over 600,000 deaths in the US per annum. DYRK and CLK kinase inhibition affords opportunities across a spectrum of malignancies. Moreover, small molecule inhibition of DYRK and CLK kinase may play a role in mitigating disease progression of autoimmune disease and inflammatory disorders, exemplified by osteo-arthritis. DYRK1A has been revealed to play a key role in dementia and down syndrome pathogenesis. With >40 million patients suffering, dementia is currently a leading unmet medical need and costly burden on public health. Seventy percent of these cases have been attributed to Alzheimer's disease (AD), a neurodegenerative pathology whose most evident symptom is a progressive decline in cognitive functions. The underlying treatment of learning and/or memory disorders is a huge and significantly unmet medical need and also includes learning and memory repair after incidents of stroke or significant brain damage.

The present invention addresses these needs.

### SUMMARY OF THE INVENTION

The proteasome (immuno- and constitutive), heat shock factor 1, and mammalian target of rapamycin (mTOR) are essential protein complexes responsible for maintaining growth, division, and survival of cells in eukaryotes and are required for almost all cellular activities. Any impairment of any one or more of the complexes often underlies neurodegenerative diseases, cancer, immune disorders, and the aging process. Targeting these complexes have been clinically proven to be effective against all forms of cancers. RNA interference, kinome-wide screen, and biochemical studies demonstrate that blocking 26S proteasome and heat shock factor 1 phosphorylations triggered by DYRK2 markedly impairs proteostasis and impedes cell proliferation (see, Guo et. al. 2016 Nature Cell Biology; Moreno et. al. 2021 Cell death and differentiation; Banerjee et. al. PNAS 2018; Banerjee et. al. PNAS 2019). Furthermore, inhibition of DYRK3 activity leads to loss of PRAS40 phosphorylation leading to loss of mTOR signaling which reduces cancer cell proliferation. Importantly, loss of DYRK2 and DYRK3 activities significantly inhibited tumor formation in mice (see, eg, Banerjee et. al. 2019 PNAS). Accordingly, small-molecule inhibitors of DYRK kinases, either used alone or in combination with existing chemotherapy and/or proteasome inhibitors, have unique therapeutic potentials in treating human cancers with deregulated growth and proliferation.

Moreover, canonical WNT signaling is a key developmental pathway that has garnered significant interest for therapeutic intervention. The ability to modulate the WNT pathway and thus restore the health of diseased tissues affords possibilities in regenerative therapeutics and oncology. Noteworthy, WNT signaling controls chondrocyte osteoblast and synovial cell functions in osteoarthritis (Tao et. al., Theranostics, 2017, 7, 180-195). Indeed, numerous biological processes and targets related to WNT activation have been reported (Zhan et. al., Oncogene 2017, 36, 1461-1473. Ahmed et. al., Cancers, 2016, 8, 66). Part of this set of targets comprises the serine/threonine kinase CLKs shown to modulate the Wnt pathway by regulating pre-mRNA splicing (Deshmukh et. al., Osteoarthritis Cartilage 2019, 27, 1347-1360, Wang et. al., Nature 2008, 456-470-476). They consist as four isoforms in mammals (CLK1 through CLK4) and belong to the CMGC group of kinases which includes DYRKS, cyclin-dependent kinases (CDKs), GSK3, serine-arginine-rich protein kinases (SRPK) and others. CLK protein over-expression affects splicing site selection of pre-mRNA and as such, several CLK family inhibitors have been reported to play roles in the control mechanisms of mRNA splicing (Bossard et al., Cancer Res., 2020, 80, 5691. Deshmukh et. al., Osteoarthritis Cartilage 2019, 27, 1347-1360). Specifically, two high profile CLK inhibitors in clinical trials are SM08502 (Indication: Colorectal cancer, NCT03355066) and SM04690 (Indication: Osteoarthritis of the knee, Phase 3, NCT03928184).

CDKs have also shown to be heavily implicated in WNT inhibition. In particular CDK7 enhances the interaction between beta-catenin and TCF4 (see Duan et al., Cell Death & Differentiation, 2019, 26, 1442-1452), CDK8 has been identified as a gene that regulates b-catenin driven reporter activity in a loss of function RNAi screen (Rosenbluh et al., Trends Pharmacol Sci. 2014, 35, 103-109). Inhibitors of CDK7, 8 and 19 have shown utility in colorectal cancer and metastatic colon cancer in the liver and have been implicated in WNT signaling inhibition. Moreover, CDK8 selectivity promotes growth of colon cancer metastases in the liver by regulating gene expression of TIMP3 and matrix metalloproteases (see Liang et. al., Cancer Res. 2018, 78(23), 6594-6606). CDK8 and its paralog CDK19 are two isoforms of the Mediator kinase, the enzymatic component of the CDK module that binds to the transcriptional Mediator complex and inhibition of the CDK8/19 Mediator kinase sensitizes HER2+ breast cancers to HER2-targeting drugs preventing resistance in vitro and in vivo (see, e.g., Ding et al., PNAS, 2022, 119 (32), 1-11, e2201073119). High profile CDK8/CDK19 inhibitors in clinical trials include RVU120 in patients with Acute Myeloid Leukemia (AML) or high-risk Myelodysplastic Syndrome (HR-MDS) (NCT04021368), TSN084 (NCT05300438) and Senexin B, the first selective CDK8/19 inhibitor to enter clinical trials (NCT03065010).

In addition to the overwhelmingly prominent β-amyloid hypothesis being evaluated in a multitude of clinical trials through small molecule modulation of γ- and β-secretases and numerous immune-based approaches, aberrant phosphorylation of the tau protein is believed to significantly contribute to the development of AD and thus affords an alternate approach for therapeutic development. Tau is a cytoplasmic protein involved in the stabilization of microtubules under normal conditions. In AD, neuronal tau has been found to be excessively phosphorylated, with subsequent generation of aggregates of phosphorylated tau protein, known as “neurofibrillary tangles” (NFTs). NFTs and amyloid plaques are considered the most common hallmarks of AD and are correlated with neurofibrillary degeneration, neuronal death, and dementia.

Interestingly, several protein kinases have been implicated in neuronal development and, in particular, their overexpression and aberrant activation have been shown to play a significant role in the development of AD via tau phosphorylation. Dual specificity tyrosine phosphorylation regulated kinase-1A (DYRK1A) is important in neuronal development and plays a variety of functional roles within the adult central nervous system. The DYRK1A gene is located within the Down syndrome critical region (DSCR) on human chromosome 21 and current research suggests that overexpression of DYRK1A may be a significant factor leading to cognitive deficits in people with Alzheimer's disease (AD) and Down syndrome (DS).

Experiments conducted during the course of developing embodiments for the present invention designed, synthesized and biologically evaluated compounds having a 6,6-heterocyclic structure (e.g., compounds having anaphthyridine, pyrido-pyridazine, pyrido-pyrazine, quinoline, pyrazino-pyridazine, pyrimido-pyrimidine, quinazoline, quinoxaline or cinnoline ring system) as inhibitors of the dual specificity tyrosine phosphorylation regulated kinases (DYRKS) and CLKs, and their potential for use as therapeutics against AD and other disorders related to DYRK1A, DYRK1B, DYRK2, DYRK3, and CLK1, CLK2, CLK3 and CLK4 activities (e.g., DS, other neuropathology, cancer including glioblastoma, prostate cancer, metastatic breast cancer, metastatic lung cancer, multiple myeloma, secondary metastatic tumors of the brain, triple negative breast cancer), diabetes, cognitive enhancement). Many of such compounds are likely to exhibit activity against dual specificity tyrosine phosphorylation regulated kinase-1B (DYRK1B), dual specificity tyrosine phosphorylation regulated kinase-2 (DYRK2) (see, Tandon, et al., J. Biol. Chem 296 (2021)), dual specificity tyrosine phosphorylation regulated kinase-3 (DYRK3) (see, Kim, et al., Intl. J. Molecular Sciences 22, 2982 (2021)), and exhibit activity against other kinases implicated in a variety of disease states (e.g., dual specificity protein kinase CLK1 (Clk-1).

The DYRK/CLK inhibitors described herein can also be considered as potential therapeutics for the treatment of developmental diseases such as Down syndrome, and neurodegenerative diseases such as Parkinson's disease, and Huntington's disease. Moreover, the DYRK inhibitors of the present invention have been also implicated as potential therapeutics for the treatment of glioblastomas and further potential utility is highlighted in the oncology arena (see, e.g., Ionescu et al., Mini-reviews in Medicinal Chemistry, 2012, 12, 1315-1329).

These novel DYRK/CLK inhibitors may also have utility as general cognitive enhancers, given the published findings that DYRK1A can phosphorylate sirtuin 1, a key regulator of learning and memory (see, e.g., Michan et al., J. Neurosci. 2010, 30(29), 9695-9707; Guo et al., J Biol. Chem. 2010, 285 (17), 13223-13232). Moreover, the effectiveness of small molecule inhibition of DYRK1A in mitigating both insoluble tau aggregates and amyloid plaques has been demonstrated (see, e.g., Branca et al., Aging Cell, 2017, 16(5), 1146-1154). The mechanistic rational for this was detailed previously (Smith et al., ACS Chem. Neuroscience, 2012, 3(11), 857-872). These novel DYRK/CLK inhibitors may also have further utility as results identify DYRK1A as a physiologically relevant regulator of T_{reg} cell differentiation and suggest a broader role for other DYRK family members in immune homeostasis. As such, new roles may be found in autoimmune diseases such as inflammatory bowel disease and type 1 diabetes (see, e.g., Khor B, et al., eLife 2015; 4:e05920).

Accordingly, this invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

In a particular embodiment, compounds encompassed within the following formulas are provided:

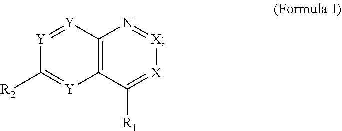

including pharmaceutically acceptable salts, solvates, and/or prodrugs thereof.

Formula I is not limited to a particular chemical moiety for X, Y, R1 and R2. In some embodiments, the particular chemical moiety for X, Y, R1 and R2 independently include any chemical moiety that permits the resulting compound to inhibit DYRK1A activity. In some embodiments, the particular chemical moiety for X, Y, R1 and R2 independently include any chemical moiety that permits the resulting compound to inhibit one or more of DYRK1A related PI3K/Akt signaling; DYRK1A related tau phosphorylation; DYRK1A related NFAT phosphorylation; DYRK1A related ASK1/JNK1 pathway activation; DYRK1A related p53 phosphorylation; DYRK1A related Amph 1 phosphorylation; DYRK1A related Dynamin 1 phosphorylation; DYRK1A related Synaptojanin phosphorylation; DYRK1A related presenilin 1 (the catalytic sub-unit of γ-secretase) activity; DYRK1A related amyloid precursor protein phosphorylation; DYRK1A related SIRT1 activation; DYRK2 related heat shock factor 1 and 26S proteasome activities; DYRK3 related mTOR activity; DYRK3 phosphorylation (e.g., PRAS40); DYRK1B activity; CMGC/CLK kinase activity; CLK1 activity; CLK2 activity; CLK3 activity; CLK4 activity; CDK7 activity; CDK8 activity; CDK19 activity; PI3K activity; PI3K mutant activity; PDGFrA/B activity; mTOR activity; c-KIT activity; RYK activity; and WNT signaling.

Such embodiments are not limited to a particular definition for each of the “X” and “Y” substituents.

In some embodiments, one of the “X” substituents is carbon and the other is nitrogen, or both of the “X” substituents are carbon; and one of the “Y” substituents is nitrogen and the other “Y” substituents are carbon, or two of the “Y” substituents are nitrogen and one “Y” substituent is carbon, or all of the “Y” substituents are carbon; such that the resulting structure is one of the following formulas:

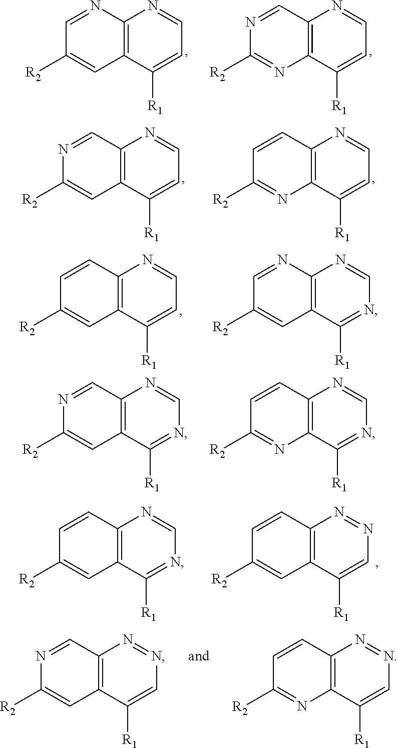

In some embodiments, R1 is an aryl or heteroaryl ring.

In some embodiments, R1 is selected from hydrogen, halogen (e.g., fluorine, bromine, iodine, chlorine).

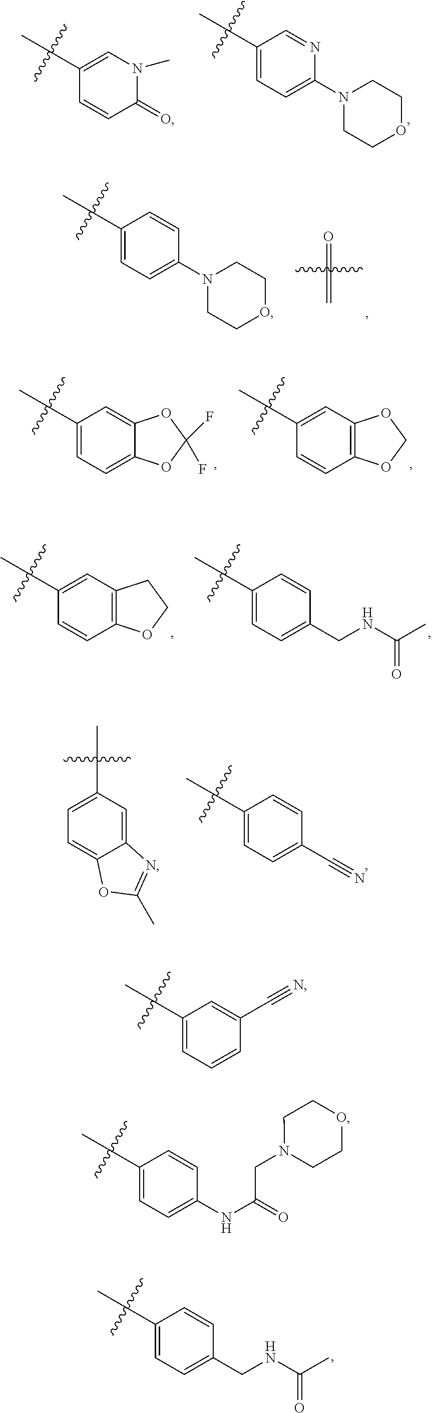

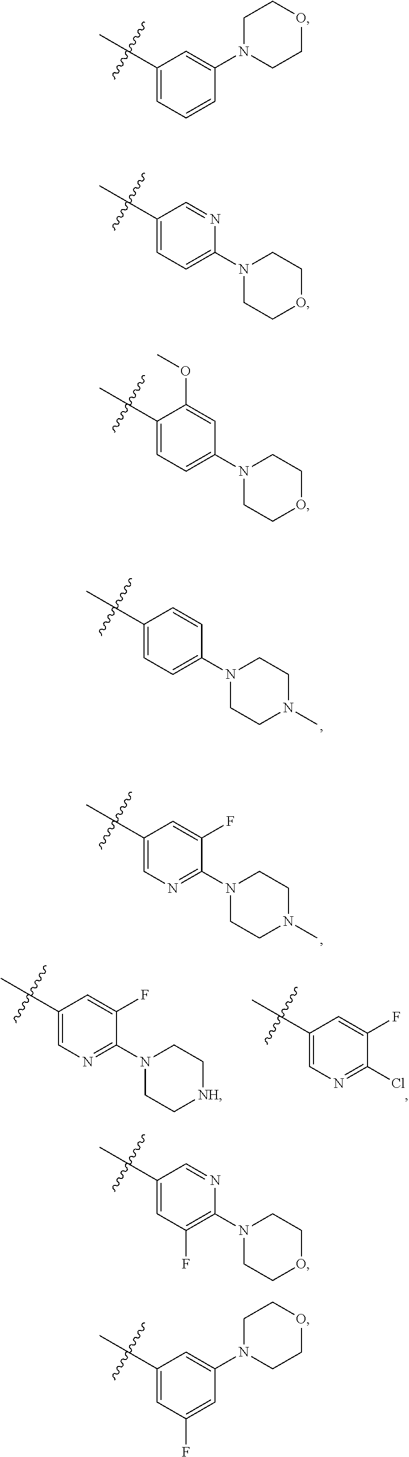

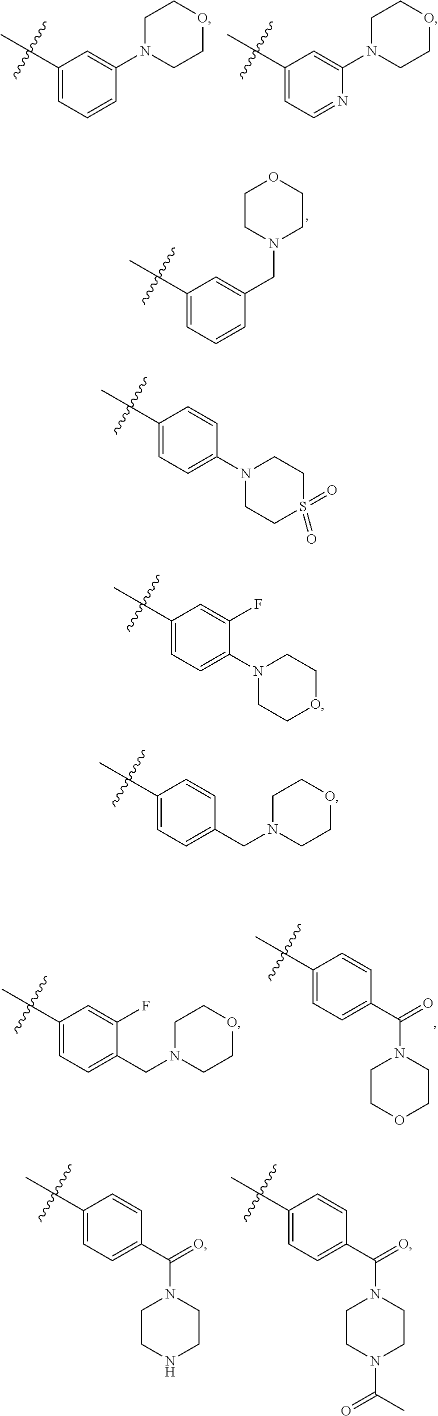

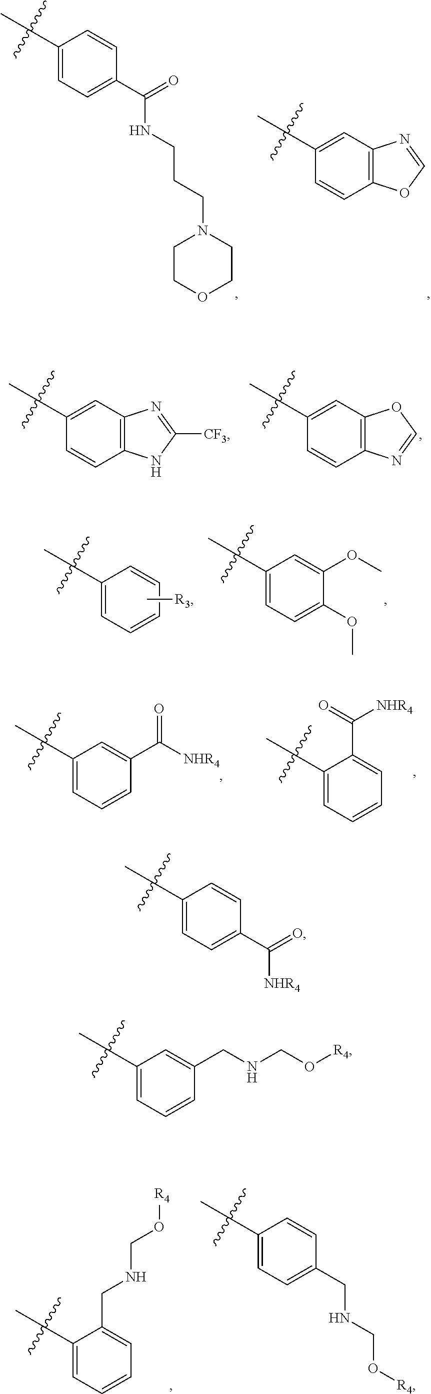

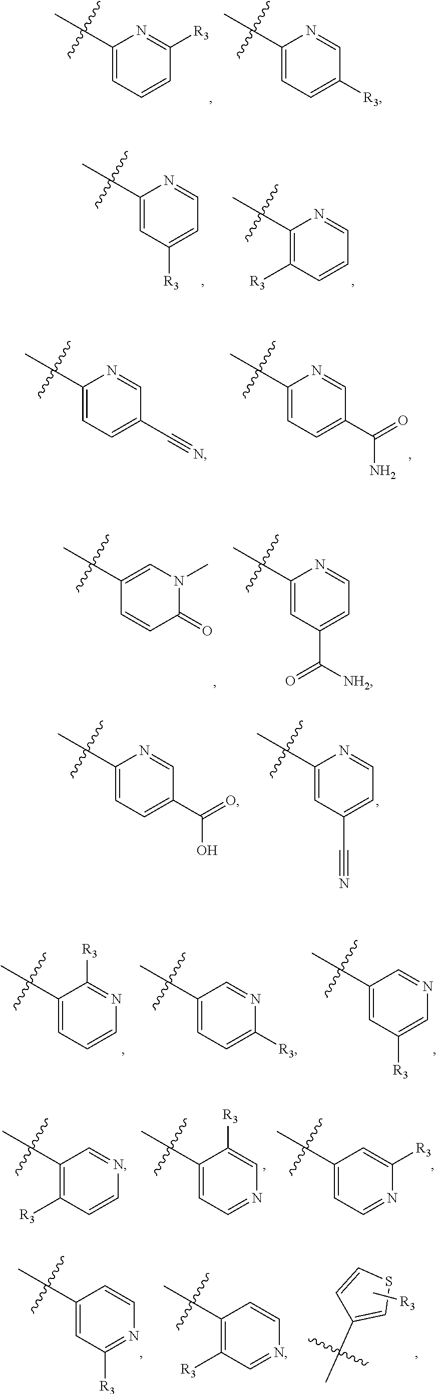

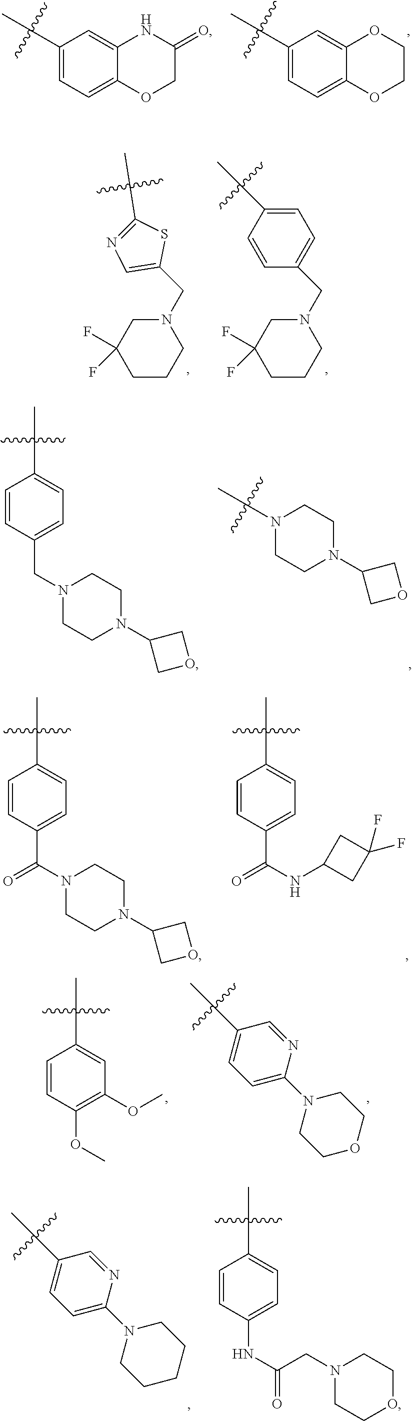

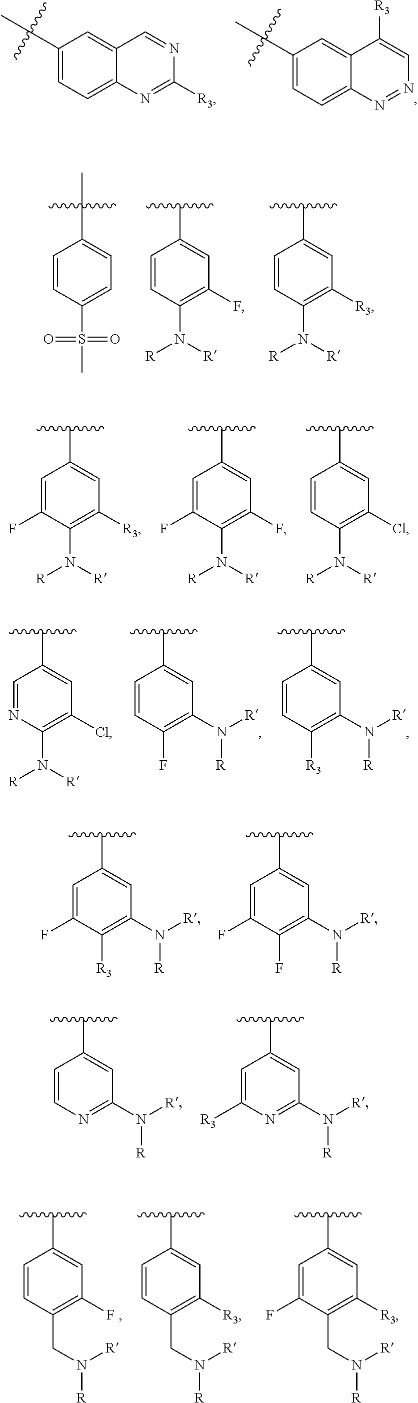

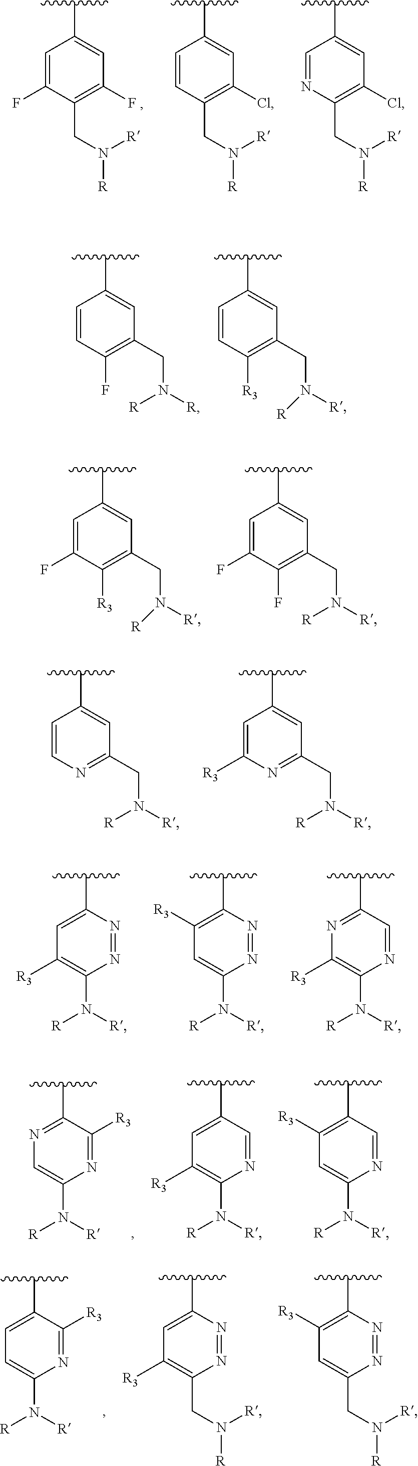

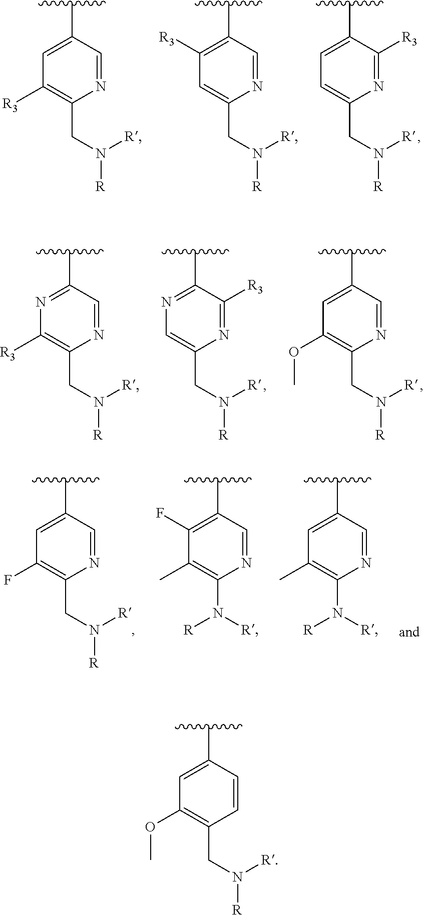

In some embodiments, R2 is selected from hydrogen, halogen (e.g., fluorine, bromine, iodine, chlorine), aryl, substituted aryl, heteroaryl, substituted heteroaryl,

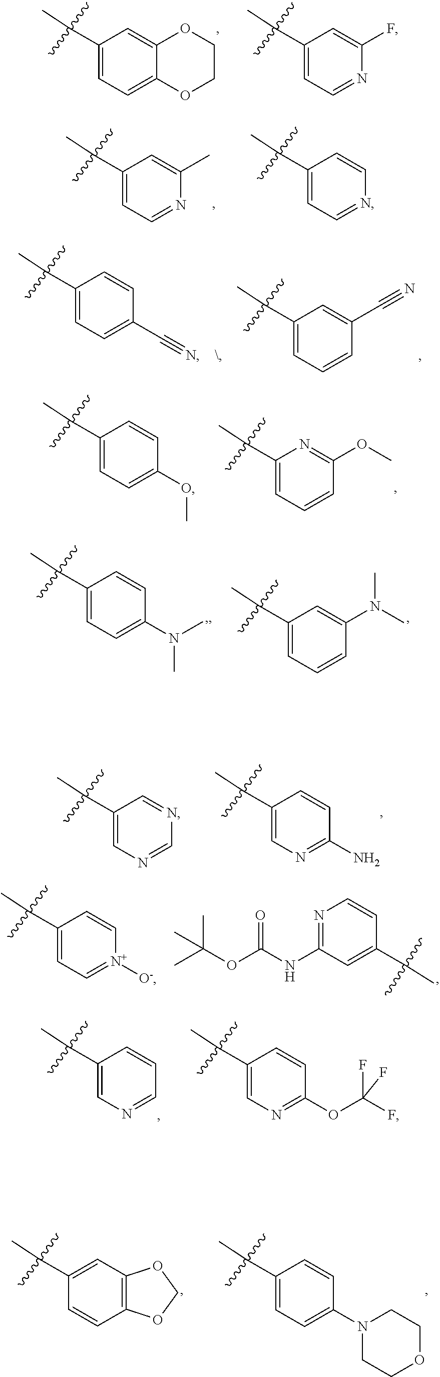

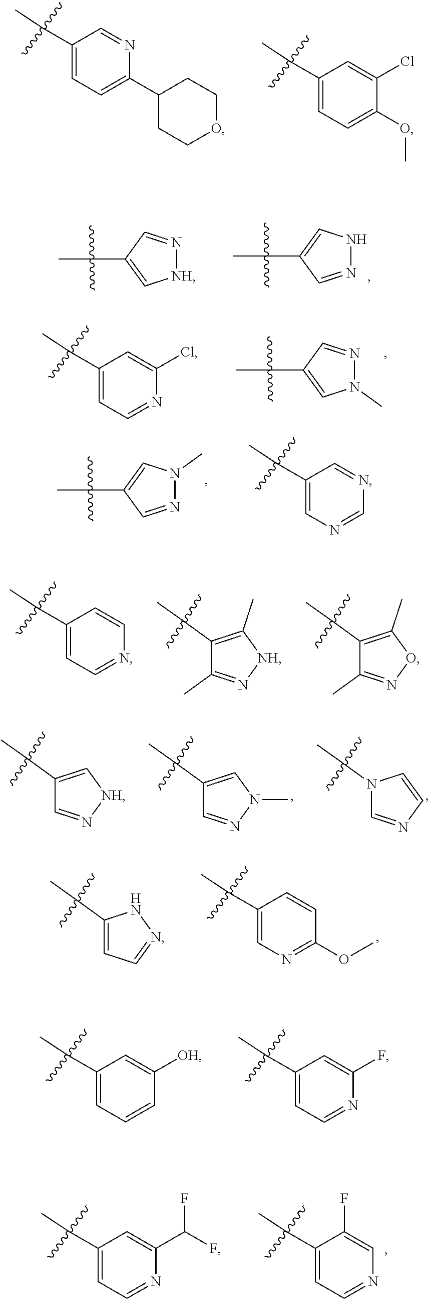

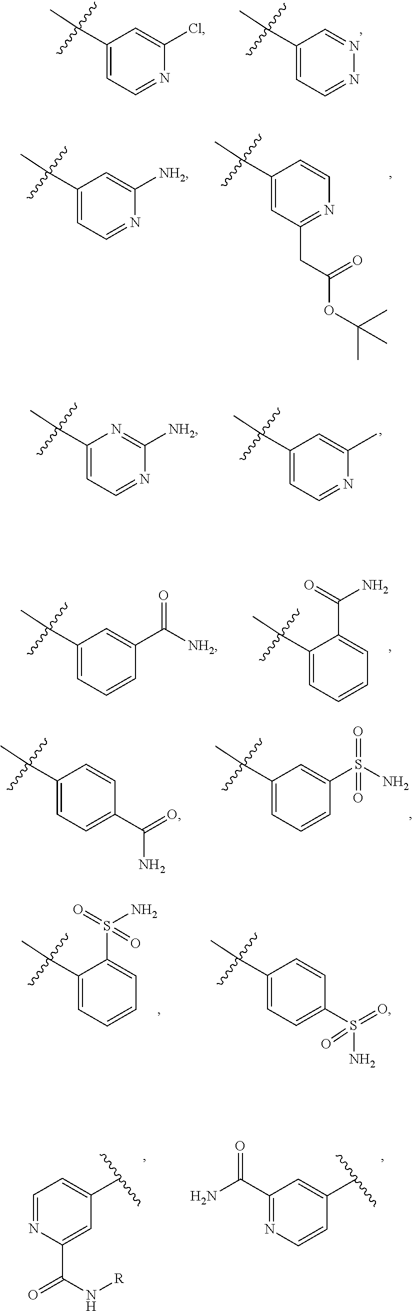

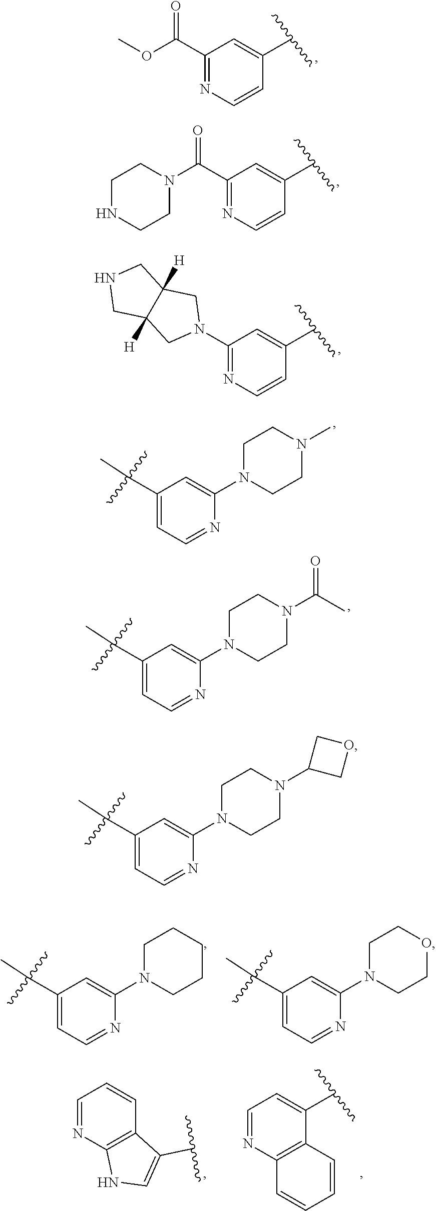

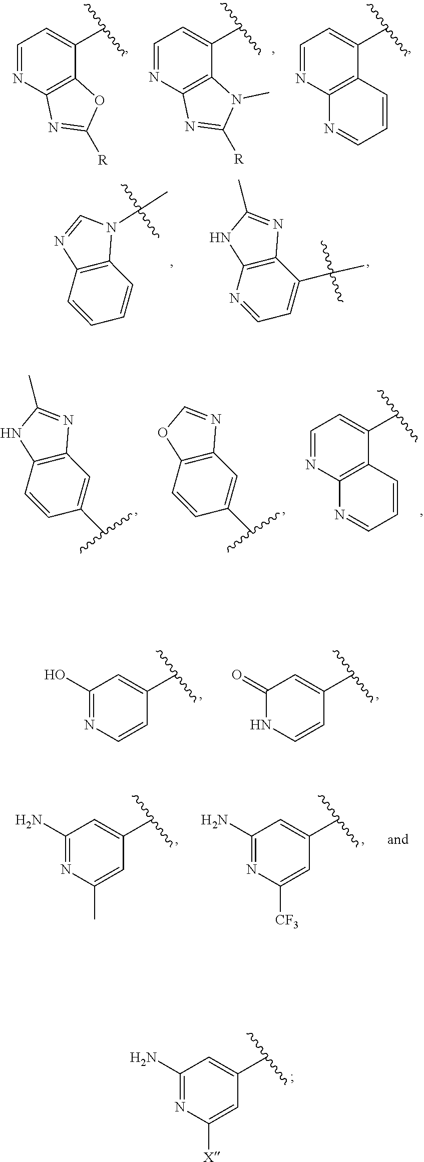

wherein X″ is selected from alkyl, haloalkyl, amino, alkylamino, hydroxy, fluoro, chloro, bromo, and cyano groups.

In some embodiments, R, R′ and R″ are independently selected from hydrogen, halogen (e.g., fluorine, bromine, chlorine, iodine), di-halogen (di-fluorine, di-bromine, di-chlorine, di-iodine), CF3, OCH3, CHF2H, OCF3, methyl, di-methyl, alkoxy, alkylsulfonyl, cyano, carboxy, ester, amido, substituted amido, sulfonamide, substituted sulfonamide, methylenedioxy, heterocyclyl alkyl, heterocyclyl, heterocyclyl alkyl amido, a lipophilic moiety comprising ether, a secondary or tertiary amine moiety consisting of a heterocycloalkyl group that is bioisosteric to secondary amines (e.g., morpholine, piperidine, piperazine).

In some embodiments, R3 and R4 are independently selected from hydrogen, halogen (e.g., fluorine, bromine, chlorine, iodine), methyl, ethyl, and methoxy.

In some embodiments, the compounds are compounds 1-64 as recited in Table 1. Each of the compounds presented in Table 1 have KD values between 0.5 nM to 10 uM (DYRK1A) and exhibit pan-DYRK and pan-CLK inhibitory profiles. Moreover, many of the exemplified compounds exhibit the ability to inhibit WNT signaling as judged by data from a WNT reporter assay (see Table 1). WNT Reporter Assay: Human Colonic Epithelial Cells (HCEC) were cultured using 1×DMEM supplemented with 1% penicillin/streptomycin, 1% Glutamax, and 10% fetal bovine serum in 5% CO2 at 37° C. These cells were previously engineered to express the TopGFP reporter (Addgene #24304) using second generation lentiviral techniques. For the Wnt reporter assay, cells were seeded at 2000 cells per well in a 384-well black screenstar imaging microplates (Greiner #781866) and allowed to adhere overnight. The following day cells were stimulated to induce the Wnt pathway using 10 μM CHIR99021 (Selleck #S1263). Simultaneously, DYR compounds were given in a dose-response using a Tecan d300e digital dispenser ranging from 0 M to 30 μM concentrations. Cells were incubated for 24 hours before fixing for 30 minutes with 4% paraformaldehyde/sucrose solution. Cells were permeabilized with 0.10% triton-x in PBS for 10 minutes and stained for DAPI for 30 minutes. Plates were imaged on a Nikon Ti2 Eclipse fluorescent microscope for DAPI, GFP, and mCherry. Using Nikon Elements software for analysis, nuclei were segmented based on DAPI and mean object intensity per cell for both TopGFP and the internal control (mCherry) was measured. To calculate the amount of Wnt activity, we took the mean intensity of TopGFP and divided it by the mean intensity of mCherry per cell to normalize individual cells. Curves and EC50s were plotted and calculated using Graphpad Prism software. KDs were obtained from Eurofins. The KdELECT Kinase Assay Panel enables quantitated compound binding affinity against any kinase assay. Inhibitor binding constants (Kd values) are calculated from duplicate 11-point dose-response curves. Measurements are made under optimized conditions that generate true thermodynamic Kd values which facilitate direct comparison of inhibitor affinity across kinases.

TABLE 1

| 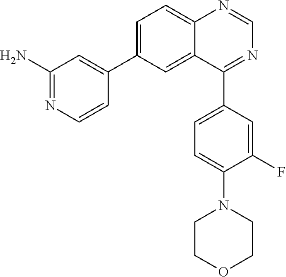 [SMILES: Nc1cc(-c2ccc3ncnc(-c4ccc(N5CCOCC5)c(F)c4)c3c2)ccn1] | WNT EC₅₀: ++ DYRK1A K_{d}: +++ |
|---|---|
| 1 | |
| 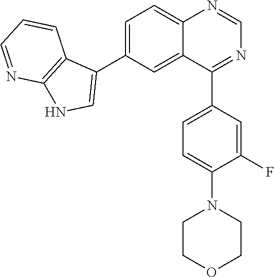 [SMILES: Fc1cc(-c2ncnc3ccc(-c4c[nH]c5ncccc45)cc23)ccc1N1CCOCC1] | WNT EC₅₀: +++ DYRK1A K_{d}: +++ |
| 2 | |
| 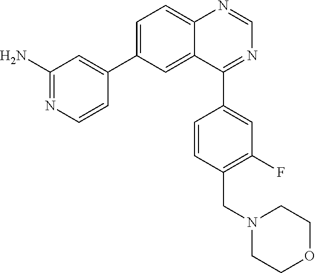 [SMILES: Nc1cc(-c2ccc3ncnc(-c4ccc(CN5CCOCC5)c(F)c4)c3c2)ccn1] | WNT EC₅₀: ++ DYRK1A K_{d}: +++ |
| 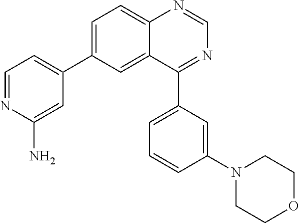 [SMILES: Nc1cc(-c2ccc3ncnc(-c4cccc(N5CCOCC5)c4)c3c2)ccn1] | WNT EC₅₀: + DYRK1A K_{d}: +++ |
| 4 | |
| 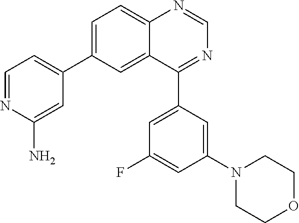 [SMILES: Nc1cc(-c2ccc3ncnc(-c4cc(F)cc(N5CCOCC5)c4)c3c2)ccn1] | WNT EC₅₀: N/A DYRK1A K_{d}: +++ |
| 5 | |
| 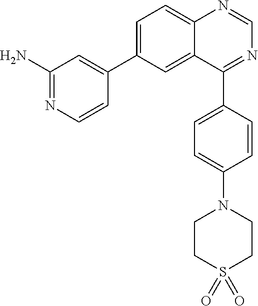 [SMILES: Nc1cc(-c2ccc3ncnc(-c4ccc(N5CCS(=O)(=O)CC5)cc4)c3c2)ccn1] | WNT EC₅₀: N/A DYRK1A K_{d}: +++ |
| 6 | |
| 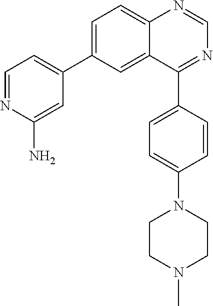 [SMILES: CN1CCN(c2ccc(-c3ncnc4ccc(-c5ccnc(N)c5)cc34)cc2)CC1] | WNT EC₅₀: N/A DYRK1A K_{d}: +++ |
| 7 | |
| 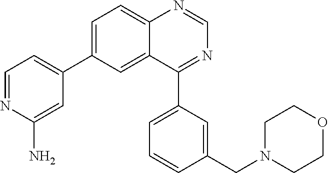 [SMILES: Nc1cc(-c2ccc3ncnc(-c4cccc(CN5CCOCC5)c4)c3c2)ccn1] | WNT EC₅₀: + DYRK1A K_{d}: +++ |
| 8 | |
| 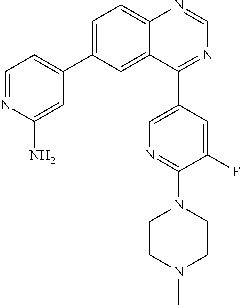 [SMILES: CN1CCN(c2ncc(-c3ncnc4ccc(-c5ccnc(N)c5)cc34)cc2F)CC1] | WNT EC₅₀: ++ DYRK1A K_{d}: +++ |
| 9 | |
| 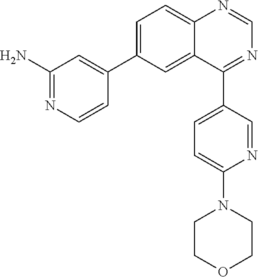 [SMILES: Nc1cc(-c2ccc3ncnc(-c4ccc(N5CCOCC5)nc4)c3c2)ccn1] | WNT EC₅₀: N/A DYRK1A K_{d}: +++ |
| 10 | |
| 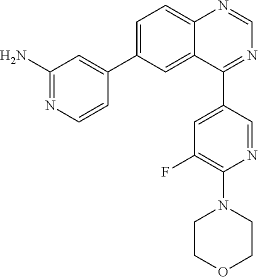 [SMILES: Nc1cc(-c2ccc3ncnc(-c4cnc(N5CCOCC5)c(F)c4)c3c2)ccn1] | WNT EC₅₀: ++ DYRK1A K_{d}: +++ |
| 11 | |
| 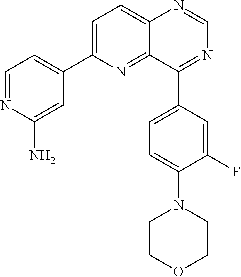 [SMILES: Nc1cc(-c2ccc3ncnc(-c4ccc(N5CCOCC5)c(F)c4)c3n2)ccn1] | WNT EC₅₀: +++ DYRK1A K_{d}: +++ |
| 12 | |
| 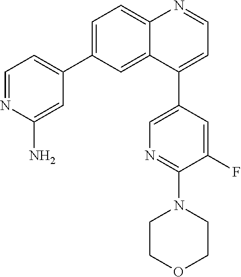 [SMILES: Nc1cc(-c2ccc3nccc(-c4cnc(N5CCOCC5)c(F)c4)c3c2)ccn1] | WNT EC₅₀: ++ DYRK1A K_{d}: +++ |
| 13 | |
| 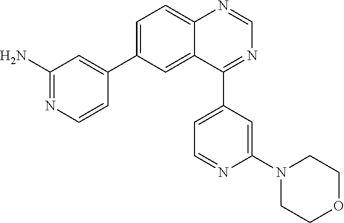 [SMILES: Nc1cc(-c2ccc3ncnc(-c4ccnc(N5CCOCC5)c4)c3c2)ccn1] | WNT EC₅₀: + DYRK1A K_{d}: +++ |
| 14 | |
| 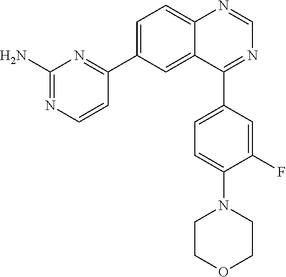 [SMILES: Nc1nccc(-c2ccc3ncnc(-c4ccc(N5CCOCC5)c(F)c4)c3c2)n1] | WNT EC₅₀: ++ DYRK1A K_{d}: +++ |
| 15 | |
| 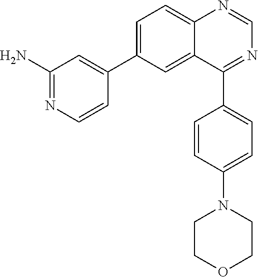 [SMILES: Nc1cc(-c2ccc3ncnc(-c4ccc(N5CCOCC5)cc4)c3c2)ccn1] | WNT EC₅₀: ++ DYRK1A K_{d}: +++ |
| 16 | |
| 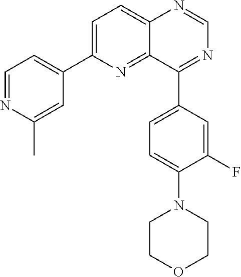 [SMILES: Cc1cc(-c2ccc3ncnc(-c4ccc(N5CCOCC5)c(F)c4)c3n2)ccn1] | WNT EC₅₀: + DYRK1A K_{d}: +++ |
| 17 | |
| 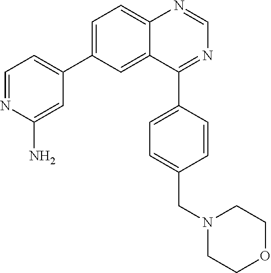 [SMILES: Nc1cc(-c2ccc3ncnc(-c4ccc(CN5CCOCC5)cc4)c3c2)ccn1] | WNT EC₅₀: N/A DYRK1A K_{d}: + |
| 18 | |
| 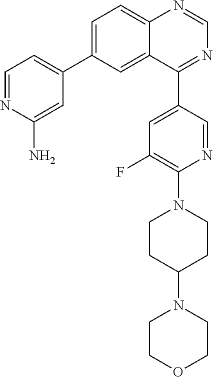 [SMILES: Nc1cc(-c2ccc3ncnc(-c4cnc(N5CCC(N6CCOCC6)CC5)c(F)c4)c3c2)ccn1] | WNT EC₅₀: ++ DYRK1A K_{d}: +++ |
| 19 | |
| 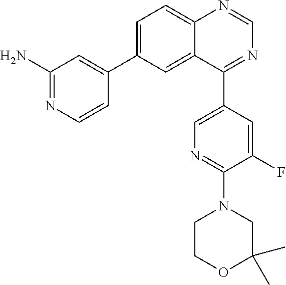 [SMILES: CC1(C)CN(c2ncc(-c3ncnc4ccc(-c5ccnc(N)c5)cc34)cc2F)CCO1] | WNT EC₅₀: + DYRK1A K_{d}: +++ |
| 20 | |
| 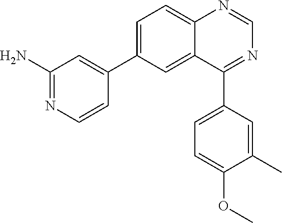 [SMILES: COc1ccc(-c2ncnc3ccc(-c4ccnc(N)c4)cc23)cc1C] | WNT EC₅₀: ++ DYRK1A K_{d}: +++ |
| 21 | |
| 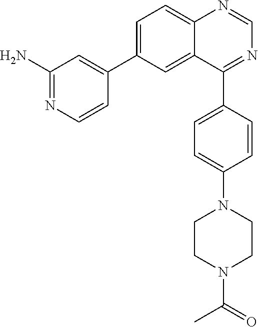 [SMILES: CC(=O)N1CCN(c2ccc(-c3ncnc4ccc(-c5ccnc(N)c5)cc34)cc2)CC1] | WNT EC₅₀: +++ DYRK1A K_{d}: +++ |
| 22 | |
| 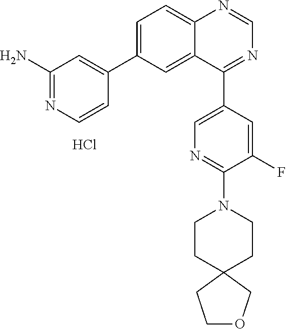 [SMILES: Nc1cc(-c2ccc3ncnc(-c4cnc(N5CCC6(CCOC6)CC5)c(F)c4)c3c2)ccn1 ; Cl] | WNT EC₅₀: ++ DYRK1A K_{d}: +++ |
| 23 | |
| 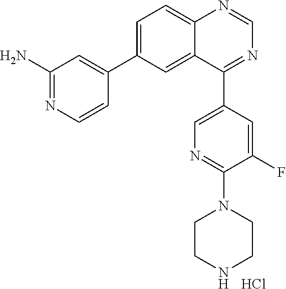 [SMILES: Nc1cc(-c2ccc3ncnc(-c4cnc(N5CCNCC5)c(F)c4)c3c2)ccn1 ; Cl] | WNT EC₅₀: + DYRK1A K_{d}: +++ |
| 24 | |
| 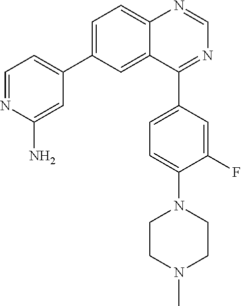 [SMILES: CN1CCN(c2ccc(-c3ncnc4ccc(-c5ccnc(N)c5)cc34)cc2F)CC1] | WNT EC₅₀: +++ DYRK1A K_{d}: +++ |
| 25 | |
| 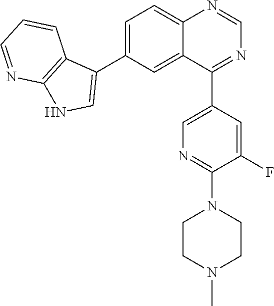 [SMILES: CN1CCN(c2ncc(-c3ncnc4ccc(-c5c[nH]c6ncccc56)cc34)cc2F)CC1] | WNT EC₅₀: ++ DYRK1A K_{d}: +++ |
| 26 | |
| 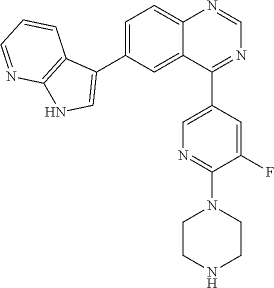 [SMILES: Fc1cc(-c2ncnc3ccc(-c4c[nH]c5ncccc45)cc23)cnc1N1CCNCC1] | WNT EC₅₀: ++ DYRK1A K_{d}: +++ |
| 27 | |
| 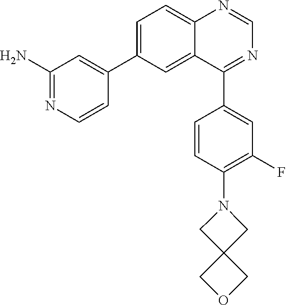 [SMILES: Nc1cc(-c2ccc3ncnc(-c4ccc(N5CC6(COC6)C5)c(F)c4)c3c2)ccn1] | WNT EC₅₀: ++ DYRK1A K_{d}: +++ |
| 28 | |
| 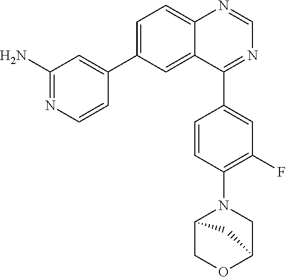 [SMILES: Nc1cc(-c2ccc3ncnc(-c4ccc(N5C[C@@H]6C[C@H]5CO6)c(F)c4)c3c2)ccn1] | WNT EC₅₀: ++ DYRK1A K_{d}: +++ |
| 29 | |
| 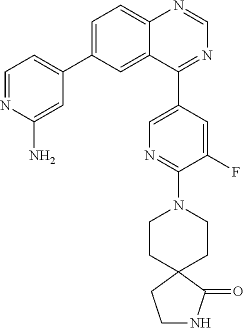 [SMILES: Nc1cc(-c2ccc3ncnc(-c4cnc(N5CCC6(CCNC6=O)CC5)c(F)c4)c3c2)ccn1] | WNT EC₅₀: ++ DYRK1A K_{d}: +++ |
| 30 | |
| 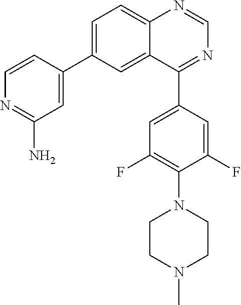 [SMILES: CN1CCN(c2c(F)cc(-c3ncnc4ccc(-c5ccnc(N)c5)cc34)cc2F)CC1] | WNT EC₅₀: ++ DYRK1A K_{d}: +++ |
| 31 | |
| 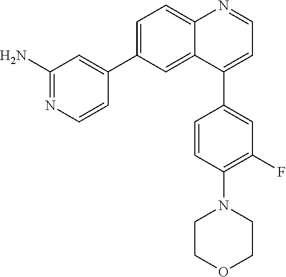 [SMILES: Nc1cc(-c2ccc3nccc(-c4ccc(N5CCOCC5)c(F)c4)c3c2)ccn1] | WNT EC₅₀: ++ DYRK1A K_{d}: +++ |
| 32 | |
| 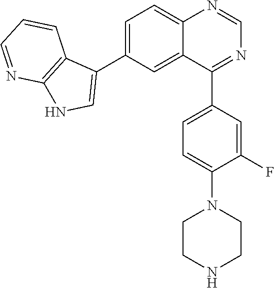 [SMILES: Fc1cc(-c2ncnc3ccc(-c4c[nH]c5ncccc45)cc23)ccc1N1CCNCC1] | WNT EC₅₀: ++ DYRK1A K_{d}: +++ |
| 33 | |
| 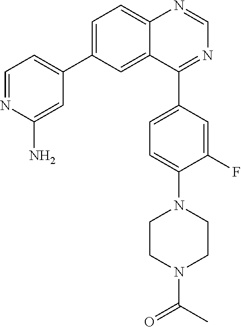 [SMILES: CC(=O)N1CCN(c2ccc(-c3ncnc4ccc(-c5ccnc(N)c5)cc34)cc2F)CC1] | WNT EC₅₀: ++ DYRK1A K_{d}: +++ |
| 34 | |
| 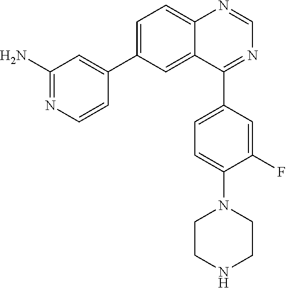 [SMILES: Nc1cc(-c2ccc3ncnc(-c4ccc(N5CCNCC5)c(F)c4)c3c2)ccn1] | WNT EC₅₀: ++ DYRK1A K_{d}: +++ |
| 35 | |
| 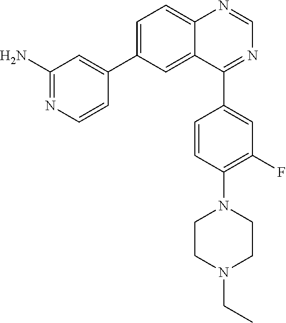 [SMILES: CCN1CCN(c2ccc(-c3ncnc4ccc(-c5ccnc(N)c5)cc34)cc2F)CC1] | WNT EC₅₀: ++ DYRK1A K_{d}: +++ |
| 36 | |
| 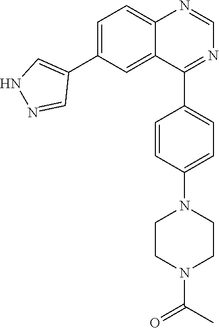 [SMILES: CC(=O)N1CCN(c2ccc(-c3ncnc4ccc(-c5cn[nH]c5)cc34)cc2)CC1] | WNT EC₅₀: +++ DYRK1A K_{d}: +++ |
| 37 | |
| 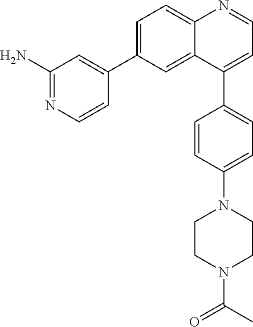 [SMILES: CC(=O)N1CCN(c2ccc(-c3ccnc4ccc(-c5ccnc(N)c5)cc34)cc2)CC1] | WNT EC₅₀: +++ DYRK1A K_{d}: +++ |
| 38 | |
| 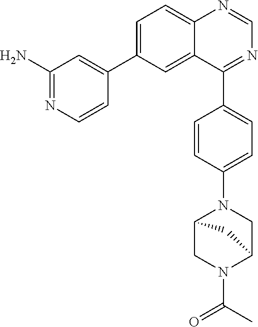 [SMILES: CC(=O)N1C[C@@H]2C[C@H]1CN2c1ccc(-c2ncnc3ccc(-c4ccnc(N)c4)cc23)cc1] | WNT EC₅₀: ++ DYRK1A K_{d}: +++ |
| 39 | |
| 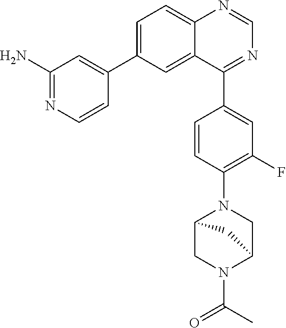 [SMILES: CC(=O)N1C[C@@H]2C[C@H]1CN2c1ccc(-c2ncnc3ccc(-c4ccnc(N)c4)cc23)cc1F] | WNT EC₅₀: ++ DYRK1A K_{d}: +++ |
| 40 | |
| 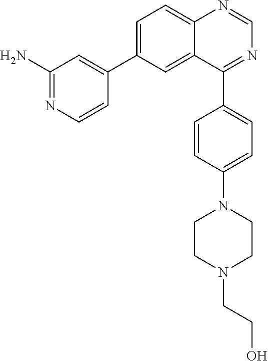 [SMILES: Nc1cc(-c2ccc3ncnc(-c4ccc(N5CCN(CCO)CC5)cc4)c3c2)ccn1] | WNT EC₅₀: +++ DYRK1A K_{d}: +++ |
| 41 | |
| 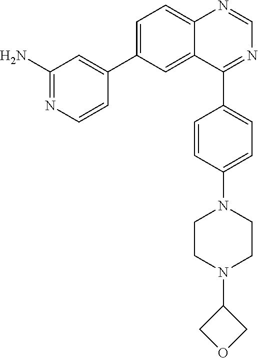 [SMILES: Nc1cc(-c2ccc3ncnc(-c4ccc(N5CCN(C6COC6)CC5)cc4)c3c2)ccn1] | WNT EC₅₀: +++ DYRK1A K_{d}: +++ |
| 42 | |
| 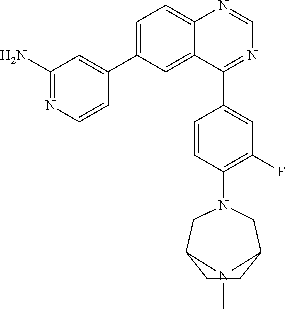 [SMILES: CN1C2CCC1CN(c1ccc(-c3ncnc4ccc(-c5ccnc(N)c5)cc34)cc1F)C2] | WNT EC₅₀: ++ DYRK1A K_{d}: +++ |
| 43 | |
| 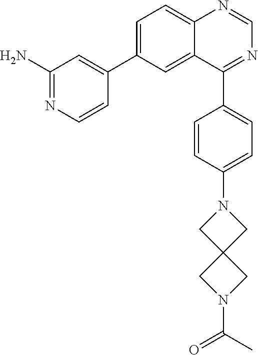 [SMILES: CC(=O)N1CC2(C1)CN(c1ccc(-c3ncnc4ccc(-c5ccnc(N)c5)cc34)cc1)C2] | WNT EC₅₀: ++ DYRK1A K_{d}: +++ |
| 44 | |
| 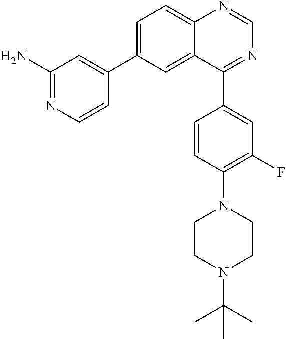 [SMILES: CC(C)(C)N1CCN(c2ccc(-c3ncnc4ccc(-c5ccnc(N)c5)cc34)cc2F)CC1] | WNT EC₅₀: ++ DYRK1A K_{d}: +++ |
| 45 | |
| 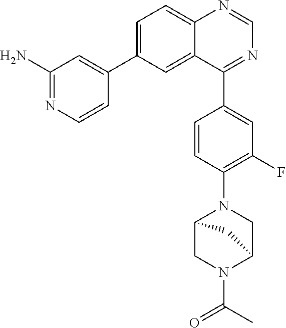 [SMILES: CC(=O)N1C[C@@H]2C[C@H]1CN2c1ccc(-c2ncnc3ccc(-c4ccnc(N)c4)cc23)cc1F] | WNT EC₅₀: N/A DYRK1A K_{d}: +++ |
| 46 | |
| 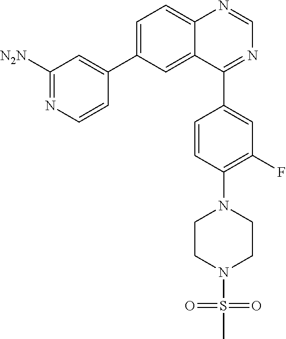 | WNT EC₅₀: +++ DYRK1A K_{d}: +++ |
| 47 | |
| 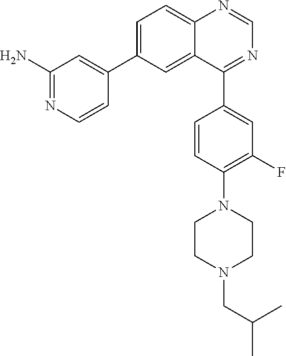 [SMILES: CC(C)CN1CCN(c2ccc(-c3ncnc4ccc(-c5ccnc(N)c5)cc34)cc2F)CC1] | WNT EC₅₀: +++ DYRK1A K_{d}: +++ |
| 48 | |
| 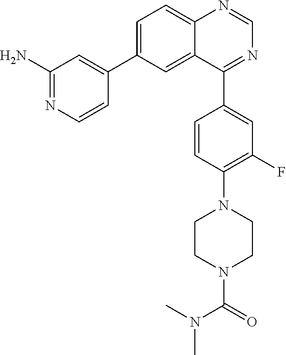 [SMILES: CN(C)C(=O)N1CCN(c2ccc(-c3ncnc4ccc(-c5ccnc(N)c5)cc34)cc2F)CC1] | WNT EC₅₀: +++ DYRK1A K_{d}: +++ |
| 49 | |
| 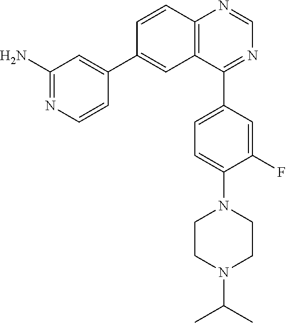 [SMILES: CC(C)N1CCN(c2ccc(-c3ncnc4ccc(-c5ccnc(N)c5)cc34)cc2F)CC1] | WNT EC₅₀: ++ DYRK1A K_{d}: +++ |
| 50 | |
| 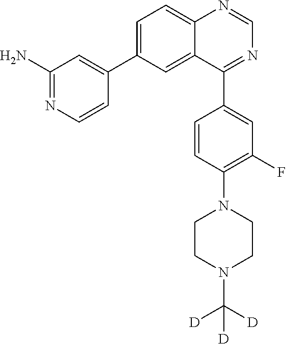 [SMILES: [2H]C([2H])([2H])N1CCN(c2ccc(-c3ncnc4ccc(-c5ccnc(N)c5)cc34)cc2F)CC1] | WNT EC₅₀: +++ DYRK1A K_{d}: +++ |
| 51 | |
| 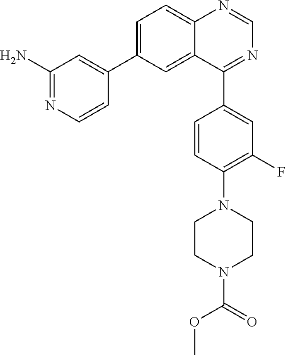 [SMILES: COC(=O)N1CCN(c2ccc(-c3ncnc4ccc(-c5ccnc(N)c5)cc34)cc2F)CC1] | WNT EC₅₀: +++ DYRK1A K_{d}: +++ |
| 52 | |
| 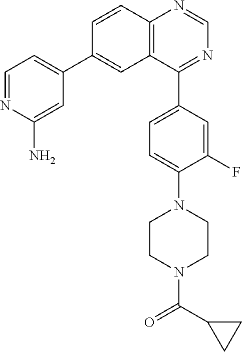 [SMILES: Nc1cc(-c2ccc3ncnc(-c4ccc(N5CCN(C(=O)C6CC6)CC5)c(F)c4)c3c2)ccn1] | WNT EC₅₀: +++ DYRK1A K_{d}: +++ |
| 53 | |
| 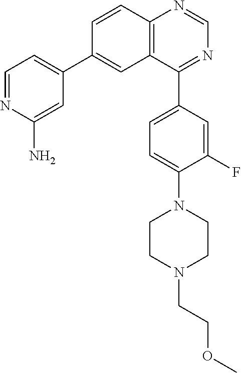 [SMILES: COCCN1CCN(c2ccc(-c3ncnc4ccc(-c5ccnc(N)c5)cc34)cc2F)CC1] | WNT EC₅₀: +++ DYRK1A K_{d}: +++ |
| 54 | |
| 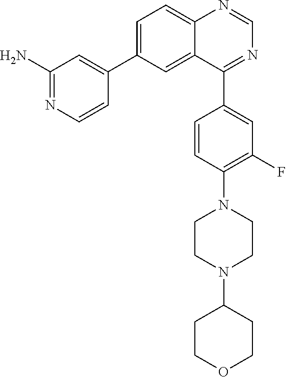 [SMILES: Nc1cc(-c2ccc3ncnc(-c4ccc(N5CCN(C6CCOCC6)CC5)c(F)c4)c3c2)ccn1] | WNT EC₅₀: +++ DYRK1A K_{d}: +++ |
| 55 | |
| 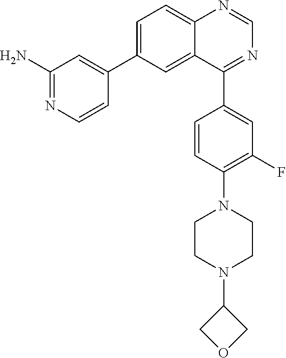 [SMILES: Nc1cc(-c2ccc3ncnc(-c4ccc(N5CCN(C6COC6)CC5)c(F)c4)c3c2)ccn1] | WNT EC₅₀: +++ DYRK1A K_{d}: +++ |
| 56 | |
| 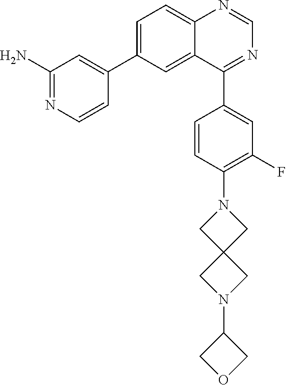 [SMILES: Nc1cc(-c2ccc3ncnc(-c4ccc(N5CC6(C5)CN(C5COC5)C6)c(F)c4)c3c2)ccn1] | WNT EC₅₀: ++ DYRK1A K_{d}: N/A |
| 57 | |
| 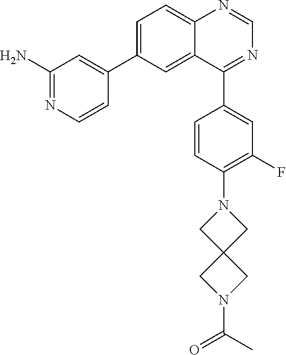 [SMILES: CC(=O)N1CC2(C1)CN(c1ccc(-c3ncnc4ccc(-c5ccnc(N)c5)cc34)cc1F)C2] | WNT EC₅₀: +++ DYRK1A K_{d}: N/A |
| 58 | |
| 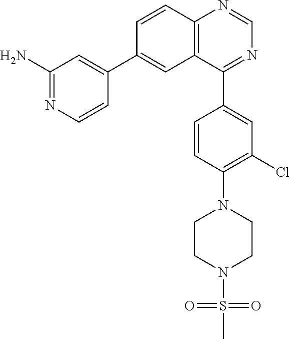 [SMILES: CS(=O)(=O)N1CCN(c2ccc(-c3ncnc4ccc(-c5ccnc(N)c5)cc34)cc2Cl)CC1] | WNT EC₅₀: +++ DYRK1A K_{d}: N/A |
| 59 | |
| 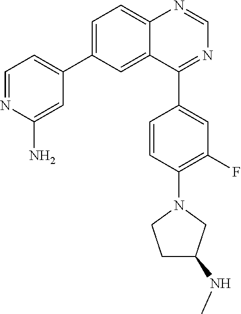 [SMILES: CN[C@H]1CCN(c2ccc(-c3ncnc4ccc(-c5ccnc(N)c5)cc34)cc2F)C1] | WNT EC₅₀: + DYRK1A K_{d}: N/A |
| 60 | |
| 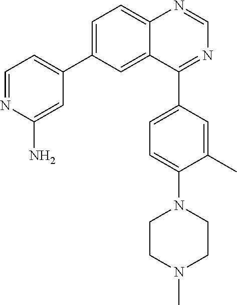 [SMILES: Cc1cc(-c2ncnc3ccc(-c4ccnc(N)c4)cc23)ccc1N1CCN(C)CC1] | WNT EC₅₀: ++ DYRK1A K_{d}: N/A |
| 61 | |
| 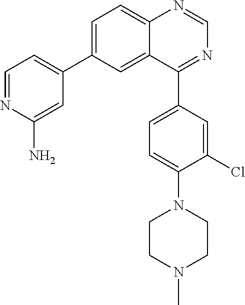 [SMILES: CN1CCN(c2ccc(-c3ncnc4ccc(-c5ccnc(N)c5)cc34)cc2Cl)CC1] | WNT EC₅₀: +++ DYRK1A K_{d}: N/A |
| 62 | |
| 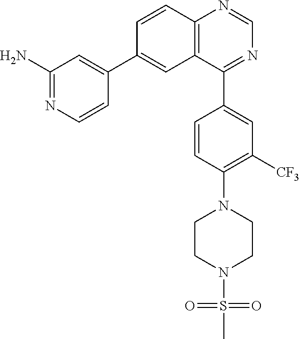 [SMILES: CS(=O)(=O)N1CCN(c2ccc(-c3ncnc4ccc(-c5ccnc(N)c5)cc34)cc2C(F)(F)F)CC1] | WNT EC₅₀: +++ DYRK1A K_{d}: N/A |
| 63 | |
| 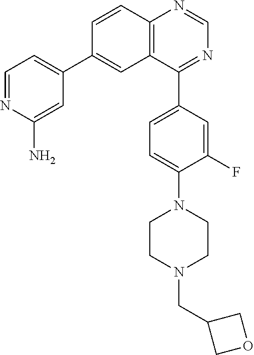 [SMILES: Nc1cc(-c2ccc3ncnc(-c4ccc(N5CCN(CC6COC6)CC5)c(F)c4)c3c2)ccn1] | WNT EC₅₀: +++ DYRK1A K_{d}: N/A |
| 64 | |

Activity Key in WNT reporter (EC50) +++ <100 nM, ++ 100 nM − 1 uM, + >1 uM. 2.
Activity key for DYRK1A affinity (KD) +++ <100 nM; ++ 100 mN − 1 uM; + >1 uM

The invention further provides processes for preparing any of the compounds of the present invention.

The invention also provides the use of compounds to not only inhibit DYRK1A activity but also signaling pathways dependent upon DYRK1A phosphorylation (e.g., Tau, PI3K/AKT, APP, PSI, ASF, RCAN-1, NFAT, p53, ASK1/JNK1, SIRT1, GluN2A and other NMDA receptors), DYRK2 phosphorylation (e.g., 26S proteasome, heat shock factor 1, p53, MYC, and JUN), and DYRK3 phosphorylation (e.g., PRAS40). The invention also relates to the use of compounds for sensitizing cells to additional agent(s), such as agents known to be effective in the treatment of neurodegenerative disorders.

In certain embodiments, the compounds are used as DYRK protein degraders (see, Valazquez, et al, 2019 Molecular Neurobiology 1-12).

The compounds of the invention are useful for the treatment, amelioration, or prevention of disorders associated with DYRK1A, DYRK1B, DYRK2, DYRK3, CLK1, CLK2, CLK3, CLK4, homeodomain-interacting kinases (HIPKs), and/or CMGC kinases leading to inhibition of WNT signaling (e.g., Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases), such as those responsive to DYRK isoform activity inhibition. In certain embodiments, the compounds can be used to treat, ameliorate, or prevent cancer that is associated with DYRK2 and DYRK3 activities (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)). In certain embodiments, the compounds can be used to treat, ameliorate, or prevent autoimmune diseases. In certain embodiments, the compounds can be used to treat, ameliorate, or prevent inflammatory disorders (e.g., airway inflammation, osteoarthritis (e.g., knee related osteoarthritis)).

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents, e.g., agents useful in treating neurodegenerative disorders and/or anticancer agents.

### DETAILED DESCRIPTION OF THE INVENTION

The DYRK family contains 5 kinases (DYRK1A, DYRK1B, DYRK2, DYRK3 and DYRK4). DYRKs belong to the CMGC group of proline-directed kinases, which also includes cyclin-dependent kinases (CDKs), mitogen-activated protein kinases (MAPKs), glycogen synthase kinases (GSKs) and CDC2-like kinases (CLKs). While the signaling pathways of CDK and MAPK families have been extensively studied, much less is known on how DYRKs and CLKs are linked to other proteins and various physiological or pathological processes. The CLK family comprises CLK1 through CLK4.

The DYRK1A gene is located on chromosome 21 (21q22.2), a region known as the Down-Syndrome Critical Region (DSCR) (see, e.g., Hämmerle et al., 2011 Development 138, 2543-2554). The under- or over-expression of the Dyrk1a gene in mammals or of its orthologous gene minibrain (mnb) in Drosophila causes severe retardation of central nervous system development and maturation. At the molecular level, DYRK1A phosphorylates the nuclear factor of activated T cells (NFAT), counteracting the effect of calcium signaling and maintaining inactive NFAT (see, e.g., Arron et al., 2006 Nature 411, 595-600). DYRK1A has been identified as a negative regulator of the cell cycle that promotes the switch to a quiescent state or differentiation (see, e.g., Chen et al., 2013 Mol. Cell 52, 87-100). In malignant cells, DYRK1A promotes survival via inhibition of pro-apoptotic proteins (see, e.g., Guo et al., 2010 J. Bio. Chem. 285, 13223-13232; Seifert et al., 2008 FEBS J. 275, 6268-6280).

Experiments conducted during the course of developing embodiments for the present invention designed, synthesized and biologically evaluated compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) as inhibitors of the dual specificity tyrosine phosphorylation regulated kinases (DYRKs: 1A, 1B, 2, 3, 4) and CLK family members (1, 2, 3, 4) for use as therapeutics against AD, down syndrome, multiple malignancies, in particular those associated with inhibition of WNT signaling and other disorders related to DYRK/CLK activity (e.g., DS, other neuropathology, glioblastoma, prostate cancer, metastatic breast cancer, metastatic lung cancer, multiple myeloma, secondary metastatic tumors of the brain). Note DYRK1B plays roles in survival of certain cancer cells and myoblast differentiation and has been validated as a promising target for CRPC.

Moreover, the DYRK/CLK inhibitors of the present invention can be used for treating other cellular pathways involved in mental impairment and neurodegenerative dementia. Specifically, the DYRK/CLK inhibitors of the present invention can be used for inhibiting DYRK1A activated PI3K/Akt signaling, a pathway largely involved in neuronal development, growth, and survival. The DYRK1A inhibitors of the present invention DYRK1A can be used for inhibiting DYRK1A stimulated ASK1/JNK1 activity, thereby inducing neuronal death and apoptosis. In addition, the DYRK1A inhibitors of the present invention DYRK1A can be used to inhibit DYRK1A phosphorylation of p53 during embryonic brain development, thereby preventing neuronal proliferation alteration. The DYRK1A inhibitors of the present invention can be used to inhibit DYRK1A phosphorylation of synaptic proteins Amph 1, Dynamin 1, and Synaptojanin, involved in the regulation of endocytosis, thereby retaining synaptic plasticity through preventing alteration of the number, size, and morphology of dendritic spines. The DYRK1A inhibitors of the present invention can be used to inhibit presenilin 1 (the catalytic sub-unit of γ-secretase). The DYRK1A inhibitors of the present invention can be used to inhibit DYRK2/3 & 4 activity. The DYRK1A inhibitors of the present invention can be used to inhibit DYRK1B activity. The DYRK1A inhibitors of the present invention can be used to inhibit CMGC CLK1-4 kinase activity.

As such, the present invention addresses the need for effective therapies for GBM, AD and DS by providing potent and pan-selective DYRK/CLK inhibitors able to permeate the blood-brain barrier (BBB) and elicit on-mechanism therapeutic responses in animal models. Disease states in the periphery include colorectal cancer, castration-resistant prostate cancer and malignancies associated with inhibition of WNT signaling.

Accordingly, the present invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

The CDC2-like kinase (CLK) family contains four isoforms which are important in regulating the function of the spliceosome complex (see, e.g., Fedorov et al, Chem Biol. 2011; 18(1):67-76). This complex, comprised of small nuclear RNAs (snRNA) and a large number of associated proteins, regulates the splicing of pre-mRNAs to give mature protein-encoding mRNAs. CLK1 is known to regulate the activity of the spliceosome via phosphorylation of the constituent serine-arginine-rich (SR) proteins (see, e.g., Bullock et al. Structure. 2009; 17(3):352-62). By controlling the activity of the spliceosome in this way, many genes are able express more than one mRNA leading to diversity in the translated proteins. The alternative protein iso forms transcribed from the same gene will often have different activities and physiological functions. Deregulation of alternative splicing has been linked to cancer, where a number of cancer-related proteins are known to be alternatively spliced (see, e.g., Druillennec et al, J Nucleic Acids. 2012; 2012:639062). An example of an alternatively spliced protein in cancer is Cyclin D1, important for the progression of cancer cells through the cell cycle (see, e.g., Wang et al, Cancer Res. 2008; 68(14):5628-38).

Alternative splicing regulated by CLK1 has also been described to play a role in neurodegenerative diseases, including Alzheimer's and Parkinson's, via phosphorylation of the SR proteins of the spliceosome (see, e.g., Jain et al, Curr Drug Targets. 2014; 15(5):539-50). In the case of Alzheimer's, CLK1 is known to regulate the alternative splicing of the microtubule-associated protein TAU leading to an imbalance between TAU iso forms which is sufficient to cause neurodegeneration and dementia (see, e.g., Liu et al, Mol Neurodegener. 2008; 3:8).

In the treatment of both cancer and neurological disease, there is thus undoubtedly an urgent need for compounds which potently inhibit DYRK and CLK kinases whilst not affecting other closely-related kinases. The compounds described herein address this need.

In a particular embodiment, compounds encompassed within the following formulas are provided:

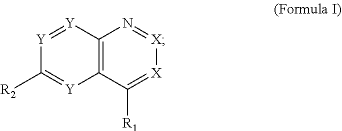

including pharmaceutically acceptable salts, solvates, and/or prodrugs thereof.

Formula I is not limited to a particular chemical moiety for X, Y, R1 and R2. In some embodiments, the particular chemical moiety for X, Y, R1 and R2 independently include any chemical moiety that permits the resulting compound to inhibit DYRK1A activity. In some embodiments, the particular chemical moiety for X, Y, R1 and R2 independently include any chemical moiety that permits the resulting compound to inhibit one or more of DYRK1A related PI3K/Akt signaling; DYRK1A related tau phosphorylation; DYRK1A related NFAT phosphorylation; DYRK1A related ASK1/JNK1 pathway activation; DYRK1A related p53 phosphorylation; DYRK1A related Amph 1 phosphorylation; DYRK1A related Dynamin 1 phosphorylation; DYRK1A related Synaptojanin phosphorylation; DYRK1A related presenilin 1 (the catalytic sub-unit of γ-secretase) activity; DYRK1A related amyloid precursor protein phosphorylation; DYRK1A related SIRT1 activation; DYRK2 related heat shock factor 1 and 26S proteasome activities; DYRK3 related mTOR activity; DYRK3 phosphorylation (e.g., PRAS40); DYRK1B activity; CMGC/CLK kinase activity; CLK1 activity; CLK2 activity; CLK3 activity; CLK4 activity; CDK7 activity; CDK8 activity; CDK19 activity; PI3K activity; PI3K mutant activity; PDGFrA/B activity; mTOR activity; c-KIT activity; RYK activity; and WNT signaling.

Such embodiments are not limited to a particular definition for each of the “X” and “Y” substituents.

In some embodiments, one of the “X” substituents is carbon and the other is nitrogen, or both of the “X” substituents are carbon; and one of the “Y” substituents is nitrogen and the other “Y” substituents are carbon, or two of the “Y” substituents are nitrogen and one “Y” substituent is carbon, or all of the “Y” substituents are carbon; such that the resulting structure is one of the following formulas:

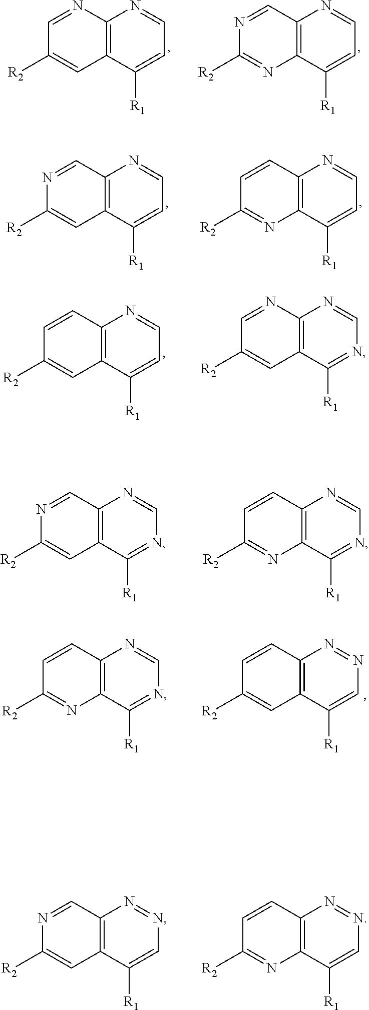

In some embodiments, R1 is an aryl or heteroaryl ring.

In some embodiments, R1 is selected from hydrogen, halogen (e.g., fluorine, bromine, iodine, chlorine),

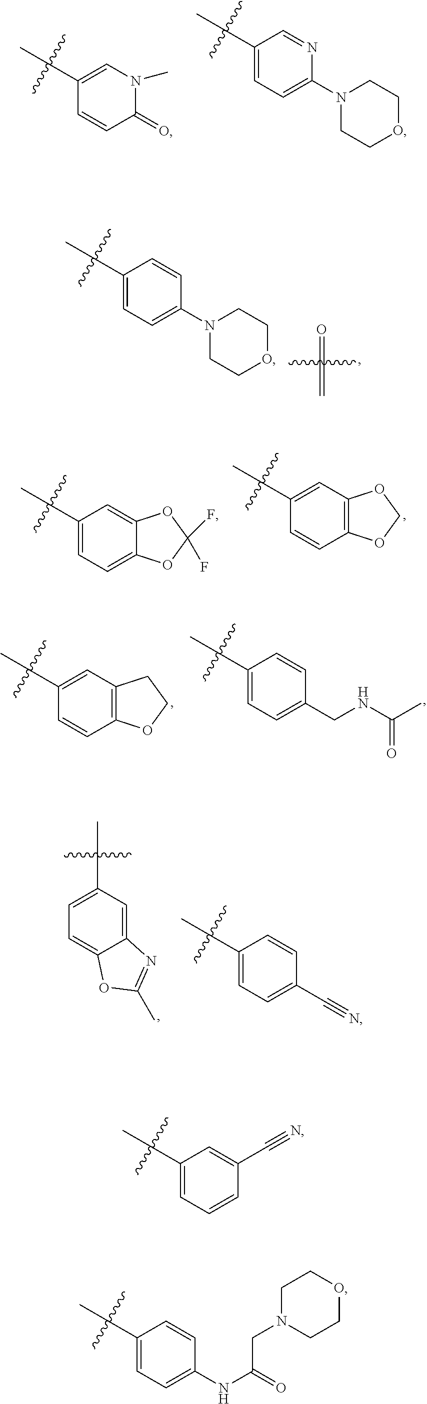

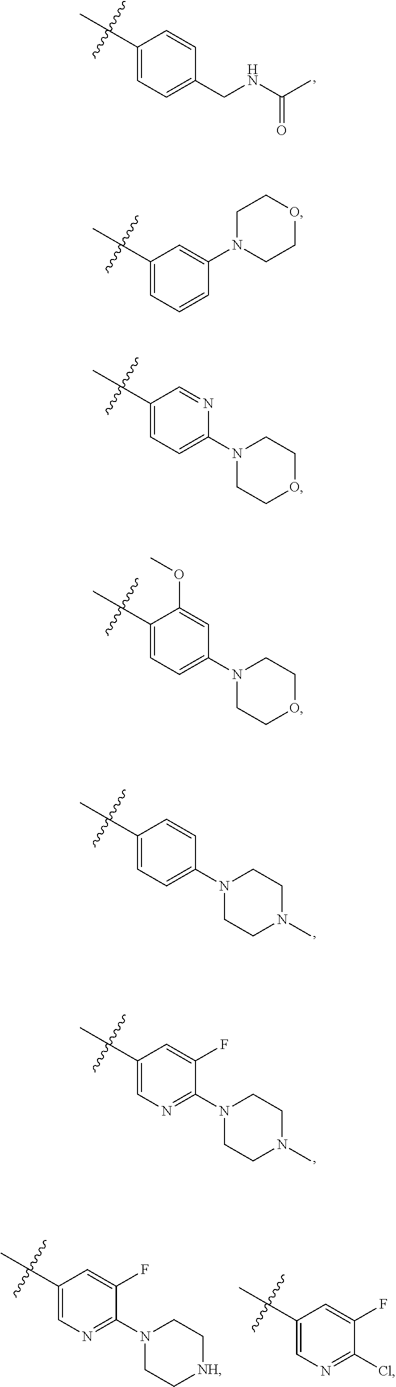

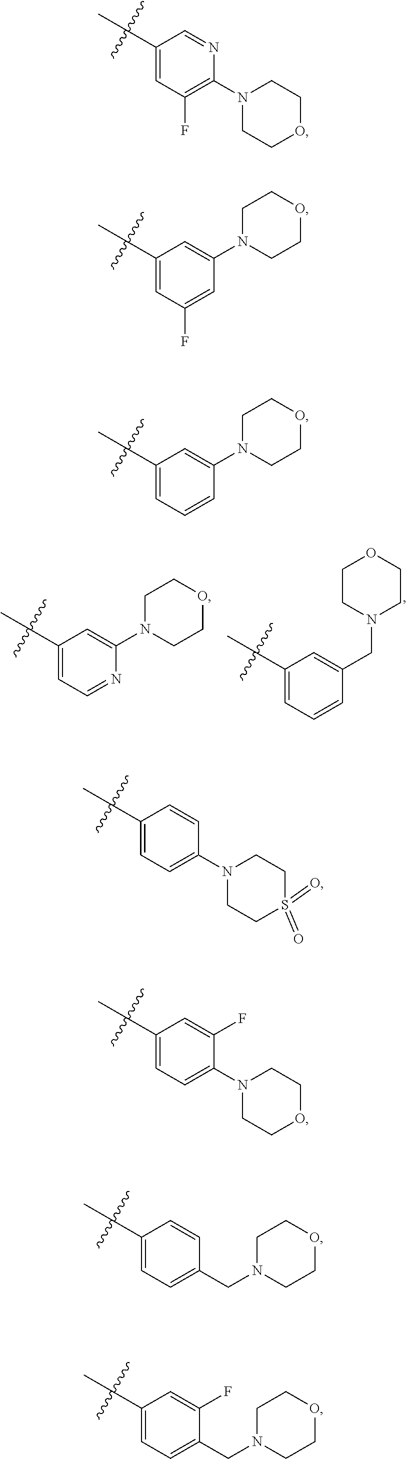

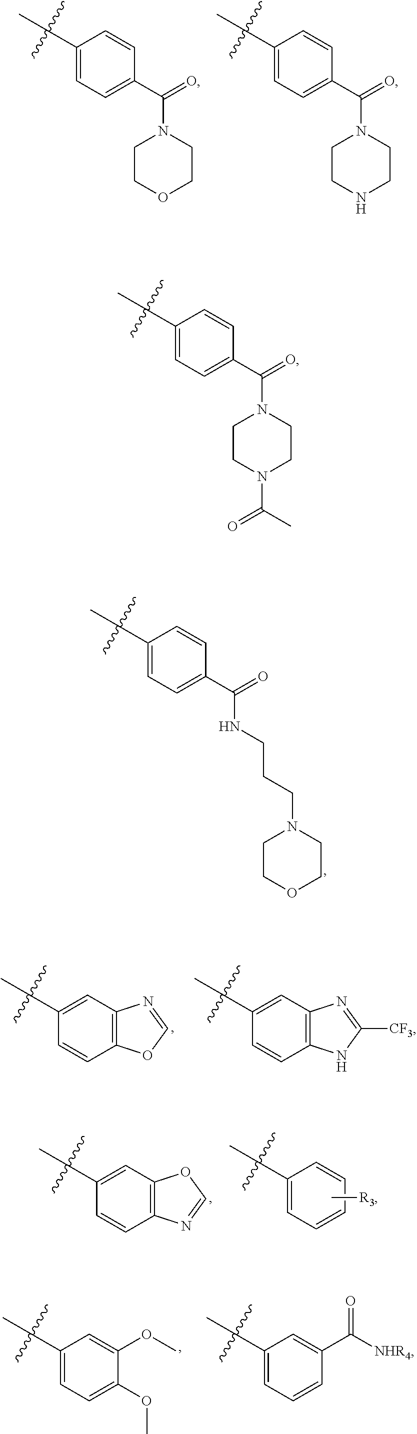

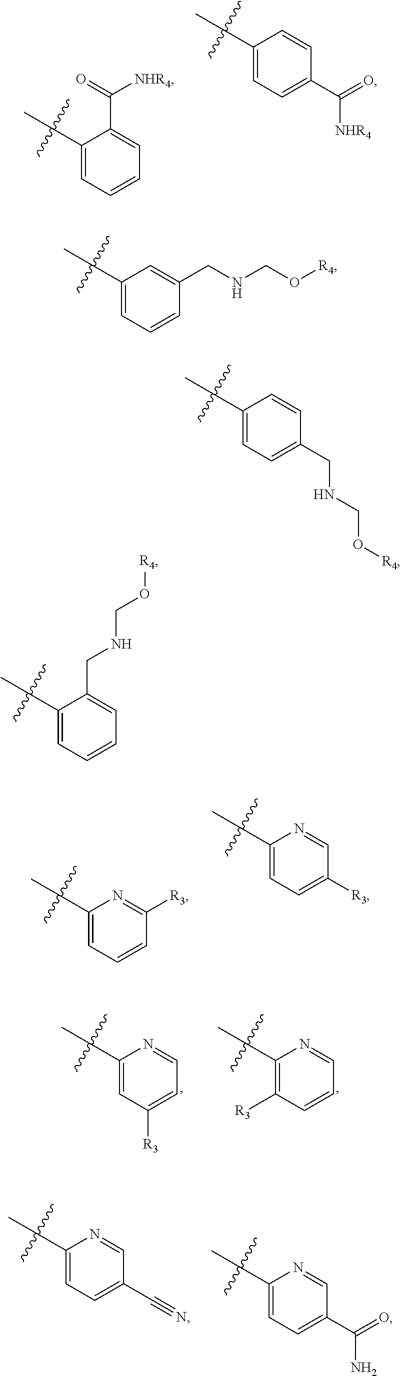

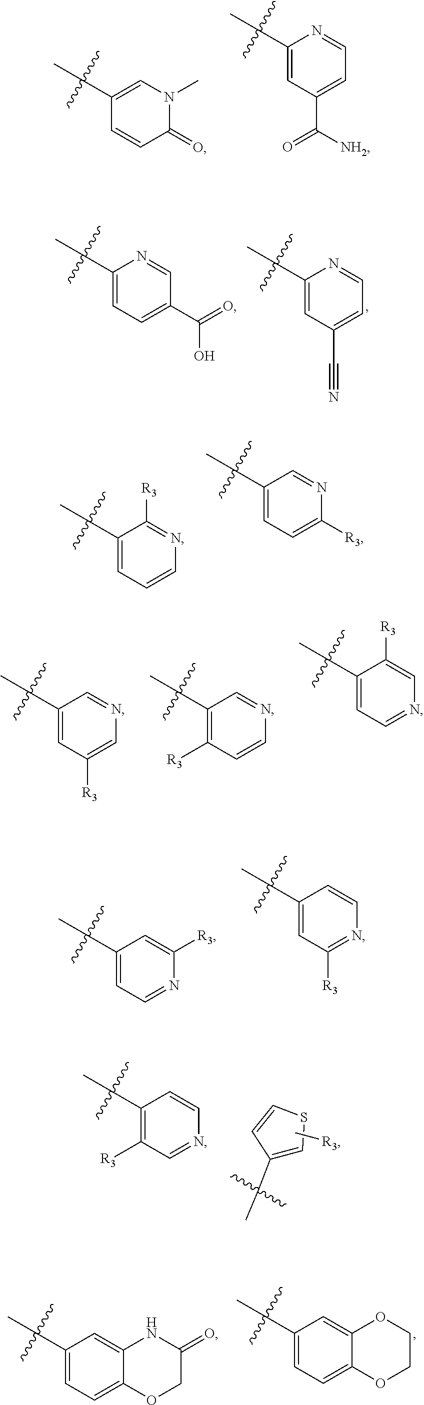

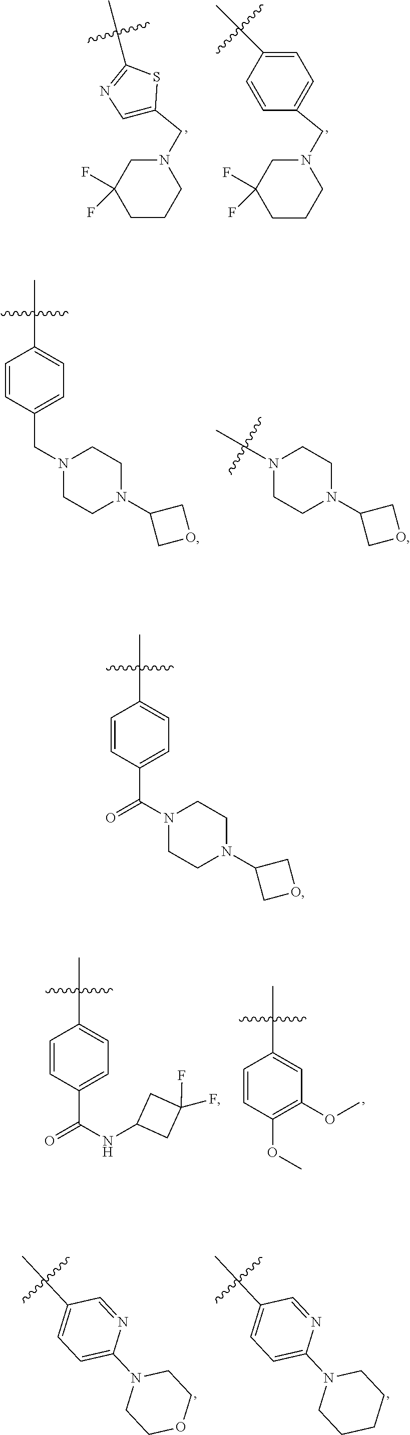

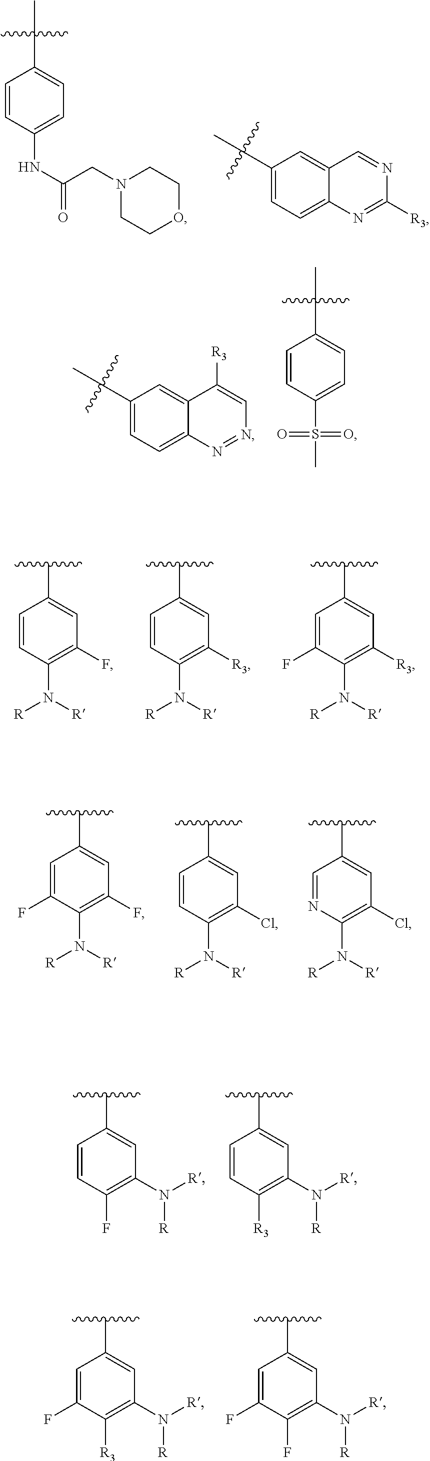

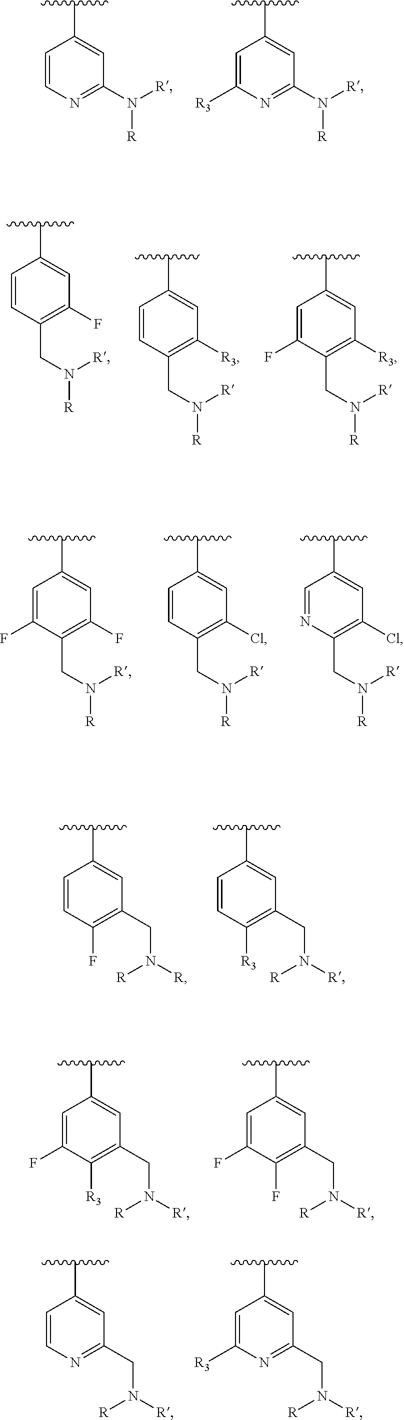

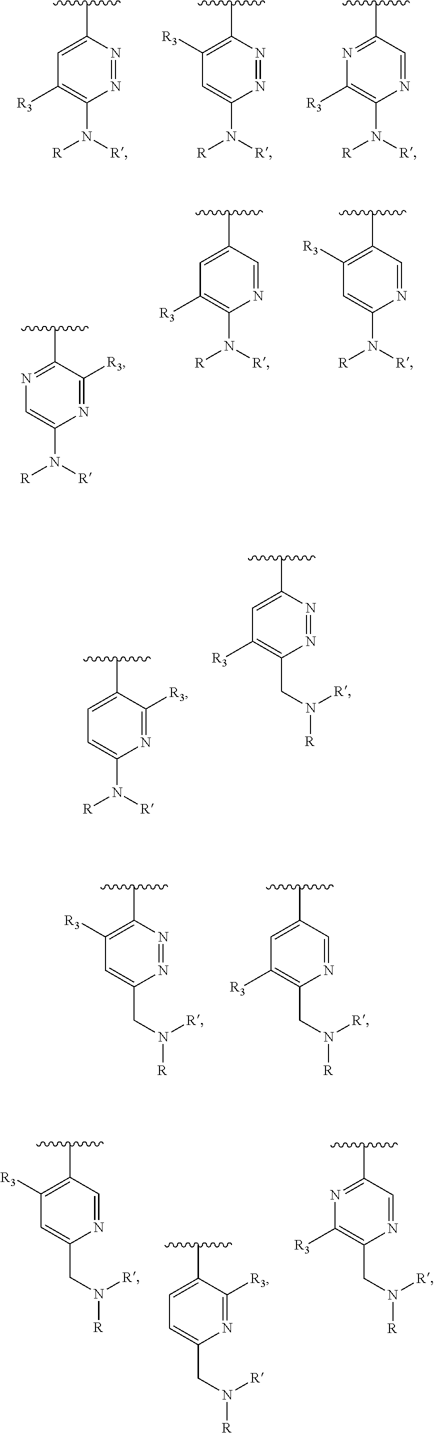

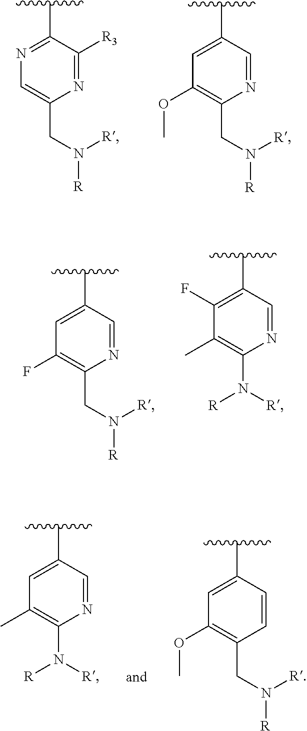

In some embodiments, R2 is selected from hydrogen, halogen (e.g., fluorine, bromine, iodine, chlorine), aryl, substituted aryl, heteroaryl, substituted heteroaryl,

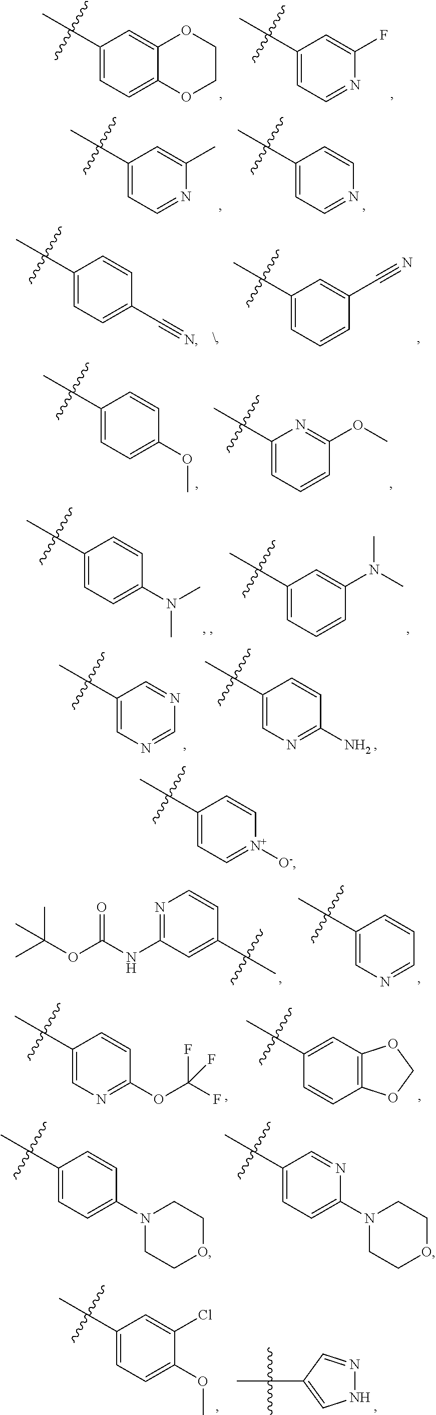

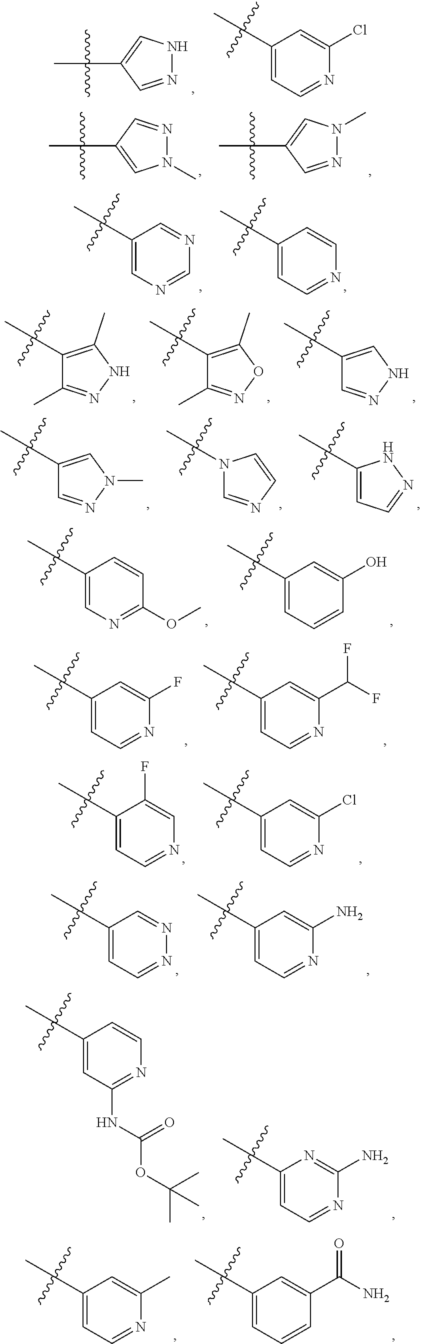

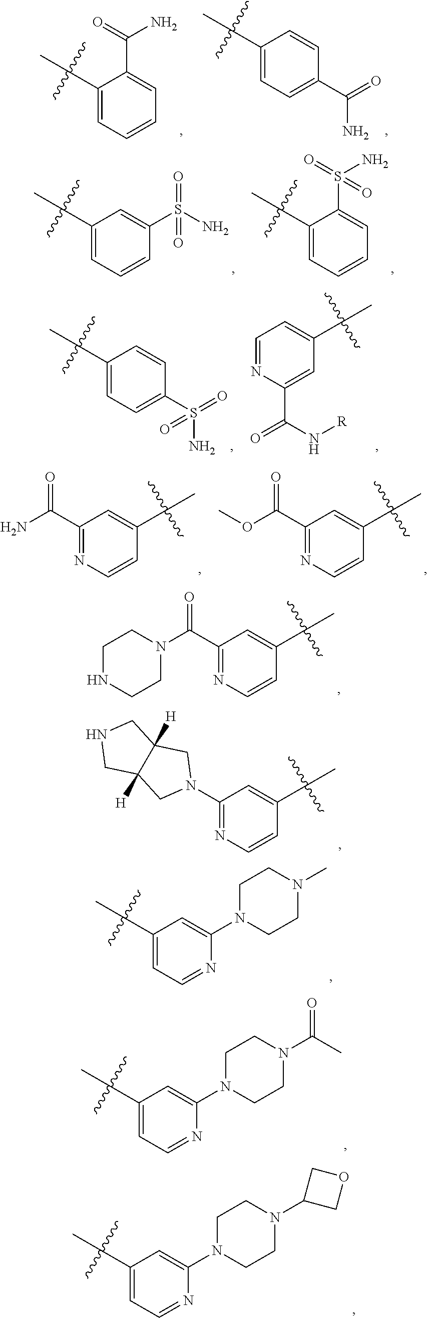

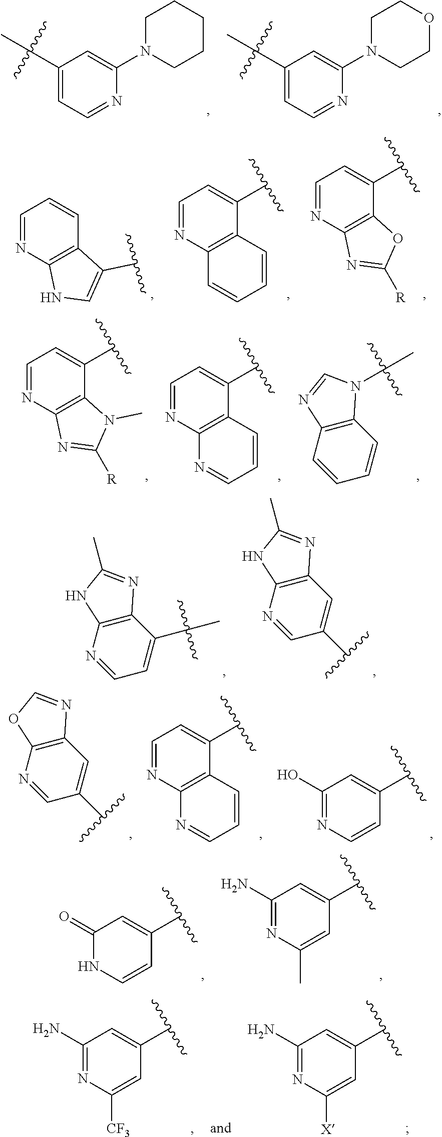

wherein X″ is selected from alkyl, haloalkyl, amino, alkylamino, hydroxy, fluoro, chloro, bromo, and cyano groups.

In some embodiments, R, R′ and R″ are independently selected from hydrogen, halogen (e.g., fluorine, bromine, chlorine, iodine), di-halogen (di-fluorine, di-bromine, di-chlorine, di-iodine), CF3, OCH3, CHF2H, OCF3, methyl, di-methyl, alkoxy, alkylsulfonyl, cyano, carboxy, ester, amido, substituted amido, sulfonamide, substituted sulfonamide, methylenedioxy, heterocyclyl alkyl, heterocyclyl, heterocyclyl alkyl amido, a lipophilic moiety comprising ether, a secondary or tertiary amine moiety consisting of a heterocycloalkyl group that is bioisosteric to secondary amines (e.g., morpholine, piperidine, piperazine).

In some embodiments, R3 and R4 are independently selected from hydrogen, halogen (e.g., fluorine, bromine, chlorine, iodine), methyl, ethyl, and methoxy.

In some embodiments, the compounds are compounds 1-64 as recited in Table 1.

The invention further provides processes for preparing any of the compounds of the present invention.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, Alzheimer's disease, Down syndrome, Huntington's disease, Parkinson's disease, autoimmune diseases, cancer (e.g., glioblastoma, prostate cancer, metastatic breast cancer, metastatic lung cancer, multiple myeloma, secondary metastatic tumors of the brain), inflammatory disorders (e.g., airway inflammation), any neurodegenerative disorder related to DYRK1A, DYRK1B, DYRK2, DYRK3, and/or CLK1, CLK2, CLK3 and CLK4 activity, and any type of cancer related to DYRK1A, DYRK1B, DYRK2, DYRK3, and/or CLK1, CLK2, CLK3 or CLK4 activity, in particular those associated with disruption of WNT signaling.

Some embodiments of the present invention provide methods for administering an effective amount of a compound of the invention and at least one additional therapeutic agent (including, but not limited to, any agent useful in treating Alzheimer's disease, Down syndrome, Huntington's disease, Parkinson's disease, autoimmune diseases, inflammatory disorders (e.g., airway inflammation), any neurodegenerative disorder related to DYRK1A, DYRK1B, DYRK2, DYRK3, and/or CLK1, CLK2, CLK3 or CLK4 activity, in particular those associated with disruption of WNT signaling.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### Examples

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

This example provides synthesis and characterization information for compounds of the present invention.

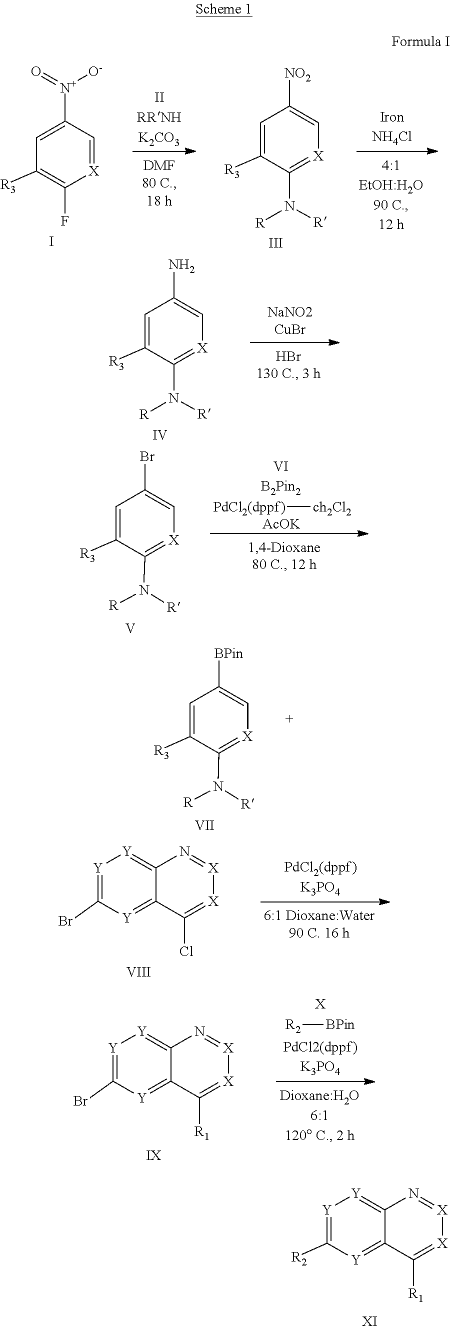

Scheme 1 describes the synthesis of general product (XI), from commercially available nitrobenzenes and nitropyridines (I). The first step of the reaction scheme is an S_{N}Ar reaction with commercially available amines (II), to furnish (III). The substituted nitrobenzene can be reduced in a Bechamp style reaction to yield anilines (IV). The anilines are subsequently converted into (hetero)aryl bromides (V) via a Sandmeyer reaction. The (hetero)aryl bromides (V) are utilized in a Miyaura borylation to produce the pinacol boronic esters (VII). The boronic esters are coupled with polyhalogenated heterocycles (VIII) in a Suzuki reaction to afford the R₁ substituted heterocyle (IX) which is reacted in an additional Suzuki reaction resulting in R₂, R₁ substituted heterocycle (XI).

In some embodiments, compounds of Formula I of this present disclosure can be prepared as depicted in scheme 2.

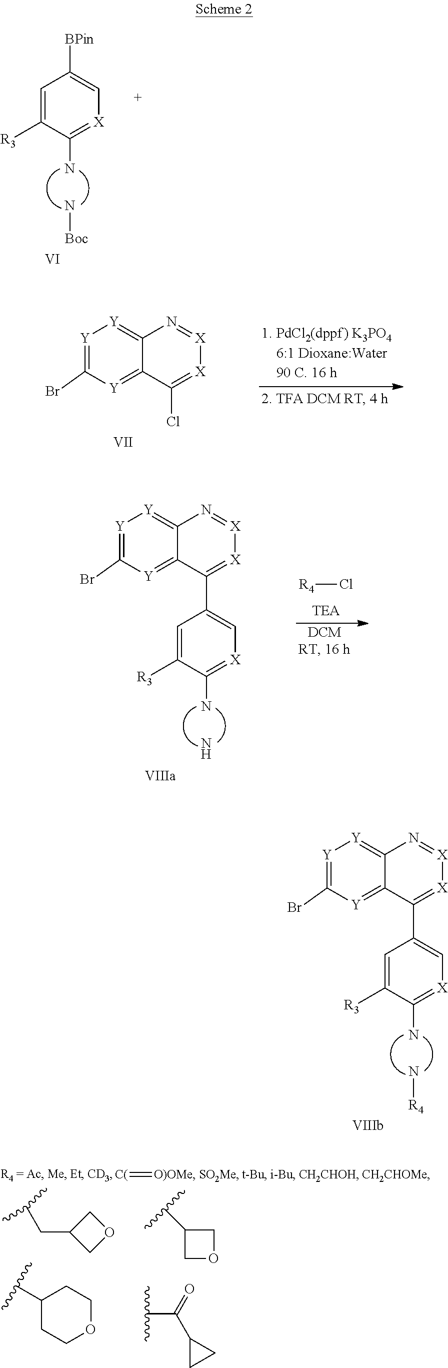

Scheme 2 describes the synthesis of advance intermediate (VIIIb), which can be prepared with commercially available, or readily prepared (hetero)aryl pinacol boronates (VI) and polyhalogenated bicyclic heteroaryl by a Suzuki reaction, followed by an acid mediated deprotection, to be further functionalized to produce General Intermediate (VIIIb).

In some embodiments, compounds of Formula I of this present disclosure can be prepared as depicted in scheme 3.

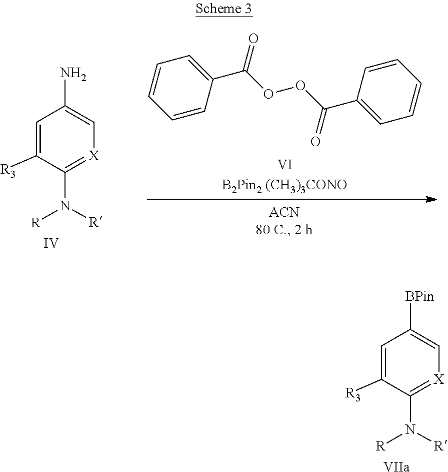

Scheme 3 describes the preparations of Boronic pinacol esters (VIIa) via a Sandmeyer-type borylation using aniline (IV).

In some embodiments, compounds of Formula I of this present disclosure can be prepared as depicted in scheme 4.

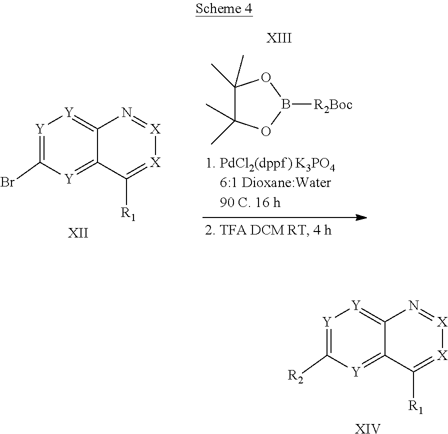

Scheme 4 describes the synthesis of product (XIV) via Suzuki coupling followed by an acid mediated deprotection.

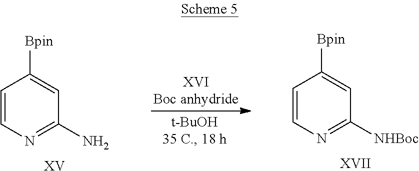

Preparation of intermediate tert-butyl (4-(4,4,5,5-tetramethyl-1,3,2-dioxaborolan-2-yl)pyridin-2-yl)carbamate (XVII) is depicted below in Scheme 5.

4-(4,4,5,5-tetramethyl-1,3,2-dioxaborolan-2-yl)pyridin-2-amine (XV) (206 mg, 1 eq, 0.937 mmol) was stirred in t-BuOH (4.5 mL) under an argon atmosphere. After 10 min di-tert-butyl decarbonate (XVI) (225 mg, 221 μL, 1.1 eq, 1.03 mmol) in 6.5 mL of t-BuOH was slowly added to the reaction flask. The flask was placed under argon for an additional 10 min. The reaction was then placed in an oil bath at 35° C. for 18 h. Upon completion the reaction was cooled, and the solvent was removed under vacuum. The crude material was stirred in water for 5 min and then collected through filtration to produce tert-butyl (4-(4,4,5,5-tetramethyl-1,3,2-dioxaborolan-2-yl)pyridin-2-yl)carbamate (XVII) (300 mg, 937 μmol, 99%) as a white solid. ¹H NMR (500 MHz, DMSO-d₆) δ 9.70 (s, 1H), 8.20 (d, J=4.7 Hz, 1H), 8.02 (s, 1H), 7.11 (d, J=4.7 Hz, 1H), 1.41 (s, 9H), 1.24 (s, 12H). ¹³C NMR (126 MHz, DMSO-d₆) δ 153.22, 152.67, 147.91, 123.13, 117.53, 84.80, 80.05, 28.49, 25.11.

Preparation of intermediate 6-bromo-4-(6-chloro-5-fluoropyridin-3-yl)quinazoline (XX) is depicted below in Scheme 6.

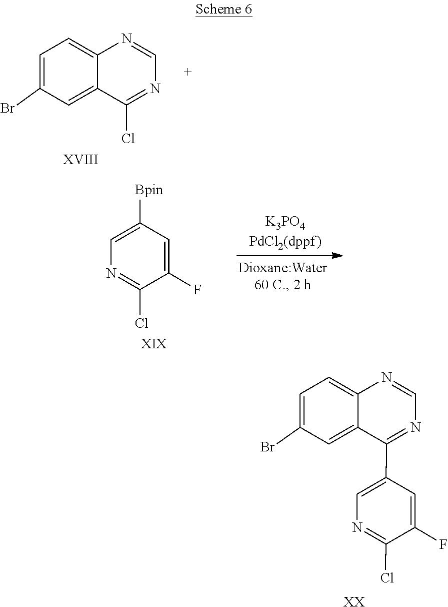

A 100-mL-round-bottle mixture of 6-bromo-4-chloroquinazoline (XVIII) (284 mg, 2 eq, 1.17 mmol), 2-chloro-3-fluoro-5-(4,4,5,5-tetramethyl-1,3,2-dioxaborolan-2-yl)pyridine (XIX) (150 mg, 1 eq, 583 μmol), and tirpotassium phosphate (185 mg, 1.5 eq, 874 μmol) and PdCl2(dppf) (46 mg, 0.1 eq, 58.3 μmol) in 1,4-Dioxane (20 mL) and water (3.3 mL) was heated at 60° C. under argon atmosphere for 2 hrs. The reaction was cooled to the room temperature, diluted with ethyl acetate (40 ml) and washed with saturated sodium bicarbonate (10 ml) and brine (10 ml). The organic layer was dried over sodium sulfate, evaporated in vacuo to get the crude product. The crude product was purified with silica gel chromatography (40 g silica gel, eluent EtOAc/Hex=0-100%) to afford the product 6-bromo-4-(6-chloro-5-fluoropyridin-3-yl)quinazoline (XX) (152 mg, 449 μmol, 77%). ¹H NMR (500 MHz, DMSO-d₆) δ 9.39 (s, 1H), 8.62 (d, J=1.8 Hz, 1H), 8.36 (dd, J=9.0, 1.9 Hz, 1H), 8.25 (d, J=2.0 Hz, 1H), 8.16 (dd, J=9.0, 2.1 Hz, 1H), 8.02 (d, J=9.0 Hz, 1H). ¹³C NMR (126 MHz, DMSO-d₆) δ 162.52, 155.04, 149.55, 146.03, 138.59, 134.18, 131.04, 128.97, 127.18, 124.29, 122.29.

Preparation of intermediate 4-(4-chloroquinolin-6-yl)pyridin-2-amine (XXIII) is depicted below in Scheme 7.

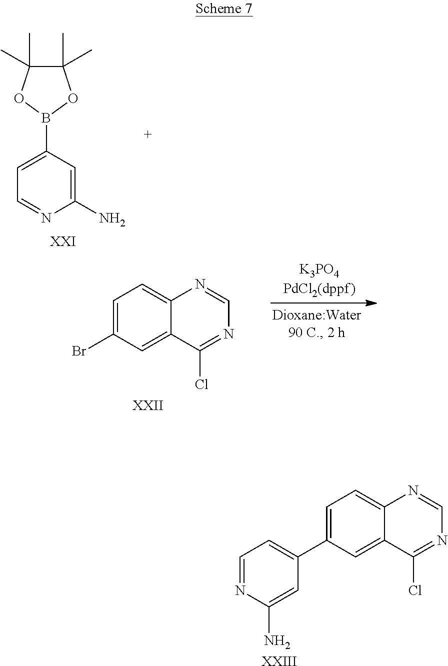

A mixture of 6-bromo-4-chloroquinoline (XXI) (485 mg, 1 eq, 2.000 mmol), 4-(4,4,5,5-tetramethyl-1,3,2-dioxaborolan-2-yl)pyridin-2-amine (XXI) (440 mg, 1 eq, 2.000 mmol), potassium phosphate (637 mg, 1.5 eq, 3.000 mmol), and PdCl2(dppf) (158 mg, 0.1 eq, 200.0 μmol) in 1,4-Dioxane (0.8 mL) and water (0.1 mL) was heated at 90° C. under argon atmosphere for 2 hrs. The reaction mixture was cooled to room temperature, and diluted with ethyl acetate (10 ml), washed with water (5 ml). The aqueous solution was washed with ethyl acetate (10 ml). The combined organic layer was washed with brine (10 ml), dried over sodium sulfate, filtered, and evaporated in vacuo to get the crude product. The crude product was purified with silica gel chromatography (24 g, ethyl acetate/MeOH=0 10 10%) to afford 4-(4-chloroquinolin-6-yl)pyridin-2-amine (XXIII) (381 mg, 1.49 mmol, 75%). LCMS [M+H]⁺=256. ¹H NMR (500 MHz, CDCl₃) δ 9.03 (d, J=4.7 Hz, 1H), 8.63 (s, 1H), 8.43 (t, J=7.8 Hz, 2H), 8.20 (d, J=8.6 Hz, 1H), 7.90-7.58 (m, 2H), 7.07 (s, 1H), 4.98 (brs, 2H). ¹³C NMR (126 MHz, CDCl₃) δ 159.11, 150.35, 149.12, 149.03, 148.81, 142.92, 138.02, 130.60, 129.28, 126.53, 122.26, 121.80, 112.84, 106.70.

Preparation of intermediate 6-bromo-4-(3-fluoro-4-(piperazin-1-yl)phenyl)quinazoline (XXV) is depicted below in Scheme 8.

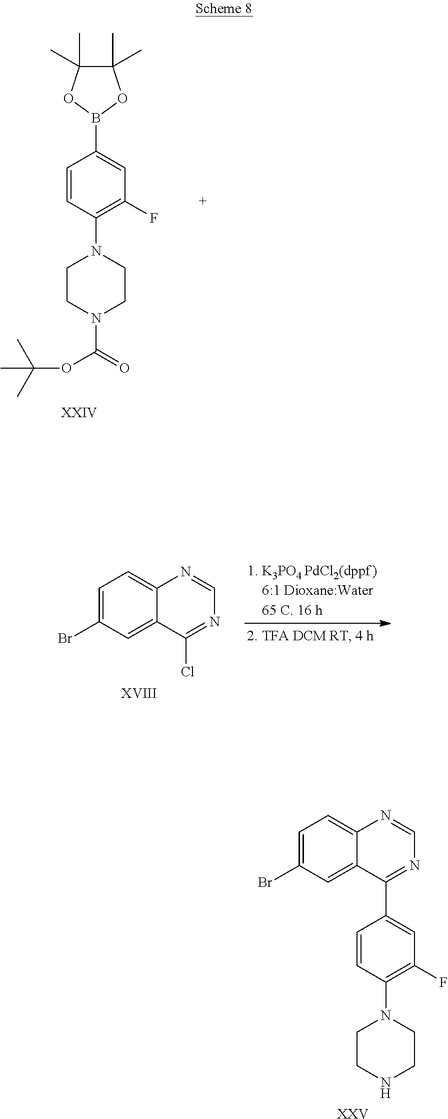

In a vial 6-bromo-4-chloroquinazoline (XVIII) (2.10 g, 1 eq, 8.61 mmol), tert-butyl 4-(2-fluoro-4-(4,4,5,5-tetramethyl-1,3,2-dioxaborolan-2-yl)phenyl)piperazine-1-carboxylate (XXIV) (3.50 g, 1 eq, 8.61 mmol), PdCl2dppf (315 mg, 0.05 eq, 431 μmol), K3PO4 (5.49 g, 3 eq, 25.8 mmol) and purged for 10 min. dioxane (72 mL), Water (12 mL) (6:1) were added to the vial and purged for an additional 10 min. The reaction was placed in the oil bath at 65° C. for 16 hour. Upon completion, the reaction was cooled and diluted with DCM (19 mL), and 2,2,2-trifluoroacetic acid (7.28 g, 4.890 mL, 20 eq, 63.85 mmol) were added to a vial. The mixture stirred at 25° C. for 4 h. After this time, the mixture was poured into DCM and washed with water to afford 6-bromo-4-(3-fluoro-4-(piperazin-1-yl)phenyl)quinazoline (XXV) (1.56 g, 1 eq, 3.193 mmol) solid, as a yellow solid. LCMS [M+H]⁺=388. ¹H NMR (400 MHz, DMSO-d₆) δ 9.27 (s, 1H), 8.29 (d, J=2.0 Hz, 1H), 8.26-8.21 (m, 2H), 7.98 (d, J=5.4 Hz, 1H), 7.66-7.60 (m, 2H), 7.18 (t, J=8.8 Hz, 1H), 6.81 (dd, J=5.3, 1.8 Hz, 1H), 6.71 (d, J=1.7 Hz, 1H), 6.02 (s, 1H), 3.07 (t, J=4.9 Hz, 4H), 2.84 (q, J=7.0 Hz, 4H), 1.03 (s, 1H). ¹³C NMR (101 MHz, CDCl₃) δ 167.13, 159.24, 156.49, 154.87, 154.32, 151.19, 149.06, 148.71, 138.37, 132.74, 129.65, 126.87, 124.97, 123.38, 122.99, 118.84, 117.93, 112.59, 106.76, 50.40, 45.96

### Example 1.1: Preparation of 4-(4-(3-fluoro-4-morpholinophenyl)quinazolin-6-yl)pyridin-2-amine (1)

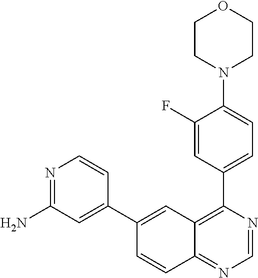 [SMILES: Nc1cc(-c2ccc3ncnc(-c4ccc(N5CCOCC5)c(F)c4)c3c2)ccn1]

### Step A. Preparation of 4-(4-bromo-2-fluorophenyl)morpholine

A mixture of 3-fluoro-4-morpholinoaniline (5 g, 30 mmol) and 48% HBr (50 ml) was stirred at 0° C., NaNO₂ (5 g, 80 mmol) in water (50 ml) was added dropwise to the solution and stirred for another 30 mins. CuBr (8 g, 50 mmol) was added to the reaction solution, and the resulting mixture was heated at 130° C. for 3 hrs. The reaction mixture was cooled down to the room temperature, NaOH (5.0 N, 50 ml) was added to the solution and extracted with ethyl acetate (150 ml). The solution was filtered with Celite to remove greenish copper salts. The organic layer in filtrate was further collected through separatory funnel. The aqueous solution was washed with ethyl acetate (60 ml). The combined organic layer was washed with brine (10 ml), dried over sodium sulfate, evaporated in vacuo to obtain the crude product. The crude product was purified with silica gel (liquid, 80 g, eluent 0-60% ethyl acetate in hexanes) to afford the desired product. LCMS [M+H]⁺=259.

### Step B. Preparation of 4-(2-fluoro-4-(4,4,5,5-tetramethyl-1,3,2-dioxaborolan-2-yl)phenyl)morpholine

A mixture of 4-(4-bromo-2-fluorophenyl)morpholine (5 g, 1 eq, 0.02 mol), 4,4,4′,4′,5,5,5′,5′-octamethyl-2,2′-bi(1,3,2-dioxaborolane) (8 g, 1.7 eq, 0.03 mol), potassium acetate (6 g, 3 eq, 0.06 mol), and PdCl2(dppf) (1 g, 0.1 eq, 2 mmol) in 1,4-dioxane (50 ml) was heated at 120° C. in argon atmosphere for 2.5 hrs. (Note: The product and starting material are similar in polarity by TLC plate analysis, 20% ethyl acetate in hexanes indicated 0.3 Rf value, Iodine indicator reveals different stained color). The reaction was cooled to the room temperature, and diluted with ethyl acetate (100 ml), then washed with water (50 ml). The aqueous solution was washed with ethyl acetate (80 ml), and the combined organic layer was filtered with Celite (due to dark red color mixed with water). The organic layer was collected and dried over sodium sulfate, filtered, and evaporated in vacuo, to obtain the crude product (note: The residue contained a great deal of HOAc from KOAc due to weak vacuum system). The crude product was purified with silica gel chromatography (80 g, 0-60-% ethyl acetate in hexanes) to afford 4-(2-fluoro-4-(4,4,5,5-tetramethyl-1,3,2-dioxaborolan-2-yl)phenyl)morpholine (6 g, 20 mmol) as a white solid. LCMS [M+H]⁺=307.

### Step C. Preparation of 4-(4-(6-bromoquinazolin-4-yl)-2-fluorophenyl)morpholine

A mixture of 6-bromo-4-chloroquinazoline (595 mg, 1.5 eq, 2.44 mmol), 4-(2-fluoro-4-(4,4,5,5-tetramethyl-1,3,2-dioxaborolan-2-yl)phenyl)morpholine (500 mg, 1 eq, 1.63 mmol). PdCl₂(dppf) (128 mg, 0.1 eq, 163 μmol), and tripotassium phosphate (518 mg, 1.50 eq, 2.44 mmol) in 1,4-dioxane (6 mL) and Water (1 mL) was heated at 90° C. in argon atmosphere for 16 hrs. The reaction solution was cooled to the room temperature, and diluted with ethyl acetate (20 ml), washed with water (5 ml), and brine (5 ml). The organic layer was dried over Na₂SO₄, evaporated in vacuo to obtain the crude product. The crude product was purified with silica gel chromatography (12 g, eluent 0-10% MeOH in DCM) to afford the desired product. However, the fractions were not pure based on TLC plate analysis. Thus, evaporation of combined fractions were further washed with MeOH (5-10 ml), filtered off and collected the pure product. Repeated one more time to collect the rest of product. Combined product gave rise to a yellow solid 4-(4-(6-bromoquinazolin-4-yl)-2-fluorophenyl)morpholine (338 mg, 871 μmol, 53%). LCMS [M+H]⁺=389.

### Step D. Preparation of 4-(4-(3-fluoro-4-morpholinophenyl)quinazolin-6-yl)pyridin-2-amine (1)

A mixture of 4-(4-(6-bromoquinazolin-4-yl)-2-fluorophenyl)morpholine (115 mg. 1 eq, 296 μmol), tert-butyl (4-(4,4,5,5-tetramethyl-1,3,2-dioxaborolan-2-yl)pyridin-2-yl)carbamate (104 mg, 1.1 eq, 326 μmol), potassium phosphate (94 mg, 1.5 eq, 444 μmol), and PdCl2(dppf) (23 mg, 0.1 eq, 29.6 μmol) in 1,4-dioxane (6 mL) and Water (1 mL) solution in argon atmosphere was heated at 110° C. for 2 hrs. The reaction mixture was cooled to room temperature, diluted with ethyl acetate (30 ml), and washed with water (10 ml). The aqueous layer was washed with ethyl acetate (20 ml). The combined organic layer was washed with brine (20 ml), dried over sodium sulfate, evaporated in vacuo to obtain a brown solid. The crude product was dissolved in DCM (2 ml), loaded and in silica gel (24 g, eluent 0-10% MeOH in DCM) to afford the desired product tert-butyl (4-(4-(3-fluoro-4-morpholinophenyl)quinazolin-6-yl)pyridin-2-yl)carbamate (96 mg, 0.19 mmol, 65%), which was carried forward and 2,2,2-trifluoroacetic acid (1.5 g, 1.0 mL, 75 eq, 13 mmol) in DCM (3 mL) was stirred at rt for 16 hrs. The reaction mixture was diluted with ethyl acetate (50 ml), washed with TN NaOH (20 ml), and brine (20 ml). The organic layer was dried over sodium sulfate, evaporated in vacuo to obtain the crude product. TFA (0.24 mL):DCM (0.71 mL), (1:3 v/v), was added to the flask and stirred at 25° C. for 2 hour. When completed the reaction was diluted with DCM, basified with 1N NaOH (4.5 g NaOH in 100 mL of water), washed with H₂O, brine, and the organic layer was dried over Na2SO4. Mixture was reduced under pressure and then purified crude product was purified via column chromatography (DCM/MeOH 0-5%) to afford 4-(4-(3-fluoro-4-morpholinophenyl)quinazolin-6-yl)pyridin-2-amine with a yellow solid (46.3 mg, 63% yield, 98% purity). Mp 184-186° C. LCMS [M+H]⁺=402. ¹HNMR (500 MHz, CDCl₃) δ 9.39 (d, J=9.5 Hz, 1H), 8.39 (d, J=1.9 Hz, 1H), 8.26-8.18 (m, 1H), 8.09 (dd, J=8.8, 2.1 Hz, 1H), 7.89 (d, J=6.4 Hz, 1H), 7.57 (dt, J=5.2, 2.1 Hz, 2H), 7.13 (t, J=8.6 Hz, 1H), 7.06-6.96 (m, 3H), 3.98-3.81 (m, 4H), 3.26 (m, 4H). ¹³CNMR (126 MHz, CDCl₃) δ 166.96, 158.63, 156.20, 154.23, 151.36, 149.63, 147.36, 141.95, 137.94, 132.47, 130.67, 129.90, 126.78, 124.98, 122.94, 118.49, 118.16, 112.54, 107.05, 66.87, 50.49.

### Example 1.2: Preparation of 4-(4-(6-(1H-pyrrolo[2,3-b]pyridin-4-yl)quinazolin-4-yl)-2-fluorophenyl)morpholine (2)

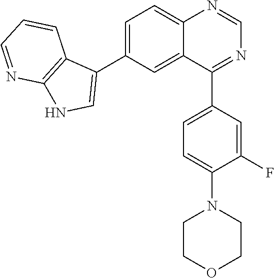 [SMILES: Fc1cc(-c2ncnc3ccc(-c4c[nH]c5ncccc45)cc23)ccc1N1CCOCC1]

### Step A. Preparation of 4-(4-(6-(1H-pyrrolo[2,3-b]pyridin-4-yl)quinazolin-4-yl)-2-fluorophenyl)morpholine (2)

From 4-(4-(6-bromoquinazolin-4-yl)-2-fluorophenyl)morpholine, the title compound was isolated in a similar manner as depicted in Example 1.1, Step D, as a yellow solid. MP=264-270° C. ¹H NMR (400 MHz, CDCl₃) δ 10.85 (s, 1H), 9.45 (s, 1H), 8.57 (s, 1H), 8.42 (s, 1H), 8.33 (d, J=1.1 Hz, 2H), 7.69-7.57 (m, 3H), 7.42 (d, J=4.8 Hz, 1H), 7.13 (1, J=8.5 Hz, 1H), 6.80 (d, J=3.3 Hz, 1H), 3.98-3.87 (m, 4H), 3.30-3.19 (m, 4H). 13C NMR (201 MHz, CDCl₃) δ 172.77, 167.13, 155.82, 155.51, 154.59, 151.58, 142.10, 137.15, 136.62, 133.56, 130.40, 130.27, 128.11, 127.31, 126.79, 123.07, 118.51, 118.18, 118.06, 115.28, 101.15, 66.86, 50.45.

### Example 1.3: Preparation of 4-(4-(3-fluoro-4-(morpholinomethyl)phenyl)quinazolin-6-yl)pyridin-2-amine (3)

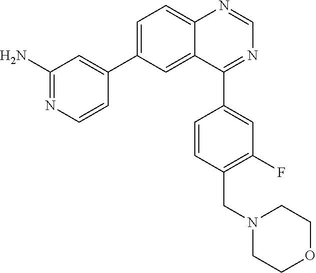 [SMILES: Nc1cc(-c2ccc3ncnc(-c4ccc(CN5CCOCC5)c(F)c4)c3c2)ccn1]

### Step A. Preparation of 4-(4-(6-bromoquinazolin-4-yl)-2-fluorobenzyl)morpholine

From 3-Fluoro-4-(4-morpholinylmethyl)phenylboronic acid pinacol ester, the title compound was isolated in a similar manner as depicted in Example 1.1, Step C, as a yellow solid. ¹H NMR (500 MHz, CDCl₃) δ 9.37 (s, 1H), 8.24-8.18 (m, 1H), 8.13 (dd, J=8.8, 2.0 Hz, 1H), 7.59 (dd, J=7.7, 1.7 Hz, 1H), 7.53 (dd, J=10.2, 1.6 Hz, 1H), 7.41-7.34 (m, 1H), 7.29 (dd, J=10.7, 1.8 Hz, 1H), 3.76 (s, 2H), 3.72 (m, 4H), 2.67-2.51 (m, 4H).

### Step B. Preparation of 4-(4-(3-fluoro-4-(morpholinomethyl)phenyl)quinazolin-6-yl)pyridin-2-amine (3)

From 4-(4-(6-bromoquinazolin-4-yl)-2-fluorobenzyl)morpholine, the title compound was isolated in a similar manner as depicted in Example 1.1, Step D, as a light brown solid. MP=110-116° C. ¹H NMR (400 MHz, CDCl₃) δ 9.42 (d, J=0.9 Hz, 1H), 8.31 (d, J=1.9 Hz, 1H), 8.26-8.17 (m, 2H), 8.16 (dd, J=8.5, 1.9 Hz, 1H), 7.69 (t, J=7.5 Hz, 1H), 7.63-7.59 (m, 1H), 7.57 (dd, J=10.2, 1.4 Hz, 1H), 6.93-6.88 (m, 1H), 6.75 (s, 1H), 4.72 (s, 2H), 3.78 (q, J=5.9, 5.2 Hz, 4H), 3.74 (s, 2H), 2.65-2.51 (m, 4H). ¹³C NMR (101 MHz, CDCl₃) δ 167.07, 162.59, 160.13, 159.00, 154.96, 151.23, 148.85, 138.60, 137.80, 132.86, 131.84, 129.91, 127.00, 125.59, 124.59, 122.96, 116.95, 112.82, 106.70, 66.94, 55.47, 53.52.

### Example 1.4: Preparation of 4-(4-(3-morpholinophenyl)quinazolin-6-yl)pyridin-2-amine (4)

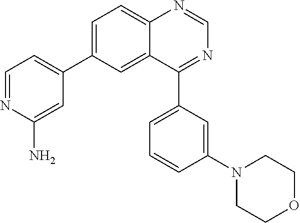 [SMILES: Nc1cc(-c2ccc3ncnc(-c4cccc(N5CCOCC5)c4)c3c2)ccn1]

### Step A. Preparation of 4-(3-bromophenyl)morpholine

From 3-morpholinoaniline, the title compound was isolated in a similar manner as depicted in Example 1.1, Step A, as a yellow solid. ¹H NMR (500 MHz, CDCl₃) δ 6.97-6.92 (m, 1H), 6.86-6.79 (m, 1H), 6.64 (ddd, J=8.4, 2.5, 0.9 Hz, OH), 6.51 (dd, J=8.9, 2.9 Hz, 1H), 3.75-3.62 (m, 4H), 3.00-2.90 (m, 4H).

### Step B. Preparation of 4-(3-(4,4,5,5-tetramethyl-1,3,2-dioxaborolan-2-yl)phenyl)morpholine

From 4-(3-Bromo-phenyl)-morpholine, the title compound was isolated in a similar manner as depicted in Example 1.1, Step B, as an orange oil. ¹H NMR (500 MHz, CDCl₃) δ 7.30-7.22 (m, 1H), 7.23-7.14 (m, 1H), 6.91 (ddd, J=8.2, 2.8, 1.1 Hz, 1H), 6.66 (s, 1H), 3.76-3.70 (m, 4H), 3.10-2.97 (m, 4H), 2.94 (s, 1H), 1.19-1.06 (m, 12H). ¹³C NMR (126 MHz, CDCl₃) δ 150.58, 129.55, 128.55, 126.66, 121.94, 118.92, 83.28, 66.80, 49.46, 24.65.

### Step C. Preparation of 4-(3-(6-bromoquinazolin-4-yl)phenyl)morpholine

From 4-(3-(4,4,5,5-tetramethyl-1,3,2-dioxaborolan-2-yl)phenyl)morpholine, the title compound was in a similar manner as depicted in Example 1.1, Step C, as a yellow solid. LCMS [M+H]⁺=372. ¹H NMR (500 MHz, CDCl₃) δ 9.36 (s, 1H), 8.28 (d, J=1.7 Hz, 1H), 7.99-7.95 (m, 2H), 7.47 (t, J=7.9 Hz, 1H), 7.28 (s, 1H), 7.20 (d, J=7.5 Hz, 1H), 7.16 (d, J=8.3 Hz, 1H), 3.92-3.84 (m, 4H), 3.30-3.22 (m, 4H).

### Step D. Preparation of 4-(4-(3-morpholinophenyl)quinazolin-6-yl)pyridin-2-amine (4)

From 4-(3-(6-bromoquinazolin-4-yl)phenyl)morpholine, the title compound was in a similar manner as depicted in Example 1.1, Step D, as a yellow/orange solid. MP=100-105° C. LCMS [M+H]⁺=384. ¹H NMR (500 MHz, CDCl₃): δ 9.37 (s, 1H), 8.31 (d, J=2.0 Hz, 1H), 8.17 (d, J=8.7 Hz, 1H), 8.14-8.07 (m, 2H), 7.46 (t, J=7.9 Hz, 1H), 7.32 (t, J=2.0 Hz, 1H), 7.21 (dt, J=7.6, 1.1 Hz, 1H), 7.12 (dd, J=8.2, 2.6 Hz, 1H), 6.87 (dd, J=5.4, 1.6 Hz, 1H), 6.70 (d, J=1.6 Hz, 1H), 4.71 (s, 2H), 3.91-3.78 (m, 4H), 3.28-3.21 (m, 4H). ¹³C NMR (126 MHz, CDCl₃): δ 169.25, 158.80, 155.81, 155.05, 151.79, 151.15, 149.18, 148.34, 137.98, 137.81, 132.53, 129.71, 129.38, 125.30, 123.30, 121.46, 117.36, 116.53, 112.68, 106.66, 66.83, 49.02.

### Example 1.5: Preparation of 4-(4-(3-fluoro-5-morpholinophenyl)quinazolin-6-yl)pyridin-2-amine (5)

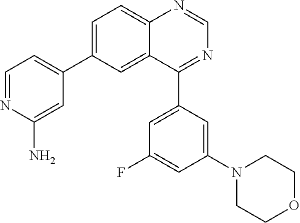 [SMILES: Nc1cc(-c2ccc3ncnc(-c4cc(F)cc(N5CCOCC5)c4)c3c2)ccn1]

### Step A. Preparation of 4-(3-fluoro-5-(4,4,5,5-tetramethyl-1,3,2-dioxaborolan-2-yl)phenyl)morpholine

From 4-(3-bromo-5-fluorophenyl)morpholine, the title compound was in a similar manner as depicted in Example 1.1, Step B, as a white solid. ¹H NMR (500 MHz, CDCl₃) δ 7.11 (s, 1H), 6.97 (dd, J=8.3, 2.3 Hz, 1H), 6.66 (dt, J=12.1, 2.5 Hz, 1H), 3.87-3.78 (m, 4H), 3.18 (dd, J=6.0, 3.8 Hz, 4H), 1.32 (s, 12H). ¹³C NMR (126 MHz, CDCl₃) δ 164.46, 162.51, 152.49, 117.04, 111.94, 105.32, 84.06, 66.75, 48.99, 24.86.

### Step B. Preparation of 4-(3-(6-bromoquinazolin-4-yl)-5-fluorophenyl)morpholine

From 4-(3-fluoro-5-(4,4,5,5-tetramethyl-1,3,2-dioxaborolan-2-yl)phenyl)morpholine, the title compound was in a similar manner as depicted in Example 1.1, Step C, as a yellow solid. LCMS [M+H]'⁰=388. ¹H NMR (500 MHz, CDCl₃) δ 9.59 (s, 1H), 8.49 (d, J=1.6 Hz, 1H), 8.22 (d, J=2.1 Hz, 2H), 7.24 (d, J=1.7 Hz, 1H), 7.11 (dt, J=8.3, 1.6 Hz, 1H), 7.04-6.99 (m, 1H), 4.13-4.06 (m, 4H), 3.52-3.46 (m, 4H). ¹³C NMR (126 MHz, CDCl₃) δ 166.87, 164.61, 162.66, 154.64, 153.10, 149.69, 138.64, 137.55, 130.72, 128.91, 124.01, 121.90, 112.04, 107.67, 103.94, 66.57, 48.58.

### Step C. Preparation of 4-(4-(3-fluoro-5-morpholinophenyl)quinazolin-6-yl)pyridin-2-amine (5)

From 4-(3-(6-bromoquinazolin-4-yl)-5-fluorophenyl)morpholine, the title compound was in a similar manner as depicted in Example 1.1, Step D, as a yellow solid. MP=130-135° C. ¹H NMR (500 MHz, CDCl₃) δ 9.55 (s, 1H), 8.45 (d, J=1.9 Hz, 1H), 8.37 (d, J=8.7 Hz, 1H), 8.34-8.29 (m, 2H), 7.25-7.22 (m, 1H), 7.10-7.07 (m, 1H), 7.05 (dd, J=5.3, 1.5 Hz, 1H), 6.96 (dt, J=11.9, 2.4 Hz, 1H), 6.88 (s, 1H), 4.88 (s, 2H), 4.03 (t, J=4.9 Hz, 4H), 3.43 (t, J=4.9 Hz, 4H). ¹³C NMR (126 MHz, CDCl₃) δ 168.11, 164.56, 162.61, 158.94, 154.90, 153.28, 153.19, 151.11, 148.92, 139.13, 139.06, 138.40, 132.82, 129.80, 124.76, 123.09, 112.65, 111.99, 111.97, 107.77, 106.61, 103.83, 66.60, 48.50.

### Example 1.6: Preparation of 4-(4-(6-(2-aminopyridin-4-yl)quinazolin-4-yl)phenyl)thiomorpholine 1,1-dioxide (6)

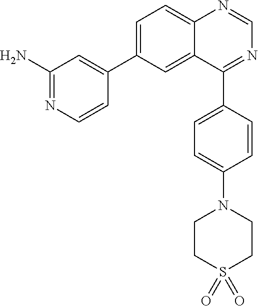 [SMILES: Nc1cc(-c2ccc3ncnc(-c4ccc(N5CCS(=O)(=O)CC5)cc4)c3c2)ccn1]

### Step A. Preparation of 4-(4-(4,4,5,5-tetramethyl-1,3,2-dioxaborolan-2 yl)phenyl)thiomorpholine 1,1-dioxide

From 4-(4-bromophenyl)thiomorpholine 1,1-dioxide, the title compound was in a similar manner as depicted in Example 1.1, Step B, as a white solid. ¹H NMR (500 MHz, CDCl₃) δ 7.73 (d, J=8.5 Hz, 2H), 6.87 (d, J=8.6 Hz, 2H), 4.00-3.76 (m, 4H), 3.18-2.98 (m, 4H), 1.31 (s, 12H), 1.24 (d, J=3.1 Hz, 8H), 1.21 (s, 4H). ¹³C NMR (126 MHz, CDCl₃) δ 149.15, 136.76, 114.50, 83.67, 83.11, 75.08, 50.35, 46.79, 24.85, 24.55.

### Step B. Preparation of 4-(4-(6-bromoquinazolin-4-yl)phenyl)thiomorpholine 1,1-dioxide

From 4-(4-(4,4,5,5-tetramethyl-1,3,2-dioxaborolan-2-yl)phenyl)thiomorpholine 1,1-dioxide, the title compound was in a similar manner as depicted in Example 1.1, Step C, as a yellow solid. ¹H NMR (500 MHz, CDCl₃) δ 9.31 (s, 1H), 8.32 (d, J=1.0 Hz, 1H), 8.02-7.92 (m, 2H), 7.85-7.74 (m, 2H), 7.08 (d, J=8.9 Hz, 2H), 4.12-3.95 (m, 4H), 3.23-3.12 (m, 4H). 13C NMR (126 MHz, CDCl₃) δ 166.70, 154.44, 149.93, 148.63, 137.23, 131.80, 130.59, 129.15, 128.08, 124.34, 121.68, 115.19, 50.63, 46.48.

### Step C. Preparation of 4-(4-(6-(2-aminopyridin-4-yl)quinazolin-4-yl)phenyl)thiomorpholine 1,1-dioxide (6)

From 4-(4-(6-bromoquinazolin-4-yl)phenyl)thiomorpholine 1,1-dioxide, the title compound was in a similar manner as depicted in Example 1.1, Step D, as a yellow solid. ¹H NMR (500 MHz, DMSO-d₆) δ 9.32 (d, J=15.8 Hz, 1H), 8.38 (dd, J=9.1, 7.3 Hz, 1H), 8.32-8.01 (m, 2H), 7.86 (d, J=8.8 Hz, 1H), 7.67 (t, J=10.5 Hz, 1H), 7.29 (d, J=8.8 Hz, 1H), 7.19 (d, J=8.8 Hz, 1H), 6.94-6.83 (m, 2H), 6.72 (dt, J=8.9, 6.9 Hz, 1H), 6.29 (s, 1H), 6.08 (d, J=16.2 Hz, 1H), 3.98-3.91 (m, 4H), 3.24-3.05 (m, 4H). ¹³C NMR (126 MHz, DMSO-d₆) δ 167.47, 166.86, 160.82, 155.24, 154.62, 149.41, 145.48, 140.28, 138.39, 133.86, 132.82, 132.21, 128.66, 122.02, 116.18, 115.37, 112.88, 110.60, 108.94, 106.01, 50.13, 46.81.

### Example 1.7: Preparation of 4-(4-(4-(4-methylpiperazin-1-yl)phenyl)quinazolin-6-yl)pyridin-2-amine (7)

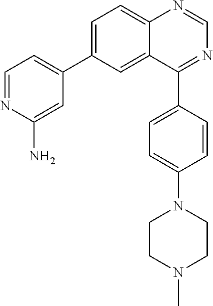 [SMILES: CN1CCN(c2ccc(-c3ncnc4ccc(-c5ccnc(N)c5)cc34)cc2)CC1]

### Step A. Preparation of 6-bromo-4-(4-(4-methylpiperazin-1-yl)phenyl)quinazoline

From 1-methyl-4-(4-(4,4,5,5-tetramethyl-1,3,2-dioxaborolan-2-yl)phenyl)piperazine, the title compound was in a similar manner as depicted in Example 1.1, Step C, as a yellow solid. ¹H NMR (500 MHz, CDCl₃): δ 6 9.46 (s, 1H), 8.52 (t, J=1.4 Hz, 1H), 8.09 (d, J=1.3 Hz, 2H), 7.92-7.88 (m, 2H), 7.26-7.21 (m, 2H), 3.59-3.54 (m, 4H), 2.81-2.76 (m, 4H), 2.55 (s, 3H).

### Step B. Preparation of 4-(4-(4-(4-methylpiperazin-1-yl)phenyl)quinazolin-6-yl)pyridin-2-amine (7)

From 6-bromo-4-(4-(4-methylpiperazin-1-yl)phenyl)quinazoline, the title compound was in a similar manner as depicted in Example 1.1, Step D, as a yellow solid. LCMS [M+H]⁺=397. ¹H NMR (500 MHz, CDCl₃) δ 9.31 (s, 1H), 8.39 (d, J=1.9 Hz, 1H), 8.14 (d, J=8.6 Hz, 2H), 8.07 (dd, J=8.8, 1.9 Hz, 1H), 7.78 (d, J=8.4 Hz, 2H), 7.08 (d, J=8.5 Hz, 2H), 6.90 (d, J=5.1 Hz, 1H), 6.72 (s, 1H), 4.73 (s, 2H), 3.42 (t, J=5.0 Hz, 4H), 2.68 (t, J=4.9 Hz, 4H), 2.42 (s, 3H). ¹³C NMR (126 MHz, CDCl₃) δ 168.16, 155.11, 152.42, 151.31, 137.68, 132.18, 131.59, 129.63, 127.32, 125.44, 123.12, 115.12, 114.99, 112.76, 110.84, 54.70, 47.70, 45.88

### Example 1.8: Preparation of 4-(4-(3-(morpholinomethyl)phenyl)quinazolin-6-yl)pyridin-2-amine (8)

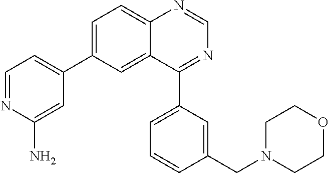 [SMILES: Nc1cc(-c2ccc3ncnc(-c4cccc(CN5CCOCC5)c4)c3c2)ccn1]

### Step A. Preparation 4-(3-(4,4,5,5-tetramethyl-1,3,2-dioxaborolan-2-yl)benzyl)morpholine

From 4-(3-bromobenzyl)morpholine, the title compound was in a similar manner as depicted in Example 1.1, Step B, as an orange oil. ¹H NMR (500 MHz, CDCl₃) δ 7.63 (dd, J=4.9, 2.6 Hz, 2H), 7.39-7.36 (m, 1H), 7.26-7.20 (m, 1H), 3.62 (q, J=6.8, 5.7 Hz, 4H), 3.44 (s, 2H), 2.38 (t, J=4.6 Hz, 4H), 1.30 (d, J=3.4 Hz, 13H), 1.25 (s, 12H). ¹³C NMR (126 MHz, CDCl₃) δ 136.16, 135.82, 133.86, 132.43, 127.72, 83.75, 66.63, 63.15, 53.32, 24.61.

### Step B. Preparation 4-(3-(6-bromoquinazolin-4-yl)benzyl)morpholine

From, 4-(3-(4,4,5,5-tetramethyl-1,3,2-dioxaborolan-2-yl)benzyl)morpholine, the title compound was in a similar manner as depicted in Example 1.1, Step C, as a brown solid. LCMS [M+H]⁺=386. ¹H NMR (500 MHz, CDCl₃) δ 9.36 (s, 1H), 8.23 (d, J=1.8 Hz, 1H), 7.96 (d, J=2.9 Hz, 2H), 7.74 (s, 1H). 7.64 (d, J=7.2 Hz, 1H). 7.59-7.50 (m, 2H), 3.73 (t, J=4.7 Hz, 4H), 3.63 (s, 2H), 2.59-2.42 (m, 4H). ¹³C NMR (126 MHz, CDCl₃) δ 167.44, 154.86, 149.81, 138.52, 137.25, 136.55, 131.22, 130.77, 130.59, 129.19, 128.91, 128.85, 124.18, 121.59, 66.90, 63.01, 53.59.

### Step C. Preparation 4-(4-(3-(morpholinomethyl)phenyl)quinazolin-6-yl)pyridin-2-amine (8)

From, 4-(3-(6-bromoquinazolin-4-yl)benzyl)morpholine, the title compound was in a similar manner as depicted in Example 1.1, Step D, as a tan solid. ¹H NMR (500 MHz, CDCl₃) δ 9.38 (s, 1H), 8.26 (d, J=2.0 Hz, 1H), 8.19 (d, J=8.7 Hz, 1H), 8.12-8.09 (m, 2H), 7.77 (d, J=1.9 Hz, 1H), 7.70-7.68 (m, 1H), 7.58-7.54 (m, 2H), 6.86 (dd, J=5.4, 1.6 Hz, 1H), 6.70 (d, J=1.5 Hz, 1H), 4.73 (s, 2H), 3.70-3.65 (m, 4H), 3.64 (s, 2H), 3.46 (s, 1H), 2.51 (t, J=4.7 Hz, 4H). ¹³C NMR (126 MHz, CDCl₃) δ 168.77, 158.88, 155.04, 151.16, 149.18, 148.42, 138.40, 138.19, 136.95, 132.63, 131.21, 130.69, 129.79, 128.93, 128.84, 125.13, 123.26, 112.63, 106.60, 66.86, 63.09, 53.54.

### Example 1.9: Preparation of 4-(4-(5-fluoro-6-(4-methylpiperazin-1-yl)pyridin-3-yl)quinazolin-6-yl)pyridin-2-amine (9)

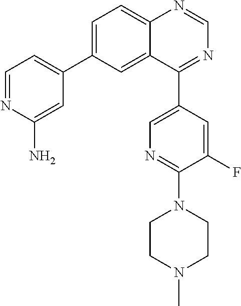 [SMILES: CN1CCN(c2ncc(-c3ncnc4ccc(-c5ccnc(N)c5)cc34)cc2F)CC1]

### Step A. Preparation 6-bromo-4-(5-fluoro-6-(4-methylpiperazin-1-yl)pyridin-3-yl)quinazoline

A mixture of 6-bromo-4-(6-chloro-5-fluoropyridin-3-yl)quinazoline (XX) (223 mg, 1 eq, 659 μmol), 1-methylpiperazine (198 mg, 0.2 mL, 3 eq, 1.98 mmol), and potassium carbonate (182 mg, 2 eq, 1.32 mmol) in THF (10 ml) was heated at 80° C. for 48 hrs. The reaction mixture was cooled to the room temperature, diluted with ethyl acetate (20 ml), washed with brine (20 ml). The organic layer was dried with sodium sulfate, filtered, and evaporated in vacuo to afford 6-bromo-4-(5-fluoro-6-(4-methylpiperazin-1-yl)pyridin-3-yl)quinazoline (175 mg, 435 μmol, 66%) without further purification. ¹H NMR (500 MHz, DMSO-d₆) δ 9.27 (s, 1H), 8.40 (d, J=1.6 Hz, 1H), 8.24 (d, J=2.0 Hz, 1H), 8.11 (dd, J=8.9, 2.1 Hz, 1H), 7.96 (d, J=8.9 Hz, 1H), 7.92 (dd, J=14.6, 1.8 Hz, 1H), 3.62-3.52 (m, 4H), 2.43-2.38 (m, 4H), 2.17 (s, 3H). ¹³C NMR (126 MHz, DMSO-d₆) δ 163.21, 155.06, 150.04, 149.78, 147.57, 144.73, 137.84, 131.40, 129.16, 125.00, 123.72, 121.45, 95.47, 55.16, 47.32, 46.27.

### Step B. Preparation 4-(4-(5-fluoro-6-(4-methylpiperazin-1-yl)pyridin-3-yl)quinazolin-6-yl)pyridin-2-amine (9)

A mixture of 6-bromo-4-(5-fluoro-6-(4-methylpiperazin-1-yl)pyridin-3-yl)quinazoline (85 mg, 1 eq, 0.21 mmol), 4-(4,4,5,5-tetramethyl-1,3,2-dioxaborolan-2-yl)pyridin-2-amine (56 mg, 1.2 eq, 0.25 mmol), potassium phosphate (67 mg, 1.5 eq, 0.32 mmol), and PdCl2(dppf) (17 mg, 0.1 eq, 21 mol) in 1,4-Dioxane (0.8 mL) and water (0.1 mL) was heated at 90° C. under argon atmosphere for 2 hrs. The reaction mixture was cooled to room temperature, and diluted with ethyl acetate (10 ml), washed with water (5 ml). The aqueous solution was washed with ethyl acetate (10 ml). The combined organic layer was washed with brine (10 ml), dried over sodium sulfate, filtered, and evaporated in vacuo to get the crude product. The crude product was purified with pre-HPLC (5-95% ACN) to afford 4-(4-(5-fluoro-6-(4-methylpiperazin-1-yl)pyridin-3-yl)quinazolin-6-yl)pyridin-2-amine (9) (38 mg, 91 μmol, 43%). ¹H NMR (500 MHz, DMSO-d₆) δ 9.25 (s, 1H), 8.47 (d, J=1.3 Hz, 1H), 8.24 (s, 1H), 8.21 (d, J=8.8 Hz, 1H), 8.10 (d, J=8.7 Hz, 1H), 7.97 (d, J=14.6 Hz, 2H), 6.82 (s, 1H), 6.73 (s, 1H), 6.02 (s, 2H), 3.59-3.54 (m, 4H), 2.42-2.36 (m, 4H), 2.17 (s, 3H). ¹³C NMR (126 MHz, DMSO-d₆) δ 164.40, 161.05, 155.08, 150.88, 149.98, 149.63, 149.25, 147.46, 144.84, 138.71, 133.33, 129.74, 125.27, 124.73, 124.40, 122.88, 110.79, 106.08, 55.17, 47.31, 46.48

### Example 1.10: 4-(4-(6-morpholinopyridin-3-yl)quinazolin-6-yl)pyridin-2-amine (10)

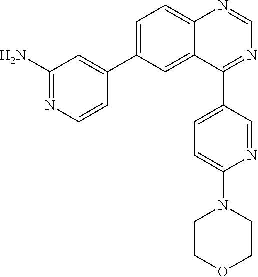 [SMILES: Nc1cc(-c2ccc3ncnc(-c4ccc(N5CCOCC5)nc4)c3c2)ccn1]

### Step A. Preparation 4-(5-(4,4,5,5-tetramethyl-1,3,2-dioxaborolan-2-yl)pyridin-2-yl)morpholine

From 4-(5-bromopyridin-2-yl)morpholine, the title compound was in a similar manner as depicted in Example 1.1, Step B, as a white solid. ¹H NMR (500 MHz, CDCl₃) δ 8.50 (d, J=0.8 Hz, 1H), 7.79 (dd, J=8.6, 1.6 Hz, 1H), 6.53 (d, J=8.6 Hz, 1H), 3.88-3.68 (m, 4H), 3.61-3.46 (m, 4H), 1.26 (s, 6H), 1.18 (s, 6H). ¹³C NMR (125 MHz, CDCl₃) δ 160.48, 154.95, 143.66, 113.75, 105.68, 83.40, 66.82, 45.20, 24.55.

### Step B. Preparation 4-(5-(6-bromoquinazolin-4-yl)pyridin-2-yl)morpholine

From 4-(5-(4,4,5,5-tetramethyl-1,3,2-dioxaborolan-2-yl)pyridin-2-yl)morpholine, the title compound was in a similar manner as depicted in Example 1.1, Step C, as a yellow solid. ¹H NMR (500 MHz, CDCl₃) δ 9.30 (s, 1H), 8.64 (d, J=2.2 Hz, 1H), 8.32 (s, 1H), 8.12-8.01 (m, 1H), 7.95 (s, 2H), 6.81 (d, J=8.9 Hz, 1H), 3.95-3.80 (m, 4H), 3.75-3.65 (m, 4H). ¹³C NMR (125 MHz, CDCl₃) δ 164.84, 159.41, 154.85, 149.85, 149.509, 139.37, 137.20, 130.73, 128.68, 123.95, 121.97, 121.53, 106.57, 66.62, 45.27.

### Step C. Preparation 4-(4-(6-morpholinopyridin-3-yl)quinazolin-6-yl)pyridin-2-amine (10)

From 4-(5-(6-bromoquinazolin-4-yl)pyridin-2-yl)morpholine, the title compound was in a similar manner as depicted in Example 1.1, Step D, as a yellow solid. ¹H NMR (500 MHz, CDCl₃) δ 9.31 (s, 1H), 8.44 (s, 1H), 8.32 (d, J=5.2 Hz, 1H), 7.97 (s, 2H), 7.79 (d, J=14.0 Hz, 2H), 3.99-3.77 (m, 6H), 3.79-3.64 (m, 6H). ¹³C NMR (126 MHz, CDCl₃) δ 163.57, 154.90, 149.92, 148.09, 144.49, 137.60, 130.58, 128.32, 124.62, 124.45, 124.25, 123.89, 121.99, 67.24, 47.47.

### Example 1.11: 4-(4-(5-fluoro-6-morpholinopyridin-3-yl)quinazolin-6-yl)pyridin-2-amine (11)

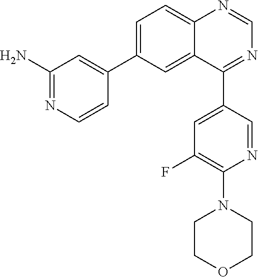 [SMILES: Nc1cc(-c2ccc3ncnc(-c4cnc(N5CCOCC5)c(F)c4)c3c2)ccn1]

### Step A. Preparation 4-(3-fluoro-5-(4,4,5,5-tetramethyl-1,3,2-dioxaborolan-2-yl)pyridin-2-yl)morpholine

From 4-(5-bromo-3-fluoropyridin-2-yl)morpholine, the title compound was in a similar manner as depicted in Example 1.1, Step B, as a white solid. ¹H NMR (500 MHz, CDCl₃) δ 8.30 (t, J=1.6 Hz, 1H), 7.50 (dt, J=20.8, 10.4 Hz, 1H), 3.90-3.69 (m, 4H), 3.67-3.48 (m, 4H), 1.29 (s, 6H), 1.23 (s, 6H). ¹³C NMR (126 MHz, CDCl₃) δ 149.45, 148.26, 128.44, 83.79, 83.53, 67.02, 47.92, 25.02, 24.86.

### Step B. Preparation 4-(5-(6-bromoquinazolin-4-yl)-3-fluoropyridin-2-yl)morpholine

From 4-(3-fluoro-5-(4,4,5,5-tetramethyl-1,3,2-dioxaborolan-2-yl)pyridin-2-yl)morpholine, the title compound was in a similar manner as depicted in Example 1.1, Step C, as a yellow solid. ¹H NMR (500 MHz, CDCl₃) δ 9.31 (s, 1H), 8.44 (s, 1H), 8.32 (d, J=5.2 Hz, 1H), 7.97 (s, 2H), 7.79 (d, J=14.0 Hz, 2H), 3.99-3.77 (m, 6H), 3.79-3.64 (m, 6H). ¹³C NMR (126 MHz, CDCl₃) δ 163.57, 154.90, 149.92, 148.09, 144.49, 137.60, 130.58, 128.32, 124.62, 124.45, 124.25, 123.89, 121.99, 67.24, 47.47.

### Step C. Preparation 4-(4-(5-fluoro-6-morpholinopyridin-3-yl)quinazolin-6-yl)pyridin-2-amine (11)

From 4-(3-fluoro-5-(4,4,5,5-tetramethyl-1,3,2-dioxaborolan-2-yl)pyridin-2-yl)morpholine, the title compound was in a similar manner as depicted in Example 1.1, Step D, as a yellow solid. ¹H NMR (500 MHz, CDCl₃) δ 9.31 (s, 1H), 8.44 (s, 1H), 8.32 (d, J=5.2 Hz, 1H), 7.97 (s, 2H), 7.79 (d, J=14.0 Hz, 2H), 3.99-3.77 (m, 6H), 3.79-3.64 (m, 6H). ¹³C NMR (126 MHz, CDCl₃) δ 163.57, 154.90, 149.92, 148.09, 144.49, 137.60, 130.58, 128.32, 124.62, 124.45, 124.25, 123.89, 121.99, 67.24, 47.47.

### Example 1.12: 4-(4-(3-fluoro-4-morpholinophenyl)pyrido[3,2-d]pyrimidin-6-yl)pyridin-2-amine (12)

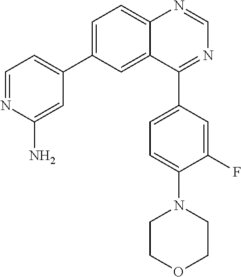 [SMILES: Nc1cc(-c2ccc3ncnc(-c4ccc(N5CCOCC5)c(F)c4)c3c2)ccn1]

### Step A. Preparation 4-(4-(6-chloropyrido[3,2-d]pyrimidin-4-yl)-2-fluorophenyl)morpholine

From 4-(2-fluoro-4-(4,4,5,5-tetramethyl-1,3,2-dioxaborolan-2-yl)phenyl)morpholine, the title compound was in a similar manner as depicted in Example 1.1, Step C, as a yellow solid. ¹H NMR (500 MHz, CDCl₃) δ 9.51 (d, J=3.1 Hz, 1H), 8.60 (dd, J=8.5, 1.9 Hz, 1H), 8.52 (dd, J=14.9, 2.0 Hz, 1H), 8.47 (d, J=8.8 Hz, 1H), 7.93 (t, J=8.5 Hz, 1H), 7.24 (t, J=8.7 Hz, 1H), 4.21-3.91 (m, 4H), 3.55-3.27 (m, 4H). ¹³C NMR (126 MHz, CDCl₃) δ 163.19, 163.05, 155.54, 151.26, 146.76, 142.75, 140.18, 138.35, 137.37, 129.94, 128.84, 119.57, 117.79, 66.84, 50.31.

### Step B. Preparation 4-(4-(3-fluoro-4-morpholinophenyl)pyrido[3,2-d]pyrimidin-6-yl)pyridin-2-amine (12)

From 4-(4-(6-chloropyrido[3,2-d]pyrimidin-4-yl)-2-fluorophenyl)morpholine, the title compound was in a similar manner as depicted in Example 1.9, Step B, as a yellow solid. ¹H NMR (500 MHz, DMSO-d₆) δ 9.31 (s, 1H), 8.49 (dd, J=8.6, 5.8 Hz, 2H), 8.45 (t, J=9.9 Hz, 1H), 8.34 (dd, J=15.5, 1.8 Hz, 1H), 8.28 (s, 1H), 8.07 (d, J=5.4 Hz, 1H), 7.23 (dd, J=11.4, 6.5 Hz, 1H), 7.17 (s, 1H), 6.15 (s, 2H), 3.81-3.65 (m, 4H), 3.21-3.13 (m, 4H). ¹³C NMR (126 MHz, DMSO-d₆) δ 163.53, 161.27, 159.15, 156.88, 155.69, 149.41, 149.11, 147.43, 146.10, 142.57, 138.61, 138.20, 129.61, 119.48, 118.65, 118.11, 110.14, 106.21, 66.75, 50.82.

### Example 1.13: 4-(4-(5-fluoro-6-morpholinopyridin-3-yl)quinolin-6-yl)pyridin-2-amine (13)

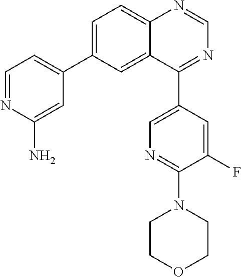 [SMILES: Nc1cc(-c2ccc3ncnc(-c4cnc(N5CCOCC5)c(F)c4)c3c2)ccn1]

### Step A. Preparation 4-(4-(5-fluoro-6-morpholinopyridin-3-yl)quinolin-6-yl)pyridin-2-amine (13)

A mixture of 4-(4-chloroquinolin-6-yl)pyridin-2-amine (XXII) (80 mg, 1 eq, 312.9 μmol), 4-(3-fluoro-5-(4,4,5,5-tetramethyl-1,3,2-dioxaborolan-2-yl)pyridin-2-yl)morpholine (96 mg, 1 eq, 312.9 μmol), potassium phosphate (100 mg, 1.5 eq, 469.3 μmol), and PdCl2(dppf) (25 mg, 0.1 eq, 31.29 μmol) in 1,4-Dioxane (0.8 mL) and water (0.1 mL) was heated at 90° C. under argon atmosphere for 2 hrs. The reaction mixture was cooled to the room temperature, and diluted with ethyl acetate (10 ml), washed with water (5 ml). The aqueous solution was washed with ethyl acetate (10 ml). The combined organic layer was washed with brine (10 ml), dried over sodium sulfate, filtered, and evaporated in vacuo to get the crude product. The crude product was purified with silica gel chromatography (0-10% MeOH in ethyl acetate) to afford 4-(4-(5-fluoro-6-morpholinopyridin-3-yl)quinolin-6-yl)pyridin-2-amine. LCMS [M+H]⁺=402. ¹HNMR (500 MHz, CDCl₃) δ 8.86 (d, J=4.4 Hz, 1H), 8.21-8.08 (m, 2H), 8.00 (dd, J=7.6, 3.7 Hz, 2H), 7.83 (dd, J=8.8, 2.0 Hz, 1H), 7.51-7.26 (m, 2H), 6.83-6.75 (m, 1H), 6.63 (s, 1H), 4.99 (d, J=36.4 Hz, 2H), 3.88-3.72 (m, 4H), 3.56 (dd, J=5.6, 3.8 Hz, 4H). ¹³CNMR (126 MHz, CDCl₃) δ 171.12, 158.99, 150.49, 149.83, 149.41, 148.71, 148.21, 147.86, 144.08, 142.93, 137.38, 130.82, 128.53, 126.55, 124.34, 123.30, 121.97, 112.64, 106.92, 66.87, 47.76.

### Example 1.14: 4-(4-(2-morpholinopyridin-4-yl)quinazolin-6-yl)pyridin-2-amine (14)

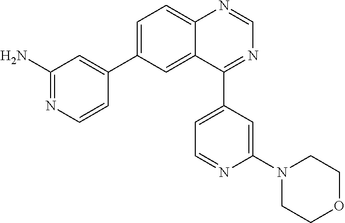 [SMILES: Nc1cc(-c2ccc3ncnc(-c4ccnc(N5CCOCC5)c4)c3c2)ccn1]

### Step A. Preparation 4-(4-(6-bromoqunazoli-4-yl)pyridi-2-yl)morpholine

From 4-(4-(4,4,5,5-tetramethyl-1,3,2-dioxaborolan-2-yl)pyridin-2-yl)morpholine, the title compound was in a similar manner as depicted in Example 1.1, Step C, as a yellow solid. ¹H NMR (500 MHz, CDCl₃) δ 9.35 (s, 1H), 8.40 (dd, J=36.6, 3.8 Hz, 1H), 8.26-8.15 (m, 1H), 7.96 (d, J=7.4 Hz, 2H), 6.89 (d, J=4.0 Hz, 2H), 3.80 (dd, J=13.0, 8.0 Hz, 4H), 3.57 (dd, J=17.2, 12.5 Hz, 4H). ³C NMR (126 MHz, CDCl₃) δ 166.15, 159.79, 154.81, 149.71, 148.37, 145.61, 137.70, 130.86, 128.55, 123.82, 122.04, 113.85, 106.96, 66.69, 45.46.

### Step B. Preparation 4-(4-(2-morpholinopyridin-4-yl)quinazolin-6-yl)pyridin-2-amine (14)

From 4-(4-(6-bromoquinazolin-4-yl)pyridin-2-yl)morpholine, the title compound was in a similar manner as depicted in Example 1.1, Step D, as a yellow solid. ¹H NMR (500 MHz, DMSO-d₆) δ 9.47 (s, 1H), 8.40 (dd, J=8.8, 2.0 Hz, 1H), 8.37 (d, J=5.0 Hz, 1H), 8.31 (d, J=1.9 Hz, 1H), 8.30 (d, J=8.8 Hz, 1H), 8.07 (d, J=6.6 Hz, 1H), 8.02 (d, J=6.5 Hz, 2H), 7.24 (dd, J=6.6, 1.6 Hz, 1H), 7.22-7.19 (m, 2H), 7.08 (dd, J=5.0, 0.8 Hz, 1H), 3.79-3.68 (m, 4H), 3.63-3.53 (m, 4H). ¹³C NMR (126 MHz, DMSO-d₆) δ 167.95, 159.81, 155.93, 155.16, 152.97, 151.33, 148.45, 145.84, 138.39, 135.88, 133.29, 130.26, 126.02, 122.64, 114.26, 111.30, 110.68, 107.89, 66.40, 45.50 Example 1.15: 4-(4-(3-fluoro-4-morpholinophenyl)quinazolin-6-yl)pyrimidin-2-amine (15)

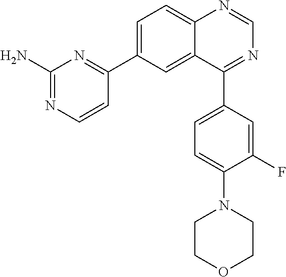 [SMILES: Nc1nccc(-c2ccc3ncnc(-c4ccc(N5CCOCC5)c(F)c4)c3c2)n1]

### Step A. Preparation 4-(2-fluoro-4-(6-(4,4,5,5-tetramethyl-1,3,2-dioxaborolan-2-yl)quinazolin-4-yl)phenyl)morpholine

A mixture of 4-(4-(6-bromoquinazolin-4-yl)-2-fluorophenyl)morpholine (250 mg, 1 eq, 644 μmol), 4,4,4′,4′,5,5,5′,5′-octamethyl-2,2′-bi(1,3,2-dioxaborolane) (278 mg, 1.7 eq, 1.09 mmol), potassium acetate (190 mg, 3 eq, 1.93 mmol), and PdCl2(dppf) (51 mg, 0.1 eq, 64.4 μmol) in 1,4-Dioxane (10 mL) was heated at 100° C. for 16 hrs. The reaction mixture was diluted with ethyl acetate, washed with brine. The organic layer was dried over sodium sulfate, filtered, evaporated in vacuo to obtain the crude product 4-(2-fluoro-4-(6-(4,4,5,5-tetramethyl-1,3,2-dioxaborolan-2-yl)quinazolin-4-yl)phenyl)morpholine (137 mg, 315 μmol, 49%). ¹H NMR (500 MHz, CDCl₃) δ 9.30 (s, 1H), 8.59 (s, 1H), 8.25 (d, J=8.3 Hz, 1H), 8.05 (d, J=8.4 Hz, 1H), 7.57 (s, 1H), 7.56-7.53 (m, 1H), 7.09 (t, J=8.5 Hz, 1H), 3.88 (dd, J=15.9, 11.2 Hz, 4H), 3.32-3.16 (m, 4H), 1.23 (s, 6H), 1.20 (s, 6H). ¹³C NMR (126 MHz, CDCl₃) δ 167.25, 156.18, 154.98, 154.21, 152.46, 141.83, 138.79, 134.59, 131.02, 130.96, 127.73, 127.02, 122.27, 118.49, 84.47, 82.85, 75.05, 66.90, 50.54, 50.51, 24.87, 24.57.

### Step B. Preparation 4-(4-(3-fluoro-4-morpholinophenyl)quinazolin-6-yl)pyrimidin-2-amine (15)

A mixture of 4-(2-fluoro-4-(6-(4,4,5,5-tetramethyl-1,3,2-dioxaborolan-2-yl)quinazolin-4-yl)phenyl)morpholine (50 mg, 1 eq, 0.11 mmol), 4-bromopyrimidin-2-amine (26 mg, 1.3 eq, 0.15 mmol), potassium triphosphate (41 mg, 1.7 eq, 0.20 mmol), and PdCl₂(dppf) (9.0 mg, 0.1 eq, 11 μmol) in 1,4-dioxane/water (6/1, 5 ml) was heated at 90° C. for 4 hrs. The reaction mixture was cooled to the room temperature, diluted with ethyl acetate, washed with brine. The organic layer was dried over sodium sulfate, filtered, and evaporated in vacuo to obtain the crude product. The crude product was further purified to get the desired product 4-(4-(3-fluoro-4-morpholinophenyl)quinazolin-6-yl)pyrimidin-2-amine (24.5 mg, 60.9 μmol, 53%). LCMS [M+H]⁺=403. ¹H NMR (500 MHz, CDCl₃) δ 9.35 (s, 1H), 8.81 (s, 1H), 8.53 (d, J=8.7 Hz, 1H), 8.38 (s, 1H), 8.18 (d, J=8.7 Hz, 1H), 7.60 (dd, J=18.6, 12.6 Hz, 2H), 7.18-7.00 (m, 2H), 5.31 (d, J=32.8 Hz, 2H), 3.91 (s, 4H), 3.28 (m, 4H). ¹³C NMR (126 MHz, CDCl₃) δ 167.35, 164.27, 162.77, 158.33, 156.21, 155.46, 154.24, 152.41, 141.95, 136.21, 131.94, 130.66, 129.66, 126.94, 126.04, 122.70, 118.45, 118.09, 107.87, 66.87, 50.49.

### Example 1.16: 4-(4-(4-morpholinophenyl)quinazolin-6-yl)pyridin-2-amine (16)

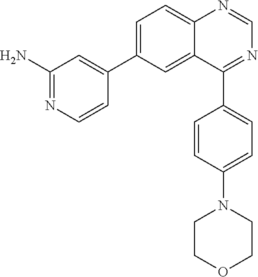 [SMILES: Nc1cc(-c2ccc3ncnc(-c4ccc(N5CCOCC5)cc4)c3c2)ccn1]

### Step A. Preparation 4-(4-(4,4,5,5-tetramethyl-1,3,2-dioxaborolan-2-yl)phenyl)morpholine

From 4-(4-bromophenyl)morpholine, the title compound was in a similar manner as depicted in Example 1.1, Step B, as a white solid. LCMS [M+H]⁺=390. ¹H NMR (500 MHz, CDCl₃) δ 7.73-7.67 (m, 2H), 6.88 (d, J=8.1 Hz, 2H), 3.84 (t, J=4.8 Hz, 4H), 3.24-3.16 (m, 4H), 1.30 (s, 12H).

### Step B. Preparation 4-(4-(6-bromoquinazolin-4-yl)phenyl)morpholine

From 4-(4-(4,4,5,5-tetramethyl-1,3,2-dioxaborolan-2-yl)phenyl)morpholine, the title compound was in a similar manner as depicted in Example 1.1, Step C, as a yellow solid. LCMS [M+H]⁺=370. ¹H NMR (500 MHz CD₂Cl₃): δ 9.47 (s, 1H), 8.52 (d, J=1.7 Hz, 1H), 8.11 (d, J=2.0 Hz, 2H), 7.96-7.88 (m, 2H), 7.26-7.20 (m, 2H), 4.11-4.03 (m, 4H), 3.49 (dd, J=6.0, 3.8 Hz, 4H). ¹³C NMR (126 MHz, CDCl₃) δ 166.85, 154.81, 152.73, 149.84, 136.90, 131.49, 130.59, 129.37, 127.09, 124.05, 121.15, 114.73, 77.32, 77.27, 77.07, 76.82, 66.69, 48.17.

### Step C. Preparation 4-(4-(4-morpholinophenyl)quinazolin-6-yl)pyridin-2-amine (16)

From 4-(4-(6-bromoquinazolin-4-yl)phenyl)morpholine, the title compound was in a similar manner as depicted in Example 1.9, Step B, as a yellow solid. MP=190-200° C. LCMS [M+H]'⁰=384. ¹H NMR (500 MHz, CDCl₃) δ 9.48 (s, 1H), 8.54 (s, 1H), 8.30 (d, J=7.6 Hz, 2H), 8.23 (d, J=8.9 Hz, 1H), 7.96 (d, J=8.4 Hz, 2H), 7.77-7.69 (m, 1H), 7.64-7.59 (m, 1H), 7.55 (d, J=7.9 Hz, 1H), 7.23 (d, J=8.3 Hz, 2H), 7.06 (d, J=5.1 Hz, 1H), 6.88 (s, 1H), 4.89 (s, 2H), 4.05 (t, J=4.8 Hz, 4H), 3.48 (t, J=4.7 Hz, 4H). ¹³C NMR (126 MHz, CDCl₃) δ 168.13, 158.94, 155.09, 152.67, 151.30, 149.28, 148.52, 137.71, 132.21, 131.58, 129.63, 127.55, 125.40, 123.10, 114.75, 112.71, 106.65, 77.29, 77.04, 76.78, 66.72, 48.20.

### Example 1.17: 4-(2-fluoro-4-(6-(2-methylpyridin-4-yl)pyrido[3,2-d]pyrimidin-4-yl)phenyl)morpholine (17)

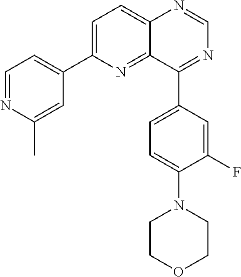 [SMILES: Cc1cc(-c2ccc3ncnc(-c4ccc(N5CCOCC5)c(F)c4)c3n2)ccn1]

### Step A. Preparation 4-(2-fluoro-4-(6-(2-methylpyridin-4-yl)pyrido[3,2-d]pyrimidin-4-yl)phenyl)morpholine (17)

From 4-(4-(6-chloropyrido[3,2-d]pyrimidin-4-yl)-2-fluorophenyl)morpholine with 2-methyl-4-(4,4,5,5-tetramethyl-1,3,2-dioxaborolan-2-yl)pyridine the title compound was in a similar manner as depicted in Example 1.9, Step B, as a yellow solid. Mp 218-219° C. LCMS [M+H]⁺=402. ¹HNMR (500 MHz, CDCl₃) δ 9.35 (d, J=2.2 Hz, 1H), 8.69 (d, J=4.7 Hz, 1H), 8.54-8.38 (m, 3H), 8.29 (d, J=8.7 Hz, 1H), 7.92 (s, 1H), 7.86 (d, J=4.3 Hz, 1H), 7.07 (td, J=8.7, 2.1 Hz, 1H), 3.90 (dd, J=5.7, 3.5 Hz, 4H), 3.27 (dd, J=5.7, 3.7 Hz, 4H), 2.72 (s, 3H). ¹³CNMR (126 MHz, CDCl₃) δ 164.25, 159.33, 155.72, 155.49, 155.31, 153.54, 149.67, 147.24, 145.93, 142.64, 138.38, 129.39, 128.76, 125.01, 121.38, 120.22, 120.03, 118.88, 117.57, 66.87, 50.35, 24.50.

### Example 1.18: 4-(4-(4-(morpholinomethyl)phenyl)quinazolin-6-yl)pyridin-2-amine (18)

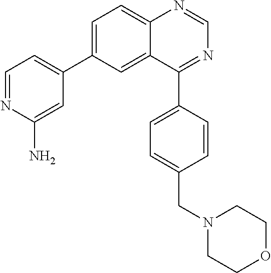 [SMILES: Nc1cc(-c2ccc3ncnc(-c4ccc(CN5CCOCC5)cc4)c3c2)ccn1]

### Step A. Preparation 4-(4-bromobenzyl)morpholine

From 4-(morpholinomethyl) aniline, the title compound was isolated in a similar manner as depicted in Example 1.1, Step A, as a cream solid. ¹H NMR (500 MHz, CDCl₃) δ 7.27-7.22 (m, 2H), 6.81-6.77 (m, 2H), 3.88-3.82 (m, 4H), 3.55 (s, 2H), 2.58 (t, J=4.7 Hz, 4H). ¹³C NMR (126 MHz, CDCl₃) δ 145.58, 130.49, 127.12, 114.91, 66.96, 53.58.

### Step B. Preparation 4-(4-(4,4,5,5-tetramethyl-1,3,2-dioxaborolan-2-yl)benzyl)morpholine

From 4-(4-bromobenzyl)morpholine, the title compound was isolated in a similar manner as depicted in Example 1.1, Step B, as a white solid. ¹H NMR (500 MHz, CDC3) δ 7.68-7.64 (m, 2H), 7.23 (d, J=7.8 Hz, 2H), 3.60 (t, J=4.7 Hz, 5H), 3.41 (s, 2H), 2.34 (d, J=9.7 Hz, 4H), 1.23 (s, 12H). ¹³C NMR (126 MHz, CDCl₃) δ 140.65, 134.78, 131.37, 130.88, 128.67, 83.61, 66.82, 63.33, 53.51, 24.89.

### Step C. Preparation 4-(4-(6-bromoquinazolin-4-yl)benzyl)morpholine

From 4-(4-(4,4,5,5-tetramethyl-1,3,2-dioxaborolan-2-yl)benzyl)morpholine, the title compound was isolated in a similar manner as depicted in Example 1.1, Step C, as a yellow solid. LCMS [M+H]⁺=383.85. ¹H NMR (500 MHz CD₂Cl₃): δ 6 9.38 (s, 1H), 8.28-8.23 (m, 1H), 8.02-7.98 (m, 1H), 7.67 (t, J=7.5 Hz, 1H), 7.60 (dd, 1=7.8, 1.7 Hz, 1H), 7.53 (dd, J=7.7, 1.7 Hz, 1H), 7.49 (dd, J=10.2, 1.6 Hz, 1H), 3.77 (t, J=4.7 Hz, 4H), 3.71 (s, 2H), 2.59 (s, 4H). ¹³C NMR δ 165.86, 162.61, 154.82, 149.93, 137.49, 130.91, 128.70, 125.55, 123.92, 121.94, 116.99, 116.76, 77.35, 77.03, 76.71, 75.01, 66.92, 55.40, 53.48, 24.87.

### Step D. Preparation 4-(4-(4-(morpholinomethyl)phenyl)quinazolin-6-yl)pyridin-2-amine (18)

From 4-(4-(6-bromoquinazolin-4-yl)benzyl)morpholine, the title compound was isolated in a similar manner as depicted in Example 1.1, Step D, as a tan solid. LCMS [M+H]⁺=398. ¹H NMR (500 MHz, CDCl₃) δ 9.33 (s, 1H), 8.36 (d, J=1.9 Hz, 1H), 8.17 (t, J=8.3 Hz, 3H), 7.96-7.92 (m, 1H), 7.57 (d, J=7.8 Hz, 2H), 7.05 (d, J=5.1 Hz, 1H), 6.91 (s, 1H), 4.94 (s, 2H), 3.75 (t, J=4.6 Hz, 4H), 3.63 (d, J=4.8 Hz, 2H), 2.53 (s, 4H). ¹³C NMR (126 MHz, CDCl₃) δ 159.06, 152.33, 148.98, 148.95, 143.75, 142.36, 141.53, 140.66, 140.63, 135.53, 129.97, 129.75, 128.36, 127.56, 127.53, 112.87, 106.61, 67.02, 63.08, 53.69.

### Example 1.19: 4-(4-(5-fluoro-6-(4-morpholinopiperidin-1-yl)pyridin-3-yl)quinazolin-6-yl)pyridin-2-amine (19)

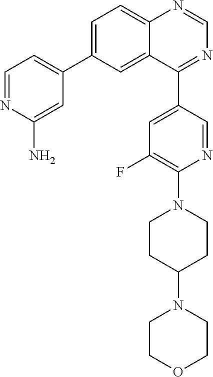 [SMILES: Nc1cc(-c2ccc3ncnc(-c4cnc(N5CCC(N6CCOCC6)CC5)c(F)c4)c3c2)ccn1]

### Step A. Preparation 4-(1-(5-(6-bromoquinazolin-4-yl)-3-fluoropyridin-2-yl)piperidin-4-yl)morpholine

From 6-bromo-4-(6-chloro-5-fluoropyridin-3-yl)quinazoline (XX), the title compound was isolated in a similar manner as depicted in Example 1.9, Step A, as a yellow solid. ¹HNMR (500 MHz, CDCl₃) δ 9.29 (s, 1H), 8.41 (t, J=1.6 Hz, 1H), 8.33 (t, J=1.3 Hz, 1H), 7.95 (d, J=1.7 Hz, 2H), 7.76 (dd, J=14.1, 2.0 Hz, 1H), 4.45 (dq, J=11.8, 2.9, 2.5 Hz, 2H), 3.77 (brs, 4H), 3.01 (td, J=12.9, 2.3 Hz, 2H), 2.65 (brs, 4H), 2.55 (brs, 1H), 2.02 (dd, J=8.8, 4.8 Hz, 2H), 1.67 (dd, J=12.6, 4.0 Hz, 2H). ¹³CNMR (126 MHz, CDCl₃) δ 163.21, 154.85, 149.98, 147.94, 144.41, 137.32, 130.86, 128.48, 128.45, 124.39, 123.86, 121.80, 62.39, 49.66, 46.66, 44.84, 34.42, 28.13.

### Step B. Preparation 4-(4-(5-fluoro-6-(4-morpholinopiperidin-1-yl)pyridin-3-yl)quinazolin-6-yl)pyridin-2-amine (19)

From 4-(1-(5-(6-bromoquinazolin-4-yl)-3-fluoropyridin-2-yl)piperidin-4-yl)morpholine, the title compound was isolated in a similar manner as depicted in Example 1.9, Step B, as a yellow solid. Mp 202-204° C. LCMS [M+H]⁺=486. ¹H NMR (500 MHz, CDCl₃) δ 9.27 (s, 1H), 8.44 (t, J=1.7 Hz, 1H), 8.31 (d, J=1.8 Hz, 1H), 8.18-8.08 (m, 2H), 8.05 (dd, J=8.7, 2.0 Hz, 1H), 7.77 (dd, J=14.1, 1.9 Hz, 1H), 6.86 (dd, J=5.2, 1.5 Hz, 1H), 6.70 (s, 1H), 4.70 (s, 2H), 4.41 (dq, J=11.9, 2.3 Hz, 2H), 3.70 (t, J=4.7 Hz, 4H), 2.98 (td, J=12.9, 2.3 Hz, 2H), 2.57 (t, J=4.7 Hz, 4H), 2.46 (ddd, J=11.2, 7.3, 3.8 Hz, 1H), 1.98-1.92 (m, 2H), 1.61 (qd, J=12.2, 3.9 Hz, 2H). ¹³C NMR (126 MHz, CDCl₃) δ 164.41, 159.12, 154.96, 151.25, 150.18, 150.04, 148.93, 147.99, 144.50, 144.46, 138.56, 132.72, 129.80, 124.47, 124.34, 124.13, 122.90, 112.75, 106.58, 67.15, 62.16, 60.37, 49.80, 46.72, 28.36.

### Example 1.20: 4-(4-(6-(2,2-dimethylmorpholino)-5-fluoropyridin-3-yl)quinazolin-6-yl)pyridin-2-amine (20)

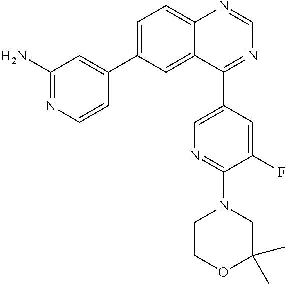 [SMILES: CC1(C)CN(c2ncc(-c3ncnc4ccc(-c5ccnc(N)c5)cc34)cc2F)CCO1]

### Step A. Preparation 4-(5-(6-bromoquinazolin-4-yl)-3-fluoropyridin-2-yl)-2,2-dimethylmorpholine

From 6-bromo-4-(6-chloro-5-fluoropyridin-3-yl)quinazoline (XX), the title compound was isolated in a similar manner as depicted in Example 1.9, Step A, as a yellow solid. LCMS [M+H]⁺=416.9. ¹HNMR (500 MHz, CDCl₃) δ 9.30 (d, J=2.7 Hz, 1H), 8.42 (s, 1H), 8.33 (s, 1H), 7.96 (d, J=2.4 Hz, 2H), 7.77 (d, J=14.1 Hz, 1H), 3.89 (p, J=2.9 Hz, 2H), 3.68 (t, J=4.4 Hz, 3H), 3.52 (s, 2H), 1.31 (d, J=2.7 Hz, 6H). ¹³CNMR (126 MHz, CDCl₃) δ 163.16, 154.87, 150.49, 144.43, 137.36, 130.90, 128.42, 124.49, 124.32, 123.86, 121.83, 71.50, 60.92, 56.48, 56.43, 46.97, 46.92, 24.42.

### Step B. Preparation 4-(4-(6-(2,2-dimethylmorpholino)-5-fluoropyridin-3-yl)quinazolin-6-yl)pyridin-2-amine (20)

From 4-(5-(6-bromoquinazolin-4-yl)-3-fluoropyridin-2-yl)-2,2-dimethylmorpholine, the title compound was isolated in a similar manner as depicted in Example 1.9, Step B, as a yellow solid. MP 175-177° C. ¹HNMR (500 MHz, CDCl₃) δ 9.58-9.13 (m, 1H), 8.46 (s, 1H), 8.34 (s, 1H), 8.26-8.01 (m, 3H), 7.82 (d, J=14.1 Hz, 1H), 6.88 (d, J=44.4 Hz, 2H), 5.19 (d, J=78.6 Hz, 2H), 3.95-3.84 (m, 2H), 3.68 (d, J=4.8 Hz, 2H), 3.51 (s, 2H), 1.31 (d, J=2.7 Hz, 6H). ¹³CNMR (126 MHz, CDCl₃) δ 164.46, 155.15, 151.43, 150.52, 149.96, 149.77, 147.91, 144.52, 138.09, 132.67, 130.04, 124.57, 124.45, 124.25, 122.92, 71.49, 60.92, 56.53, 56.47, 47.01, 46.97, 24.89, 24.42.

### Example 1.21: 4-(4-(4-methoxy-3-methylphenyl)quinazolin-6-yl)pyridin-2-amine (21)

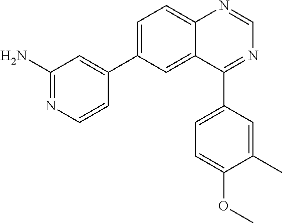 [SMILES: COc1ccc(-c2ncnc3ccc(-c4ccnc(N)c4)cc23)cc1C]

### Step A. Preparation 2-(4-methoxy-3-methylphenyl)-4,4,5,5-tetramethyl-1,3,2-dioxaborolane

From 4-bromo-1-methoxy-2-methylbenzene, the title compound was isolated in a similar manner as depicted in Example 1.1, Step B, as a brown oil. LCMS [M+H]⁺=249. ¹H NMR (500 MHz, CDCl₃) δ 7.53 (d, J=8.2 Hz, 1H), 7.47 (s, 1H), 6.69 (d, J=8.0 Hz, 1H), 3.71 (d, J=2.6 Hz, 3H), 2.09 (s, 3H), 1.21 (s, 12H). ¹³C NMR (126 MHz, CDCl₃) δ 160.44, 137.15, 134.34, 125.88, 109.24, 83.50, 55.17, 24.89, 16.03.

### Step B. Preparation 6-bromo-4-(4-methoxy-3-methylphenyl)quinazoline

From 2-(4-methoxy-3-methylphenyl)-4,4,5,5-tetramethyl-1,3,2-dioxaborolane, the title compound was isolated in a similar manner as depicted in Example 1.1, Step C, as a white solid. LCMS [M+H]⁺=328. ¹H NMR (500 MHz, CDCl₃) δ 9.55 (s, 1H), 8.56 (s, 1H), 8.18 (s, 2H), 7.85-7.78 (m, 2H), 7.26-7.21 (m, 1H), 4.16 (d, J=2.3 Hz, 3H), 2.55 (s, 3H). ¹³C NMR (126 MHz, CDCl₃) δ 167.32, 159.88, 154.74, 149.76, 137.04, 132.18, 130.58, 129.45, 129.31, 128.40, 127.68, 124.19, 121.30, 109.87, 55.57, 16.38.

### Step C. Preparation 4-(4-(4-methoxy-3-methylphenyl)quinazolin-6-yl)pyridin-2-amine (21)

From 6-bromo-4-(4-methoxy-3-methylphenyl)quinazoline, the title compound was isolated in a similar manner as depicted in Example 1.9, Step B, as a gray solid. LCMS [M+H]⁺=342. ¹H NMR (500 MHz, DMSO-d₆) δ 9.33 (s, 1H), 8.35-8.26 (m, 2H), 8.18 (d, J=8.7 Hz, 1H), 8.03 (d, J=5.3 Hz, 1H), 7.73 (d, J=8.2 Hz, 2H), 7.21 (d, J=8.2 Hz, 1H), 6.86 (dd, J=5.4, 1.7 Hz, 1H), 6.74 (d, J=1.7 Hz, 1H), 6.09 (s, 2H), 3.93 (s, 3H), 2.30 (s, 3H). ¹³C NMR (126 MHz, DMSO-d₆) δ 159.9, 159.88, 154.74, 151.98, 149.76, 148.50, 137.04, 132.18, 130.58, 129.45, 129.31, 128.40, 127.68, 124.19, 121.30, 109.87, 107.52, 106.89, 56.05, 16.53.

### Example 1.22: 1-(4-(4-(6-(2-aminopyridin-4-yl)quinazolin-4-yl)phenyl)piperazin-1-yl)ethan-1-one (22)

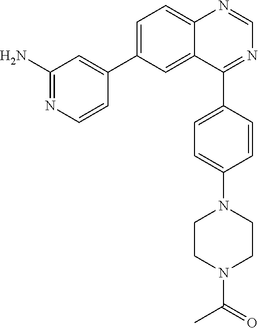 [SMILES: CC(=O)N1CCN(c2ccc(-c3ncnc4ccc(-c5ccnc(N)c5)cc34)cc2)CC1]

### Step A. Preparation 1-(4-(4-bromophenyl)piperazin-1-yl)ethan-1-one

1-(4-Bromophenyl)piperazine (0.50 g, 1 eq, 2.07 mmol) and triethylamine (0.56 g, 0.77 mL, 2.7 eq, 5.5 mmol) were added to DCM (7.2 mL). The reaction was cooled to 0 C and then Acetyl chloride (0.33 g, 0.29 mL, 2.0 eq, 4.2 mmol) was added slowly. Once completely added, the reaction was warmed to 25° C. and stirred for 16 h. Upon completion, the reaction was quenched with brine solution and diluted with DCM. Org. phase was washed additionally with brine and water, and then dried using sodium sulfate. The mixture was reduced under pressure and purified through column chromatography (EtOAc/Hex). 1-(4-(4-bromophenyl)piperazin-1-yl)ethan-1-one (405 mg, 1.43 mmol, 69%) was isolated as a white solid. LCMS [M+H]⁺=282. ¹H NMR (500 MHz, CDCl₃) δ 7.27-7.21 (m, 2H), 6.71-6.66 (m, 2H), 3.68-3.64 (m, 2H), 3.54-3.48 (m, 2H), 3.07-2.98 (m, 4H), 2.03 (s, 3H). ¹³C NMR (126 MHz, CDCl₃) δ 168.98, 149.90, 132.04, 118.24, 112.79, 49.54, 49.20, 46.05, 41.16, 21.37.

### Step B. Preparation 1-(4-(4-(4,4,5,5-tetramethyl-1,3,2-dioxaborolan-2-yl)phenyl)piperazin-1-yl)ethan-1-one

From 1-(4-(4-bromophenyl)piperazin-1-yl)ethan-1-one, the title compound was isolated in a similar manner as depicted in Example 1.1, Step B, as a tan solid. LCMS [M+H]⁺=331. ¹H NMR (500 MHz, CDCl₃) δ 7.71 (d, J=7.9 Hz, 2H), 6.89 (d, J=8.0 Hz, 2H), 3.76 (t, J=5.1 Hz, 2H), 3.63-3.58 (m, 2H), 3.26 (t, J=5.0 Hz, 2H), 3.23 (d, J=5.2 Hz, 2H), 2.12 (s, 3H), 1.31 (s, 12H). ¹³C NMR (126 MHz, CDCl₃) δ 169.11, 152.82, 136.24, 122.18, 114.94, 83.52, 45.99, 41.12, 24.86, 21.41.

### Step C. Preparation 6-bromo-4-(4-(4-(prop-1-en-2-yl)piperazin-1-yl)phenyl)quinazoline

From 1-(4-(4-(4,4,5,5-tetramethyl-1,3,2-dioxaborolan-2-yl)phenyl)piperazin-1-yl)ethan-1-one, the title compound was isolated in a similar manner as depicted in Example 1.1, Step C, as a yellow solid. LCMS [M+H]⁺=410. ¹H NMR (500 MHz, CDCl₃) δ 9.47 (s, 1H), 8.50 (s, 1H), 8.10 (s, 2H), 7.91 (d, J=8.3 Hz, 2H), 7.23 (d, J=8.3 Hz, 2H), 3.98 (t, J=5.0 Hz, 2H), 3.83 (t, J=4.9 Hz, 2H), 3.52 (dt, J=17.9, 5.0 Hz, 4H), 2.33 (s, 3H). ¹³C NMR (126 MHz, CDCl₃) δ 169.12, 166.80, 154.82, 152.25, 149.86, 136.97, 131.53, 130.64, 129.31, 127.43, 124.05, 121.23, 115.45, 48.20, 45.93, 41.09, 21.24.

### Step D. 1-(4-(4-(6-(2-aminopyridin-4-yl)quinazolin-4-yl)phenyl)piperazin-1-yl)ethan-1-one (22)

From 1-(4-(4-(6-bromoquinazolin-4-yl)phenyl)piperazin-1-yl)ethan-1-one, the title compound was isolated in a similar manner as depicted in Example 1.9, Step B, as a yellow solid. MP=120-130° C. LCMS [M+H]⁺=425. ¹H NMR (500 MHz, CDCl₃) δ 9.48 (d, J=4.6 Hz, 1H), 8.53 (s, 1H), 8.37-8.22 (m, 3H), 7.97 (d, J=8.0 Hz, 2H), 7.25 (d, J=8.1 Hz, 2H), 7.06 (d, J=5.6 Hz, 1H), 6.88 (s, 1H), 4.83 (s, 2H), 3.97 (d, J=6.5 Hz, 2H), 3.88-3.81 (m, 2H), 3.57-3.46 (m, 4H), 2.32 (s, 3H). ¹³C NMR (126 MHz, CDCl₃) δ 169.09, 168.01, 159.04, 155.06, 152.19, 151.28, 149.09, 148.86, 137.90, 132.29, 131.61, 129.64, 127.93, 125.27, 123.09, 115.45, 112.73, 106.49, 48.24, 45.95, 41.10, 21.37.

### Example 1.23: 4-(4-(5-fluoro-6-(2-oxa-8-azaspiro[4.5]decan-8-yl)pyridin-3-yl)quinazolin-6-yl)pyridin-2-amine hydrochloride (23)

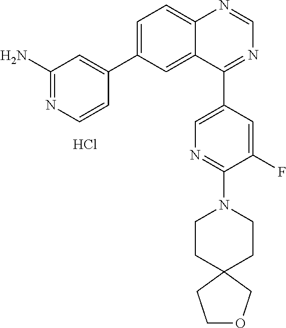 [SMILES: Nc1cc(-c2ccc3ncnc(-c4cnc(N5CCC6(CCOC6)CC5)c(F)c4)c3c2)ccn1 ; Cl]

### Step A. Preparation 8-(5-(6-bromoquinazolin-4-yl)-3-fluoropyridin-2-yl)-2-oxa-8-azaspiro[4.5]decane

From 6-bromo-4-(6-chloro-5-fluoropyridin-3-yl)quinazoline (XX), the title compound was isolated in a similar manner as depicted in Example 1.9, Step A, as a yellow solid. LCMS [M+H]⁺=416.9.

### Step B. Preparation 4-(4-(5-fluoro-6-(2-oxa-8-azaspiro[4.5]decan-8-yl)pyridin-3-yl)quinazolin-6-yl)pyridin-2-amine hydrochloride (23)

A mixture of 8-(5-(6-bromoquinazolin-4-yl)-3-fluoropyridin-2-yl)-2-oxa-8-azaspiro[4.5]decane (90 mg, 1 eq, 0.20 mmol), 4-(4,4,5,5-tetramethyl-1,3,2-dioxaborolan-2-yl)pyridin-2-amine (54 mg, 1.2 eq, 0.24 mmol), potassium phosphate (65 mg, 1.5 eq, 0.30 mmol), and PdCl₂(dppf) (16 mg, 0.1 eq, 20 μmol) in 1,4-Dioxane (0.8 mL) and water (0.1 mL) was heated at 90° C. under argon atmosphere for 2 hrs. The reaction mixture was cooled to the room temperature, and diluted with ethyl acetate, washed with water. The aqueous solution was washed with ethyl acetate. The combined organic layer was washed with brine, dried over sodium sulfate, filtered, and evaporated in vacuo to get the crude product. The crude product was purified with silica gel flash column (eluent 0-10% MeOH in ethyl acetate) to collect free base product, followed by dissolving in DCM (3 ml) and purged with HCl gas to afford 4-(4-(5-fluoro-6-(2-oxa-8-azaspiro[4.5]decan-8-yl)pyridin-3-yl)quinazolin-6-yl)pyridin-2-amine hydrochloride (23) (56 mg, 56%). LCMS [M+H]⁺=457. ¹HNMR (500 MHz, DMSO-d₆) δ 14.16 (s, 1H), 9.38 (s, 1H), 8.56 (s, 1H), 8.47 (d, J=1.9 Hz, 1H), 8.36 (dd, J=8.8, 1.9 Hz, 1H), 8.29 (s, 1H), 8.24 (d, J=8.8 Hz, 1H), 8.14-8.02 (m, 2H), 7.36 (s, 1H), 7.33 (d, J=6.7 Hz, 1H), 5.74 (s, 1H), 3.78 (t, J=7.0 Hz, 2H), 3.71 (dt, J=13.6, 5.2 Hz, 2H), 3.66-3.56 (m, 2H), 3.52 (s, 2H), 1.79 (t, J=7.1 Hz, 2H), 1.65 (t, J=5.6 Hz, 4H). ¹³CNMR (126 MHz, DMSO-d₆) δ 164.95, 155.61, 154.77, 153.27, 151.37, 147.47, 145.11, 136.85, 135.71, 133.13, 129.90, 126.46, 125.32, 123.81, 122.58, 114.99, 111.47, 111.18, 77.48, 66.93, 45.44, 42.11, 37.19, 34.89.

### Example 1.24: 4-(4-(5-fluoro-6-(piperazin-1-yl)pyridin-3-yl)quinazolin-6-yl)pyridin-2-amine hydrochloride (24)

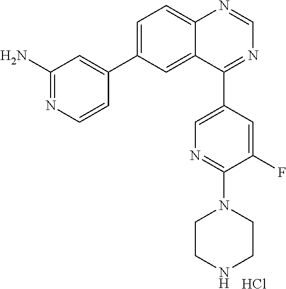 [SMILES: Nc1cc(-c2ccc3ncnc(-c4cnc(N5CCNCC5)c(F)c4)c3c2)ccn1 ; Cl]

### Step A. Preparation 6-bromo-4-(5-fluoro-6-(piperazin-1-yl)pyridin-3-yl)quinazoline hydrochloride

From 6-bromo-4-(6-chloro-5-fluoropyridin-3-yl)quinazoline (XX), the title compound was isolated in a similar manner as depicted in Example 1.9, Step A. followed by dissolving in DCM (3 ml) and purged with HCl gas. LCMS [M−HCl+1]⁺ 388. ¹H NMR (500 MHz, DMSO-d₆) δ 9.54 (s, 2H), 9.37 (s, 1H), 8.57-8.46 (m, 1H), 8.29 (d, J=2.1 Hz, 1H), 8.19 (dd, J=9.0, 2.2 Hz, 1H), 8.11-8.02 (m, 2H), 3.87 (t, J=5.1 Hz, 4H), 3.25 (t, J=4.9 Hz, 4H). ¹³C NMR (126 MHz, DMSO-d₆) δ 163.35, 155.08, 149.62, 147.80, 144.58, 138.04, 131.11, 128.93, 125.59, 125.42, 123.97, 121.78, 49.04, 44.25, 42.83.

### Step B. Preparation 4-(4-(5-fluoro-6-(piperazin-1-yl)pyridin-3-yl)quinazolin-6-yl)pyridin-2-amine hydrochloride (24)

From 6-bromo-4-(5-fluoro-6-(piperazin-1-yl)pyridin-3-yl)quinazoline hydrochloride, the title compound was isolated in a similar manner as depicted in Example 1.9, Step B, as an orange solid. LCMS [M−HCl+1]⁺ 402. ¹HNMR (500 MHz, d₄-MeOH) δ 9.35 (s, 1H), 8.60 (t, J=1.5 Hz, 1H), 8.51 (d, J=1.9 Hz, 1H), 8.38 (dd, J=8.8, 2.0 Hz, 1H), 8.25 (d, J=8.8 Hz, 1H), 8.07 (dd, J=13.7, 1.9 Hz, 1H), 8.00 (d, J=6.4 Hz, 1H), 7.31-7.26 (m, 1H), 7.23 (dd, J=6.4, 1.7 Hz, 1H), 4.00 (t, J=5.1 Hz, 4H), 3.45 (t, J=5.2 Hz, 4H). ¹³CNMR (126 MHz, d₄-MeOH) δ 165.04, 156.03, 154.88, 152.83, 151.30, 150.18, 149.20, 148.13, 144.34, 138.84, 136.60, 132.74, 129.32, 125.98, 125.38, 125.05, 124.88, 122.73, 111.30, 109.77, 44.05, 43.11.

### Example 1.25: 4-(4-(3-fluoro-4-(4-methylpiperazin-1-yl)phenyl)quinazolin-6-yl)pyridin-2-amine (25)

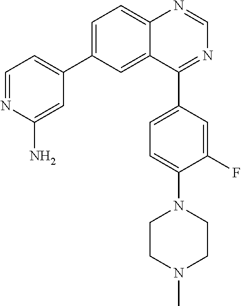 [SMILES: CN1CCN(c2ccc(-c3ncnc4ccc(-c5ccnc(N)c5)cc34)cc2F)CC1]

### Step A. Preparation 1-(4-bromo-2-fluorophenyl)-4-methylpiperazine

From 3-fluoro-4-(4-methylpiperazin-1-yl) aniline, the title compound was isolated in a similar manner as depicted in Example 1.1, Step A, as a brown solid. LCMS [M+H]⁺=274. ¹H NMR (500 MHz, CDCl₃) δ 7.24 (s, 2H), 6.88 (t, J=9.0 Hz, 1H), 3.17 (t, J=5.1 Hz, 4H), 2.71-2.64 (m, 4H), 2.43 (s, 3H). ¹³C NMR (126 MHz, CDCl₃) δ 156.32, 154.33, 139.41, 127.41, 120.00, 119.51, 113.45, 55.01, 50.28, 46.07 Step B. Preparation 1-(2-fluoro-4-(4,4,5,5-tetramethyl-1,3,2-dioxaborolan-2-yl)phenyl)-4-methylpiperazine.

From 1-(4-bromo-2-fluorophenyl)-4-methylpiperazine, the title compound was isolated in a similar manner as depicted in Example 1.1, Step B, as a brown solid. LCMS [M+H]⁺=321. ¹H NMR (500 MHz, CDCl₃) δ 7.26 (dd, J=33.9, 10.7 Hz, 2H), 6.97 (d, J=10.0 Hz, 1H), 6.71 (t, J=8.2 Hz, 1H), 2.94 (dt, J=46.3, 4.8 Hz, 4H), 2.44-2.36 (m, 4H), 2.16 (s, 3H), 1.12 (s, 12H). ¹³C NMR (126 MHz, CDCl₃) δ 156.13, 154.15, 131.36, 127.45, 121.83, 118.01, 83.74, 55.03, 50.05, 46.05, 24.75.

### Step C. Preparation 6-bromo-4-(3-fluoro-4-(4-methylpiperazin-1-yl)phenyl)quinazoline

From 1-(2-fluoro-4-(4,4,5,5-tetramethyl-1,3,2-dioxaborolan-2-yl)phenyl)-4-methylpiperazine, the title compound was isolated in a similar manner as depicted in Example 1.1, Step C, as a tan solid. LCMS [M+H]⁺=402. ¹H NMR (500 MHz, CDCl₃) δ 9.31 (s, 1H), 8.30 (d, J=1.4 Hz, 1H), 7.95 (d, J=1.3 Hz, 2H), 7.54-7.48 (m, 2H), 7.10 (t, J=8.6 Hz, 1H), 3.31 (t, J=4.7 Hz, 3H), 2.76-2.68 (m, 4H), 2.42 (s, 3H). ¹³C NMR (126 MHz, CDCl₃) δ 165.69, 154.82, 149.98, 137.17, 130.81, 128.92, 126.66, 126.64, 123.92, 121.57, 118.84, 118.81, 117.98, 117.80, 54.97, 54.89, 49.72, 45.87.

### Step D. Preparation 4-(4-(3-fluoro-4-(4-methylpiperazin-1-yl)phenyl)quinazolin-6-yl)pyridin-2-amine (25)

From 6-bromo-4-(3-fluoro-4-(4-methylpiperazin-1-yl)phenyl)quinazoline, the title compound was isolated in a similar manner as depicted in Example 1.9, Step B, as a tan solid. LCMS [M+H]⁺=415. ¹H NMR (500 MHz, CDCl₃) δ 9.37 (d, J=10.1 Hz, 1H), 8.33 (d, J=1.9 Hz, 1H), 8.22-8.11 (m, 3H), 7.64-7.56 (m, 2H), 7.15 (t, J=8.6 Hz, 1H), 6.91 (d, J=5.0 Hz, 1H), 6.74 (s, 1H), 4.69 (s, 1H), 3.47 (dt, J=12.9, 6.4 Hz, 4H), 2.92 (s, 4H), 2.58 (s, 3H). ¹³C NMR (126 MHz, CDCl₃) δ 166.87, 158.97, 156.19, 154.98, 154.22, 151.32, 148.94, 141.60, 138.31, 132.55, 129.82, 126.75, 124.81, 122.96, 118.92, 118.02, 112.77, 106.51, 54.81, 49.46, 45.64.

### Example 1.26: 4-(5-fluoro-6-(4-methylpiperazin-1-yl)pyridin-3-yl)-6-(1H-pyrrolo[2,3-b]pyridin-3-yl)quinazoline (26)

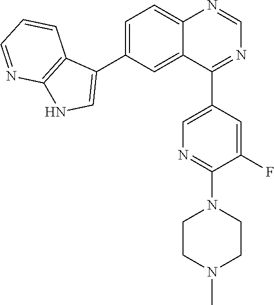 [SMILES: CN1CCN(c2ncc(-c3ncnc4ccc(-c5c[nH]c6ncccc56)cc34)cc2F)CC1]

### Step A. Preparation 4-(5-fluoro-6-(4-methylpiperazin-1-yl)pyridin-3-yl)-6-(4,4,5,5-tetramethyl-1,3,2-dioxaborolan-2-yl)quinazoline

From 6-bromo-4-(5-fluoro-6-(4-methylpiperazin-1-yl)pyridin-3-yl)quinazoline, the title compound was isolated in a similar manner as depicted in Example 1.15, Step A. LCMS [M+H]⁺=450.

### Step B. Preparation 4-(5-fluoro-6-(4-methylpiperazin-1-yl)pyridin-3-yl)-6-(1H-pyrrolo[2,3-b]pyridin-3-yl)quinazoline (26)

From 6-bromo-4-(5-fluoro-6-(4-methylpiperazin-1-yl)pyridin-3-yl)quinazoline, the title compound was isolated in a similar manner as depicted in Example 1.15, Step B. MP 263-265 C. LCMS [M+H]⁺=443.

### Example 1.27: 4-(5-fluoro-6-(piperazin-1-yl)pyridin-3-yl)-6-(1H-pyrrolo[2,3-b]pyridin-3-yl)quinazoline (27)

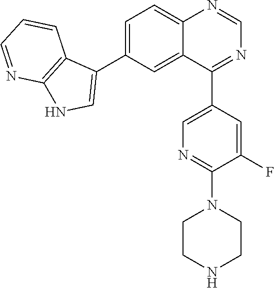 [SMILES: Fc1cc(-c2ncnc3ccc(-c4c[nH]c5ncccc45)cc23)cnc1N1CCNCC1]

### Step A. Preparation 4-(5-fluoro-6-(piperazin-1-yl)pyridin-3-yl)-6-(1H-pyrrolo[2,3-b]pyridin-3-yl)quinazoline (27)

From 6-bromo-4-(5-fluoro-6-(piperazin-1-yl)pyridin-3-yl)quinazoline hydrochloride and tert-butyl 3-(4,4,5,5-tetramethyl-1,3,2-dioxaborolan-2-yl)-1H-pyrrolo[2,3-b]pyridine-1-carboxylate, the title compound was isolated in a similar manner as depicted in Example 1.1, Step D. LCMS [M+H]⁺=425. ¹H NMR (400 MHz, DMSO-d₆) δ 12.19 (s, 1H), 9.28 (s, 1H), 8.64 (t, J=1.6 Hz, 1H), 8.46 (dd, J=8.8, 2.0 Hz, 1H), 8.38-8.31 (m, 2H), 8.28 (dd, J=8.0, 1.5 Hz, 1H), 8.24-8.08 (m, 3H), 7.20 (dd, J=8.0, 4.6 Hz, 1H), 3.80 (t, J=4.9 Hz, 4H), 3.23 (d, J=5.1 Hz, 4H). ¹³C NMR (101 MHz, DMSO-d₆) δ 163.18, 154.07, 149.69, 149.64, 144.57, 143.81, 135.65, 134.08, 129.41, 127.57, 126.26, 123.41, 121.62, 117.56, 116.90, 113.52, 45.23, 43.54.

### Example 1.28: 8-(5-(6-(2-aminopyridin-4-yl)quinazolin-4-yl)-3-fluoropyridin-2-yl)-2,8-diazaspiro[4.5]decan-1-one (30)

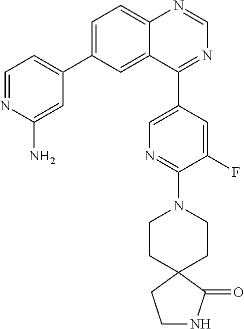 [SMILES: Nc1cc(-c2ccc3ncnc(-c4cnc(N5CCC6(CCNC6=O)CC5)c(F)c4)c3c2)ccn1]

### Step A. Preparation 8-(5-(6-bromoquinazolin-4-yl)-3-fluoropyridin-2-yl)-2,8-diazaspiro[4.5]decan-1-one

From 6-bromo-4-(6-chloro-5-fluoropyridin-3-yl)quinazoline (XX), the title compound was isolated in a similar manner as depicted in Example 1.9, Step A, as a yellow solid. LCMS [M+H]⁺=457. ¹H NMR (500 MHz, DMSO-d₆) δ 9.33 (s, 1H), 8.47 (t, J=1.8 Hz, 1H), 8.32 (d, J=2.2 Hz, 1H), 8.17 (dd, J=8.9, 2.2 Hz, 1H), 8.02 (d, J=9.0 Hz, 1H), 7.98 (dd, J=14.6, 1.9 Hz, 1H), 7.63 (s, 1H), 4.19 (dt, J=13.5, 4.0 Hz, 2H), 3.29-3.21 (m, 4H), 2.07 (t, J=6.8 Hz, 2H), 1.81 (ddd, J=13.2, 11.5, 4.1 Hz, 2H), 1.49 (dt, J=13.6, 3.2 Hz, 2H). ¹³C NMR (126 MHz, DMSO-d₆) δ 180.25, 163.29, 155.11, 149.97, 149.61, 147.39, 144.84, 137.83, 131.13, 128.97, 125.07, 124.90, 123.72, 121.59, 44.22, 42.30, 38.41, 32.01, 31.71.

### Step B. Preparation 8-(5-(6-(2-aminopyridin-4-yl)quinazolin-4-yl)-3-fluoropyridin-2-yl)-2,8-diazaspiro[4.5]decan-1-one (30)

From 8-(5-(6-bromoquinazolin-4-yl)-3-fluoropyridin-2-yl)-2,8-diazaspiro[4.5]decan-1-one, the title compound was isolated in a similar manner as depicted in Example 1.9, Step B, as a yellow oil. LCMS [M+H]⁺=470. ¹H NMR (500 MHz, DMSO-d₆) δ 9.32 (s, 1H), 8.55 (t, J=1.7 Hz, 1H), 8.33 (d, J=1.9 Hz, 1H), 8.28 (dd, J=8.8, 1.9 Hz, 1H), 8.16 (d, J=8.7 Hz, 1H), 8.08-7.93 (m, 2H), 7.64 (s, 1H), 6.89 (dd, J=5.3, 1.6 Hz, 1H), 6.79 (d, J=1.5 Hz, 1H), 6.11 (s, 2H), 4.20 (dt, J=13.5, 4.1 Hz, 2H), 3.29-3.20 (m, 4H), 2.07 (t, J=6.8 Hz, 2H), 1.86-1.76 (m, 2H), 1.57-1.44 (m, 2H). ¹³C NMR (126 MHz, DMSO-d₆) δ 180.28, 164.32, 160.93, 155.07, 150.94, 149.97, 149.23, 147.49, 147.47, 144.96, 138.66, 133.29, 129.72, 125.19, 124.31, 122.79, 110.77, 106.00, 44.22, 44.17, 42.30, 38.42, 32.04, 31.70.

### Example 1.29: 4-(4-(3-fluoro-4-morpholinophenyl)quinolin-6-yl)pyridin-2-amine (32)

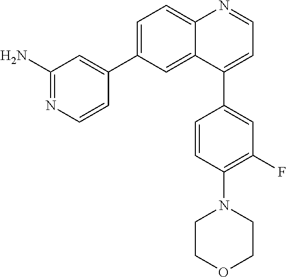 [SMILES: Nc1cc(-c2ccc3nccc(-c4ccc(N5CCOCC5)c(F)c4)c3c2)ccn1]

### Step A. Preparation 4-(4-(3-fluoro-4-morpholinophenyl)quinolin-6-yl)pyridin-2-amine (32)

From 4-(4-chloroquinolin-6-yl)pyridin-2-amine (XXIII), the title compound was isolated in a similar manner as depicted in Example 1.13, Step A, as a gray solid. LCMS [M+H]⁺=400. ¹H NMR (500 MHz, DMSO-d₆) δ 8.95 (d, J=4.4 Hz, 1H), 8.20 (d, J=8.7 Hz, 1H), 8.12 (d, J=2.0 Hz, 1H), 8.07-8.03 (m, 1H), 8.00 (d, J=5.3 Hz, 1H), 7.50 (d, J=4.5 Hz, 2H), 7.40 (dd, J=8.3, 2.0 Hz, 1H), 7.24 (t, J=8.7 Hz, 1H), 6.82-6.79 (m, 1H), 6.70 (d, J=2.0 Hz, 1H), 6.05 (s, 2H), 3.82-3.78 (m, 5H), 3.15 (dd, J=4.2, 2.2 Hz, 4H). ¹³C NMR (126 MHz, DMSO-d₆) δ 159.83, 153.99, 150.00, 148.11, 147.55, 147.08, 145.67, 139.31, 136.30, 130.28, 129.82, 127.52, 125.63, 125.21, 122.21, 121.51, 118.56, 116.60, 109.63, 104.73, 65.56, 49.64.

### Example 1.30: 1-(4-(4-(6-(1H-pyrazol-4-yl)quinazolin-4-yl)phenyl)piperazin-1-yl)ethan-1-one (37)

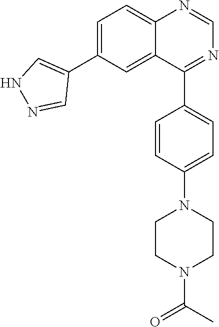 [SMILES: CC(=O)N1CCN(c2ccc(-c3ncnc4ccc(-c5cn[nH]c5)cc34)cc2)CC1]

### Step A. Preparation 1-(4-(4-(6-(1H-pyrazol-4-yl)quinazolin-4-yl)phenyl)piperazin-1-yl)ethan-1-one (37)

From 1-(4-(4-(6-bromoquinazolin-4-yl)phenyl)piperazin-1-yl)ethan-1-one with tert-butyl 4-(4,4,5,5-tetramethyl-1,3,2-dioxaborolan-2-yl)-1H-pyrazole-1-carboxylate, the title compound was isolated in a similar manner as depicted in Example 1.1, Step D, as a yellow solid. LCMS [M+H]⁺=399. ¹H NMR (500 MHz, CDCl₃) δ 9.25 (s, 1H), 8.27 (s, 1H), 8.12 (dd, J=7.0, 4.5 Hz, 1H), 8.05 (d, J=8.7 Hz, 1H), 7.93 (s, 2H), 7.82 (d, J=8.2 Hz, 2H), 7.08 (d, J=8.3 Hz, 2H), 3.84-3.79 (m, 2H), 3.69-3.65 (m, 2H), 3.43-3.34 (m, 4H), 3.17 (dd, J=9.7, 5.7 Hz, 1H), 2.16 (s, 3H). ¹³C NMR (126 MHz, CDCl₃) δ 169.22, 167.45, 153.46, 152.28, 149.57, 132.53, 132.14 131.76, 131.55, 129.01, 127.70, 123.36, 122.45, 115.46, 115.38, 48.35, 48.08, 45.96, 41.14, 21.37.

### Example 1.31: 1-(4-(4-(6-(2-aminopyridin-4-yl)quinolin-4-yl)phenyl)piperazin-1-yl)ethan-1-one (38)

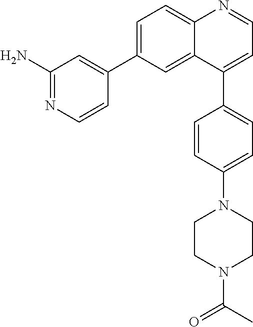 [SMILES: CC(=O)N1CCN(c2ccc(-c3ccnc4ccc(-c5ccnc(N)c5)cc34)cc2)CC1]

### Step A. Preparation 1-(4-(4-(6-(2-aminopyridin-4-yl)quinolin-4-yl)phenyl)piperazin-1-yl)ethan-1-one (38)

From 4-(4-chloroquinolin-6-yl)pyridin-2-amine (XXIII) with 1-(4-(4-(4,4,5,5-tetramethyl-1,3,2-dioxaborolan-2-yl)phenyl)piperazin-1-yl)ethan-1-one, the title compound was isolated in a similar manner as depicted in Example 1.13, Step A, as a gray solid. LCMS [M+H]⁺=424. ¹H NMR (500 MHz, CDCl₃) δ 8.99 (dd, J=13.5, 4.5 Hz, 1H), 8.28 (d, J=7.2 Hz, 1H), 7.95-7.77 (m, 2H), 7.56 (d, J=8.4 Hz, 1H), 7.48 (d, J=8.1 Hz, 2H), 7.40 (dd, J=8.3, 4.4 Hz, 1H), 7.25-7.20 (m, 1H), 7.12 (d, J=8.2 Hz, 3H), 6.98 (s, 1H), 6.87 (dt, J=17.5, 8.5 Hz, 1H), 3.86-3.61 (m, 5H), 3.38-3.15 (m, 4H), 2.17 (s, 3H). ¹³C NMR (126 MHz, CDCl₃) δ 169.14, 158.09, 151.03, 150.77, 148.95, 148.77, 145.87, 136.07, 130.78, 130.66, 128.92, 127.92, 126.91, 124.52, 121.89, 116.12, 112.71, 107.45, 75.98, 46.12, 41.27, 21.37.

### Example 1.32: 2-(4-(4-(6-(2-aminopyridin-4-yl)quinazolin-4-yl)phenyl)piperazin-1-yl)ethan-1-ol (41)

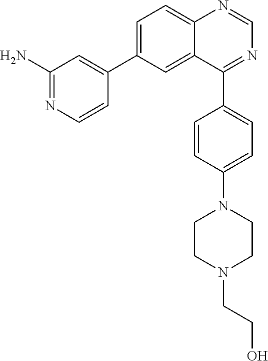 [SMILES: Nc1cc(-c2ccc3ncnc(-c4ccc(N5CCN(CCO)CC5)cc4)c3c2)ccn1]

### Step A. Preparation 2-(4-(4-bromophenyl)piperazin-1-yl)ethan-1-ol

From 1-(4-Bromophenyl)piperazine with ethylene bromohydrin, the title compound was isolated in a similar manner as depicted in Example 1.22, Step A, as a white solid. LCMS [M+H]⁺=284. ¹H NMR (500 MHz, CDCl₃) δ 7.37-7.31 (m, 2H), 6.81-6.75 (m, 2H), 3.69 (t, J=5.3 Hz, 2H), 3.23-3.17 (m, 4H), 2.87 (s, 1H), 2.72 (t, J=5.0 Hz, 4H), 2.65 (t, J=5.3 Hz, 2H). ¹³C NMR (126 MHz, CDCl₃) δ 150.12, 131.93, 117.76, 112.10, 59.41, 57.68, 52.76, 48.93.

### Step B. Preparation 1-(4-bromophenyl)-4-(2-((tert-butyldimethylsilyl)oxy)ethyl)piperazine

2-(4-(4-bromophenyl)piperazin-1-yl)ethan-1-ol (200 mg, 1 eq, 701 μmol), Imidazole (91 mg, 88.1 μL, 1.9 eq, 1.33 mmol), and DCM (3.5 mL) were added to a vial and allowed to stir for 10 min. tert-Butyldimethylchlorosilane (159 mg, 175 μL, 1.5 eq, 1.05 mmol) was added still and continued to stir for 16 hour at 25° C. Upon completion the reaction was diluted with DCM, washed with water, brine, and dried with magnesium sulfated. The reaction was purified via column chromatography, to afford 1-(4-bromophenyl)-4-(2-((tert-butyldimethylsilyl)oxy)ethyl)piperazine (280 mg, 701 mol, 99%). LCMS [M+H]⁺=400. ¹H NMR (500 MHz, CDCl₃) δ 7.27-7.22 (m, 2H), 6.73-6.66 (m, 2H), 3.73 (t, J=6.2 Hz, 2H), 3.12-3.06 (m, 4H), 2.62 (dd, J=6.2, 4.1 Hz, 4H), 2.52 (t, J=6.2 Hz, 2H), 0.83 (s, 9H), 0.00 (s, 6H). ¹³C NMR (126 MHz, CDCl₃) δ 150.33, 131.84, 116.67, 111.71, 61.31, 60.48, 53.64, 48.90, 25.96, 18.30.

### Step C. Preparation 1-(2-((tert-butyldimethylsilyl)oxy)ethyl)-4-(4-(4,4,5,5-tetramethyl-1,3,2-dioxaborolan-2-yl)phenyl)piperazine

From 1-(4-bromophenyl)-4-(2-((tert-butyldimethylsilyl)oxy)ethyl)piperazine, the title compound was isolated in a similar manner as depicted in Example 1.1, Step B, as a white solid. LCMS [M+H]⁺=446. ¹H NMR (500 MHz, CDCl₃) δ 7.69-7.64 (m, 2H), 6.86-6.83 (m, 2H), 3.78 (t, J=6.2 Hz, 2H), 3.27-3.18 (m, 4H), 2.74-2.64 (m, 4H), 2.57 (t, J=6.2 Hz, 2H), 1.20 (s, 12H), 0.87 (s, 9H), 0.04 (s, 6H). ¹³C NMR (126 MHz, CDCl₃) δ 153.30, 136.11, 129.09, 114.34, 83.37, 61.09, 60.42, 53.53, 47.86, 25.93, 24.85, 24.60, 18.27.

### Step D. Preparation 6-bromo-4-(4-(4-(2-((tert-butyldimethylsilyl)oxy)ethyl)piperazin-1-yl)phenyl)quinazoline

From 1-(2-((tert-butyldimethylsilyl)oxy)ethyl)-4-(4-(4,4,5,5-tetramethyl-1,3,2-dioxaborolan-2-yl)phenyl)piperazine, the title compound was isolated in a similar manner as depicted in Example 1.1, Step C, as a yellow solid. LCMS [M+H]⁺=528. ¹H NMR (500 MHz, CDCl₃) δ 9.46 (s, 1H), 8.53 (t, J=1.3 Hz, 1H), 8.13-8.07 (m, 2H), 7.91 (d, J=8.7 Hz, 1H), 7.32-7.28 (m, 1H), 7.23 (d, J=8.5 Hz, 1H), 7.18-7.14 (m, 1H), 4.00 (t, J=6.1 Hz, 2H), 3.56 (t, J=5.0 Hz, 2H), 2.96-2.88 (m, 3H), 2.80 (t, J=6.3 Hz, 2H), 1.07 (d, J=2.6 Hz, 9H), 0.24 (d, J=2.5 Hz, 6H). ¹³C NMR (126 MHz, CDCl₃) δ 154.93, 152.68, 149.95, 136.83, 131.47, 130.63, 129.46, 126.17, 124.11, 122.09, 121.05, 114.91, 61.29, 60.49, 53.56, 47.86, 29.71, 25.95, 18.31.

### Step E. Preparation 2-(4-(4-(6-(2-aminopyridin-4-yl)quinazolin-4-yl)phenyl)piperazin-1-yl)ethan-1-ol (41)

A 5 mL MWV was loaded with 6-bromo-4-(4-(4-(2-((tert-butyldimethylsilyl)oxy)ethyl)piperazin-1-yl)phenyl)quinazoline (30 mg, 1 eq, 57 μmol), 4-(4,4,5,5-tetramethyl-1,3,2-dioxaborolan-2-yl)pyridin-2-amine (14 mg, 1.1 eq, 63 μmol), PdCl2(dppf) (5 mg, 0.1 eq, 5.7 μmol), and tripotassium phosphate (18 mg, 1.5 eq, 85 mol), the vial capped then degassed for 10 minutes then injected with solvents degassed 1,4-Dioxane (0.19 mL):Water (32 μL), (6:1 v/v), degassed further for 10 min and heated to 110° C. for 3 hour in an oil bath. The mixture was stirring in THF (0.45 mL) at 25° C., and tetrabutylammonium fluoride (14 mg, 1 eq, 52 mol) was added. The solution was allowed to stir for 1.5 hour. Upon completion, the reaction was bacified with 1N NaOH and diluted with DCM. The organics were purified through column chromatography and produced 2-(4-(4-(6-(2-aminopyridin-4-yl)quinazolin-4-yl)phenyl)piperazin-1-yl)ethan-1-ol (41) as a yellow solid. LCMS [M+H]⁺=427. ¹H NMR (400 MHz, CDCl₃) δ 9.32 (s, 1H), 8.39 (d, J=2.0 Hz, 1H), 8.16 (dd, J=8.8, 3.9 Hz, 2H), 8.09 (dd, J=8.8, 2.0 Hz, 2H), 7.84-7.76 (m, 2H), 7.09 (d, J=8.7 Hz, 3H), 6.92 (dd, J=5.4, 1.6 Hz, 1H), 6.73 (d, J=1.6 Hz, 2H), 4.67 (s, 1H), 3.73 (t, J=5.3 Hz, 2H), 3.45 (t, J=5.1 Hz, 4H), 2.80 (t, J=5.1 Hz, 4H), 2.71 (t, J=5.3 Hz, 2H).

### Example 1.33: 4-(4-(3-fluoro-4-((1R,5S)-8-methyl-3,8-diazabicyclo[3.2.1]octan-3-yl)phenyl)quinazolin-6-yl)pyridin-2-amine (43)

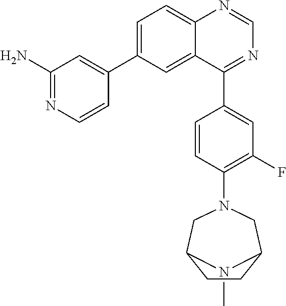 [SMILES: CN1C2CCC1CN(c1ccc(-c3ncnc4ccc(-c5ccnc(N)c5)cc34)cc1F)C2]

### Step A. Preparation tert-butyl (1R,5S)-3-(2-fluoro-4-nitrophenyl)-3,8-diazabicyclo[3.2.1]octane-8-carboxylate

1,2-difluoro-4-nitrobenzene (1.1 g, 0.78 mL, 1 eq, 7.1 mmol), tert-butyl (1R,5S)-3,8-diazabicyclo[3.2.1]octane-8-carboxylate (1.5 g, 1 eq, 7.1 mmol) was stirred in DMF (9.0 mL) at 90° C. for 16 h. The reaction was then diluted with of ethyl acetate, washed with water, and concentrated under reduced pressure to afford tert-butyl (1R,5S)-3-(2-fluoro-4-nitrophenyl)-3,8-diazabicyclo[3.2.1]octane-8-carboxylate (1.839 g, 5.234 mmol, 74%) as a yellow solid. LCMS [M+H]⁺=351. ¹H NMR (500 MHz, DMSO-d₆) δ 8.04-7.95 (m, 2H), 7.14 (t, J=9.2 Hz, 1H), 4.22 (s, 2H), 3.50-3.42 (m, 2H), 3.12-3.03 (m, 2H), 1.91-1.79 (m, 3H), 1.42 (d, J=8.1 Hz, 9H). ¹³C NMR (126 MHz, DMSO-d₆) δ 152.33, 150.41, 145.02, 138.73, 120.58, 117.63, 111.67, 78.37, 60.90, 49.95, 27.47.

### Step B. Preparation 3-(4-amino-2-fluorophenyl)-3,8-diazabicyclo[3.2.1]octane-8-carboxylate

Tert-butyl 3-(2-fluoro-4-nitrophenyl)-3,8-diazabicyclo[3.2.1]octane-8-carboxylate (1.84 g, 1 eq, 5.234 mmol) was added to Water (8.8 mL):Ethanol (35 mL) and stirred for 10 min. iron (877 mg, 3 eq, 15.70 mmol) and ammonia hydrochloride (560 mg, 2 eq, 10.47 mmol) were added to the reaction flask and placed in an oil bath at 90° C. for 12 hour. Upon completion the reaction was diluted with EtOAc and filtered over celite. Organics were collected and reduced under pressure to afford a solid. The solid was diluted with water and filtered to afford tert-butyl 3-(4-amino-2-fluorophenyl)-3,8-diazabicyclo[3.2.1]octane-8-carboxylate (1.508 g, 4.692 mmol, 90%) a light purple solid, which was carried forward to the next reaction. LCMS [M+H]⁺=322.

### Step C. Preparation tert-butyl (1R,5S)-3-(2-fluoro-4-(4,4,5,5-tetramethyl-1,3,2-dioxaborolan-2-yl)phenyl)-3,8-diazabicyclo[3.2.1]octane-8-carboxylate

Tert-butyl (1R,5S)-3-(4-amino-2-fluorophenyl)-3,8-diazabicyclo[3.2.1]octane-8-carboxylate (1.00 g, 1 eq, 3.11 mmol), 4,4,4′,4′,5,5,5′,5′-octamethyl-2,2′-bi(1,3,2-dioxaborolane) (869 mg, 1.1 eq, 3.42 mmol), benzoic peroxyanhydride (15 mg. 0.02 eq, 62.2 gmol), and tert-butyl nitrite (481 mg, 1.5 eq, 4.67 mmol) were added in a vial in ACN (10 mL). The reaction stirred at 80° C. for 2 hours. The reaction was diluted with EtOAc, washed with water, brine, and dried with sodium sulfate. The reaction was purified via column chromatography (EtOAc/Hex 0-10%) to afford tert-butyl (1R,5S)-3-(2-fluoro-4-(4,4,5,5-tetramethyl-1,3,2-dioxaborolan-2-yl)phenyl)-3,8-diazabicyclo[3.2.1]octane-8-carboxylate (364 mg, 842 mol, 27%). LCMS [M+H]⁺=433. ¹H NMR (500 MHz, CDCl₃) δ 7.45 (dd, J=8.0, 1.5 Hz, 1H), 7.39 (dd, J=13.8, 1.5 Hz, 1H), 6.82 (dd, J=9.5, 6.9 Hz, 1H), 4.26 (s, 2H), 3.26 (d, J=11.1 Hz, 2H), 2.99 (s, 2H), 2.01-1.86 (m, 4H), 1.45 (s, 9H), 1.29 (s, 12H). ¹³C NMR (126 MHz, CDCl₃) δ 155.76, 153.63, 142.24, 131.37, 121.85, 118.20, 83.78, 79.64, 54.99, 49.9, 28.50, 27.73, 25.03.

### Step D. Preparation 4-(4-((1R,5S)-3,8-diazabicyclo[3.2.1]octan-3-yl)-3-fluorophenyl)-6-bromoquinazoline

In a vial 6-bromo-4-chloroquinazoline (194 mg, 1 eq, 796 μmol), tert-butyl (1R,5S)-3-(2-fluoro-4-(4,4,5,5-tetramethyl-1,3,2-dioxaborolan-2-yl)phenyl)-3,8-diazabicyclo[3.2.1]octane-8-carboxylate (344 mg, 1 eq, 796 mol), PdCl2dppf (29 mg, 0.05 eq, 39.8 μmol), K3PO4 (507 mg, 3 Eq, 2.39 mmol) and purged for 10 min. dioxane (8 mL), Water (1 mL) (6:1) were added to the vial and purged for an additional 10 min. The reaction was placed in the oil bath at 90° C. for 16 h. Upon completion, the reaction was cooled and in the 5 mL microwave vial DCM (2.5 mL), and 2,2,2-trifluoroacetic acid (888 mg, 597 μL, 20 eq, 7.79 mmol) were added to a vial. The mixture stirred at 25° C. for 4 h. After this time, the mixture was poured into DCM and washed with water to afford 4-(4-((1R,5S)-3,8-diazabicyclo[3.2.1]octan-3-yl)-3-fluorophenyl)-6-bromoquinazoline solid. LCMS [M+H]⁺=414. ¹H NMR (500 MHz, CDCl₃) δ 9.54 (s, 1H), 8.55 (t, J=1.4 Hz, 1H), 8.19 (d, J=1.4 Hz, 2H), 7.75-7.70 (m, 2H), 7.24 (t, J=8.5 Hz, 1H), 3.91-3.84 (m, 2H), 3.65 (dd, J=11.5, 2.7 Hz, 2H), 3.33 (d, J=11.0 Hz, 2H), 2.78 (s, 2H), 2.29 (t, J=6.7 Hz, 2H), 2.10 (dt, J=7.3, 2.8 Hz, 2H). ¹³C NMR (126 MHz, CDCl₃) δ 165.77, 154.82, 153.94, 149.95, 142.10, 137.14, 130.76, 128.99, 126.70, 123.92, 121.52, 118.52, 118.01, 117.83, 56.27, 54.69, 28.69.

### Step E. Preparation 6-bromo-4-(3-fluoro-4-((1R,5S)-8-methyl-3,8-diazabicyclo[3.2.1]octan-3-yl)phenyl)quinazoline

4-(4-((1R,5S)-3,8-diazabicyclo[3.2.1]octan-3-yl)-3-fluorophenyl)-6-bromoquinazoline (132 mg, 1 eq, 319 μmol), formaldehyde (14 mg, 13.2 μL, 1.5 eq, 479 gmol), acetic acid (38 mg, 36.6 μL, 2 eq, 639 μmol) was added to DCM (3 mL) in a vial. The reaction was allowed to stir at 25° C. for 1 hour. The reaction was cooled, diluted with EtOAc, washed with water, and dried with sodium sulfate. The organic layer was concentrated under reduced pressure to afford 6-bromo-4-(3-fluoro-4-((1R,5S)-8-methyl-3,8-diazabicyclo[3.2.1]octan-3-yl)phenyl)quinazoline (80 mg, 0.19 mmol, 59%). LCMS [M+H]⁺=428. ¹H NMR (500 MHz, CDCl₃) δ 9.52 (s, 1H), 8.51 (t, J=1.3 Hz, 1H), 8.16 (d, J=1.4 Hz, 2H), 7.73-7.67 (m, 2H), 7.24 (t, J=8.6 Hz, 1H), 3.66 (dd, J=7.4, 4.1 Hz, 2H), 3.59 (d, J=2.3 Hz, 4H), 2.69 (s, 3H), 2.35-2.25 (m, 4H). ¹³C NMR (126 MHz, CDCl₃) δ 165.68, 154.80, 149.94, 141.45, 137.19, 130.76, 129.67, 128.94, 126.71, 123.91, 121.58, 118.80, 117.98, 117.80, 61.50, 54.34, 40.06, 25.03.

### Step F. Preparation 4-(4-(3-fluoro-4-((1R,5S)-8-methyl-3,8-diazabicyclo[3.2.1]octan-3-yl)phenyl)quinazolin-6-yl)pyridin-2-amine (43)

From 6-bromo-4-(3-fluoro-4-((1R,5S)-8-methyl-3,8-diazabicyclo[3.2.1]octan-3-yl)phenyl)quinazoline, the title compound was isolated in a similar manner as depicted in Example 1.9, Step D, as a brown solid. LCMS [M+H]⁺=441. ¹H NMR (500 MHz, CDCl₃) δ 9.32 (s, 1H), 8.35 (d, J=2.0 Hz, 1H), 8.18-8.13 (m, 2H), 8.09 (dd, J=8.8, 2.0 Hz, 1H), 7.56-7.53 (m, 1H), 7.52 (s, 1H), 7.00 (t, J=8.5 Hz, 1H), 6.91-6.88 (m, 1H), 6.71 (s, 1H), 4.55 (s, 2H), 3.36 (dd, J=11.3, 2.8 Hz, 2H), 3.23 (s, 2H), 3.15 (d, J=10.7 Hz, 2H), 2.34 (s, 3H), 2.05 (dd, J=8.8, 4.3 Hz, 2H), 1.94 (t, J=6.5 Hz, 2H). ¹³C NMR (126 MHz, CDCl₃) δ 167.04, 159.02, 155.01, 153.84, 151.32, 149.12, 149.00, 138.24, 132.50, 129.75, 126.77, 124.98, 123.86, 122.98, 118.50, 118.09, 117.91, 112.87, 106.48, 61.28, 55.61, 41.06, 25.25.

### Example 1.34: 4-(4-(4-(4-(tert-butyl)piperazin-1-yl)-3-fluorophenyl)quinazolin-6-yl)pyridin-2-amine (45)

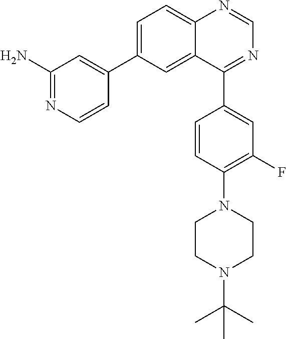 [SMILES: CC(C)(C)N1CCN(c2ccc(-c3ncnc4ccc(-c5ccnc(N)c5)cc34)cc2F)CC1]

### Step A. Preparation 1-(tert-butyl)-4-(2-fluoro-4-nitrophenyl)piperazine

From 1-(tert-butyl)piperazine, the title compound was isolated in a similar manner as depicted in Example 1.43, Step A, as a yellow solid. LCMS [M+H]⁺=281. ¹H NMR (400 MHz, CDCl₃) δ 7.98-7.93 (m, 1H), 7.87 (dd, J=13.3, 2.6 Hz, 1H), 6.88 (t, J=8.8 Hz, 1H), 3.35-3.26 (m, 4H), 2.74 (t, J=4.9 Hz, 4H), 1.10 (s, 9H). C NMR (101 MHz, CDCl₃) δ 154.31, 151.83, 145.79, 121.13, 117.01, 112.47, 50.29, 45.71, 25.91.

### Step B. Preparation 4-(4-(tert-butyl)piperazin-1-yl)-3-fluoroaniline

From 1-(tert-butyl)-4-(2-fluoro-4-nitrophenyl)piperazine, the title compound was isolated in a similar manner as depicted in Example 1.43, Step B, as a purple solid. LCMS [M+H]⁺=251. ¹H NMR (400 MHz, CDCl₃) δ 6.80 (t, J=8.9 Hz, 1H), 6.42 (d, J=2.6 Hz, 1H), 6.38 (d, J=8.1 Hz, 1H), 3.51 (s, 2H), 3.03 (s, 4H), 2.79 (s, 4H), 1.13 (s, 9H). ¹³C NMR (101 MHz, CDCl₃) δ 157.98, 155.54, 142.62, 120.38, 110.77, 103.82, 51.75, 46.04, 25.88.

### Step C. Preparation 1-(tert-butyl)-4-(2-fluoro-4-(4,4,5,5-tetramethyl-1,3,2-dioxaborolan-2-yl)phenyl)piperazine

From 4-(4-(tert-butyl)piperazin-1-yl)-3-fluoroaniline, the title compound was isolated in a similar manner as depicted in Example 1.43, Step C. LCMS [M+H]'⁰=363. ¹H NMR (400 MHz, CDCl₃) δ 8.04 (d, J=7.5 Hz, 1H), 7.48 (d, J=8.0 Hz, 1H), 7.05-7.00 (m, 1H), 3.31-3.30 (m, 4H), 2.97 (s, 5H), 1.28 (s, 9H), 1.22 (s, 12H).

### Step D. Preparation 6-bromo-4-(4-(4-(tert-butyl)piperazin-1-yl)-3-fluorophenyl)quinazoline

From 1-(tert-butyl)-4-(2-fluoro-4-(4,4,5,5-tetramethyl-1,3,2-dioxaborolan-2-yl)phenyl)piperazine, the title compound was isolated in a similar manner as depicted in Example 1.1, Step C, as a yellow solid. LCMS [M+H]⁺=442. ¹H NMR (400 MHz, CDCl₃) δ 9.31 (s, 1H), 8.30 (s, 1H), 7.96 (s, 1H), 7.54-7.47 (m, 2H), 7.46-7.33 (m, 1H), 7.11 (t, J=8.5 Hz, 1H), 3.46 (s, 1H), 3.42-3.36 (m, 4H), 2.96 (m, 4H), 1.25 (s, 9H).

### Step E. Preparation 4-(4-(4-(4-(tert-butyl)piperazin-1-yl)-3-fluorophenyl)quinazolin-6-yl)pyridin-2-amine (45)

From 6-bromo-4-(4-(4-(tert-butyl)piperazin-1-yl)-3-fluorophenyl)quinazoline, the title compound was isolated in a similar manner as depicted in Example 1.9, Step D, as a yellow solid. LCMS [M+H]⁺=457. ¹H NMR (500 MHz, CDCl₃) δ 9.33 (s, 1H), 8.32 (d, J=2.0 Hz, 1H), 8.18-8.14 (m, 2H), 8.10 (dd, J=8.8, 2.0 Hz, 1H), 7.59-7.52 (m, 2H), 7.11 (t, J=8.5 Hz, 1H), 6.89 (d, J=5.3 Hz, 1H), 6.70 (s, 1H), 4.61 (s, 2H), 3.40 (m, 4H), 2.95 (m, 4H), 1.23 (s, 9H).

### Example 1.35: 1-(5-(4-(6-(2-aminopyridin-4-yl)quinazolin-4-yl)-2-fluorophenyl)hexahydropyrrolo[3,4-c]pyrrol-2(1H)-yl)ethan-1-one (46)

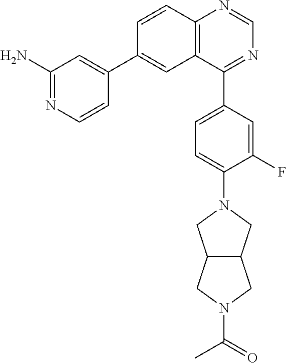 [SMILES: CC(=O)N1CC2CN(c3ccc(-c4ncnc5ccc(-c6ccnc(N)c6)cc45)cc3F)CC2C1]

### Step A. Preparation tert-butyl (3aR,6aS)-5-(2-fluoro-4-nitrophenyl)hexahydropyrrolo[3,4-c]pyrrole-2(1H)-carboxylate

From tert-butyl (3aR,6aS)-hexahydropyrrolo[3,4-c]pyrrole-2(1H)-carboxylate, the title compound was isolated in a similar manner as depicted in Example 1.43, Step A, as a yellow solid. LCMS [M+H]⁺=351. ¹H NMR (400 MHz, CDCl₃) δ 7.94-7.87 (m, 1H), 7.87-7.82 (m, 1H), 6.52 (t, J=8.9 Hz, 1H), 3.81 (tt, J=7.2, 2.9 Hz, 2H), 3.65 (dd, J=11.4, 6.9 Hz, 2H), 3.49 (dt, J=11.0, 3.4 Hz, 2H), 3.30 (d, J=11.4 Hz, 2H), 2.99 (dp, J=8.0, 2.9 Hz, 2H), 1.44 (s, 9H). ¹³C NMR (101 MHz, CDCl₃) δ 154.55, 150.39, 147.96, 142.03, 136.90, 121.87, 113.04, 79.85, 54.00, 49.89, 41.21, 28.56.

### Step B. Preparation tert-butyl 5-(4-amino-2-fluorophenyl)hexahydropyrrolo[3,4-c]pyrrole-2(1H)-carboxylate

From tert-butyl (3aR,6aS)-5-(2-fluoro-4-nitrophenyl)hexahydropyrrolo[3,4-c]pyrrole-2(1H)-carboxylate, the title compound was isolated in a similar manner as depicted in Example 1.43, Step B, as a purple solid. LCMS [M+H]⁺=321.

### Step C. Preparation tert-butyl 5-(4-amino-2-fluorophenyl)hexahydropyrrolo[3,4-c]pyrrole-2(1H)-carboxylate

From tert-butyl 5-(2-fluoro-4-(4,4,5,5-tetramethyl-1,3,2-dioxaborolan-2-yl)phenyl)hexahydropyrrolo[3,4-c]pyrrole-2(1H)-carboxylate, the title compound was isolated in a similar manner as depicted in Example 1.43, Step C. LCMS [M+H]⁺=433. ¹H NMR (400 MHz, CDCl₃) δ 7.43-7.33 (m, 1H), 7.00-6.95 (m, 1H), 6.72-6.64 (m, 1H), 3.67-3.60 (m, 2H), 3.56 (ddd, J=9.0, 7.0, 1.9 Hz, 2H), 3.39-3.30 (m, 2H), 3.28 (dq, J=10.0, 2.1 Hz, 2H), 2.93 (h, J=5.9 Hz, 2H), 1.44 (d, J=1.5 Hz, 9H), 1.22 (s, 12H).

### Step D. Preparation 6-bromo-4-(3-fluoro-4-(hexahydropyrrolo[3,4-c]pyrrol-2(1H)-yl)phenyl)quinazoline

From tert-butyl 5-(2-fluoro-4-(4,4,5,5-tetramethyl-1,3,2-dioxaborolan-2-yl)phenyl)hexahydropyrrolo[3,4-c]pyrrole-2(1H)-carboxylate, the title compound was isolated in a similar manner as depicted in Example 1.43, Step D. LCMS [M+H]⁺=412. ¹H NMR (400 MHz, CDCl₃) δ 9.29 (d, J=5.7 Hz, 1H), 8.36-8.33 (m, 1H), 7.95 (d, J=1.4 Hz, 2H), 7.57-7.46 (m, 3H), 6.88 (t, J=8.6 Hz, 1H), 3.52 (t, J=7.4 Hz, 2H), 3.44 (d, J=10.1 Hz, 2H), 3.27 (d, J=7.9 Hz, 2H), 2.99-2.94 (m, 2H), 1.46 (d, J=3.6 Hz, 1H), 0.91-0.78 (m, 2H). ¹³C NMR (101 MHz, CDCl₃) δ 156.21, 154.91, 150.06, 137.14, 130.81, 129.17, 126.99, 124.11, 124.01, 123.52, 121.49, 118.10, 117.22, 116.45, 55.64, 53.58, 42.74.

### Step E. Preparation 1-(5-(4-(6-bromoquinazolin-4-yl)-2 fluorophenyl)hexahydropyrrolo[3,4-c]pyrrol-2(1H)-yl)ethan-1-one

From 6-bromo-4-(3-fluoro-4-(hexahydropyrrolo[3,4-c]pyrrol-2(1H)-yl)phenyl)quinazoline, the title compound was isolated in a similar manner as depicted in Example 1.22, Step A. LCMS [M+H]⁺=456. ¹H NMR (500 MHz, CDCl₃) δ 9.28 (s, 1H), 8.36 (d, J=2.1 Hz, 1H), 8.02-7.93 (m, 3H), 7.56 (dd, J=14.7, 2.1 Hz, 1H), 7.54-7.50 (m, 1H), 6.77 (t, J=8.7 Hz, 1H), 3.84-3.76 (m, 4H), 3.53 (ddd, J=13.1, 6.9, 3.7 Hz, 2H), 3.50-3.41 (m, 3H), 3.12 (dt, J=7.6, 4.9 Hz, 1H), 3.03 (dtd, J=10.4, 7.6, 3.8 Hz, 1H), 2.07 (s, 3H).

### Step F. Preparation 1-(5-(4-(6-(2-aminopyridin-4-yl)quinazolin-4-yl)-2-fluorophenyl)hexahydropyrrolo[3,4-c]pyrrol-2(1H)-yl)ethan-1-one (46)

From 1-(5-(4-(6-bromoquinazolin-4-yl)-2-fluorophenyl)hexahydropyrrolo[3,4-c]pyrrol-2(1H)-yl)ethan-1-one, the title compound was isolated in a similar manner as depicted in Example 1.9, Step B, as a yellow solid. LCMS [M+H]⁺=469. ¹H NMR (500 MHz, CDCl₃) δ 9.31 (s, 1H), 8.35 (d, J=2.0 Hz, 1H), 8.18-8.13 (m, 2H), 8.09 (dd, J=8.8, 2.0 Hz, 1H), 7.56-7.53 (m, 1H), 7.52 (s, 1H), 7.00 (t, J=8.5 Hz, 1H), 6.91-6.88 (m, 1H), 6.72 (s, 1H), 4.63 (s, 2H), 3.84-3.76 (m, 4H), 3.53 (ddd, J=13.1, 6.9, 3.7 Hz, 2H), 3.50-3.41 (m, 3H), 3.12 (dt, J=7.6, 4.9 Hz, 1H), 3.03 (dtd, J=10.4, 7.6, 3.8 Hz, 1H). 2.11 (s, 3H).

### Example 1.36: 4-(4-(4-(4-ethylpiperazin-1-yl)-3-fluorophenyl)quinazolin-6-yl)pyridin-2-amine (36)

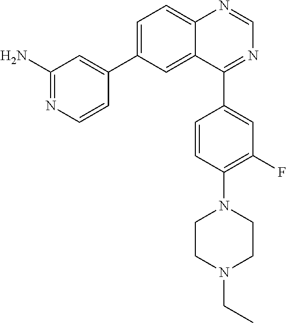 [SMILES: CCN1CCN(c2ccc(-c3ncnc4ccc(-c5ccnc(N)c5)cc34)cc2F)CC1]

### Step A. Preparation 6-bromo-4-(4-(4-ethylpiperazin-1-yl)-3-fluorophenyl)quinazoline

6-bromo-4-(3-fluoro-4-(piperazin-1-yl)phenyl)quinazoline (XXV) (218 mg, 1 eq, 563 μmol) and potassium carbonate (156 mg, 2 eq, 1.13 mmol) were added to Acetone (5.6 mL) and stirred under argon for 10 min. iodoethane (97 mg, 49.8 μL, 1.1 eq, 619 μmol) was added and allowed to stir at 25° C. for 24 hour. Upon completion, the reaction was reduced under pressure then dissolved in DCM. Org layer was washed with water, brine, and dried with sodium sulfate. Collected organic layer was reduced under pressure and purified via column chromatography (DCM/MeOH 0-10%) to afford 6-bromo-4-(4-(4-ethylpiperazin-1-yl)-3-fluorophenyl)quinazoline (25 mg, 60 μmol, 11%). LCMS [M+H]⁺=414. ¹H NMR (500 MHz, CDCl₃) δ 9.33 (s, 1H), 8.32 (t, J=1.4 Hz, 1H), 7.97 (d, J=1.3 Hz, 2H), 7.56-7.49 (m, 2H), 7.12 (t, J=8.7 Hz, 1H), 3.32 (t, J=4.9 Hz, 4H), 2.72 (t, J=4.8 Hz, 4H), 2.55 (d, J=7.2 Hz, 2H), 1.17 (t, J=7.2 Hz, 3H). ¹³C NMR (126 MHz, CDCl₃) δ 165.76, 156.18, 154.83, 154.21, 149.97, 142.10, 137.19, 130.80, 130.00, 128.97, 126.67, 123.94, 121.58, 118.76, 117.87, 52.54, 49.95, 29.71, 11.83.

### Step B. Preparation 4-(4-(4-(4-ethylpiperazin-1-yl)-3-fluorophenyl)quinazolin-6-yl)pyridin-2-amine (36)

From 6-bromo-4-(4-(4-ethylpiperazin-1-yl)-3-fluorophenyl)quinazoline, the title compound was isolated in a similar manner as depicted in Example 1.9, Step B, as a brown solid. LCMS [M+H]⁺=429. ¹H NMR (500 MHz, DMSO-d₆) δ 9.33 (s, 1H), 8.34-8.27 (m, 2H), 8.19 (s, 1H), 8.03 (d, J=5.3 Hz, 1H), 7.72-7.65 (m, 2H), 7.25 (t, J=8.7 Hz, 1H), 6.87 (dd, J=5.3, 1.6 Hz, 1H), 6.76 (d, J=1.6 Hz, 1H), 6.09 (s, 2H), 3.22 (t, J=4.8 Hz, 5H), 2.58 (t, J=4.7 Hz, 4H), 2.43 (q, J=7.2 Hz, 2H), 1.06 (t, J=7.2 Hz, 3H). ¹³C NMR (101 MHz, DMSO-d₆) δ 160.95, 155.08, 151.03, 149.31, 147.49, 144.34, 141.92, 138.55, 133.18, 129.78, 127.65, 124.49, 122.71, 119.35, 118.17, 117.94, 114.25, 110.69, 105.93, 52.72, 52.05, 50.06, 12.33.

### Example 1.37: 4-(4-(3-fluoro-4-(4-(methylsulfonyl)piperazin-1-yl)phenyl)quinazolin-6-yl)pyridin-2-amine (47)

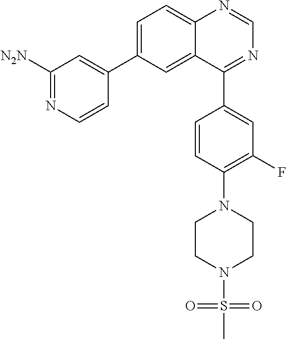

### Step A. Preparation 6-bromo-4-(3-fluoro-4-(4-(methylsulfonyl)piperazin-1-yl)phenyl)quinazoline

6-bromo-4-(3-fluoro-4-(piperazin-1-yl)phenyl)quinazoline (XXV) (60 mg, 1 eq, 0.15 mmol), K2CO3 (43 mg, 2 eq, 0.31 mmol), and methanesulfonyl chloride (18 mg, 1 eq, 0.15 mmol) were added to a vial in DMF (1.5 mL). The reaction was allowed to stir at 60° C. for 16 h. Upon completion, the reaction was diluted with EtOAC, washed with water, and dried with sodium sulfate. The organic layer was removed under reduced pressure and purified via column chromatography (DCM:MeOH 0-5%) to afford 6-bromo-4-(3-fluoro-4-(4-(methylsulfonyl)piperazin-1-yl)phenyl)quinazoline (45 mg, 97 mol, 62%). LCMS [M+H]⁺=466. ¹H NMR (500 MHz, CDCl₃) δ 9.32 (s, 1H), 8.27 (t, J=1.4 Hz, 1H), 7.96 (d, J=1.3 Hz, 2H), 7.55-7.49 (m, 2H), 7.11 (t, J=8.6 Hz, 1H), 3.44 (dd, J=6.1, 3.7 Hz, 4H), 3.33 (dd, J=6.4, 3.5 Hz, 4H), 2.84 (s, 3H). ¹³C NMR (126 MHz, CDCl₃) δ 165.51, 156.29, 154.81, 154.32, 149.97, 141.26, 137.32, 130.88, 128.79, 126.70, 123.89, 121.74, 119.19, 117.93, 49.87, 45.91, 34.47.

### Step B. Preparation 4-(4-(3-fluoro-4-(4-(methylsulfonyl)piperazin-1-yl)phenyl)quinazolin-6-yl)pyridin-2-amine (47)

From 6-bromo-4-(3-fluoro-4-(4-(methylsulfonyl)piperazin-1-yl)phenyl)quinazoline, the title compound was isolated in a similar manner as depicted in Example 1.9, Step B, as a yellow solid. LCMS [M+H]⁺=478. ¹H NMR (400 MHz, CDCl₃) δ 9.38 (s, 1H), 8.34 (d, J=1.6 Hz, 1H), 8.21 (d, J=8.7 Hz, 2H), 8.14 (d, J=8.8 Hz, 1H), 7.66-7.58 (m, 2H), 7.16 (t, J=8.5 Hz, 1H), 6.93 (d, J=4.8 Hz, 1H), 6.79 (s, 1H), 4.87 (s, 2H), 3.48 (dd, J=6.4, 3.3 Hz, 4H), 3.40-3.32 (m, 4H), 2.88 (s, 3H). ¹³C NMR (101 MHz, CDCl₃) δ 166.75, 156.55, 155.02, 154.09, 151.36, 149.31, 148.05, 141.26, 138.23, 132.62, 131.62, 129.92, 128.27, 126.79, 124.80, 122.91, 119.24, 118.00, 112.72, 49.89, 45.91, 34.54.

### Example 1.38: 4-(4-(3-fluoro-4-(4-isobutylpiperazin-1-yl)phenyl)quinazolin-6-yl)pyridin-2-amine (48)

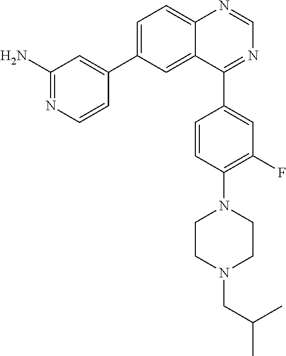 [SMILES: CC(C)CN1CCN(c2ccc(-c3ncnc4ccc(-c5ccnc(N)c5)cc34)cc2F)CC1]

### Step A. Preparation 6-bromo-4-(3-fluoro-4-(4-isobutylpiperazin-1-yl)phenyl)quinazoline

6-bromo-4-(3-fluoro-4-(piperazin-1-yl)phenyl)quinazoline (XXV) (150 mg, 1 eq. 387 μmol) in THF (3.0 mL) was treated with DIPEA (200 mg, 270 μL, 4 eq, 1.55 mmol) and isobutyraldehyde (140 mg, 0.18 mL, 5 eq, 1.94 mmol) and stirred for 1 hour at room temperature. STAB (sodium triacetoxyborohydride) (328 mg. 4 eq, 1.55 mmol) was then added and the resulting mixture was stirred for 18 hours at room temperature. The reaction mixture was diluted with an aqueous solution of sodium hydrogen carbonate (10%) and ethyl acetate.

The aqueous layer was extracted twice with ethyl acetate. The combined organic layers were washed with an aqueous solution of sodium chloride, dried over sodium sulphate and evaporated. The residue was purified by flash chromatography (MeOH:DCM 0-5%) affording 6-bromo-4-(3-fluoro-4-(4-isobutylpiperazin-1-yl)phenyl)quinazoline (139 mg, 314 μmol, 81%). LCMS [M+H]⁺=444. ¹H NMR (400 MHz, CDCl₃) δ 9.29 (s, 1H), 8.29 (d, J=1.5 Hz, 1H), 7.92 (d, J=1.4 Hz, 2H), 7.55-7.45 (m, 2H), 7.07 (dd, J=9.8, 7.4 Hz, 1H), 3.26 (s, 4H), 2.62 (s, 4H), 2.33-2.12 (m, 2H), 1.83 (p, J=6.9 Hz, 1H), 0.92 (d, J=6.6 Hz, 6H).

### Step B. Preparation 4-(4-(3-fluoro-4-(4-isobutylpiperazin-1-yl)phenyl)quinazolin-6-yl)pyridin-2-amine (48)

From 6-bromo-4-(3-fluoro-4-(4-isobutylpiperazin-1-yl)phenyl)quinazoline, the title compound was isolated in a similar manner as depicted in Example 1.9, Step B. LCMS [M+H]⁺=457 ¹H NMR (400 MHz, CDC3) δ 9.36 (s, 1H), 8.36 (d, J=1.9 Hz, 1H), 8.25-8.15 (m, 2H), 8.12 (dd, J=8.8, 2.0 Hz, 1H), 7.64-7.55 (m, 2H), 7.13 (t, J=8.7 Hz, 1H), 6.91 (dd, J=5.4, 1.5 Hz, 1H), 6.75 (s, 1H), 3.32 (t, J=5.0 Hz, 4H), 2.69 (q, J=5.8 Hz, 4H), 2.24 (d, J=7.3 Hz, 2H), 1.88 (m, J=13.6, 6.8 Hz, 1H), 0.97 (d, J=6.5 Hz, 6H).

### Example 1.39: 4-(4-(6-(2-aminopyridin-4-yl)quinazolin-4-yl)-2-fluorophenyl)-N,N-dimethylpiperazine-1-carboxamide (49)

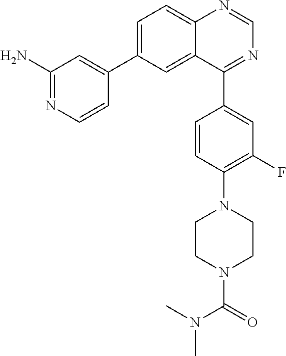 [SMILES: CN(C)C(=O)N1CCN(c2ccc(-c3ncnc4ccc(-c5ccnc(N)c5)cc34)cc2F)CC1]

### Step A. Preparation 4-(4-(6-bromoquinazolin-4-yl)-2-fluorophenyl)-N,N-dimethylpiperazine-1-carboxamide

From 6-bromo-4-(3-fluoro-4-(piperazin-1-yl)phenyl)quinazoline (XXV), the title compound was isolated in a similar manner as depicted in Example 1.22, Step A. LCMS [M+H]⁺=459. ¹H NMR (400 MHz, CDCl₃) δ 9.27 (s, 1H), 8.26 (s, 1H), 7.92 (s, 2H), 7.53-7.44 (m, 2H), 7.06 (t, J=8.5 Hz, 1H), 3.45-3.38 (m, 4H), 3.20 (t, J=4.8 Hz, 4H), 2.84 (s, 6H).

### Step B. Preparation 4-(4-(6-(2-aminopyridin-4-yl)quinazolin-4-yl)-2-fluorophenyl)-N,N-dimethylpiperazine-1-carboxamide (49)

From 4-(4-(6-bromoquinazolin-4-yl)-2-fluorophenyl)-N,N-dimethylpiperazine-1-carboxamide, the title compound was isolated in a similar manner as depicted in Example 1.9, Step B. LCMS [M+H]⁺=472. ¹H NMR (500 MHz, CDCl₃) δ 9.36 (d, J=3.0 Hz, 1H), 8.38 (s, 1H), 8.20 (d, J=8.2 Hz, 1H), 8.10 (s, 1H), 7.97 (s, 1H), 7.68-7.41 (m, 2H), 7.15 (s, 1H), 7.08-6.99 (m, 1H), 6.95 (s, 1H), 3.44 (s, 4H), 3.24 (s, 4H), 2.86 (d, J=6.6 Hz, 6H).

### Example 1.40: 4-(4-(3-fluoro-4-(4-isopropylpiperazin-1-yl)phenyl)quinazolin-6-yl)pyridin-2-amine (50)

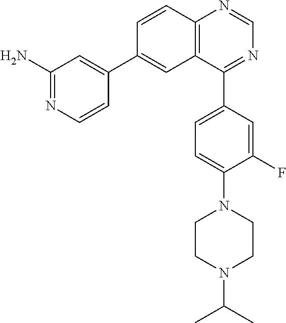 [SMILES: CC(C)N1CCN(c2ccc(-c3ncnc4ccc(-c5ccnc(N)c5)cc34)cc2F)CC1]

### Step A. Preparation 6-bromo-4-(3-fluoro-4-(4-isopropylpiperazin-1-yl)phenyl)quinazoline

From 6-bromo-4-(3-fluoro-4-(piperazin-1-yl)phenyl)quinazoline (XXV), the title compound was isolated in a similar manner as depicted in Example 1.47, Step A. LCMS [M+H]⁺=430. ¹H NMR (500 MHz, CDCl₃) δ 9.32 (d, J=6.2 Hz, 1H), 8.32-8.28 (m, 1H), 7.98-7.94 (m, 2H), 7.55-7.48 (m, 2H), 7.11 (t, J=8.5 Hz, 1H), 3.43-3.32 (m, 3H), 2.95-2.80 (m, 4H), 2.04 (m, 1H), 1.22-1.15 (m, 6H). ¹³C NMR (126 MHz, CDCl₃) δ 165.71, 156.18, 154.84, 154.21, 149.98, 141.93, 137.21, 130.82, 128.94, 126.66, 123.93, 121.60, 118.86, 117.80, 48.4, 40.0, 18.1.

### Step B. Preparation 4-(4-(3-fluoro-4-(4-isopropylpiperazin-1-yl)phenyl)quinazolin-6-yl)pyridin-2-amine (50)

From 6-bromo-4-(3-fluoro-4-(4-isopropylpiperazin-1-yl)phenyl)quinazoline, the title compound was isolated in a similar manner as depicted in Example 1.1, Step D. LCMS [M+H]⁺=442. ¹H NMR (500 MHz, CDCl₃) δ 9.35 (s, 1H), 8.30 (d, J=1.9 Hz, 1H), 8.18 (d, J=8.7 Hz, 1H), 8.12 (dd, J=8.8, 2.0 Hz, 1H), 7.59-7.56 (m, 2H), 7.16 (t, J=8.3 Hz, 1H), 6.91-6.87 (m, 1H), 6.74 (s, 1H), 4.83 (s, 2H), 3.64 (m, 4H), 3.20 (m, 4H), 1.41 (s, 7H).

### Example 1.41: 4-(4-(3-fluoro-4-(4-(methyl-d₃)piperazin-1-yl)phenyl)quinazolin-6-yl)pyridin-2-amine (51)

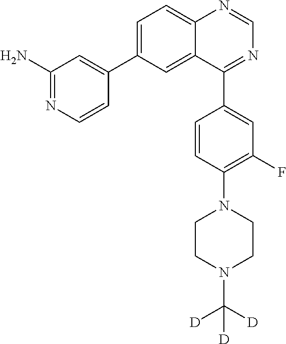 [SMILES: [2H]C([2H])([2H])N1CCN(c2ccc(-c3ncnc4ccc(-c5ccnc(N)c5)cc34)cc2F)CC1]

### Step A. Preparation 6-bromo-4-(3-fluoro-4-(4-(methyl-d₃)piperazin-1-yl)phenyl)quinazoline

From 6-bromo-4-(3-fluoro-4-(piperazin-1-yl)phenyl)quinazoline (XXV), the title compound was isolated in a similar manner as depicted in Example 1.47, Step A. LCMS [M+H]⁺=403. ¹H NMR (500 MHz, CDCl₃) δ 9.31 (s, 1H), 8.30 (t, J=1.4 Hz, 1H), 7.95 (d, J=1.3 Hz, 2H), 7.54-7.48 (m, 2H), 7.10 (t, J=8.6 Hz, 1H), 3.31 (t, J=5.0 Hz, 5H), 2.71 (t, J=4.4 Hz, 4H). ¹³C NMR (126 MHz, CDCl₃) δ 165.71, 156.18, 154.84, 154.21, 149.98, 141.93, 137.21, 130.82, 128.94, 126.66, 123.93, 121.60, 118.86, 117.80, 54.86, 49.69.

### Step B. Preparation 4-(4-(3-fluoro-4-(4-(methyl-d₃)piperazin-1-yl)phenyl)quinazolin-6-yl)pyridin-2-amine (51)

From 6-bromo-4-(3-fluoro-4-(4-(methyl-d₃)piperazin-1-yl)phenyl)quinazoline, the title compound was isolated in a similar manner as depicted in Example 1.9, Step B, as a yellow solid. LCMS [M+H]⁺=418. ¹H NMR (500 MHz, CDCl₃) δ 9.32 (s, 1H), 8.32 (d, J=1.9 Hz, 1H), 8.18-8.13 (m, 2H), 8.09 (dd, J=8.8, 2.0 Hz, 1H), 7.59-7.53 (m, 2H), 7.11 (t, J=8.6 Hz, 1H), 6.88 (dd, J=5.3, 1.5 Hz, 1H), 6.70 (d, J=1.5 Hz, 1H), 4.66-4.52 (m, 2H), 3.32 (t, J=4.9 Hz, 4H), 2.72 (t, J=4.8 Hz, 4H). ¹³C NMR (126 MHz, CDCl₃) δ 166.93, 159.02, 156.18, 154.98, 154.2, 151.29, 148.97, 141.74, 138.33, 132.60, 130.64, 129.79, 126.74, 124.86, 122.97, 118.88, 118.10, 112.77, 106.52, 54.83, 49.69, 41.03.

### Example 1.52: methyl 4-(4-(6-(2-aminopyridin-4-yl)quinazolin-4-yl)-2-fluorophenyl)piperazine-1-carboxylate (52)

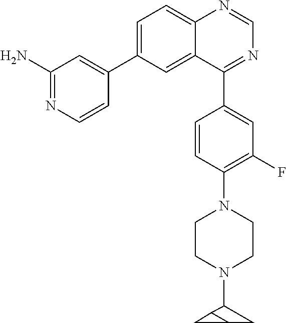 [SMILES: Nc1cc(-c2ccc3ncnc(-c4ccc(N5CCN(C6CC7CC76)CC5)c(F)c4)c3c2)ccn1]

### Step A. Preparation 6-bromo-4-(3-fluoro-4-(4-(methyl-d₃)piperazin-1-yl)phenyl)quinazoline

From 6-bromo-4-(3-fluoro-4-(piperazin-1-yl)phenyl)quinazoline (XXV), the title compound was isolated in a similar manner as depicted in Example 1.47, Step A. LCMS [M+H]⁺=446. ¹H NMR (500 MHz, CDCl₃) δ 9.32 (s, 1H), 8.29 (dd, J=1.8, 0.9 Hz, 1H), 7.97 (t, J=1.6 Hz, 2H), 7.55-7.52 (m, 1H), 7.50 (d, J=2.1 Hz, 1H), 7.09 (t, J=8.6 Hz, 1H), 3.73 (s, 3H), 3.68 (d, J=5.1 Hz, 4H), 3.19 (t, J=5.2 Hz, 4H). ¹³C NMR (126 MHz, CDCl₃) δ 165.71, 156.26, 155.90, 154.70, 154.29, 149.84, 141.96, 137.34, 130.75, 128.90, 126.70, 123.90, 121.73, 118.99, 117.92, 52.80, 50.07, 43.77.

### Step B. Preparation methyl 4-(4-(6-(2-aminopyridin-4-yl)quinazolin-4-yl)-2-fluorophenyl)piperazine-1-carboxylate (52)

From with methyl 4-(4-(6-bromoquinazolin-4-yl)-2-fluorophenyl)piperazine-1-carboxylate, the title compound was isolated in a similar manner as depicted in Example 1.9, Step B, as a yellow solid. LCMS [M+H]⁺=458. ¹H NMR (500 MHz, CDCl₃) δ 9.48 (s, 1H), 8.46 (s, 1H), 8.38-8.19 (m, 3H), 7.72 (t, J=11.2 Hz, 2H), 7.25 (d, J=8.5 Hz, 1H), 7.03 (s, 1H), 6.86 (s, 1H), 4.89 (s, 2H), 3.88 (s, 3H), 3.83 (m, 4H), 3.34 (m, 4H). ¹³C NMR (126 MHz, CDCl₃) δ 166.89, 158.91, 155.91, 154.97, 151.29, 149.13, 148.54, 141.77, 138.29, 132.62, 131.01, 129.83, 126.74, 124.84, 122.94, 119.02, 118.16, 112.72, 106.69, 52.80, 50.09, 43.83.

### Example 1.42: (4-(4-(6-(2-aminopyridin-4-yl)quinazolin-4-yl)-2-fluorophenyl)piperazin-1-yl)(cyclopropyl)methanone (53)

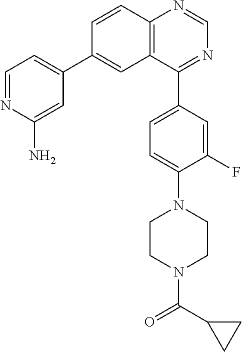 [SMILES: Nc1cc(-c2ccc3ncnc(-c4ccc(N5CCN(C(=O)C6CC6)CC5)c(F)c4)c3c2)ccn1]

### Step A. Preparation (4-(4-(6-bromoquinazolin-4-yl)-2-fluorophenyl)piperazin-1-yl)(cyclopropyl)methanone

From 6-bromo-4-(3-fluoro-4-(piperazin-1-yl)phenyl)quinazoline (XXV), the title compound was isolated in a similar manner as depicted in Example 1.47, Step A. LCMS [M+H]⁺=456. ¹H NMR (500 MHz, CDCl₃) δ 9.47 (s, 1H), 8.44 (t, J=1.3 Hz, 1H), 8.12 (d, J=1.7 Hz, 2H), 7.71-7.64 (m, 2H), 7.24 (t, J=8.4 Hz, 1H), 4.08-3.96 (m, 4H), 3.45-3.31 (m, 4H), 1.93 (tt, J=8.0, 4.7 Hz, 1H), 1.18-1.14 (m, 2H), 0.95 (dt, J=7.9, 3.4 Hz, 2H). ¹³C NMR (126 MHz, CDCl₃) δ 172.20, 165.67, 156.25, 154.74, 149.87, 141.69, 137.33, 130.76, 128.89, 126.73, 123.90, 121.73, 118.99, 118.11, 45.56, 42.06, 11.01, 7.61.

### Step B. Preparation (4-(4-(6-(2-aminopyridin-4-yl)quinazolin-4-yl)-2-fluorophenyl)piperazin-1-yl)(cyclopropyl)methanone (53)

From with methyl (4-(4-(6-bromoquinazolin-4-yl)-2-fluorophenyl)piperazin-1-yl)(cyclopropyl)methanone, the title compound was isolated in a similar manner as depicted in Example 1.9, Step B, as a yellow solid. LCMS [M+H]⁺=469. ¹H NMR (400 MHz, CDCl₃) δ 9.38 (s, 1H), 8.37 (d, J=1.6 Hz, 1H), 8.21 (d, J=8.5 Hz, 1H), 8.17-8.07 (m, 2H), 7.65-7.59 (m, 2H), 7.15 (t, J=8.5 Hz, 1H), 6.94 (s, 1H), 6.86 (s, 1H), 5.52 (s, 2H), 3.90 (d, J=16.2 Hz, 4H), 3.37-3.19 (m, 4H), 1.82 (tt, J=7.9, 4.7 Hz, 1H), 1.09-1.02 (m, 2H), 0.88-0.80 (m, 2H). ¹³C NMR (101 MHz, CDCl₃) δ 172.20, 166.94, 158.36, 156.52, 155.15, 154.06, 151.46, 150.11, 141.71, 137.69, 132.41, 131.02, 130.00, 128.37, 126.83, 125.07, 122.91, 119.04, 60.37, 11.00, 7.57.

### Example 1.43: 4-(4-(3-fluoro-4-(4-(2-methoxyethyl)piperazin-1-yl)phenyl)quinazolin-6-yl)pyridin-2-amine (54)

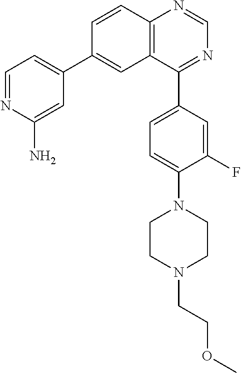 [SMILES: COCCN1CCN(c2ccc(-c3ncnc4ccc(-c5ccnc(N)c5)cc34)cc2F)CC1]

### Step A. 6-bromo-4-(3-fluoro-4-(4-(2-methoxyethyl)piperazin-1-yl)phenyl)quinazoline

From 6-bromo-4-(3-fluoro-4-(piperazin-1-yl)phenyl)quinazoline (XXV) (200 mg, (0940) 0.501 mmol) in DMF (3 mL) were added potassium carbonate (139 mg, 1.006 mmol), potassium iodide (83 mg, 0.500 mmol) and 2-bromoethyl methyl ether (70 mg, 0.504 mmol) under a nitrogen atmosphere. The resulting mixture was stirred for 2 h at 80° C. under a nitrogen atmosphere. The resulting mixture was cooled down to room temperature, diluted with 50 mL of ethyl acetate and washed twice with water. The organic layer was dried with sodium sulfate, filtered and concentrated. the resulting residue was purified via column chromatography. (0-5% MeOH/DCM) affording 6-bromo-4-(3-fluoro-4-(4-(2-methoxyethyl)piperazin-1-yl)phenyl)quinazoline (125 mg, 281 μmol, 73%) LCMS [M+H]⁺=446. ¹H NMR (400 MHz, CDCl₃) δ 9.29 (s, 1H), 8.28 (t, J=1.3 Hz, 1H), 7.92 (d, J=1.3 Hz, 2H), 7.53-7.44 (m, 2H), 7.07 (t, J=8.7 Hz, 1H), 3.56 (t, J=5.5 Hz, 2H), 3.36 (s, 3H), 3.29 (t, J=5.0 Hz, 4H), 2.73 (t, J=4.9 Hz, 4H), 2.67 (t, J=5.5 Hz, 2H).

### Step B. Preparation 4-(4-(3-fluoro-4-(4-(2-methoxyethyl)piperazin-1-yl)phenyl)quinazolin-6-yl)pyridin-2-amine (54)

From with methyl 6-bromo-4-(3-fluoro-4-(4-(2-methoxyethyl)piperazin-1-yl)phenyl)quinazoline, the title compound was isolated in a similar manner as depicted in Example 1.9, Step B, as a yellow solid. LCMS [M+H]⁺=459. ¹H NMR (400 MHz, CDCl₃) δ 9.34 (d, J=1.4 Hz, 1H), 8.37-8.32 (m, 1H), 8.21-8.05 (m, 3H), 7.62-7.53 (m, 2H), 7.16-7.07 (m, 1H), 6.90 (dd, J=5.4, 1.5 Hz, 1H), 6.72 (s, 1H), 3.60 (td, J=5.5, 1.5 Hz, 2H), 3.39 (d, J=1.0 Hz, 3H), 3.32 (d, J=5.1 Hz, 4H), 2.78 (dd, J=6.4, 3.3 Hz, 4H), 2.71 (td, J=5.5, 1.8 Hz, 2H).

### Example 1.44: 4-(4-(3-fluoro-4-(4-(tetrahydro-2H-pyran-4-yl)piperazin-1-yl)phenyl)quinazolin-6-yl)pyridin-2-amine (55)

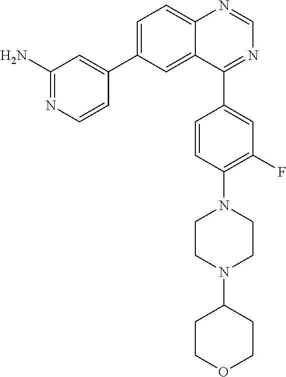 [SMILES: Nc1cc(-c2ccc3ncnc(-c4ccc(N5CCN(C6CCOCC6)CC5)c(F)c4)c3c2)ccn1]

### Step A. 6-bromo-4-(3-fluoro-4-(4-(tetrahydro-2H-pyran-4-yl)piperazin-1-yl)phenyl)quinazoline

From 6-bromo-4-(3-fluoro-4-(piperazin-1-yl)phenyl)quinazoline (XXV), the title compound was isolated in a similar manner as depicted in Example 1.48, Step A. LCMS [M+H]⁺=472. ¹H NMR (400 MHz, CDCl₃) δ 9.33 (s, 1H), 8.32 (t, J=1.4 Hz, 1H), 7.97 (d, J=1.4 Hz, 2H), 7.58-7.49 (m, 2H), 7.11 (t, J=8.7 Hz, 1H), 4.06 (dd, J=11.1, 4.4 Hz, 2H), 3.42 (td, J=11.9, 1.9 Hz, 2H), 3.30 (t, J=4.9 Hz, 4H), 2.81 (t, J=4.8 Hz, 4H), 2.66-2.46 (m, 2H), 1.92-1.76 (m, 1H), 1.65 (m, J=12.1, 4.5 Hz, 2H).

### Step B. Preparation 4-(4-(3-fluoro-4-(4-(tetrahydro-2H-pyran-4-yl)piperazin-1-yl)phenyl)quinazolin-6-yl)pyridin-2-amine (55)

From with 6-bromo-4-(3-fluoro-4-(4-(tetrahydro-2H-pyran-4-yl)piperazin-1-yl)phenyl)quinazoline, the title compound was isolated in a similar manner as depicted in Example 1.9, Step B, as a yellow solid. LCMS [M+H]⁺=471. ¹H NMR (500 MHz, CDCl₃) δ 9.32 (s, 1H), 8.32 (d, J=2.0 Hz, 1H), 8.15 (d, J=8.8 Hz, 2H), 8.08 (dd, J=8.7, 2.0 Hz, 1H), 7.58-7.52 (m, 2H), 7.08 (t, J=8.4 Hz, 1H), 6.87 (dd, J=5.3, 1.5 Hz, 1H), 6.69 (d, J=1.6 Hz, 1H), 3.99-3.87 (m, 2H), 3.79 (q, J=8.0 Hz, 1H), 3.69 (dd, J=8.6, 6.7 Hz, 1H), 3.26 (t, J=5.0 Hz, 4H), 3.05 (p, J=7.1 Hz, 1H), 2.74 (dt, J=10.4, 5.0 Hz, 2H), 2.62 (dt, =10.7, 4.9 Hz, 2H), 2.07 (dtd, J=12.1, 7.6, 4.4 Hz, 1H), 1.89 (dq, J=12.3, 8.0 Hz, 1H).

### Example 1.45: 4-(4-(3-fluoro-4-(4-(oxetan-3-yl)piperazin-1-yl)phenyl)quinazolin-6-yl)pyridin-2-amine (56). N

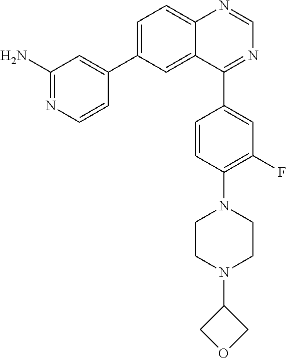 [SMILES: Nc1cc(-c2ccc3ncnc(-c4ccc(N5CCN(C6COC6)CC5)c(F)c4)c3c2)ccn1]

### Step A. 6-bromo-4-(3-fluoro-4-(4-(oxetan-3-yl)piperazin-1-yl)phenyl)quinazoline

6-bromo-4-(3-fluoro-4-(piperazin-1-yl)phenyl)quinazoline (51 ng, 1 eq, 0.13 mmol) was dissolved in DCE (0.52 mL) followed by adding of STAB (42 mg, 1.5 eq, 0.20 mmol), Oxentan-3-on (14 mg, 13 μL, 1.5 eq. 0.20 mmol), and acetic acid (1.6 mg, 1.5 μL, 2 eq, 26 μmol). The resulting suspension was stirred at 25° C. for 16 h before the solvent was removed under reduced pressure. The residue was washed with said aqNaHCO₃ and extracted with EtOAc. The combined organic layer was dried over Na₂SO₄ and concentrated under reduced pressure. 6-bromo-4-(3-fluoro-4-(4-(oxetan-3-ylpiperazin-1-yl)phenyl)quinazoline (17 mg, 38 μmol, 29%). LCIS [M+H]⁺=442. ¹H NMR (500 MHz, CDCl₃) δ 9.32 (s, 1H), 8.30 (t, J=1.4 Hz, 1H), 7.96 (d, J=1.4 Hz, 2H), 7.54-7.49 (m, 2H), 7.11 (t, J=8.7 Hz, 1H), 4.71 (d, J=6.4 Hz, 4H), 3.64 (s, 1H), 3.33 (m, 4H), 2.60 (m, 4H). ¹³C NMR (126 MHz, CDCl₃) δ 165.69, 156.20, 154.82, 154.23, 149.96, 137.25, 130.82, 128.92, 126.68, 123.93, 121.65, 118.85, 118.02, 117.84, 75.05, 59.25, 49.56.

### Step B. Preparation 4-(4-(3-fluoro-4-(4-(oxetan-3-yl)piperazin-1-yl)phenyl)quinazolin-6-yl)pyridin-2-amine (56)

From with 6-bromo-4-(3-fluoro-4-(4-(oxetan-3-yl)piperazin-1-yl)phenyl)quinazoline, the title compound was isolated in a similar manner as depicted in Example 1.9, Step B, as a yellow solid. LCMS [M+H]⁺=456. ¹H NMR (500 MHz, CDCl₃) δ 9.33 (s, 1H), 8.33 (d, J=1.8 Hz, 1H), 8.17 (d, J=8.7 Hz, 1H), 8.10 (t, J=9.2 Hz, 2H), 7.60-7.52 (m, 2H), 7.11 (t, J=8.4 Hz, 1H), 6.90 (d, J=5.1 Hz, 1H), 6.76 (s, 1H), 4.92 (s, 2H), 4.74-4.60 (m, 4H), 3.63-3.56 (m, 1H), 3.30 (t, J=4.7 Hz, 4H), 2.55 (t, J=4.8 Hz, 4H). ¹³C NMR (126 MHz, CDCl₃) δ 167.00, 158.60, 156.20, 155.08, 154.23, 151.38, 149.55, 147.63, 141.89, 137.99, 132.50, 129.91, 126.77, 125.01, 122.96, 118.84, 117.93, 112.68, 106.99, 75.41, 59.22, 49.60.

### Example 1.46: 4-(4-(3-fluoro-4-(4-(oxetan-3-ylmethyl)piperazin-1-yl)phenyl)quinazolin-6-yl)pyridin-2-amine (64)

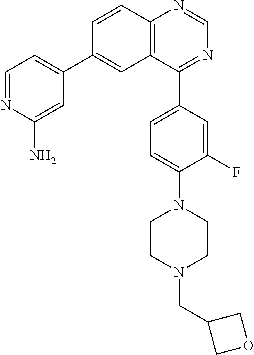 [SMILES: Nc1cc(-c2ccc3ncnc(-c4ccc(N5CCN(CC6COC6)CC5)c(F)c4)c3c2)ccn1]

### Step A. 6-bromo-4-(3-fluoro-4-(4-(oxetan-3-ylmethyl)piperazin-1-yl)phenyl)quinazoline

To a microwave vial potassium carbonate (107 mg, 2 eq, 775 μmol), 6-bromo-4-(3-fluoro-4-(piperazin-1-yl)phenyl)quinazoline (150 mg, 1 eq, 387 μmol) was added and dissolved in MeCN (1.5 mL). The solution was heated to 55° C. and stirred for 15 min, after time elapsed oxetan-3-ylmethyl 4-methylbenzenesulfonate (174 mg, 1.85 eq, 717 mol) was added. After addition, the reaction mixture stirred at 55° C. for 16 h. After time elapsed, the reaction mixture was cooled to room temperature diluted with ethyl acetate (75 mL) and washed with water (3×50 mL). The organic layer was dried with sodium sulfate, filtered, and concentrated via rotary evaporation. The resulting residue was purified via column chromatography (MeOH:DCM 0-5%) affording 6-bromo-4-(3-fluoro-4-(4-(oxetan-3-ylmethyl)piperazin-1-yl)phenyl)quinazoline (129.3 mg, 282.7 μmol, 73%). LCMS [M+H]⁺=458. ¹H NMR (400 MHz, CDCl₃) δ 9.35 (s, 1H), 8.34 (t, J=1.3 Hz, 1H), 7.99 (d, J=1.4 Hz, 2H), 7.61-7.50 (m, 2H), 7.11 (t, J=8.7 Hz, 1H), 4.92-4.80 (m, 2H), 4.48 (t, J=6.2 Hz, 2H), 3.39-3.23 (m, 5H), 2.85 (d, J=7.3 Hz, 2H), 2.69-2.61 (m, 4H).

### Step B. Preparation 4-(4-(3-fluoro-4-(4-(oxetan-3-ylmethyl)piperazin-1-yl)phenyl)quinazolin-6-yl)pyridin-2-amine (64)

From with 6-bromo-4-(3-fluoro-4-(4-(oxetan-3-yl)piperazin-1-yl)phenyl)quinazoline, the title compound was isolated in a similar manner as depicted in Example 1.9, Step B, as a yellow solid. LCMS [M+H]⁺=471. ¹H NMR (400 MHz, CDCl₃) δ 9.36 (s, 1H), 8.36 (d, J=2.0 Hz, 1H), 8.23-8.09 (m, 3H), 7.64-7.55 (m, 2H), 7.12 (t, J=8.6 Hz, 1H), 6.92 (dd, J=5.3, 1.5 Hz, 1H), 6.74 (s, 1H), 4.85 (dd, J=7.8, 6.0 Hz, 2H), 4.47 (t, J=6.1 Hz, 2H), 3.50 (s, 4H), 3.37-3.22 (m, 5H), 2.83 (d, J=7.3 Hz, 2H).

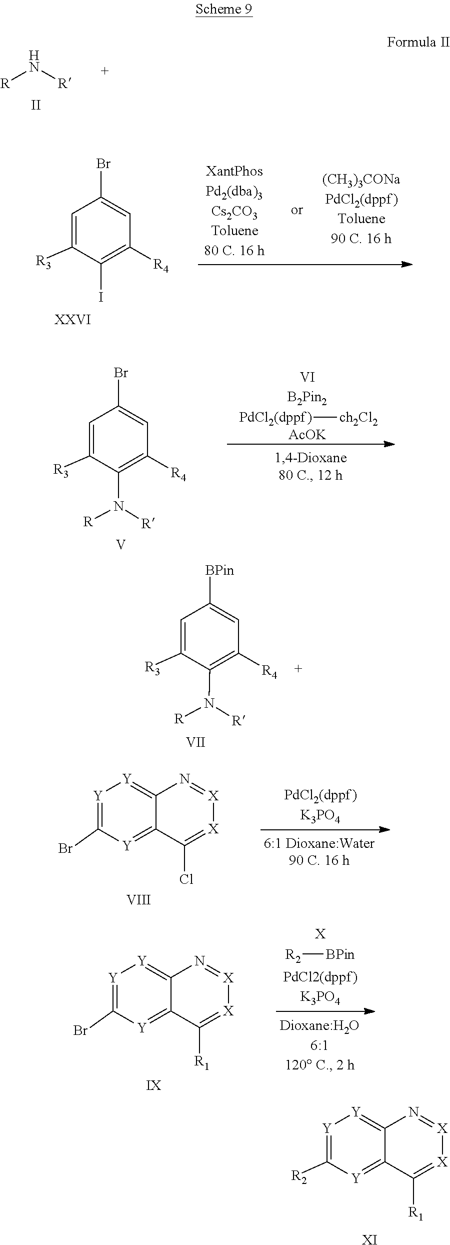

Scheme 9 describes the synthesis of general product (XI), through a Buchwald amination with commercially available amines (II) and bromo-benzenes (XXVI), to furnish (hetero)aryl bromides (V). The (hetero)aryl bromides (V) are utilized in a Miyaura borylation to produce the pinacol boronic esters (VII). The boronic esters are coupled with polyhalogenated heterocycles (VIII) in a Suzuki reaction to afford the R1 substituted heterocycle (IX) which is reacted in an additional Suzuki reaction resulting in R₂, R₁ substituted heterocycle (XI).

In some embodiments, compounds of Formula II of this present disclosure can be prepared as depicted in scheme 10.

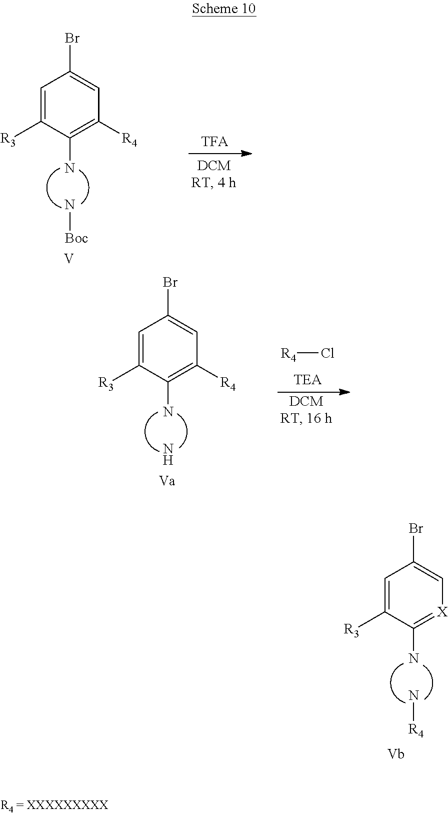

Scheme 10 describes the synthesis of advance intermediate (Vb), which can be prepared with commercially available, or readily prepared bromo- (hetero)aryl (V) and bromo-polyhalogenated bicyclic heteroaryl by an acid mediated deprotection (Va), to be further functionalized to produce General Intermediate (Vb).

### Preparation of intermediate 6-bromo-43-fluoro-4-(2,6-diazaspiro[3.3]heptan-2-yl)phenyl)quinazoline (XXVIII) is depicted below in Scheme 11

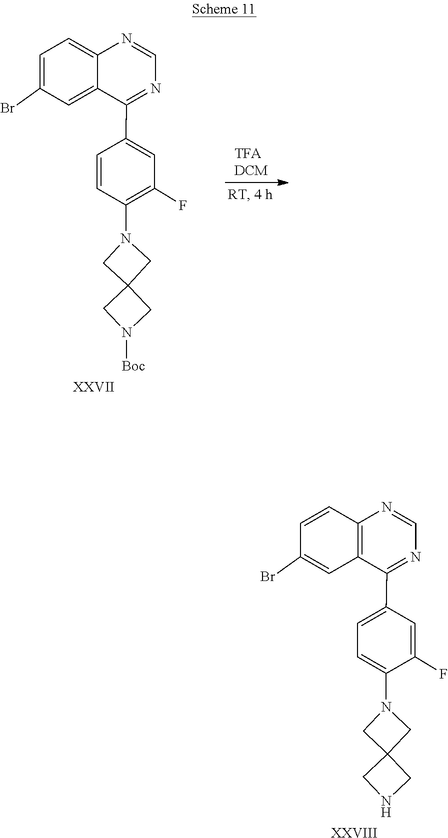

In a 5 mL microwave vial, tert-butyl 6-(4-(6-bromoquinazolin-4-yl)-2-fluorophenyl)-2,6-diazaspiro[3.3]heptane-2-carboxylate (XXVII) (469 mg, 1 Eq, 939 μmol), DCM (5.8 mL), and 2,2,2-trifluoroacetic acid (2.14 g, 1.44 mL, 20 Eq, 18.8 mmol) were added to a vial. The mixture stirred at 25° C. for 4 hour. After this time, the mixture was poured into DCM and washed with water to afford 6-bromo-4-(3-fluoro-4-(2,6-diazaspiro[3.3]heptan-2-yl)phenyl)quinazoline (XXVIII) (300 mg, 751 μmol, 80%) solid. ¹H NMR (500 MHz, CDCl₃) δ 9.30-9.27 (m, 1H), 8.30 (ddt, J=4.6, 2.7, 1.7 Hz, 1H), 7.96-7.92 (m, 2H), 7.51-7.44 (m, 2H), 6.62-6.56 (m, 1H), 4.27-4.21 (m, 4H), 4.17-4.10 (m, 4H), 1.88 (d, J=5.1 Hz, 1H).

### Example 2.1: 4-(4-(3-fluoro-4-(2-oxa-6-azaspiro[3.3]heptan-6-yl)phenyl)quinazolin-6-yl)pyridin-2-amine (28)

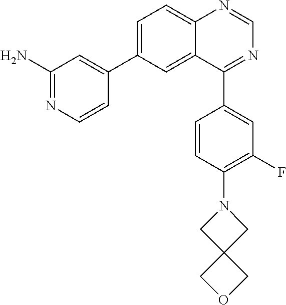 [SMILES: Nc1cc(-c2ccc3ncnc(-c4ccc(N5CC6(COC6)C5)c(F)c4)c3c2)ccn1]

### Step A. 6-(4-bromo-2-fluorophenyl)-2-oxa-6-azaspiro[3.3]heptane

In a 100 mL RBF purged and maintained with an inert atmosphere of argon, was placed a solution of 24 hour. 4-bromo-2-fluoro-1-iodobenzene (484 mg, 1 Eq, 1.61 mmol) in Toluene (9.5 mL) was added Pd2(dba)3 (44 mg, 0.03 Eq, 48.3 μmol), cesium carbonate (1.31 g, 2.5 Eq, 4.02 mmol), 2-oxa-6-azaspiro[3.3]heptane (160 mg, 1.00 Eq, 1.61 mmol), and 4,5-Bis(diphenylphosphino)-9,9-dimethyl xanthene (93 mg, 0.1 Eq, 161 μmol). The resulting solution was stirred at 100 C for. The reaction was then quenched by the addition of 25 mL of water and was extracted with 25 mL of ethyl acetate and the organized layers were combined and dried over anhydrous sodium sulfate and concentrated under reduced pressure. The crude residue was purified by automated column chromatography using a CombiFlash (0-15% EtOAc/Hex) to give 6-(4-bromo-2-fluorophenyl)-2-oxa-6-azaspiro[3.3]heptane (0.22 g, 0.81 mmol, 50%) as a yellow solid. LCMS [M+H]⁺=272.

### Step B. 6-(2-fluoro-4-(4,4,5,5-tetramethyl-1,3,2-dioxaborolan-2-yl)phenyl)-2-oxa-6-azaspiro[3.3]heptane

6-(4-bromo-2-fluorophenyl)-2-oxa-6-azaspiro[3.3]heptane (280 mg, 1 Eq, 1.03 mmol), 4,4,4′,4′,5,5,5′,5′-octamethyl-2,2′-bi(1,3,2-dioxaborolane) (392 mg, 1.5 Eq, 1.54 mmol), catalyst (17 mg, 0.02 Eq, 20.6 μmol), and potassium acetate (303 mg, 3.0 Eq, 3.09 mmol) were added to a 40 mL MW vial and placed under inert conditions for 10 min. 1,4-Dioxane (5 mL) was added to the vial and stirred for an additional 10 min under inert conditions. Reaction was placed in oil bath at 80° C. for 12 hour. When reaction was cooled contents were diluted with EtOAc, washed with brine (1×20 mL) and water (1×20 mL). Org layer was filtered through celite and dried with sodium sulfate. Drying agent was filtered out via cotton plug and the solvent was evaporated under pressure. Reaction dry loaded was purified via column chromatography (0-50% EtOAc/Hex) and 6-(2-fluoro-4-(4,4,5,5-tetramethyl-1,3,2-dioxaborolan-2-yl)phenyl)-2-oxa-6-azaspiro[3.3]heptane (276 mg, 865 μmol, 84.0%) was isolated as orange solid. LCMS [M+H]⁺=320.

### Step C. 6-(4-(6-bromoquinazolin-4-yl)-2-fluorophenyl)-2-oxa-6-azaspiro[3.3]heptane

In a vial 6-bromo-4-chloroquinazoline (73 mg, 1 Eq, 0.300 mmol), 6-(2-fluoro-4-(4,4,5,5-tetramethyl-1,3,2-dioxaborolan-2-yl)phenyl)-2-oxa-6-azaspiro[3.3]heptane (105 mg, 1.1 Eq, 330 μmol), PdCl2dppf (22 mg, 0.1 Eq, 30.0 μmol), K3PO4 (127 mg, 2 Eq, 600 μmol) and purged for 10 min. dioxane (2.58 mL), Water (430 μL) (6:1) were added to the vial and purged for an additional 10 min. The reaction was placed in the oil bath at 90° C. for 16 hour. Upon completion, the reaction was cooled and diluted with EtOAc. Org layer was washed with brine (20 mL) and water (20 mL). The recovered org layer was filtered through celite and then dried with sodium sulfate. Solvent was removed under vacuo and then separated though column chromatography (0-10% DCM/MeOH) to afford 6-(4-(6-bromoquinazolin-4-yl)-2-fluorophenyl)-2-oxa-6-azaspiro[3.3]heptane (53 mg, 0.13 mmol, 44%) as a yellow solid. LCMS [M+H]⁺=399. ¹H NMR (500 MHz, CDCl₃) δ 9.30 (s, 1H), 8.33 (s, 1H), 7.96 (s, 2H), 7.56-7.43 (m, 2H), 6.61 (t, J=8.6 Hz, 1H), 4.89 (s, 4H), 4.29 (d, J=2.1 Hz, 4H). ¹³C NMR (126 MHz, CDCl₃) δ 165.79, 154.74, 153.03, 151.10, 149.93, 140.74, 140.66, 137.04, 130.69, 129.04, 126.80, 121.94, 121.38, 117.53, 113.98, 113.95, 62.82, 39.82.

### Step D. 4-(4-(3-fluoro-4-(2-oxa-6-azaspiro[3.3]heptan-6-yl)phenyl)quinazolin-6-yl)pyridin-2-amine (28)

A 5 mL MWV was loaded with 6-(4-(6-bromoquinazolin-4-yl)-2-fluorophenyl)-2-oxa-6-azaspiro[3.3]heptane (53 mg, 1 Eq, 0.13 mmol), 4-(4,4,5,5-tetramethyl-1,3,2-dioxaborolan-2-yl)pyridin-2-amine (32 mg, 1.1 Eq, 0.15 mmol), PdCl2(dppf) (10 mg, 0.1 Eq, 13 μmol), and tripotassium phosphate (42 mg, 1.5 Eq, 0.20 mmol), the vial capped then degassed for 10 minutes then injected with solvents degassed 1,4-Dioxane (0.46 mL):Water (77 μL), (6:1 v/v), degassed further for 10 min and heated to 110° C. for 3 hour in an oil bath. The mixture was diluted with H2O and EtOAc and the aq layer was extracted 3× with EtOAc. The pooled EtOAc extracts were washed 2× with brine and then the EtOAc layer was evaporated under vacuum and re-concentrated with DCM, dry loaded onto silica gel and purified on a 12 g silica gel column (DCM/MeOH 0-10%), affording 4-(4-(3-fluoro-4-(2-oxa-6-azaspiro[3.3]heptan-6-yl)phenyl)quinazolin-6-yl)pyridin-2-amine (39 mg, 94 μmol, 71%) as a yellow solid. [M+H]⁺=414. ¹H NMR (500 MHz, CDCl₃) δ 9.30 (s, 1H), 8.32 (d, J=1.9 Hz, 1H), 8.14 (q, J=6.8 Hz, 1H), 8.07 (dd, J=8.7, 1.9 Hz, 1H), 7.59-7.51 (m, 1H), 7.51-7.42 (m, 1H), 6.89 (d, J=5.1 Hz, 1H), 6.73 (s, 1H), 6.62 (dd, J=22.3, 7.3 Hz, 1H), 6.59-6.51 (m, 1H), 4.86 (s, 4H), 4.26 (d, J=2.1 Hz, 4H), 2.71 (d, J=109.7 Hz, 2H). ¹³C NMR (126 MHz, CDCl₃) δ 167.11, 158.80, 155.04, 153.10, 151.28, 149.40, 148.10, 140.63, 138.86, 137.88, 132.36, 129.78, 127.01, 125.10, 122.94, 117.65, 114.00, 112.69, 106.80, 81.02, 62.83, 24.76.

### Example 2.2: 4-(4-(4-((1S,4S)-2-oxa-5-azabicyclo[2.2.1]heptan-5-yl)-3-fluorophenyl)quinazolin-6-yl)pyridin-2-amine (29)

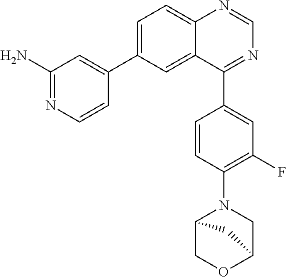 [SMILES: Nc1cc(-c2ccc3ncnc(-c4ccc(N5C[C@@H]6C[C@H]5CO6)c(F)c4)c3c2)ccn1]

### Step A. 5-(4-bromo-2-fluorophenyl)-2-oxa-5-azabicyclo[2.2.1]heptane

From with 2-oxa-5-azabicyclo[2.2.1]heptane, the title compound was isolated in a similar manner as depicted in Example 2.1, Step A, as a white solid. LCMS [M+H]⁺=271.

### Step B. (1S,4S)-5-(2-fluoro-4-(4,4,5,5-tetramethyl-1,3,2-dioxaborolan-2-yl)phenyl)-2-oxa-5-azabicyclo[2.2.1]heptane

From with 5-(4-bromo-2-fluorophenyl)-2-oxa-5-azabicyclo[2.2.1]heptane, the title compound was isolated in a similar manner as depicted in Example 2.1, Step B, as a orange solid. LCMS [M+H]⁺=319.

### Step C. (1S,4S)-5-(4-(6-bromoquinazolin-4-yl)-2-fluorophenyl)-2-oxa-5-azabicyclo[2.2.1]heptane

From with (1S,4S)-5-(2-fluoro-4-(4,4,5,5-tetramethyl-1,3,2-dioxaborolan-2-yl)phenyl)-2-oxa-5-azabicyclo[2.2.1]heptane, the title compound was isolated in a similar manner as depicted in Example 2.1, Step C, as a yellow solid. LCMS [M+H]J=399. ¹H NMR (500 MHz, CDCl₃) δ 9.27 (s, 1H), 8.36 (d, J=2.0 Hz, 1H), 8.02-7.92 (m, 2H), 7.56 (dd, J=14.5, 2.1 Hz, 1H), 7.50 (dd, J=8.4, 2.1 Hz, 1H), 6.76 (t, J=8.7 Hz, 1H), 4.74-4.64 (m, 2H), 4.09-4.01 (m, 1H), 3.91 (dd, J=7.8, 1.5 Hz, 1H), 3.74 (ddd, J=9.9, 3.1, 1.6 Hz, 1H), 3.39 (dt, J=10.2, 2.2 Hz, 1H), 2.10-2.04 (m, 1H), 2.04-1.93 (m, 2H). ¹³C NMR (126 MHz, CDCl₃) δ 165.87, 154.31, 152.61, 150.68, 149.47, 138.24, 138.17, 137.23, 130.34, 129.20, 127.29, 125.28, 123.79, 121.52, 118.36, 115.54, 76.04, 72.57, 59.48, 36.74.

### Step D. 4-(4-(4-((1S,4S)-2-oxa-5-azabicyclo[2.2.1]heptan-5-yl)-3-fluorophenyl)quinazolin-6-yl)pyridin-2-amine (29)

From with (1S,4S)-5-(4-(6-bromoquinazolin-4-yl)-2-fluorophenyl)-2-oxa-5-azabicyclo[2.2.1]heptane, the title compound was isolated in a similar manner as depicted in Example 2.1, Step D, as a yellow solid. LCMS [M+H]⁺=414. ¹H NMR (500 MHz, CDCl₃) δ 9.29 (s, 1H), 8.38 (s, 1H), 8.13 (d, J=8.7 Hz, 1H), 8.07 (d, J=8.6 Hz, 1H), 7.64-7.57 (m, 1H), 7.57-7.49 (m, 2H), 6.91 (d, J=4.9 Hz, 1H), 6.80-6.72 (m, 2H), 4.67 (d, J=12.0 Hz, 1H), 4.04 (d, J=7.7 Hz, 1H), 3.90 (d, J=7.7 Hz, 1H), 3.74 (dd, J=10.3, 2.6 Hz, 1H). 3.46 (s, 1H), 3.39 (dd, J=9.8, 2.7 Hz, 1H), 2.67-2.33 (s, 2H), 2.04 (dd, J=9.9, 2.2 Hz, 1H), 2.00-1.94 (m, 1H). ¹³C NMR (126 MHz, CDCl₃) δ 166.99, 158.63, 155.09, 152.69, 151.45, 150.76, 149.66, 148.52, 147.55, 137.97, 137.66, 132.26, 129.78, 127.18, 125.23, 122.92, 117.07, 112.65, 107.00, 72.54, 59.48, 36.73, 29.71, 28.32.

### Example 2.3: 4-(3-fluoro-4-(piperazin-1-yl)phenyl)-6-(1H-pyrrolo[2,3-b]pyridin-3-yl)quinazoline (33)

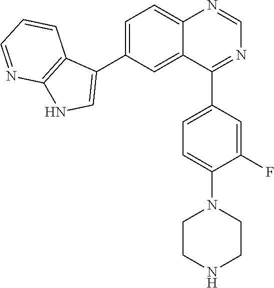 [SMILES: Fc1cc(-c2ncnc3ccc(-c4c[nH]c5ncccc45)cc23)ccc1N1CCNCC1]

### Step A. tert-butyl 4-(4-bromo-2-fluorophenyl)piperazine-1-carboxylate

From with tert-butyl piperazine-1-carboxylate, the title compound was isolated in a similar manner as depicted in Example 2.1, Step A, as a brown solid. LCMS [M+H]⁺=360.

### Step B. tert-butyl 4-(2-fluoro-4-(4,4,5,5-tetramethyl-1,3,2-dioxaborolan-2-yl)phenyl)piperazine-1-carboxylate

From with tert-butyl 4-(4-bromo-2-fluorophenyl)piperazine-1-carboxylate, the title compound was isolated in a similar manner as depicted in Example 2.1, Step B. ¹H NMR (500 MHz, CDCl₃) δ 7.28 (dd, J=7.9, 1.5 Hz, 1H), 7.26-7.20 (m, 1H), 6.68 (t, J=8.2 Hz, 1H), 3.53-3.25 (m, 4H), 2.86 (t, J=5.1 Hz, 4H), 1.26 (s, 9H), 1.10 (s, 12H). ¹³C NMR (126 MHz, CDC3) δ 155.94, 154.68, 153.99, 142.43, 142.36, 131.41, 131.38, 128.96, 128.38, 121.99, 121.84, 118.22, 118.20, 83.82, 79.85, 50.18, 50.16, 28.43, 25.02.

### Step C. tert-butyl 4-(4-(6-bromoquinazolin-4-yl)-2-fluorophenyl)piperazine-1-carboxylate

From with tert-butyl 4-(4-bromo-2-fluorophenyl)piperazine-1-carboxylate, the title compound was isolated in a similar manner as depicted in Example 2.1, Step C. LCMS [M+H]⁺=489. ¹H NMR (500 MHz, CDC3) δ 9.47 (s, 1H), 8.45 (dd, J=1.7, 0.9 Hz, 1H), 8.11 (q, J=2.3, 1.7 Hz, 2H), 7.71-7.62 (m, 2H), 7.25 (d, J=8.4 Hz, 1H), 3.78 (t, J=5.0 Hz, 4H), 3.33 (t, J=5.0 Hz, 4H), 1.64 (s, 9H). ¹³C NMR (126 MHz, CDC3) δ 155.94, 154.68, 153.99, 142.43, 142.36, 131.41, 131.38, 128.96, 128.38, 121.99, 121.84, 118.22, 118.20, 83.82, 79.85, 50.18, 28.43, 25.02.

### Step D. 4-(3-fluoro-4-(piperazin-1-yl)phenyl)-6-(1H-pyrrolo[2,3-b]pyridin-3-yl)quinazoline (33)

A mixture of tert-butyl 4-(4-(6-bromoquinazolin-4-yl)-2-fluorophenyl)piperazine-1-carboxylate (130 mg, 1 eq, 267 gmol), tert-butyl 3-(4,4,5,5-tetramethyl-1,3,2-dioxaborolan-2-yl)-1H-pyrrolo[2,3-b]pyridine-1-carboxylate (110 mg, 1.2 eq, 320 μmol), potassium phosphate (85 mg, 1.5 eq, 400 μmol), and PdCl₂(dppf) (21 mg, 0.1 eq, 26.7 μmol) in 1,4-Dioxane (0.8 mL) and water (0.1 mL) was heated at 90° C. under argon atmosphere for 2 hrs. The reaction mixture was cooled to room temperature, and diluted with ethyl acetate (10 ml), washed with water (5 ml). The aqueous solution was washed with ethyl acetate (10 ml). The combined organic layer was washed with brine (10 ml), dried over sodium sulfate, filtered, and evaporated in vacuo to get the crude product. The crude product was ran through silica gel flash column (eluent, ethyl acetate in hexanes=0 to 100%) to obtain the intermediate. The intermediate was further dissolved in DCM (3 ml) and TFA (1 ml), and the reaction solution was stirred at rt for another 4 hrs. The reaction solvent was removed, and the crude product was purified with pre-HPLC (eluent MeOH:water=8-100%) to afford 4-(3-fluoro-4-(piperazin-1-yl)phenyl)-6-(1H-pyrrolo[2,3-b]pyridin-3-yl)quinazoline (36 mg, 85 μmol, 32%). Mp 242-244° C. LCMS [M+H]⁺=425. ¹H NMR (500 MHz, DMSO-d₆) δ 12.12 (s, 1H), 9.19 (s, 1H), 8.37 (dd, J=8.7, 2.0 Hz, 1H), 8.29 (d, J=1.9 Hz, 1H), 8.25 (dt, J=5.8, 2.9 Hz, 1H), 8.17 (dd, J=8.1, 1.6 Hz, 1H), 8.09-8.02 (m, 2H), 7.80-7.63 (m, 2H), 7.26 (t, J=8.7 Hz. 1H), 7.14 (dd, J=8.0, 4.6 Hz, 1H), 3.23 (t, J=4.6 Hz, 4H), 3.10 (d, J=4.3 Hz, 4H). ¹³C NMR (126 MHz, DMSO-d₆) δ 165.41, 155.77, 154.07, 149.73, 149.70, 143.82, 141.39, 135.41, 133.89, 131.28, 129.43, 127.50, 126.16, 123.21, 121.86, 119.56, 118.19, 118.01, 117.53, 116.94, 113.54, 48.90, 44.29.

### Example 2.4: 1-(4-(4-(6-(2-aminopyridin-4-yl)quinazolin-4-yl)-2-fluorophenyl)piperazin-1-yl)ethan-1-one (34)

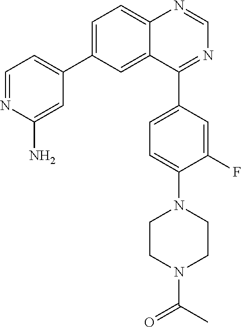 [SMILES: CC(=O)N1CCN(c2ccc(-c3ncnc4ccc(-c5ccnc(N)c5)cc34)cc2F)CC1]

### Step A. 1-(4-bromo-2-fluorophenyl)piperazine

In a 100 mL RBF purged and maintained with an inert atmosphere of argon, was placed a solution of 24 hour 4-bromo-2-fluoro-1-iodobenzene (912 mg, 1 eq, 3.03 mmol) in Toluene (18 mL) was added Pd2(dba)3 (83 mg, 0.03 eq, 90.9 μmol), cesium carbonate (2.47 g, 2.5 eq, 7.58 mmol), 4,5-Bis(diphenylphosphino)-9,9-dimethyl xanthene (175 mg, 0.1 eq, 303 μmol). The resulting solution was stirred at 100 C for 24 hours. The reaction was then quenched by the addition of 25 mL of water and was extracted with 25 mL of ethyl acetate and the organized layers were combined and dried over anhydrous sodium sulfate and concentrated under reduced pressure. The crude residue was purified by automated column chromatography using a CombiFlash (0-15% EtOAc/Hex) to give tert-butyl 4-(4-bromo-2-fluorophenyl)piperazine-1-carboxylate (748 mg, 2.08 mmol, 69%) as a light-yellow solid. LCMS [M+H]⁺=359. tert-butyl 4-(4-bromo-2-fluorophenyl)piperazine-1-carboxylate (709 mg, 1 eq, 1.97 mmol) and DCM (20 mL) were added to a vial stirring. 2,2,2-trifluoroacetic acid (15 g, 9.97 mL, 66 eq, 130 mmol) was added, with the reaction allowed to vent. The reaction stirred at 25° C. for 3 hours. Upon completion the reaction was basified with TN NaOH and diluted w/DCM (2×10 mL). Org layer was washed with water, dried with sodium sulfate, and then reduced under pressure and purified via column chromatography (DCM/MeOH 0-10%) to afford 1-(4-bromo-2-fluorophenyl)piperazine (376 mg, 1.45 mmol, 74%) as a yellow brown solid. LCMS [M+H]⁺=259. ¹H NMR (500 MHz, CDCl₃) δ 9.74 (s, 1H), 7.24-7.18 (m, 3H), 6.81 (td, J=8.7, 1.9 Hz, 1H), 3.33 (d, J=17.2 Hz, 8H). ¹³C NMR (126 MHz, CDCl₃) δ 155.47, 137.74, 127.85, 120.64, 120.03, 115.74, 77.28, 77.02, 76.77, 47.42, 43.60.

### Step B. 1-(4-(4-bromo-2-fluorophenyl)piperazin-1-yl)ethan-1-one

1-(4-bromo-2-fluorophenyl)piperazine (0.350 g, 1 eq, 1.35 mmol) and triethylamine (396 mg, 546 μL, 2.9 eq, 3.92 mmol) were added to DCM (4.5 mL). The reaction was cooled to 0 C and then Acetyl chloride (233 mg, 208 μL, 2.2 eq, 2.97 mmol) was added slowly. Once completely added, the reaction was warmed to 25° C. and stirred for 16 hour. Upon completion, the reaction was quenched with brine solution and diluted with DCM. Org. phase was washed additionally with brine and water, and then dried using sodium sulfate. The mixture was reduced under pressure and purified through column chromatography (0-5% DCM/MeOH) 1-(4-(4-bromo-2-fluorophenyl)piperazin-1-yl)ethan-1-one (257 mg, 853 μmol, 63.2%) was isolated as a yellow oil. LCMS [M+H]⁺=302. ¹H NMR (500 MHz, CDCl₃) δ 7.30-7.21 (m, 2H), 6.87-6.83 (m, 1H), 3.86-3.76 (m, 2H), 3.76-3.65 (m, 2H), 3.13-3.09 (m, 2H), 3.09-3.03 (m, 2H), 2.19 (s, 4H). ¹³C NMR (126 MHz, CDCl₃) δ 169.03, 154.38, 138.86, 127.56, 120.34, 119.70, 114.34, 50.71, 50.14, 46.31, 41.35, 21.28.

### Step C. 1-(4-(2-fluoro-4-(4,4,5,5-tetramethyl-1,3,2-dioxaborolan-2-yl)phenyl)piperazin-1-yl)ethan-1-one

From with 1-(4-(4-bromo-2-fluorophenyl)piperazin-1-yl)ethan-1-one, the title compound was isolated in a similar manner as depicted in Example 2.1, Step B, as a pink solid. LCMS [M+H]⁺=349. ¹H NMR (500 MHz, CDCl₃) δ 7.49 (dd, J=7.9, 1.5 Hz, 1H), 7.44 (dd, J=13.2, 1.4 Hz, 1H), 6.91 (t, J=8.2 Hz, 1H), 3.77 (t, J=5.2 Hz, 2H), 3.64-3.59 (m, 2H), 3.14-3.07 (m, 4H), 2.11 (s, 3H), 1.31 (s, 12H). ¹³C NMR (126 MHz, CDCl₃) δ 169.00, 155.96, 154.00, 141.81, 131.44, 122.02, 118.35, 83.91, 50.60, 50.01, 46.31, 41.34, 24.72, 21.35.

### Step D. 1-(4-(4-(6-bromoquinazolin-4-yl)-2-fluorophenyl)piperazin-1-yl)ethan-1-one

From 1-(4-(2-fluoro-4-(4,4,5,5-tetramethyl-1,3,2-dioxaborolan-2-yl)phenyl)piperazin-1-yl)ethan-1-one, the title compound was isolated in a similar manner as depicted in Example 2.1, Step C, as an orange solid. LCMS [M+H]⁺=430. ¹H NMR (500 MHz, CDCl₃) δ 9.32 (s, 1H), 8.28 (d, J=1.4 Hz, 1H), 7.96 (d, J=1.4 Hz, 2H), 7.54-7.49 (m, 2H), 7.08 (t, J=8.4 Hz, 1H), 3.82 (t, J=5.0 Hz, 2H), 3.66 (t, J=4.9 Hz, 2H), 3.21 (dt, J=21.9, 5.0 Hz, 4H), 2.15 (s, 3H). ¹³C NMR (126 MHz, CDCl₃) δ 169.14, 165.60, 156.25, 154.78, 154.28, 149.93, 141.58, 137.29, 130.79, 128.84, 126.68, 123.89, 121.70, 119.01, 118.00, 50.50, 49.91, 46.36, 41.39, 21.38.

### Step E. 1-(4-(4-(6-(2-aminopyridin-4-yl)quinazolin-4-yl)-2-fluorophenyl)piperazin-1-yl)ethan-1-one (34)

From 1-(4-(4-(6-bromoquinazolin-4-yl)-2-fluorophenyl)piperazin-1-yl)ethan-1-one, the title compound was isolated in a similar manner as depicted in Example 2.1, Step D, as a yellow solid. LCMS [M+H]⁺=443. ¹H NMR (500 MHz, CDCl₃) δ 9.34 (s, 1H), 8.31 (d, J=2.0 Hz, 1H), 8.18 (d, J=8.7 Hz, 1H), 8.11 (td, J=9.2, 3.7 Hz, 2H), 7.61-7.54 (m, 2H), 7.10 (t, J=8.4 Hz, 1H), 6.89 (d, J=5.2 Hz, 1H), 6.73 (s, 1H), 4.82 (s, 2H), 3.82 (t, J=5.1 Hz, 2H), 3.67 (t, J=5.0 Hz, 2H), 3.22 (dt, J=21.7, 5.1 Hz, 4H), 2.15 (s, 3H). ¹³C NMR (126 MHz, CDCl₃) δ 169.12, 166.86, 158.69, 155.02, 154.30, 151.34, 149.38, 147.99, 138.18, 132.60, 131.23, 129.90, 126.77, 124.87, 122.94, 119.02, 118.11, 112.70, 106.86, 50.54, 49.91, 46.36, 41.39, 21.37.

### Example 2.5: 4-(4-(3-fluoro-4-(piperazin-1-yl)phenyl)quinazolin-6-yl)pyridin-2-amine (35)

### Step A. tert-butyl 4-(4-(6-br

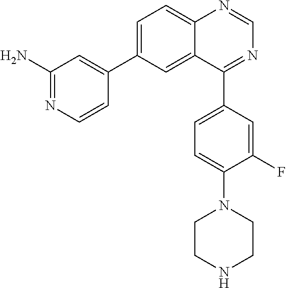 [SMILES: Nc1cc(-c2ccc3ncnc(-c4ccc(N5CCNCC5)c(F)c4)c3c2)ccn1]

l)-2-fluorophenyl)piperazine-1-carboxylate

From tert-butyl 4-(4-bromo-2-fluorophenyl)piperazine-1-carboxylate, the title compound was isolated in a similar manner as depicted in Example 2.1, Step C, as a yellow solid. LCMS [M+H]⁺=488. ¹H NMR (500 MHz, CDCl₃) δ 9.32 (s, 1H), 8.30 (dd, J=1.7, 0.9 Hz, 1H), 7.99-7.95 (m, 2H), 7.56-7.49 (m, 2H), 7.09 (t, J=8.6 Hz, 1H), 3.63 (t, J=5.0 Hz, 4H), 3.17 (t, J=5.0 Hz, 4H), 1.48 (s, 9H). ¹³C NMR (126 MHz, CDCl₃) δ 165.73, 156.23, 154.69, 154.26, 149.81, 142.04, 137.31, 130.72, 130.39, 128.92, 126.71, 123.89, 121.71, 118.96, 117.98, 80.06, 50.15, 50.12, 28.45.

### Step B. 4-(4-(3-fluoro-4-(piperazin-1-yl)phenyl)quinazolin-6-yl)pyridin-2-amine (35)

From tert-butyl 4-(4-(6-bromoquinazolin-4-yl)-2-fluorophenyl)piperazine-1-carboxylate, the title compound was isolated in a similar manner as depicted in Example 2.3, Step D, as a yellow solid. LCMS [M+H]⁺=401. ¹H NMR (500 MHz, CDCl₃) δ 9.35 (s, 1H), 8.35 (d, J=2.0 Hz, 1H), 8.19-8.16 (m, 2H), 8.11 (dd, J=8.7, 2.0 Hz, 1H), 7.60-7.56 (m, 2H), 7.12 (t, J=8.6 Hz, 1H), 6.91 (dd, J=5.4, 1.6 Hz, 1H), 6.72 (d, J=1.6 Hz, 1H), 4.60 (s, 2H), 3.28 (d, J=4.6 Hz, 1H), 3.25 (dd, J=6.2, 3.6 Hz, 4H), 3.12 (dd, J=6.4, 3.4 Hz, 4H). ¹³C NMR (126 MHz, CDCl₃) δ 166.98, 159.03, 156.22, 154.96, 154.25, 151.27, 149.00, 142.41, 138.31, 132.58, 130.45, 129.77, 126.73, 124.87, 122.97, 118.78, 118.00, 112.80, 106.48, 51.22, 46.00, 29.70.

### Example 2.6: 1-((1S,4S)-5-(4-(6-(2-aminopyridin-4-yl)quinazolin-4-yl)phenyl)-2,5-diazabicyclo[2.2.1]heptan-2-yl)ethan-1-one (39)

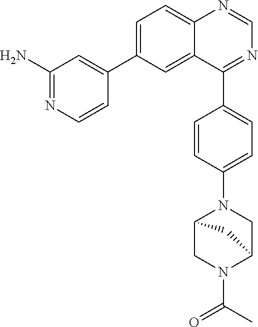 [SMILES: CC(=O)N1C[C@@H]2C[C@H]1CN2c1ccc(-c2ncnc3ccc(-c4ccnc(N)c4)cc23)cc1]

### Step A. (1S,4S)-2-(4-bromophenyl)-2,5-diazabicyclo[2.2.1]heptane

Benzene, 1-bromo-4-iodo- (400 mg, 1 eq, 1.41 mmol) and (1S,4S)-2-boc-2,5-diazabicyclo(2,2,1)heptane (140 mg, 0.5 eq, 707 mol) were suspended in Toluene (2.1 mL), and the reaction mixture was degassed with a argon stream. sodium 2-methylpropan-2-olate (543 mg, 610 μL, 4 eq, 5.66 mmol) was added, and the reaction mixture was sonicated, under a nitrogen atmosphere, until the solids were mostly dissolved, and the solution was homogeneous. 1,1′-Bis(diphenylphosphino)ferrocene-palladium(II) dichloride (52 mg, 0.05 eq, 70.7 mol) was added, and the reaction mixture was heated under nitrogen at 90° C. for 16 hour. The solution was filtered through celite, washed with ethyl acetate and the filtrate was evaporated under reduced pressure. The crude residue was purified by silica gel chromatography using a 0% to 50% Ethyl acetate in hexanes eluent gradient to afford tert-butyl (1S,4S)-5-(4-bromophenyl)-2,5-diazabicyclo[2.2.1]heptane-2-carboxylate (366 mg, 1.04 mmol, 73%). LCMS [M+H]⁺=353

### Step B. (1S,4S)-2-(4-bromophenyl)-2,5-diazabicyclo[2.2.1]heptane

tert-butyl (1S,4S)-5-(4-bromophenyl)-2,5-diazabicyclo[2.2.1]heptane-2-carboxylate (366 mg, 1 eq, 1.04 mmol) in DCM (10.5 mL) TFA (7.80 g, 5.27 mL, 66 eq, 68.4 mmol) was added unde was added under argon and allowed to stir for 10 min. The reaction was at 25° C. and allowed to stir for 4 hours. The reaction was basified with 1N NaOH and diluted with DCM to produce (1S,4S)-2-(4-bromophenyl)-2,5-diazabicyclo[2.2.1]heptane, 2,2,2-trifluoroacetate salt (223 mg, 609 mol, 59%) as a final product. [M+H]⁺=252.

### Step C. 1-((1S,4S)-5-(4-bromophenyl)-2,5-diazabicyclo[2.2.1]heptan-2-yl)ethan-1-one

From (1S,4S)-2-(4-bromophenyl)-2,5-diazabicyclo[2.2.1]heptane, the title compound was isolated in a similar manner as depicted in Example 2.4, Step B, as a brown solid. LCMS [M+H]⁺=294. ¹H NMR (500 MHz, CDCl₃) δ 7.30 (t, J=8.8 Hz, 2H), 6.43 (t, J=8.2 Hz, 2H), 4.99 (s, 1H), 4.45 (d, J=13.9 Hz, 1H), 4.39 (s, 1H), 3.66 (d, J=8.4 Hz, 1H), 3.57 (dd, J=13.0, 9.8 Hz, 1H), 3.49 (d, J=16.0 Hz, 2H), 3.19-3.08 (m, 1H), 1.92 (s, 3H). ¹³C NMR (126 MHz, CDCl₃) δ 168.74, 145.23, 132.13, 114.44, 109.31, 58.78, 57.77, 56.92, 55.46, 36.98, 29.71.

### Step D. 1-((1S,4S)-5-(4-(4,4,5,5-tetramethyl-1,3,2-dioxaborolan-2-yl)phenyl)-2,5-diazabicyclo[2.2.1]heptan-2-yl)ethan-1-one

From with 1-((1S,4S)-5-(4-bromophenyl)-2,5-diazabicyclo[2.2.1]heptan-2-yl)ethan-1-one, the title compound was isolated in a similar manner as depicted in Example 2.1, Step B, as a purple oil. LCMS [M+H]⁺=343.1H NMR (500 MHz, CDCl₃) δ 7.67 (dd, J=11.7, 7.8 Hz, 2H), 6.54 (t, J=8.8 Hz, 2H), 4.99 (d, J=6.3 Hz, 1H), 4.55-4.42 (m, 2H), 3.58 (p, J=9.0 Hz, 2H), 3.52 (s, 2H), 3.25 (d, J=8.8 Hz, 1H), 1.91 (d, J=7.6 Hz, 3H), 1.32 (s, 12H). ¹³C NMR (126 MHz, CDCl₃) δ 148.68, 136.48, 132.15, 114.23, 111.70, 83.31, 57.42, 57.15, 56.46, 55.54, 37.16, 24.87.

### Step E. 1-((1S,4S)-5-(4-(6-bromoquinazolin-4-yl)phenyl)-2,5-diazabicyclo[2.2.1]heptan-2-yl)ethan-1-one

From with 1-((1S,4S)-5-(4-(4,4,5,5-tetramethyl-1,3,2-dioxaborolan-2-yl)phenyl)-2,5-diazabicyclo[2.2.1]heptan-2-yl)ethan-1-one, the title compound was isolated in a similar manner as depicted in Example 2.1, Step C. LCMS [M+H]⁺=422. ¹H NMR (500 MHz, DMSO-d₆) δ 9.26 (s, 1H), 8.32 (d, J=2.2 Hz, 1H), 8.13 (dt, J=8.9, 1.7 Hz, 1H), 7.98 (d, J=8.9 Hz, 1H), 7.76-7.73 (m, 2H), 6.86 (dd, J=8.5, 6.2 Hz, 2H), 4.82 (d, J=12.4 Hz, 1H), 4.71 (s, 1H), 3.69-3.65 (m, 1H), 3.61 (dd, J=9.4, 1.9 Hz, 1H), 3.45 (s, 1H), 3.13 (s, 1H), 2.54 (s, 3H), 2.04 (m, 1H), 1.85 (m, 1H).

### Step F. 1-((1S,4S)-5-(4-(6-(2-aminopyridin-4-yl)quinazolin-4-yl)phenyl)-2,5-diazabicyclo[2.2.1]heptan-2-yl)ethan-1-one (39)

From 1-((1S,4S)-5-(4-(6-bromoquinazolin-4-yl)phenyl)-2,5-diazabicyclo[2.2.1]heptan-2-yl)ethan-1-one, the title compound was isolated in a similar manner as depicted in Example 2.1, Step D, as a yellow solid. LCMS [M+H]⁺=436. ¹H NMR (500 MHz, DMSO-d₆) δ 9.28 (s, 1H), 8.38 (d, J=1.9 Hz, 1H), 8.27 (d, J=8.9 Hz, 1H), 8.15 (d, J=8.7 Hz, 1H), 8.04 (d, J=5.5 Hz, 1H), 7.69 (d, J=14.9 Hz, 1H), 7.62 (dt, J=8.6, 2.2 Hz, 1H), 7.01 (td, J=9.0, 2.7 Hz, 1H), 6.92 (t, J=5.4 Hz, 1H), 6.82 (s, 1H), 6.26 (s, 2H), 4.80 (s, 1H), 4.67 (d, J=5.9 Hz, 1H), 3.88-3.83 (m, 1H), 3.79 (d, J=9.6 Hz, 1H), 3.62 (q, J=9.5 Hz, 2H), 3.27 (dd, J=9.3, 3.3 Hz, 1H), 2.03 (d, J=13.6 Hz, 3H), 1.87 (s, 1H).

### Example 2.7: 1-((1S,4S)-5-(4-(6-(2-aminopyridin-4-yl)quinazolin-4-yl)-2-fluorophenyl)-2,5-diazabicyclo[2.2.1]heptan-2-yl)ethan-1-one (40)

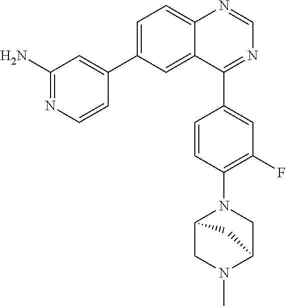 [SMILES: CN1C[C@@H]2C[C@H]1CN2c1ccc(-c2ncnc3ccc(-c4ccnc(N)c4)cc23)cc1F]

### Step A. (1S,4S)-2-(4-bromo-2-fluorophenyl)-2,5-diazabicyclo[2.2.1]heptane

From (1S,4S)-2-boc-2,5-diazabicyclo(2,2,1)heptane, the title compound was isolated in a similar manner as depicted in Example 2.4, Step A, as a yellow solid. LCMS [M+H]⁺=271.

### Step B. 1-((1S,4S)-5-(4-bromo-2-fluorophenyl)-2,5-diazabicyclo[2.2.1]heptan-2-yl)ethan-1-one

From (1S,4S)-2-(4-bromophenyl)-2,5-diazabicyclo[2.2.1]heptane, the title compound was isolated in a similar manner as depicted in Example 2.4, Step B, as a brown solid. LCMS [M+H]⁺=314. ¹H NMR (500 MHz, CDCl₃) δ 7.17-7.11 (m, 1H), 7.10 (s, 1H), 6.46 (t, J=9.1 Hz, 1H), 4.94 (s, 1H), 4.48-4.39 (m, 1H), 3.68-3.63 (m, 2H), 3.56-3.49 (m, 2H), 3.49-3.44 (m, 1H), 3.27-3.18 (m, 2H), 1.93 (s, 3H). ¹³C NMR (126 MHz, CDCl₃) δ 168.35, 151.51, 134.57, 127.52, 119.81, 116.51, 108.44, 59.39, 58.91, 58.04, 55.04, 37.18, 21.94.

### Step C. 1-((1S,4S)-5-(2-fluoro-4-(4,4,5,5-tetramethyl-1,3,2-dioxaborolan-2-yl)phenyl)-2,5-diazabicyclo[2.2.1]heptan-2-yl)ethan-1-one

From with 1-((1S,4S)-5-(4-bromo-2-fluorophenyl)-2,5-diazabicyclo[2.2.1]heptan-2-yl)ethan-1-one, the title compound was isolated in a similar manner as depicted in Example 2.1, Step B, as a green oil. LCMS [M+H]⁺=361. ¹H NMR (500 MHz, CDCl₃) δ 7.67 (dd, J=11.7, 7.8 Hz, 2H), 6.54 (t, J=8.8 Hz, 2H), 4.99 (d, J=6.3 Hz, 1H), 4.55-4.42 (m, 2H), 3.58 (p, J=9.0 Hz, 2H), 3.52 (s, 2H), 3.25 (d, J=8.8 Hz, 1H), 1.91 (d, J=7.6 Hz, 3H), 1.32 (s, 12H). ¹³C NMR (126 MHz, CDCl₃) δ 168.87, 148.68, 136.48, 132.15, 129.52, 114.23, 111.70, 83.31, 57.42, 57.15, 56.46, 55.54, 37.16, 24.87.

### Step D. 1-((1S,4S)-5-(4-(6-bromoquinazolin-4-yl)-2-fluorophenyl)-2,5-diazabicyclo[2.2.1]heptan-2-yl)ethan-1-one

From with 1-((1S,4S)-5-(2-fluoro-4-(4,4,5,5-tetramethyl-1,3,2-dioxaborolan-2-yl)phenyl)-2,5-diazabicyclo[2.2.1]heptan-2-yl)ethan-1-one, the title compound was isolated in a similar manner as depicted in Example 2.1, Step C. LCMS [M+H]⁺=440. ¹H NMR (500 MHz, DMSO-d₆) δ 9.29 (s, 1H), 8.32 (d, J=2.1 Hz, 1H), 8.15 (dt, J=9.1, 1.9 Hz, 1H), 8.01 (d, J=8.9 Hz, 1H), 7.63 (dd, J=14.8, 2.1 Hz, 1H), 7.56 (dt, J=8.7, 2.1 Hz, 1H), 7.02 (td, J=9.0, 3.3 Hz, 1H), 4.79 (s, 1H), 4.67 (d, J=8.5 Hz, 1H), 3.87-3.81 (m, 1H), 3.78 (d, J=9.5 Hz, 1H), 3.61 (d, J=5.6 Hz, 1H), 3.48 (d, J=10.9 Hz, 1H), 2.55 (s, 3H), 2.04 (m, 1H), 1.87 (m, 1H). ¹³C NMR (126 MHz, DMSO-d₆) δ 171.1, 167.08, 164.30, 154.06, 148.81, 143.97, 136.46, 130.12, 128.14, 126.80, 122.60, 120.12, 117.34, 117.17, 115.07, 57.24, 53.93, 51.97, 50.38, 35.35, 21.49.

### Step E. 1-((1S,4S)-5-(4-(6-(2-aminopyridin-4-yl)quinazolin-4-yl)-2-fluorophenyl)-2,5-diazabicyclo[2.2.1]heptan-2-yl)ethan-1-one (40)

From 1-((1S,4S)-5-(4-(6-bromoquinazolin-4-yl)-2-fluorophenyl)-2,5-diazabicyclo[2.2.1]heptan-2-yl)ethan-1-one, the title compound was isolated in a similar manner as depicted in Example 2.1, Step D, as a yellow solid. LCMS [M+H]⁺=455. ¹H NMR (500 MHz, DMSO-d₆) δ 9.28 (s, 1H), 8.38 (d, J=1.9 Hz, 1H), 8.27 (d, J=8.9 Hz, 1H), 8.15 (d, J=8.7 Hz, 1H), 8.04 (d, J=5.5 Hz, 1H), 7.69 (d, J=14.9 Hz, 1H), 7.62 (dt, J=8.6, 2.2 Hz, 1H), 7.01 (td, J=9.0, 2.7 Hz, 1H), 6.92 (t, J=5.4 Hz, 1H), 6.82 (s, 1H), 6.26 (s, 2H), 4.80 (s, 1H), 4.67 (d, J=5.9 Hz, 1H), 3.88-3.83 (m, 1H), 3.79 (d, J=9.6 Hz, 1H), 3.62 (q, J=9.5 Hz, 2H), 3.27 (dd, J=9.3, 3.3 Hz, 1H), 2.03 (d, J=13.6 Hz, 3H), 1.87 (s, 1H). ¹³C NMR (126 MHz, DMSO-d₆) δ 168.17, 166.49, 160.44, 155.15, 152.20, 151.20, 148.12, 138.03, 132.94, 129.75, 128.00, 125.63, 125.44, 124.81, 122.60, 115.99, 110.75, 106.31, 58.59, 55.01, 53.15, 51.55, 36.48, 21.95.

### Example 2.8: 4-(4-(4-(4-(oxetan-3-yl)piperazin-1-yl)phenyl)quinazolin-6-yl)pyridin-2-amine (42)

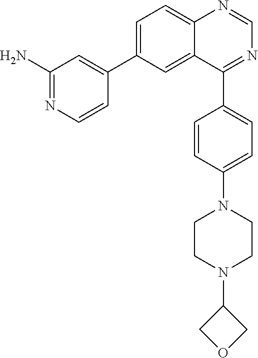 [SMILES: Nc1cc(-c2ccc3ncnc(-c4ccc(N5CCN(C6COC6)CC5)cc4)c3c2)ccn1]

### Step A. (1-(4-bromophenyl)-4-(oxetan-3-yl)piperazine

From 1-(oxetan-3-yl)piperazine, the title compound was isolated in a similar manner as depicted in Example 2.6, Step A. LCMS [M+H]⁺=297. ¹H NMR (500 MHz, CDCl₃) δ 7.35-7.32 (m, 2H), 6.81-6.76 (m, 2H), 4.69 (t, J=6.6 Hz, 2H), 4.65 (t, J=6.1 Hz, 2H), 3.22-3.16 (m, 4H), 2.50-2.46 (m, 4H). ¹³C NMR (126 MHz, CDCl₃) δ 150.21, 131.92, 117.74, 112.07, 75.42, 59.19, 49.49, 48.73.

### Step B. 1-(oxetan-3-yl)-4-(4-(4,4,5,5-tetramethyl-1,3,2-dioxaborolan-2-yl)phenyl)piperazine

From with 1-(4-bromophenyl)-4-(oxetan-3-yl)piperazine, the title compound was isolated in a similar manner as depicted in Example 2.1, Step B, as a brown solid. LCMS [M+H]⁺=345. ¹H NMR (500 MHz, CDCl₃) δ 7.70 (d, J=8.1 Hz, 2H), 6.88 (d, J=8.2 Hz, 2H), 4.73-4.67 (m, 2H), 4.65 (d, J=5.7 Hz, 2H), 3.30 (t, J=5.0 Hz, 4H), 2.53-2.44 (m, 4H), 1.22 (s, 12H). ¹³C NMR (126 MHz, CDCl₃) δ 153.17, 136.15, 129.17, 114.48, 83.79, 75.36, 59.21, 49.47, 47.81, 24.85.

### Step C. 6-bromo-4-(4-(4-(oxetan-3-yl)piperazin-1-yl)phenyl)quinazoline

From with 1-(oxetan-3-yl)-4-(4-(4,4,5,5-tetramethyl-1,3,2-dioxaborolan-2-yl)phenyl)piperazine, the title compound was isolated in a similar manner as depicted in Example 2.1, Step C, as a yellow solid. LCMS [M+H]⁺=427. ¹H NMR (500 MHz, CDCl₃) δ 9.30 (s, 1H), 8.34 (s, 1H), 7.94 (s, 2H), 7.74 (d, J=8.1 Hz, 2H), 7.07 (d, J=8.4 Hz, 2H), 4.74 (m, 4H), 3.49 (m, 4H), 2.73-2.60 (m, 3H).

### Step D. 4-(4-(4-(4-(oxetan-3-yl)piperazin-1-yl)phenyl)quinazolin-6-yl)pyridin-2-amine (42)

From with 6-bromo-4-(4-(4-(oxetan-3-yl)piperazin-1-yl)phenyl)quinazoline, the title compound was isolated in a similar manner as depicted in Example 2.1, Step D, as a yellow solid. LCMS [M+H]⁺=439. ¹H NMR (400 MHz, DMSO-d₆) δ 9.23 (s, 1H), 8.33 (d, J=2.0 Hz, 1H), 8.21 (dd, J=8.8, 2.0 Hz, 1H), 8.09 (d, J=8.7 Hz, 1H), 7.98 (d, J=5.3 Hz, 1H), 7.76 (d, J=8.5 Hz, 2H), 7.12 (d, J=8.5 Hz, 2H), 6.82 (dd, J=5.3, 1.7 Hz, 1H), 6.71 (s, 1H), 6.05 (s, 2H), 4.54 (t, J=6.5 Hz, 2H), 4.45 (t, J=6.0 Hz, 2H), 3.42 (t, J=6.3 Hz, 1H), 2.46 (t, J=5.0 Hz, 4H), 2.39 (t, J=5.0 Hz, 4H). ¹³C NMR (101 MHz, DMSO-d₆) δ 167.72, 161.06, 155.25, 152.71, 151.17, 149.41, 147.65, 138.16, 132.87, 132.09, 129.76, 126.41, 124.92, 122.76, 114.77, 110.66, 105.90, 74.89, 58.99, 49.47, 47.25.

### Example 2.9: 41-(6-(4-(6-(2-aminopyridin-4-yl)quinazolin-4-yl)phenyl)-2,6-diazaspiro[3.3]heptan-2-yl)ethan-1-one (44)

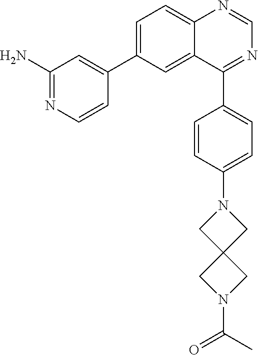 [SMILES: CC(=O)N1CC2(C1)CN(c1ccc(-c3ncnc4ccc(-c5ccnc(N)c5)cc34)cc1)C2]

### Step A. tert-butyl 6-(4-bromophenyl)-2,6-diazaspiro[3.3]heptane-2-carboxylate

From 2,6-Diazaspiro[3.3]heptane-2-carboxylic acid, the title compound was isolated in a similar manner as depicted in Example 2.6, Step A. LCMS [M+H]⁺=353.

### Step B. 2-(4-bromophenyl)-2,6-diazaspiro[3.3]heptane

From tert-butyl 6-(4-bromophenyl)-2,6-diazaspiro[3.3]heptane-2-carboxylate, the title compound was isolated in a similar manner as depicted in Example 2.6, Step B. LCMS [M+H]⁺=253.

### Step C. 1-(6-(4-bromophenyl)-2,6-diazaspiro[3.3]heptan-2-yl)ethan-1-one

From 2-(4-bromophenyl)-2,6-diazaspiro[3.3]heptane, the title compound was isolated in a similar manner as depicted in Example 2.4, Step B, as a brown solid. LCMS [M+H]⁺=294. ¹H NMR (500 MHz, CDCl₃) δ 7.25 (d, J=8.2 Hz, 2H), 6.28 (d, J=8.4 Hz, 2H), 4.25 (s, 2H), 4.11 (s, 2H), 3.92 (s, 4H), 1.84 (s, 3H). ¹³C NMR (126 MHz, CDCl₃) δ 170.52, 149.91, 131.80, 113.45, 110.44, 62.18, 60.73, 57.92, 33.04.

### Step D. 1-(6-(4-(4,4,5,5-tetramethyl-1,3,2-dioxaborolan-2-yl)phenyl)-2,6-diazaspiro[3.3]heptan-2-yl)ethan-1-one

From 1-(6-(4-bromophenyl)-2,6-diazaspiro[3.3]heptan-2-yl)ethan-1-one, the title compound was isolated in a similar manner as depicted in Example 2.1, Step B, as a brown solid. LCMS [M+H]⁺=343. ¹H NMR (500 MHz, CDCl₃) δ 7.65 (d, J=7.9 Hz, 2H), 6.41 (d, J=8.0 Hz, 2H), 4.27 (s, 2H), 4.14 (s, 2H), 4.00 (d, J=10.9 Hz, 4H), 1.86 (s, 3H), 1.22 (d, J=7.9 Hz, 12H). ¹³C NMR (126 MHz, CDCl₃) δ 170.55, 152.96, 136.03, 129.07, 110.78, 83.37, 61.91, 58.04, 33.12, 29.70, 24.85.

### Step E. 1-(6-(4-(6-bromoquinazolin-4-yl)phenyl)-2,6-diazaspiro[3.3]heptan-2-yl)ethan-1-one

From 1-(6-(4-(4,4,5,5-tetramethyl-1,3,2-dioxaborolan-2-yl)phenyl)-2,6-diazaspiro[3.3]heptan-2-yl)ethan-1-one, the title compound was isolated in a similar manner as depicted in Example 2.1, Step C, as a yellow solid. LCMS [M+H]⁺=424. ¹H NMR (400 MHz, CDCl₃) δ 9.28 (s, 1H), 8.33 (d, J=1.8 Hz, 1H), 7.96 (s, 2H), 7.73 (d, J=7.9 Hz, 2H), 6.61 (d, J=8.0 Hz, 2H), 4.35 (s, 2H), 4.21 (s, 2H), 4.15 (s, 2H), 4.04 (s, 2H), 1.90 (s, 3H).

### Step F. 41-(6-(4-(6-(2-aminopyridin-4-yl)quinazolin-4-yl)phenyl)-2,6-diazaspiro[3.3]heptan-2-yl)ethan-1-one (44)

From 1-(6-(4-(6-bromoquinazolin-4-yl)phenyl)-2,6-diazaspiro[3.3]heptan-2-yl)ethan-1-one, the title compound was isolated in a similar manner as depicted in Example 2.1, Step D, as a yellow solid. LCMS [M+H]⁺=437. ¹H NMR (400 MHz, DMSO-d₆) δ 9.21 (s, 1H), 8.30 (d, J=2.0 Hz, 1H), 8.21 (dd, J=8.9, 2.0 Hz, 1H), 8.09 (d, J=8.7 Hz, 1H), 7.98 (d, J=5.3 Hz, 1H), 7.75 (d, J=8.4 Hz, 2H), 6.81 (dd, J=5.3, 1.7 Hz, 1H), 6.70 (s, 1H), 6.62 (d, J=8.5 Hz, 2H), 6.05 (s, 2H), 4.30 (s, 2H), 4.08 (s, 4H), 4.02 (s, 3H), 1.73 (s, 3H).

### Example 2.10: 4-(4-(3-fluoro-4-(6-(oxetan-3-yl)-2,6-diazaspiro[3.3]heptan-2-yl)phenyl)quinazolin-6-yl)pyridin-2-amine (57)

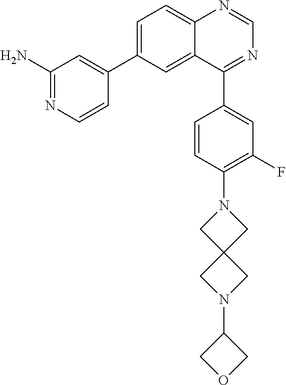 [SMILES: Nc1cc(-c2ccc3ncnc(-c4ccc(N5CC6(C5)CN(C5COC5)C6)c(F)c4)c3c2)ccn1]

### Step A. 6-bromo-4-(3-fluoro-4-(6-(oxetan-3-yl)-2,6-diazaspiro[3.3]heptan-2-yl)phenyl)quinazoline

6-bromo-4-(3-fluoro-4-(2,6-diazaspiro[3.3]heptan-2-yl)phenyl)quinazoline (XXVIII) (50 mg, 1 eq, 0.13 mmol) was dissolved in DCE (0.52 mL) followed by adding of STAB (40 mg, 1.5 eq, 0.19 mmol), Oxentan-3-on (14 mg, 12 μL, 1.5 eq, 0.19 mmol), and acetic acid (1.5 mg, 1.4 μL, 0.2 eq, 25 μmol). The resulting suspension was stirred at 25° C. for 16 hour before the solvent was removed under reduced pressure. The residue was washed with said aqNaHCO₃ and extracted with EtOAc. The combined organic layer was dried over Na₂SO₄ and concentrated under reduced pressure. 6-bromo-4-(3-fluoro-4-(6-(oxetan-3-yl)-2,6-diazaspiro[3.3]heptan-2-yl)phenyl)quinazoline (17 mg, 37 μmol, 30%) yellow solid. LCMS [M+H]⁺=456. ¹H NMR (500 MHz, CDCl₃) δ 9.27 (d, J=4.4 Hz, 1H), 8.32-8.27 (m, 1H), 7.93 (dd, J=4.0, 1.3 Hz, 2H), 7.52-7.43 (m, 2H), 6.61-6.55 (m, 1H), 4.85 (q, J=5.6 Hz, 1H), 4.80 (t, J=6.6 Hz, 1H), 4.70 (t, J=6.8 Hz, 1H), 4.57-4.48 (m, 2H), 4.21-4.17 (m, 2H), 3.53 (s, 2H), 2.04 (s, 4H). ¹³C NMR (126 MHz, CDCl₃) δ 154.71, 149.90, 137.12, 130.61, 129.11, 126.90, 123.91, 121.43, 117.43, 113.97, 74.51, 63.29, 60.28, 58.64, 29.70.

### Step B. 4-(4-(3-fluoro-4-(6-(oxetan-3-yl)-2,6-diazaspiro[3.3]heptan-2-yl)phenyl)quinazolin-6-yl)pyridin-2-amine (57)

From 6-bromo-4-(3-fluoro-4-(6-(oxetan-3-yl)-2,6-diazaspiro[3.3]heptan-2-yl)phenyl)quinazoline, the title compound was isolated in a similar manner as depicted in Example 2.1, Step D, as a yellow solid. LCMS [M+H]⁺=468. ¹H NMR (500 MHz, CDCl₃) δ 9.30 (d, J=4.5 Hz, 1H), 8.34 (d, J=2.0 Hz, 1H), 8.17-8.10 (m, 2H), 8.08 (dd, J=8.8, 2.0 Hz, 1H), 7.55-7.48 (m, 2H), 6.89 (dd, J=5.5, 1.6 Hz, 1H), 6.71 (s, 1H), 6.59 (t, J=8.5 Hz, 1H), 4.67 (d, J=6.3 Hz, 2H), 4.49 (dd, J=6.7, 5.1 Hz, 2H), 4.21 (d, J=2.1 Hz, 4H), 3.76-3.70 (m, 1H), 3.48 (s, 3H), 3.47 (d, J=3.9 Hz, 1H).

### Example 2.11: 1-(6-(4-(6-(2-aminopyridin-4-yl)quinazolin-4-yl)-2-fluorophenyl)-2,6-diazaspiro[3.3]heptan-2-yl)ethan-1-one (58)

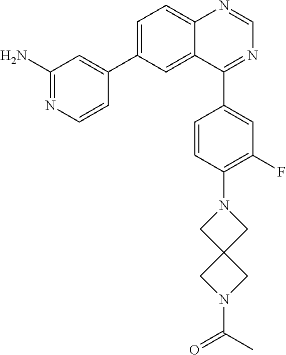 [SMILES: CC(=O)N1CC2(C1)CN(c1ccc(-c3ncnc4ccc(-c5ccnc(N)c5)cc34)cc1F)C2]

### Step A. 1-(6-(4-(6-bromoquinazolin-4-yl)-2-fluorophenyl)-2,6-diazaspiro[3.3]heptan-2-yl)ethan-1-one

From 6-bromo-4-(3-fluoro-4-(2,6-d azaspiro[3.3]heptan-2-yl)phenyl)quinazoline (XXVIII), the title compound was isolated in a similar manner as depicted in Example 2.4, Step B. LCMS [M+H]⁺=442. ¹H NMR (500 MHz, CDCl₃) δ 9.29 (s, 1H), 8.29 (t, J=1.4 Hz, 1H), 7.94 (d, J=1.4 Hz, 2H), 7.51-7.47 (m, 1H), 7.46 (d, J=1.9 Hz, 1H), 6.60 (t, J=8.6 Hz, 1H), 4.34 (s, 2H), 4.24 (d, J=2.0 Hz, 4H), 4.19 (s, 2H), 1.89 (s, 3H). ¹³C NMR (126 MHz, CDCl₃) δ 170.52, 165.76, 154.81, 153.11, 150.00, 140.57, 137.09, 130.78, 129.01, 126.86, 123.91, 121.44, 117.51, 114.07, 63.37, 57.82, 33.89, 18.87.

### Step B. 1-(6-(4-(6-(2-aminopyridin-4-yl)quinazolin-4-yl)-2-fluorophenyl)-2,6-diazaspiro[3.3]heptan-2-yl)ethan-1-one (58)

From 1-(6-(4-(6-bromoquinazolin-4-yl)-2-fluorophenyl)-2,6-diazaspiro[3.3]heptan-2-yl)ethan-1-one, the title compound was isolated in a similar manner as depicted in Example 2.1, Step D, as a yellow solid. LCMS [M+H]⁺=455. ¹H NMR (500 MHz, CDCl₃) δ 9.32 (s, 1H), 8.34 (d, J=2.0 Hz, 1H), 8.19-8.13 (m, 2H), 8.10 (dd, J=8.6, 2.0 Hz, 1H), 7.57-7.51 (m, 2H), 6.91 (d, J=5.4 Hz, 1H), 6.74 (s, 1H), 6.63 (t, J=8.5 Hz, 1H), 4.81-4.70 (m, 2H), 4.36 (s, 2H), 4.26 (d, J=2.0 Hz, 4H), 4.23 (s, 2H), 1.90 (s, 3H). ¹³C NMR (126 MHz, CDCl₃) δ 170.66, 167.18, 158.98, 155.16, 151.52, 149.42, 148.89, 148.54, 138.13, 132.54, 129.94, 127.67, 127.06, 125.16, 123.08, 117.91, 117.74, 114.20, 112.87, 106.82, 63.48, 57.93, 41.16, 18.99.

### Example 2.12: (S)-4-(4-(3-fluoro-4-(3-(methylamino)pyrrolidin-1-yl)phenyl)quinazolin-6-yl)pyridin-2-amine (60)

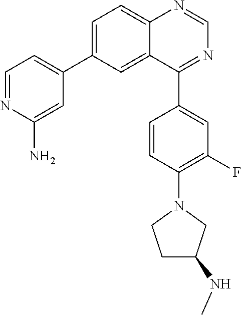 [SMILES: CN[C@H]1CCN(c2ccc(-c3ncnc4ccc(-c5ccnc(N)c5)cc34)cc2F)C1]

### Step A. tert-butyl (S)-(1-(4-bromo-2-fluorophenyl)pyrrolidin-3-yl)(methyl)carbamate

From (3S)-3-Amino-N-methylpyrrolidine, N-BOC protected, the title compound was isolated in a similar manner as depicted in Example 2.1, Step A, as a yellow solid. LCMS [M+H]⁺=373. ¹H NMR (500 MHz, CDCl₃) δ 7.23-7.21 (m, 1H), 6.65 (t, J=9.0 Hz, 1H), 3.64 (dq, J=8.4, 2.1 Hz, 1H), 3.57-3.47 (m, 2H), 3.41 (q, J=8.4 Hz, 1H), 2.97 (s, 3H), 2.32-2.25 (m, 1H), 2.19-2.12 (m, 1H), 1.61 (s, 9H). ¹³C NMR (126 MHz, CDCl₃) δ 155.72, 153.09, 136.07, 128.41, 127.31, 119.51, 116.55, 79.88, 51.86, 50.76, 48.63, 28.94, 28.48, 28.23.

### Step B. tert-butyl (S)-(1-(2-fluoro-4-(4,4,5,5-tetramethyl-1,3,2-dioxaborolan-2-yl)phenyl)pyrrolidin-3-yl)(methyl)carbamate

From tert-butyl (S)-(1-(4-bromo-2-fluorophenyl)pyrrolidin-3-yl)(methyl)carbamate, the title compound was isolated in a similar manner as depicted in Example 2.1, Step B. LCMS [M+H]⁺=421. ¹H NMR (500 MHz, CDCl₃) δ 7.43-7.38 (m, 1H), 7.38-7.33 (m, 1H), 6.59 (t, J=8.4 Hz, 1H), 3.64 (d, J=14.6 Hz, 1H), 3.56 (tdd, J=8.2, 6.4, 3.3 Hz, 1H), 3.50 (ddd, J=10.5, 7.9, 2.5 Hz, 1H), 3.42 (ddd, J=10.1, 6.6, 2.7 Hz, 1H), 3.35 (dd, J=13.1, 5.2 Hz, 1H), 2.80 (s, 3H), 2.16-2.06 (m, 1H), 2.00 (dq, J=12.6, 8.4 Hz, 1H), 1.45 (s, 9H), 1.23 (s, 12H). ¹³C NMR (126 MHz, CDCl₃) δ 155.74, 152.53, 139.15, 134.74, 131.58, 122.03, 114.73, 83.49, 79.87, 53.94, 51.59, 48.48, 28.92, 28.48, 28.33, 25.03.

### Step C. tert-butyl (S)-(1-(4-(6-bromoquinazolin-4-yl)-2-fluorophenyl)pyrrolidin-3-yl)(methyl)carbamate

From tert-butyl (S)-(1-(4-bromo-2-fluorophenyl)pyrrolidin-3-yl)(methyl)carbamate, the title compound was isolated in a similar manner as depicted in Example 2.1, Step C. LCMS [M+H]⁺=502. ¹H NMR (500 MHz, CDCl₃) δ 9.27 (s, 1H), 8.36-8.35 (m. 1H), 7.94 (t, J=1.5 Hz, 2H), 7.54 (dd, J=14.6, 2.0 Hz, 1H), 7.50 (dd, J=8.4, 2.1 Hz, 1H), 6.77 (t, J=8.7 Hz, 1H), 3.72-3.62 (m, 2H), 3.52 (ddd, J=10.1, 7.0, 2.8 Hz, 2H), 2.85 (s, 3H), 2.22-2.06 (m, 3H), 1.48 (s, 9H). ¹³C NMR (126 MHz, CDCl₃) δ 154.62, 137.28, 130.76, 130.55, 130.42, 129.88, 129.20, 129.18, 128.90, 127.23, 123.88, 121.35, 118.11, 115.08, 80.04, 54.66, 51.51, 48.55, 29.71, 29.02, 28.50.

### Step D. (S)-4-(4-(3-fluoro-4-(3-(methylamino)pyrrolidin-1-yl)phenyl)quinazolin-6-yl)pyridin-2-amine (60)

From tert-butyl (S)-(1-(4-(6-bromoquinazolin-4-yl)-2-fluorophenyl)pyrrolidin-3-yl)(methyl)carbamate, the title compound was isolated in a similar manner as depicted in Example 2.3, Step D, as a yellow solid. LCMS [M+H]⁺=414. ¹H NMR (500 MHz, CH₃OH+D₂O) δ 9.19 (t, J=4.6 Hz, 1H), 8.46 (s, 1H), 8.30 (d, J=8.8 Hz, 1H), 8.17-8.10 (m, 1H), 8.00 (d, J=4.9 Hz, 1H), 7.61 (s, 2H), 6.99 (s, 1H), 6.92 (s, 2H), 5.49 (d, J=2.8 Hz, 1H), 4.55 (s, 2H), 4.02 (s, 5H), 3.34 (d, J=2.9 Hz, 2H), 2.60 (d, J=2.8 Hz, 1H), 1.91 (s, 3H).

### Example 2.13: 4-(4-(3-methyl-4-(4-methylpiperazin-1-yl)phenyl)quinazolin-6-yl)pyridin-2-amine (61)

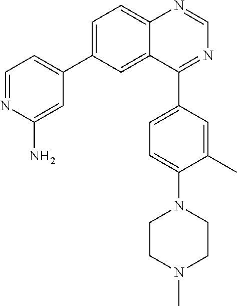 [SMILES: Cc1cc(-c2ncnc3ccc(-c4ccnc(N)c4)cc23)ccc1N1CCN(C)CC1]

### Step A. 1-(4-bromo-2-methylphenyl)-4-methylpiperazine

From (1-methylpiperazine and, 4-bromo-1-iodo-2-methylbenzene the title compound was isolated in a similar manner as depicted in Example 2.1, Step A, as a brown oil. LCMS [M+H]⁺=270. ¹H NMR (500 MHz, CDCl₃) δ 7.20 (d, J=2.3 Hz, 1H), 7.15 (dd, J=8.5, 2.5 Hz, 1H), 6.79 (d, J=8.5 Hz, 1H), 2.81 (t, J=4.9 Hz, 4H), 2.50 (s, 4H), 2.28 (s, 3H), 2.17 (s, 3H).

### Step B. 1-methyl-4-(2-methyl-4-(4,4,5,5-tetramethyl-1,3,2-dioxaborolan-2-yl)phenyl)piperazine

From 1-(4-bromo-2-methylphenyl)-4-methylpiperazine, the title compound was isolated in a similar manner as depicted in Example 2.1, Step B. LCMS [M+H]⁺=317.

### Step C. 6-bromo-4-(3-methyl-4-(4-methylpiperazin-1-yl)phenyl)quinazoline

From 1-methyl-4-(2-methyl-4-(4,4,5,5-tetramethyl-1,3,2-dioxaborolan-2-yl)phenyl)piperazine, the title compound was isolated in a similar manner as depicted in Example 2.1, Step C. LCMS [M+H]⁺=397. ¹HNMR (500 MHz, CDCl₃) δ 9.36 (s, 1H), 8.37 (d, J=1.4 Hz, 1H), 7.99 (d, J=1.4 Hz, 2H), 7.67 (d, J=2.2 Hz, 1H), 7.61 (dd, J=8.2, 2.2 Hz, 1H), 7.23 (d, J=8.2 Hz, 1H), 3.51 (s, 3H), 3.14 (t, J=4.8 Hz, 4H), 2.73 (s, 4H), 2.47 (s, 3H).

### Step D. 4-(4-(3-methyl-4-(4-methylpiperazin-1-yl)phenyl)quinazolin-6-yl)pyridin-2-amine (61)

From 6-bromo-4-(3-methyl-4-(4-methylpiperazin-1-yl)phenyl)quinazoline, the title compound was isolated in a similar manner as depicted in Example 2.1, Step D. LCMS [M+H]⁺=411. ¹H NMR (400 MHz, DMSO-d₆) δ 9.30 (s, 1H), 8.54-7.87 (m, 4H), 7.89-7.13 (m, 4H), 6.99 (d, J=5.9 Hz, 1H), 3.45-3.13 (m, 5H), 2.78 (s, 4H), 2.46 (s, 4H), 2.36 (s, 3H).

### Example 2.14: 4-(4-(4-(4-(methylsulfonyl)piperazin-1-yl)-3-(trifluoromethyl)phenyl)quinazolin-6-yl)pyridin-2-amine (63)

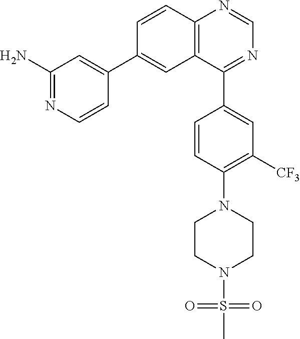 [SMILES: CS(=O)(=O)N1CCN(c2ccc(-c3ncnc4ccc(-c5ccnc(N)c5)cc34)cc2C(F)(F)F)CC1]

### Step A. 1-(4-bromo-2-(trifluoromethyl)phenyl)-4-(methylsulfonyl)piperazine

From 1-(methylsulfonyl)piperazine, HCl with 4-bromo-1-iodo-2-(trifluoromethyl)benzene the title compound was isolated in a similar manner as depicted in Example 2.1, Step A. LCMS [M+H]⁺=387. ¹H NMR (400 MHz, CDCl₃) δ 7.78 (s, 1H) 7.67 (d, 1H), 7.24 (d, 1H), 3.36 (t, 4H), 3.00 (t, 4H), 2.84 (s, 3H).

### Step B. 1-(methylsulfonyl)-4-(4-(4,4,5,5-tetramethyl-1,3,2-dioxaborolan-2-yl)-2-(trifluoromethyl)phenyl)piperazine

From 1-(4-bromo-2-(trifluoromethyl)phenyl)-4-(methylsulfonyl)piperazine, the title compound was isolated in a similar manner as depicted in Example 2.1, Step B. LCMS [M+H]⁺=435. ¹H NMR (400 MHz, CDCl₃) δ 8.08 (s, 1H), 7.96 (d, 1H), 7.31 (d, 1H), 3.37 (t, 4H), 3.05 (t, 4H), 2.84 (s, 3H), 1.34 (s, 12H).

### Step C. 6-bromo-4-(4-(4-(methylsulfonyl)piperazin-1-yl)-3-(trifluoromethyl)phenyl)quinazoline

From 1-(methylsulfonyl)-4-(4-(4,4,5,5-tetramethyl-1,3,2-dioxaborolan-2-yl)-2-(trifluoromethyl)phenyl)piperazine, the title compound was isolated in a similar manner as depicted in Example 2.1, Step C, as a cream solid. LCMS [M+H]⁺=515. ¹H NMR (400 MHz, CDCl₃) δ 9.37 (s, 1H), 8.18 (s, 1H), 8.09 (s, 1H), 8.00 (s, 2H), 7.94 (d, 1H), 7.55 (d, 1H), 3.42 (s, 4H), 3.16 (s, 4H), 2.86 (s, 3H).

### Step C. 4-(4-(4-(4-(methylsulfonyl)piperazin-1-yl)-3-(trifluoromethyl)phenyl)quinazolin-6-yl)pyridin-2-amine (63)

From 6-bromo-4-(4-(4-(methylsulfonyl)piperazin-1-yl)-3-(trifluoromethyl)phenyl)quinazoline, the title compound was isolated in a similar manner as depicted in Example 2.1, Step C. LCMS [M+H]'⁰=529. ¹H NMR (400 MHz, CDCl₃) δ 9.42 (s, 1H), 8.26 (s, 1H), 8.24 (d, 1H), 8.18 (s, 1H), 8.15 (d, 1H), 8.11 (d, 1H), 8.03 (d, 1H), 7.59 (d, 1H), 6.91 (d, 1H), 6.85 (s, 1H), 3.43 (t, 4H), 3.18 (t, 4H), 2.87 (s, 3H). ¹³CNMR (100 MHz, CDCl₃) δ 166.7, 158.3, 158.2, 155.1, 153.2, 151.4, 149.8, 138.2, 134.5, 133.9, 132.8, 130.2, 124.6, 124.5, 122.9, 112.5, 107.5, 52.9, 46.3, 34.5, 24.9.

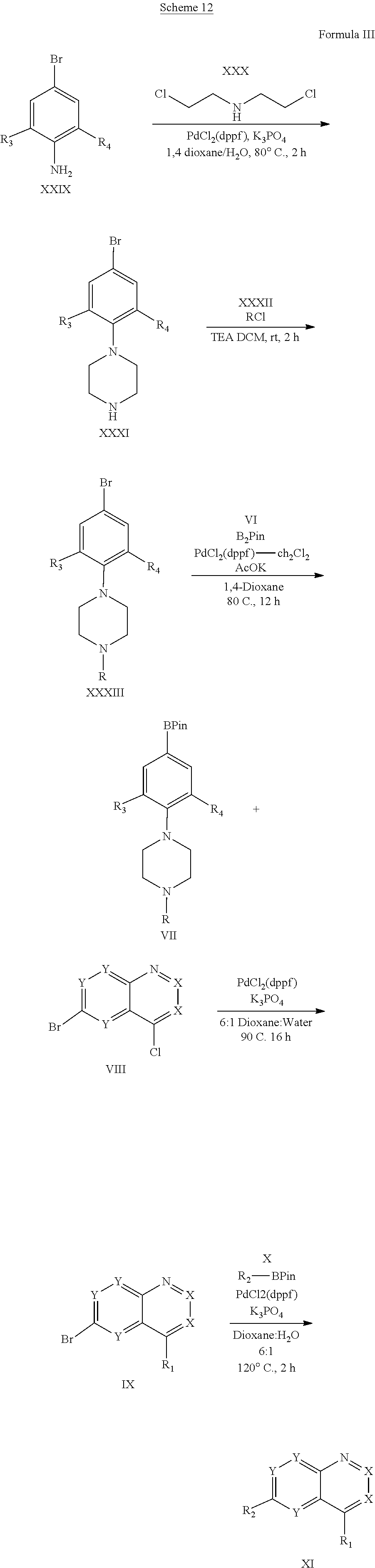

Scheme 12 describes the alternative synthesis to substituted aryl bromides (XXXIII). Anilines (XXIX) were transformed into piperazines (XXXI) via double displacement reaction with Bis(2-chloroethyl)amine (XXX). The piperazine analogues (XXXI) were then functionalized via substitution reactions, and subsequently the (hetero)aryl bromides (XXXIII) were utilized in a Miyaura borylation to produce the pinacol boronic esters (VII). The boronic esters were then coupled with polyhalogenated heterocycles (VIII) in a Suzuki reaction to afford the R₁ substituted heterocycle (IX) which is reacted in an additional Suzuki reaction resulting in R₂, R₁ substituted heterocycle (XI).

### Example 3.1: 4-(4-(3,5-difluoro-4-(4-methylpiperazin-1-yl)phenyl)quinazolin-6-yl)pyridin-2-amine (31)

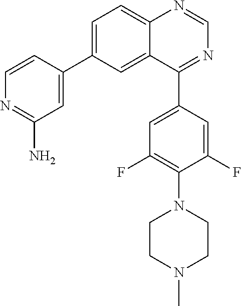 [SMILES: CN1CCN(c2c(F)cc(-c3ncnc4ccc(-c5ccnc(N)c5)cc34)cc2F)CC1]

### Step A. 1-(4-bromo-2,6-difluorophenyl)piperazine

To a 5 mL M.W. vial was added bis(2-chloroethyl)amine, HCl (1.96 g, 1.1 eq, 11.0 mmol), 4-bromo-2,6-difluoroaniline (2.08 g, 1 eq, 10.0 mmol), and diethylene glycol monomethyl ether (2.0 mL). The reaction mixture was heated to 150° C. overnight. The resulting mixture was allowed to cool and dissolved in a minimal amount of MeOH (7 mL). Ether was added until the product crashed out of solution (100 mL) and filtered. The product was washed with more ether, then extracted with DCM/water, dried over MgSO4, filtered and concentrated to yield 1-(4-bromo-2,6-difluorophenyl)piperazine (400 mg, 14.4%), which was carried over to the next step without further purification. LCMS [M+H]⁺=278.

### Step B. 1-(4-bromo-2,6-difluorophenyl)-4-methylpiperazine

A mixture of 1-(4-bromo-2,6-difluorophenyl)piperazine hydrochloride (570.0 mg, 1 eq, 1.818 mmol) and sodium hydride (0.17 g, 50% Wt, 2 eq, 3.636 mmol) in THF (10 ml) was stirred at rt for 30 mins, iodomethane (284 mg, 124 μL, 1.1 eq, 2.000 mmol) added to the solution. The resulting mixture was stirred at rt for 2 hrs. ¹H NMR (500 MHz, DMSO-d₆) δ 7.38 (d, J=8.8 Hz, 2H), 3.33 (s, 4H), 3.10 (t, J=4.9 Hz, 4H), 2.23 (s, 3H). ¹³C NMR (126 MHz, DMSO-d₆) δ 116.67, 116.45, 55.51, 50.74, 46.25, 39.84, 39.74.

### Step C. 1-(2,6-difluoro-4-(4,4,5,5-tetramethyl-1,3,2-dioxaborolan-2-yl)phenyl)-4-methylpiperazine

A mixture of 1-(4-bromo-2,6-difluorophenyl)-4-methylpiperazine (400 mg, 1 eq, 1.37 mmol), 4,4,4′,4′,5,5,5′,5′-octamethyl-2,2′-bi(1,3,2-dioxaborolane) (593 mg, 1.7 eq, 2.34 mmol), potassium acetate (405 mg, 3 Eq, 4.12 mmol), and PdCl2(dppf) (108 mg, 0.1 eq, 137 μmol) in DMF (4 mL) was heated at 80° C. for 2 hrs. The reaction mixture was diluted with ethyl acetate (20 ml), washed with brine (10 ml×2). The organic layer was dried over sodium sulfate, filtered, evaporated to obtain the crude product. The crude product was purified with silica gel chromatography (eluent ethyl acetate:hexanes=0 to 100%, then methanol:ethyl acetate=0 to 20%) to obtain 1-(2,6-difluoro-4-(4,4,5,5-tetramethyl-1,3,2-dioxaborolan-2-yl)phenyl)-4-methylpiperazine (270 mg, 798 gmol, 58.1%). LCMS [M+H]⁺=339.

### Step D. 6-bromo-4-(3,5-difluoro-4-(4-methylpiperazin-1-yl)phenyl)quinazoline

A 100-mL-round-bottle mixture of 6-bromo-4-chloroquinazoline (389 mg, 2 eq, 1.60 mmol), 1-(2,6-difluoro-4-(4,4,5,5-tetramethyl-1,3,2-dioxaborolan-2-yl)phenyl)-4-methylpiperazine (270 mg, 1 eq, 798 μmol), and tripotassium phosphate (254 mg, 1.5 eq, 1.20 mmol) and PdCl2(dppf) (63 mg, 0.1 eq, 79.8 μmol) in 1,4-Dioxane (20 mL) and water (3.3 mL) was heated at 60° C. under argon atmosphere for 2 hrs. The reaction was cooled to the room temperature, diluted with ethyl acetate (40 ml) and washed with saturated sodium bicarbonate (10 ml) and brine (10 ml). The organic layer was dried over sodium sulfate, evaporated in vacuo to get the crude product. The crude product was purified with silica gel chromatography (40 g silica gel, eluent ethyl acetate/Hexanes=0 to 100%) to afford the product 6-bromo-4-(3,5-difluoro-4-(4-methylpiperazin-1-yl)phenyl)quinazoline (192 mg, 458 μmol, 57%). ¹H NMR (500 MHz, DMSO-d₆) δ 9.37 (s, 1H), 8.26 (d, J=2.3 Hz, 1H), 8.22-8.15 (m, 1H), 8.05 (d, J=9.0 Hz, 1H), 7.52 (t, J=8.4 Hz, 2H), 3.26 (d, J=41.8 Hz, 4H), 2.58 (t, J=4.8 Hz, 4H), 2.33 (s, 3H). ¹³C NMR (126 MHz, DMSO-d₆) δ 164.68, 163.70, 158.26, 155.03, 149.72, 137.99, 131.21, 130.90, 129.91, 128.94, 123.72, 121.77, 114.79, 114.58, 55.36, 50.55, 45.97.

### Step E. 4-(4-(3,5-difluoro-4-(4-methylpiperazin-1-yl)phenyl)quinazolin-6-yl)pyridin-2-amine (31)

A mixture of 6-bromo-4-(3,5-difluoro-4-(4-methylpiperazin-1-yl)phenyl)quinazoline (192 mg, 1 eq, 458 μmol), 4-(4,4,5,5-tetramethyl-1,3,2-dioxaborolan-2-yl)pyridin-2-amine (111 mg, 1.1 eq, 504 μmol), potassium phosphate (146 mg, 1.5 eq, 687 μmol), and PdCl2(dppf) (36 mg, 0.1 eq, 45.8 μmol) in 1,4-Dioxane (9 mL) and water (1 mL) was heated at 90° C. under argon atmosphere for 2 hrs. The reaction mixture was cooled to room temperature, and diluted with ethyl acetate, washed with water. The aqueous solution was washed with ethyl acetate. The combined organic layer was washed with brine, dried over sodium sulfate, filtered, and evaporated in vacuo to get the crude product. The crude product was purified via column chromatography (DCM/MeOH 0-10%) to afford 4-(4-(3,5-difluoro-4-(4-methylpiperazin-1-yl)phenyl)quinazolin-6-yl)pyridin-2-amine (109 mg, 252 μmol, 55%). Mp 105-107° C. LCMS [M+H]⁺=433. ¹H NMR (500 MHz, DMSO-d₆) δ 9.36 (s, 1H), 8.33-8.25 (m, 2H), 8.19 (d, J=17.0 Hz, 3H), 8.04 (d, J=5.3 Hz, 1H), 7.66-7.51 (m, 2H), 6.87 (dd, J=5.4, 1.7 Hz, 1H), 6.77 (d, J=1.7 Hz, 1H), 3.32 (t, J=4.7 Hz, 4H), 2.62 (t, J=4.7 Hz, 4H), 2.35 (s, 3H). ¹³C NMR (126 MHz, DMSO-d₆) δ 165.69, 163.79, 160.90, 158.17, 155.98, 154.98, 150.96, 149.20, 147.43, 138.75, 133.43, 131.37, 129.80, 124.25, 122.64, 114.87, 110.72, 106.00, 55.28, 50.45, 45.83

### Example 3.2: 4-(4-(3-chloro-4-(4-(methylsulfonyl)piperazin-1-yl)phenyl)quinazolin-6-yl)pyridin-2-amine (59)

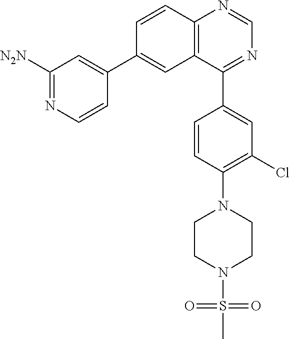

### Step A. 1-(4-bromo-2-chlorophenyl)piperazine

From bromo-2-chloroaniline, the title compound was isolated in a similar manner as depicted in Example 3.1, Step A LCMS [M+H]⁺=275.

### Step B. 1-(4-bromo-2-chlorophenyl)-4-(methylsulfonyl)piperazine

To a 100 mL RBF was added 1-(4-bromo-2-chlorophenyl)piperazine (918 mg, 1 eq, 3.33 mmol), TEA (674 mg, 929 μL, 2 eq, 6.66 mmol), and DCM (15.0 mL) The reaction was cooled to 0° C., and methanesulfonyl chloride (572 mg, 389 μL, 1.5 eq, 5.00 mmol) was added. The resulting mixture was allowed to warm to room temperature and stirred for 2 h. The mixture was diluted with more DCM and H₂O, the layers separated, and the aqueous layer washed again with DCM. The combined organic layers were then washed with brine, dried over magnesium sulfate, filtered, and concentrated. The resulting residue was purified by silica chromatography (35% EtoAc in hexanes) to grant 1-(4-bromo-2-chlorophenyl)-4-(methylsulfonyl)piperazine (407 mg, 1.15 mmol, 35%). LCMS [M+H]⁺=353. ¹H NMR (400 MHz, CDCl₃) δ 7.53 (d, 1H), 7.36 (dd, 1H), 6.91 (d, 1H), 3.41 (t, 4H), 3.12 (t, 4H), 2.84 (s, 3H).

### Step C. 1-(2-chloro-4-(4,4,5,5-tetramethyl-1,3,2-dioxaborolan-2-yl)phenyl)-4-(methylsulfonyl)piperazine

From 1-(4-bromo-2-chlorophenyl)-4-(methylsulfonyl)piperazine, the title compound was isolated in a similar manner as depicted in Example 3.1, Step C. LCMS [M+H]⁺=401. ¹H NMR (400 MHz, CDCl₃) δ 7.81 (s, 1H), 7.66 (d, 1H), 7.01 (d, 1H), 3.42 (t, 4H), 3.19 (t, 4H), 2.84 (s, 3H) 1.33 (s, 12H).

### Step D. 6-bromo-4-(3-chloro-4-(4-(methylsulfonyl)piperazin-1-yl)phenyl)quinazoline

From 1-(2-chloro-4-(4,4,5,5-tetramethyl-1,3,2-dioxaborolan-2-yl)phenyl)-4-(methylsulfonyl)piperazine, the title compound was isolated in a similar manner as depicted in Example 3.1, Step D. LCMS [M+H]⁺=481. ¹H NMR (400 MHz, CDCl₃) δ 9.36 (s, 1H), 8.26 (t, 1H), 8.00 (d, 2H), 7.87 (d, 1H), 7.67 (dd, 1H), 7.24 (d, 1H), 3.48 (t, 4H), 3.30 (t, 4H), 2.88 (s, 3H).

### Step E. 4-(4-(3-chloro-4-(4-(methylsulfonyl)piperazin-1-yl)phenyl)quinazolin-6-yl)pyridin-2-amine (59)

From 6-bromo-4-(3-chloro-4-(4-(methylsulfonyl)piperazin-1-yl)phenyl)quinazoline, the title compound was isolated in a similar manner as depicted in Example 3.1, Step E. LCMS [M+H]⁺=495. ¹H NMR (400 MHz, CDCl₃) δ 9.37 (s, 1H), 8.28 (s, 1H), 8.21-8.12 (m, 3H), 7.92 (s, 1H), 7.72 (d, 1H), 7.24 (d, 1H), 6.90 (d, 1H), 6.71 (s, 1H), 4.61 (bs, 2H), 3.48 (t, 4H), 3.30 (t, 4H), 2.87 (s, 3H). ¹³CNMR (100 MHz, CDCl₃) δ166.7, 159.0, 154.9, 151.2, 150.3, 149.1, 148.9, 138.6, 132.9, 132.8, 132.4, 129.9, 129.6, 129.3, 124.6, 123.0, 120.7, 112.8, 106.5, 75.0, 50.7, 46.1, 34.5, 24.9.

### Example 3.3: 4-(4-(3-chloro-4-(4-methylpiperazin-1-yl)phenyl)quinazolin-6-yl)pyridin-2-amine (62)

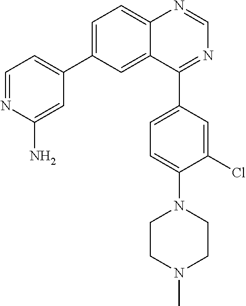 [SMILES: CN1CCN(c2ccc(-c3ncnc4ccc(-c5ccnc(N)c5)cc34)cc2Cl)CC1]

### Step A. 1-(4-bromo-2-chlorophenyl)-4-methylpiperazine

A solution of 1-(4-bromo-2-chlorophenyl)piperazine (1.28 g, 1 eq, 4.6456 mmol), aqueous formaldehyde (209 mg, 192.0 μL, 1.5 eq, 6.97 mmol) solution (37%), acetic acid (558 mg, 531.9 μL, 2 eq, 9.29 mmol) in DCM (46.5 mL) was treated with sodium triacetoxyborohydride (3.94 g, 4 eq, 18.58 mmol) at 25° C. for 1 hour. The reaction was quenched by aqueous NaHCO₃. The resulting mixture was concentrated and purified on silica to yield 1-(4-bromo-2-chlorophenyl)-4-methylpiperazine (722 mg, 2.49 mmol, 54%). LCMS [M+H]⁺=290. ¹HNMR (500 MHz, CDCl₃) δ 7.41 (d, J=2.3 Hz, 1H), 7.25 (dd, J=8.6, 2.3 Hz, 1H), 6.84 (d, J=8.6 Hz, 1H), 3.08 (s, 4H), 2.74 (s, 4H), 2.41 (s, 3H).

### Step B. 1-(2-chloro-4-(4,4,5,5-tetramethyl-1,3,2-dioxaborolan-2-yl)phenyl)-4-methylpiperazine

From 1-(4-bromo-2-chlorophenyl)-4-methylpiperazine, the title compound was isolated in a similar manner as depicted in Example 3.1, Step C. LCMS [M+H]⁺=337. ¹HNMR (500 MHz, CDCl₃) δ 8.00 (d, J=1.5 Hz, 1H), 7.86 (dd, J=8.0, 1.5 Hz, 1H), 7.23 (d, J=8.0 Hz, 1H), 3.36 (s, 4H), 2.89 (s, 4H), 2.60 (s, 3H), 1.54 (s, 12H).

### Step C. 6-bromo-4-(3-chloro-4-(4-methylpiperazin-1-yl)phenyl)quinazoline

From 1-(2-chloro-4-(4,4,5,5-tetramethyl-1,3,2-dioxaborolan-2-yl)phenyl)-4-methylpiperazine, the title compound was isolated in a similar manner as depicted in Example 3.1, Step D. LCMS [M+H]⁺=418. ¹H NMR (400 MHz, CDCl₃) δ 9.26 (s, 1H), 8.20 (t, J=1.4 Hz, 1H), 7.89 (d, J=1.4 Hz, 2H), 7.75 (d, J=2.1 Hz, 1H), 7.56 (dd, J=8.3, 2.1 Hz, 1H), 7.14 (d, J=8.3 Hz, 1H), 3.17 (t, J=4.9 Hz, 4H), 2.62 (t, J=4.8 Hz, 4H), 2.34 (s, 3H).

### Step E. 4-(4-(3-chloro-4-(4-methylpiperazin-1-yl)phenyl)quinazolin-6-yl)pyridin-2-amine (62)

From 6-bromo-4-(3-chloro-4-(4-methylpiperazin-1-yl)phenyl)quinazoline, the title compound was isolated in a similar manner as depicted in Example 3.1, Step E. LCMS [M+H]⁺=431. ¹H NMR (500 MHz, CDCl₃) δ 9.32 (s, 1H), 8.27 (d, J=2.0 Hz, 1H), 8.22-7.97 (m, 3H), 7.87 (d, J=2.0 Hz, 1H), 7.67 (dd, J=8.3, 2.1 Hz, 1H), 7.21 (d, J=8.3 Hz, 1H), 6.87 (dd, J=5.4, 1.6 Hz, 1H), 6.69 (d, J=1.1 Hz, 1H), 4.66 (s, 2H), 3.29 (s, 4H), 2.79 (s, 4H), 2.47 (s, 3H).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound encompassed within one of the following formulas: 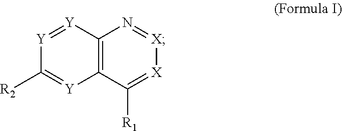
including pharmaceutically acceptable salts, solvates, and/or prodrugs thereof,
wherein each of X, Y, R1 and R2 independently include any chemical moiety that permits the resulting compound to inhibit DYRK1A, DYRK1B, DYRK2, DYRK3, CLK1, CLK2, CLK3, CLK4, homeodomain-interacting kinases (HIPKs), and/or CMGC kinases leading to inhibition of WNT signaling.

2. The compound of claim 1, wherein each of X, Y, R1 and R2 independently include any chemical moiety that permits the resulting compound to inhibit one or more of:
DYRK1A activity;
DYRK1A related PI3K/Akt signaling;
DYRK1A related tau phosphorylation;
DYRK1A related NFAT phosphorylation;
DYRK1A related ASK1/JNK1 pathway activation;
DYRK1A related p53 phosphorylation;
DYRK1A related Amph 1 phosphorylation;
DYRK1A related Dynamin 1 phosphorylation;
DYRK1A related Synaptojanin phosphorylation;
DYRK1A related presenilin 1 (the catalytic sub-unit of γ-secretase) activity;
DYRK1A related amyloid precursor protein phosphorylation;
DYRK1A related SIRT1 activation;
DYRK2 related heat shock factor 1 and 26S proteasome activities;
DYRK3 related mTOR activity;
DYRK3 phosphorylation (e.g., PRAS40);
DYRK1B activity;
CMGC/CLK kinase activity;
CLK1 activity;
CLK2 activity;
CLK3 activity;
CLK4 activity;
CDK7 activity;
CDK8 activity;
CDK19 activity;
CDK8/19 activity;
PI3K activity;
PI3K mutant activity;
PDGFrA/B activity;
mTOR activity;
c-KIT activity;
RYK activity; and
WNT signaling.

3. The compound of claim 1, wherein each of X, Y, R1 and R2 independently include any chemical moiety that permits the resulting compound capable of binding to a DYRK or CLK protein.

4. The compound of claim 1,
wherein one of the “X” substituents is carbon and the other is nitrogen, or wherein both of the “X” substituents are carbon; and
wherein one of the “Y” substituents is nitrogen and the other “Y” substituents are carbon, or wherein two of the “Y” substituents are nitrogen and one “Y” substituent is carbon, or wherein all of the “Y” substituents are carbon.

5. The compound of claim 4, wherein the resulting formula is selected from:

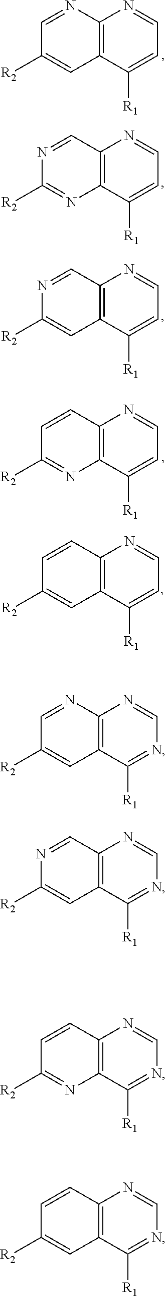

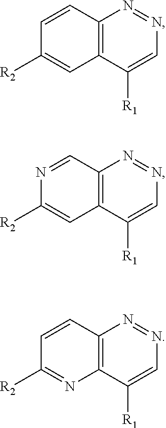

6. The compound of claim 1, wherein R1 is selected from hydrogen, halogen (e.g., fluorine, bromine, iodine, chlorine),

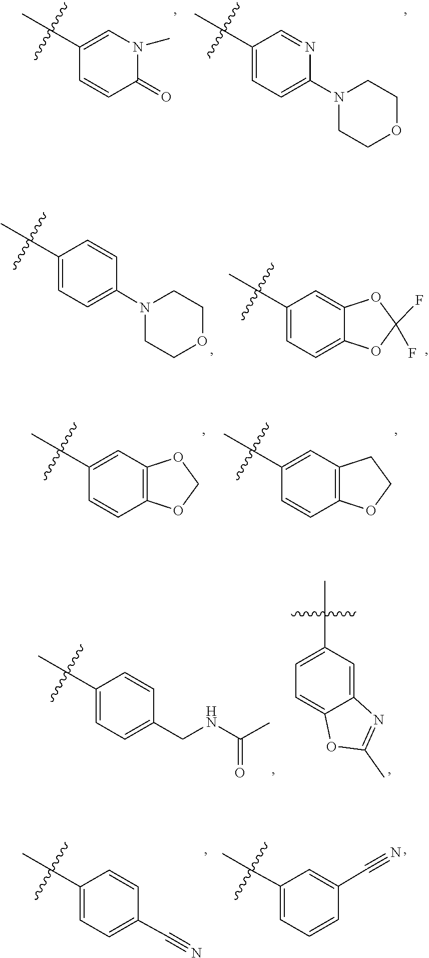

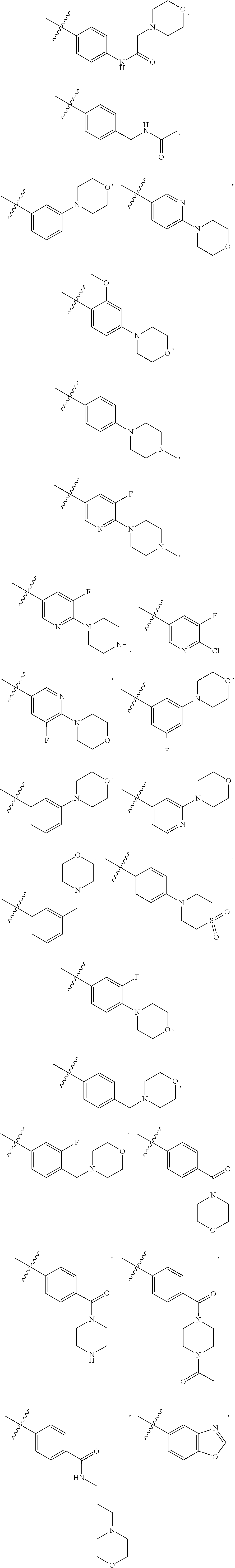

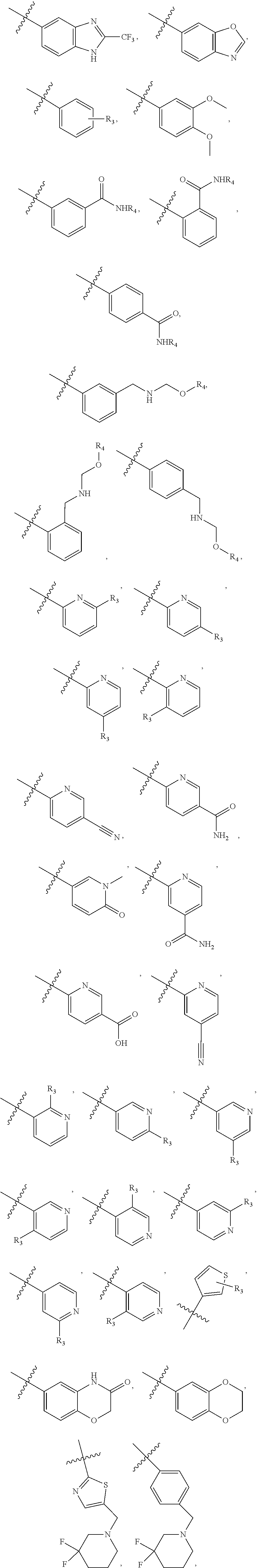

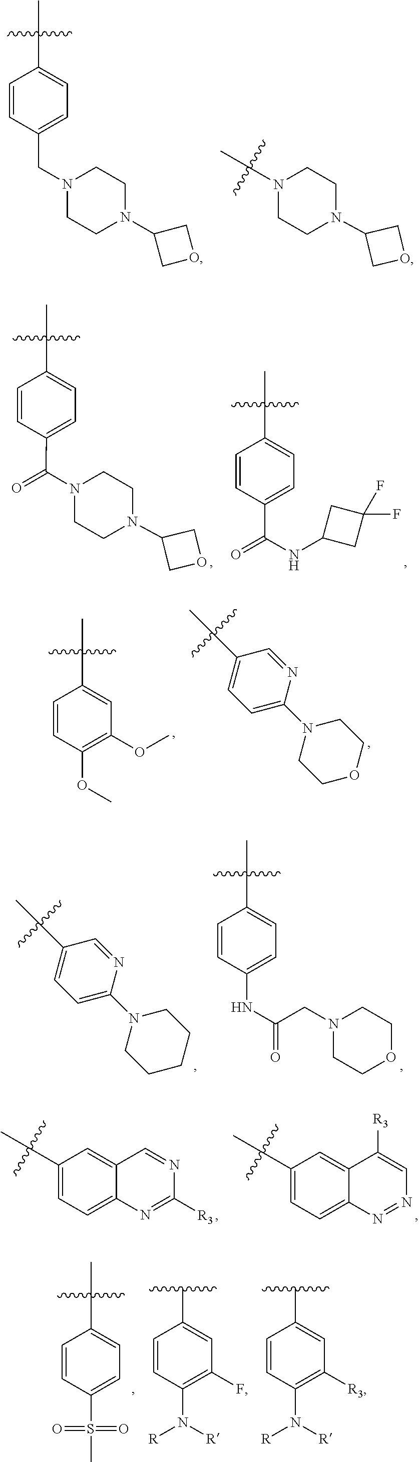

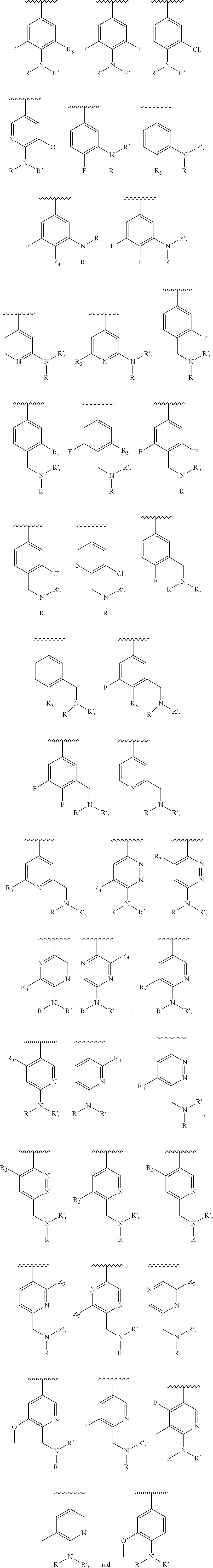

7. The compound of claim 1, wherein R2 is selected from hydrogen, halogen (e.g., fluorine, bromine, iodine, chlorine), aryl, substituted aryl, heteroaryl, substituted heteroaryl, 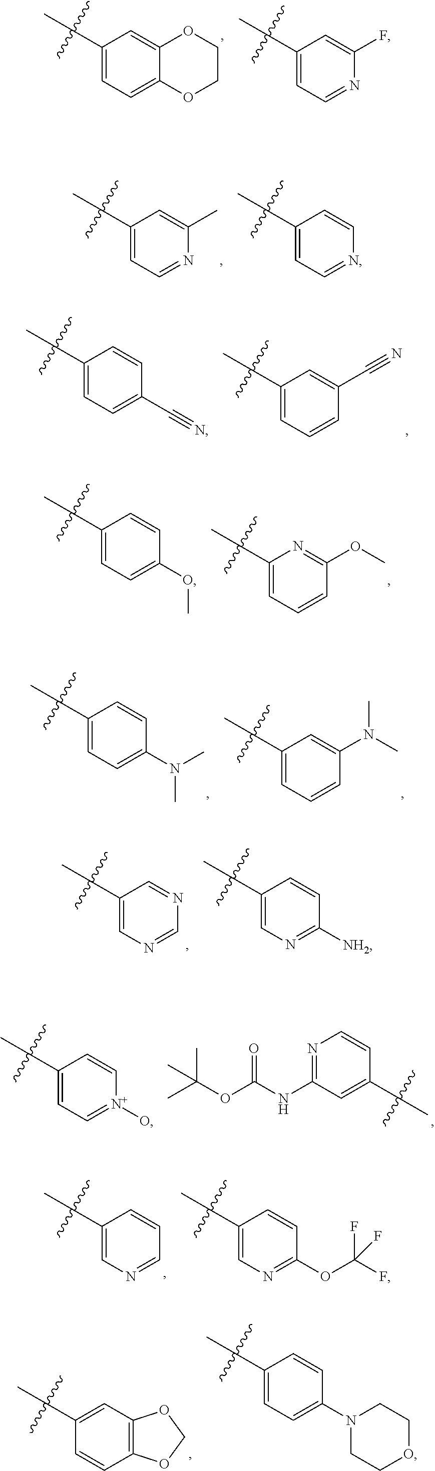 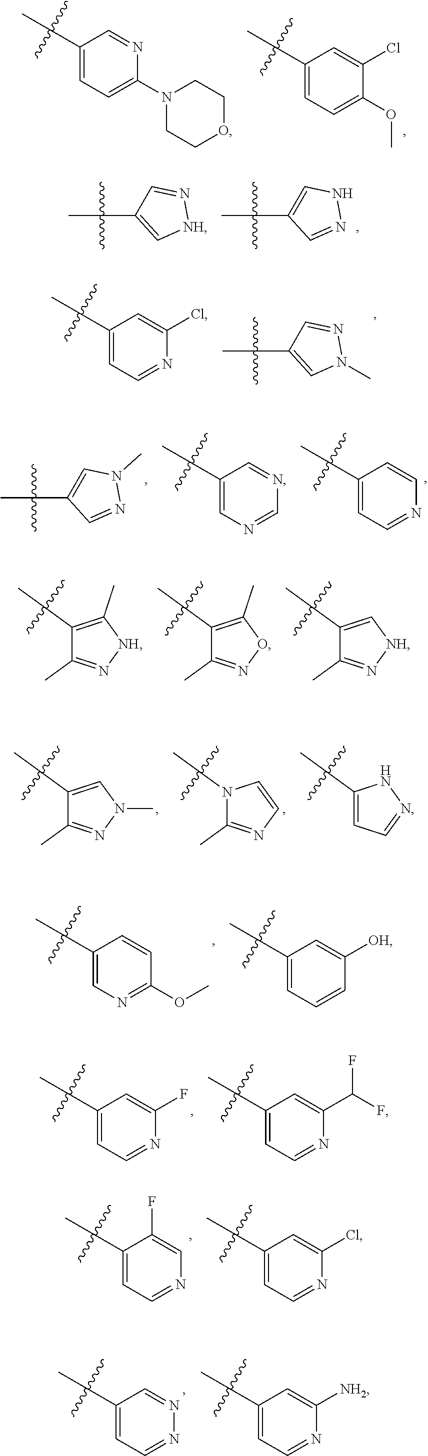 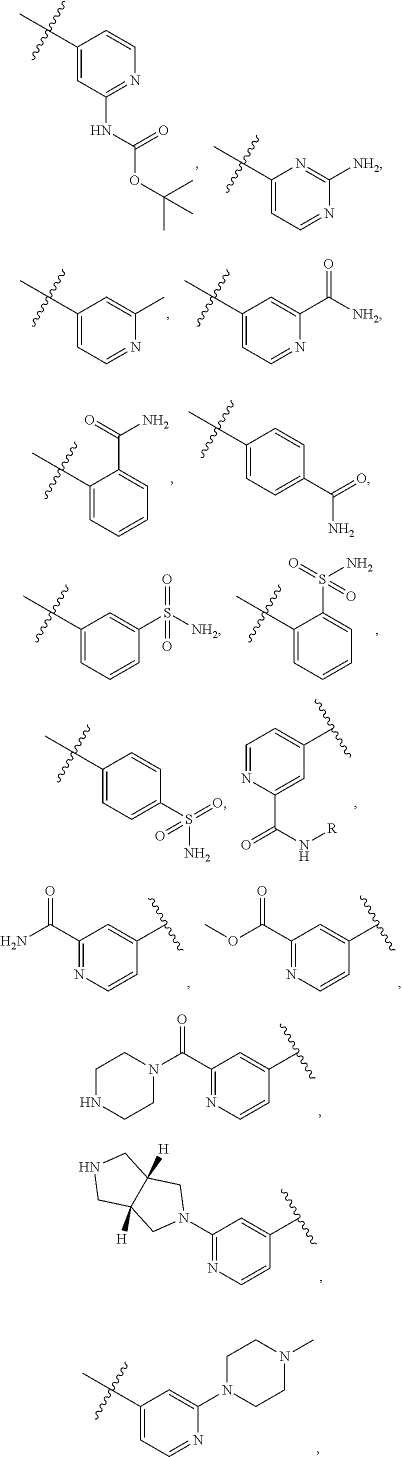 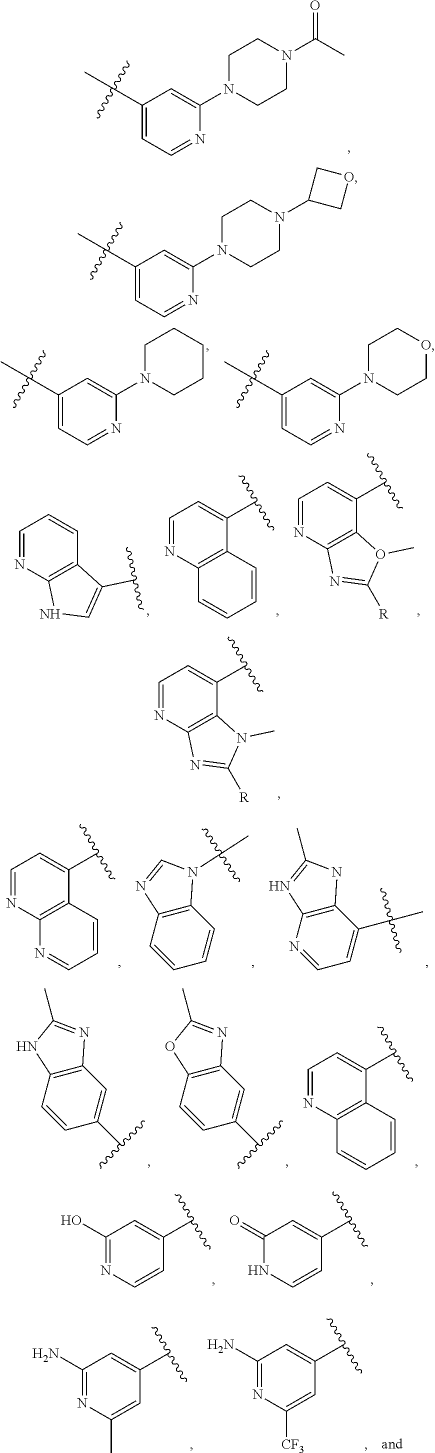 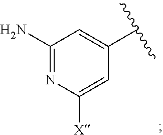
wherein X″ is selected from alkyl, haloalkyl, amino, alkylamino, hydroxy, fluoro, chloro, bromo, and cyano groups.

8. The compound of claim 6 or claim 7, wherein R, R′, and R″ are independently selected from hydrogen, halogen (e.g., fluorine, bromine, chlorine, iodine), di-halogen (di-fluorine, di-bromine, di-chlorine, di-iodine), CF3, OCH3, CHF2H, OCF3, methyl, di-methyl, alkoxy, alkylsulfonyl, cyano, carboxy, ester, amido, substituted amido, sulfonamide, substituted sulfonamide, methylenedioxy, heterocyclyl alkyl, heterocyclyl, heterocyclyl alkyl amido, a lipophilic moiety comprising ether, a secondary or tertiary amine moiety consisting of a heterocycloalkyl group that is bioisosteric to secondary amines (e.g., morpholine, piperidine, piperazine).

9. The compound of claim 6, wherein R3 and R4 are independently selected from hydrogen, halogen (e.g., fluorine, bromine, chlorine, iodine), methyl, ethyl, and methoxy.

10. The compound of claim 1, wherein said compound is selected from the group of compounds recited in Table 1 and/or Compounds 1-64.

11. A pharmaceutical composition comprising a compound of claim 1.

12. A method of treating, ameliorating, or preventing a disorder related to one or more of DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling in a patient comprising administering to said patient a therapeutically effective amount of the pharmaceutical composition of claim 11.

13. The method of claim 12, wherein said disorder is selected from Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

14. The method of claim 12, wherein said patient is a human patient.

15. The method of claim 12, further comprising administering to said patient one or more agents for treating Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

16. A kit comprising a compound of claim 1 and instructions for administering said compound to a patient having a disorder related to one or more of DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling.

17. The kit of claim 16, wherein the disorder is Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

18. The kit of claim 17, further comprising one or more agents for treating Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

19. A method for inhibiting one or more of DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling in a subject, comprising administering to the subject a compound of claim 1.

20. The method of claim 19, wherein administration of the compound results in inhibition of one or more of DYRK1A activity;
DYRK1A related PI3K/Akt signaling;
DYRK1A related tau phosphorylation;
DYRK1A related NFAT phosphorylation;
DYRK1A related ASK1/JNK1 pathway activation;
DYRK1A related p53 phosphorylation;
DYRK1A related Amph 1 phosphorylation;
DYRK1A related Dynamin 1 phosphorylation;
DYRK1A related Synaptojanin phosphorylation;
DYRK1A related presenilin 1 (the catalytic sub-unit of γ-secretase) activity;
DYRK1A related amyloid precursor protein phosphorylation;
DYRK1A related SIRT1 activation;
DYRK2 related heat shock factor 1 and 26S proteasome activities;
DYRK3 related mTOR activity;
DYRK3 phosphorylation (e.g., PRAS40);
DYRK1B activity;
CMGC/CLK kinase activity;
CLK1 activity;
CLK2 activity;
CLK3 activity;
CLK4 activity;
CDK7 activity;
CDK8 activity;
CDK19 activity;
CDK8/19 activity;
PI3K activity;
PI3K mutant activity;
PDGFrA/B activity;
mTOR activity;
c-KIT activity;
RYK activity; and
WNT signaling.

21. The method of claim 19, wherein the subject is human subject suffering from or at risk for developing a disorder related to DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling.

22. The method of claim 19, wherein the disorder is Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.
